(19) United States
(12) Patent Application Publication
(10) Pub. No.: US 2025/0241876 A1
(43) Pub. Date: Jul. 31, 2025

(54) ANTI-TUMOR COMPOUNDS AND METHODS OF USE THEREOF AND SYNTHESIS THEREOF

(71) Applicant: Atossa Therapeutics, Inc., Seattle, WA (US)

(72) Inventor: Steven Carl QUAY, Seattle, WA (US)

(21) Appl. No.: 19/005,858

(22) Filed: Dec. 30, 2024

Related U.S. Application Data
(63) Continuation-in-part of application No. PCT/US2024/038871, filed on Jul. 19, 2024.
(60) Provisional application No. 63/527,800, filed on Jul. 19, 2023.

(51) Int. Cl. A61K 31/14; A61K 31/138; A61K 31/222; A61K 31/325; A61P 35/00; C07C 217/22; C07C 217/32; C07C 271/48

(52) U.S. Cl. CPC A61K 31/14; A61K 31/138; A61K 31/222; A61K 31/325; A61P 35/00; C07C 217/22; C07C 217/32; C07C 271/48

(57) ABSTRACT

Described herein are pharmaceutical compounds and compositions and methods of making thereof. Methods of synthesis for the compounds are described herein. The compounds or compositions described herein may exhibit aromatase inhibition activity or estrogen receptor activity. The compounds and compositions described herein may be useful in the treatment of a condition in a subject.

### CROSS-REFERENCE

This application is a Continuation-in-part of International Application No. PCT/US2024/038871, entitled “ANTI-TUMOR COMPOUNDS AND METHODS OF USE THEREOF AND SYNTHESIS THEREOF”, filed Jul. 19, 2024, which claims the benefit of U.S. Provisional Application No. 63/527,800, entitled “ANTI-TUMOR COMPOUNDS AND METHODS OF USE THEREOF AND SYNTHESIS THEREOF,” filed Jul. 19, 2023, which application is incorporated herein by reference in its entirety.

### BACKGROUND

Cancer is a group of diseases characterized by uncontrolled cell growth. Cancer can be deadly and while many treatment options exist, there is a need for continuous development of new treatment therapies and regimens. New pharmaceutical drugs can allow for treatment of wider ranges of cancers and be helpful for the treatment of current therapy resistant cancers and tumors. Hence, there remains a need for new active pharmaceutical ingredients (APIs) that act as anti-tumor compounds and methods of making them.

### SUMMARY

In various aspects, the present disclosure provides a compound of Formula (I):

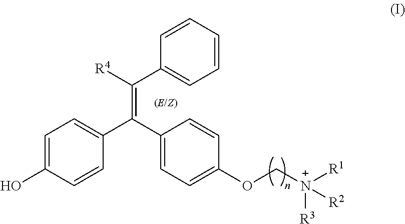

or a pharmaceutically acceptable salt, solvate, or tautomer thereof, wherein: R¹, R², R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; R⁴ is C₁-C₆ alkyl substituted with 0 to 3 instances of R⁸, C₂-C₆ alkenyl substituted with 0 to 3 instances of R⁸, or C₂-C₆ alkynyl substituted with 0 to 3 instances of R⁸; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; each instance of R⁸ is independently halogen, hydroxyl, C₁-C₃ alkoxy, C₁-C₃ alkylthio, C₁-C₃ alkyl, or C₃-C₈ cycloalkyl; and n is an integer from 1 to 10.

In some aspects, the compound of Formula (I) is a compound of Formula (Ia):

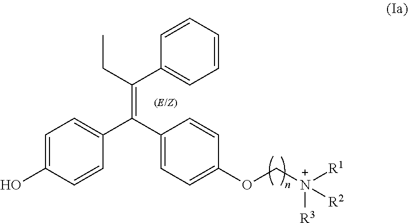

or a pharmaceutically acceptable salt, solvate, or tautomer thereof.

In some aspects, the compound of Formula (I) is a compound of Formula (Ib):

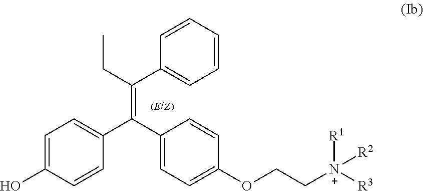

or a pharmaceutically acceptable salt, solvate, or tautomer thereof.

In some aspects, the compound of Formula (I) is a compound of Formula (II):

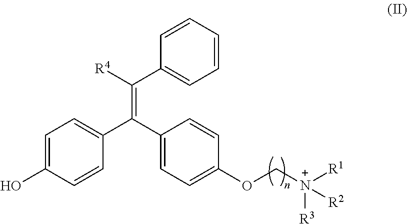

or a pharmaceutically acceptable salt, solvate, or tautomer thereof.

In some aspects, the compound of Formula (I) is a compound of Formula (IIa):

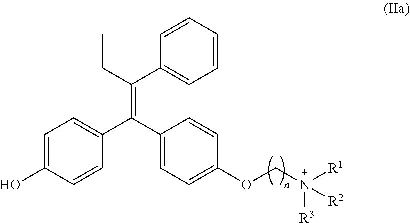

or a pharmaceutically acceptable salt, solvate, or tautomer thereof.

In some aspects, the compound of Formula (I) is a compound of Formula (IIb):

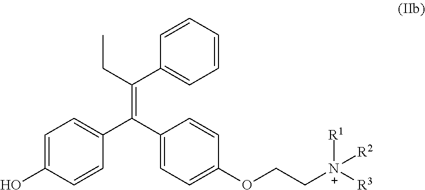

or a pharmaceutically acceptable salt, solvate, or tautomer thereof.

In some aspects, the compound is a (Z)-isomer of the compound of Formula (I). In some aspects, the compound is an (E)-isomer of the compound of Formula (I).

In some aspects, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (I) is a compound of Formula (II):

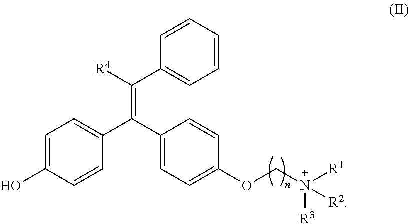

In some aspects, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (Ia) is a compound of Formula (IIa):

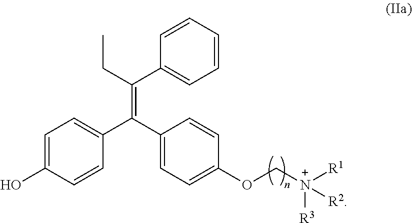

In some aspects, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (Ib) is a compound of Formula (IIb):

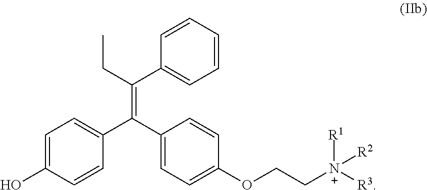

In various aspects, the present disclosure provides a compound of Formula (III):

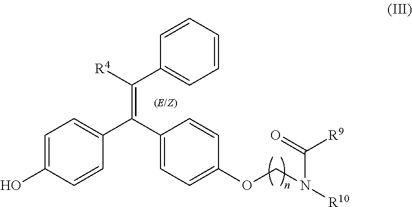

or a pharmaceutically acceptable salt, solvate, or tautomer thereof, wherein: R⁹ is hydrogen, hydroxyl, C₁-C₆ alkyl substituted with 0 to 3 instances of R¹¹, C₂-C₆ alkenyl substituted with 0 to 3 instances of R¹¹, or C₂-C₆ alkynyl substituted with 0 to 3 instances of R¹¹; R¹⁰ is hydrogen, C₁-C₆alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; R⁴ is C₁-C₆ alkyl substituted with 0 to 3 instances of R¹, C₂-C₆ alkenyl substituted with 0 to 3 instances of R⁸, or C₂-C₆ alkynyl substituted with 0 to 3 instances of R⁸; each instance of R¹¹ is independently halogen, hydroxyl, C₁-C₃ alkoxy, C₁-C₃ alkylthio, C₁-C₃ alkyl, or C₃-C₈ cycloalkyl, aryl, or —C(═O)OCH₃; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; each instance of R⁸ is independently halogen, hydroxyl, C₁-C₃ alkoxy, C₁-C₃ alkylthio, C₁-C₃ alkyl, or C₃-C₈ cycloalkyl; and n is an integer from 1 to 10.

In some aspects, the compound of Formula (III) is a compound of Formula (IIIa):

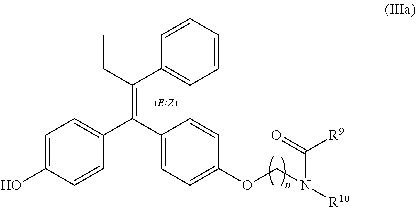

or a pharmaceutically acceptable salt, solvate, or tautomer thereof.

In some aspects, the compound of Formula (III) is a compound of Formula (IIIb):

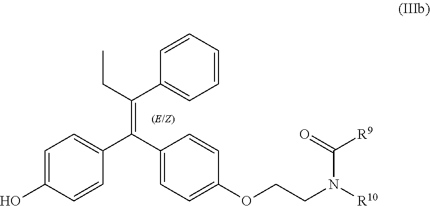

or a pharmaceutically acceptable salt, solvate, or tautomer thereof.

In some aspects, the compound of Formula (III) is a compound of Formula (IV):

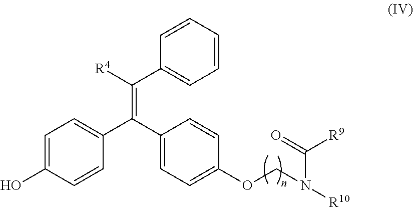

or a pharmaceutically acceptable salt, solvate, or tautomer thereof.

In some aspects, the compound of Formula (III) is a compound of Formula (IVa):

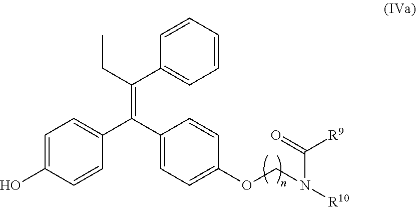

or a pharmaceutically acceptable salt, solvate, or tautomer thereof.

In some aspects, the compound of Formula (III) is a compound of Formula (IVb):

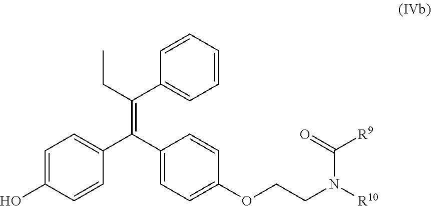

or a pharmaceutically acceptable salt, solvate, or tautomer thereof.

In some aspects, the compound is a (Z)-isomer of the compound of Formula (III). In some aspects, the compound is an (E)-isomer of the compound of Formula (III).

In some aspects, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (III) is a compound of Formula (IV):

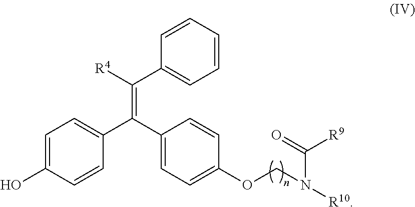

In some aspects, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (IIIa) is a compound of Formula (IVa):

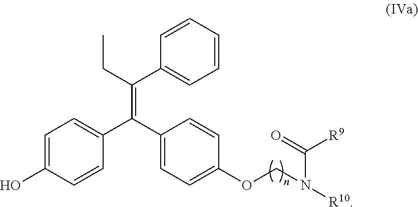

In some aspects, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (IIIb) is a compound of Formula (IVb):

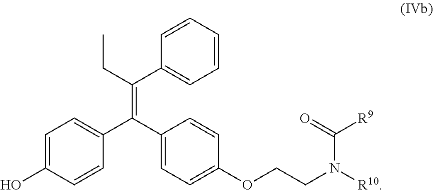

In various aspects, the present disclosure provides a compound of Formula (V):

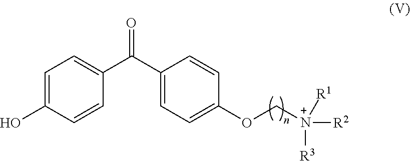

or a pharmaceutically acceptable salt, solvate, or tautomer thereof, wherein: R¹, R², R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and n is an integer from 1 to 10.

In some aspects, the compound of Formula (V) is a compound of Formula (Va):

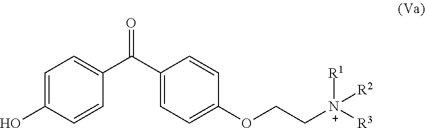

or a pharmaceutically acceptable salt, solvate, or tautomer thereof.

In some aspects, the compound of Formula (V) is a compound of Formula (Vb):

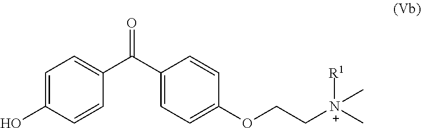

or a pharmaceutically acceptable salt, solvate, or tautomer thereof.

In various aspects, the present disclosure provides a compound of Formula (VI):

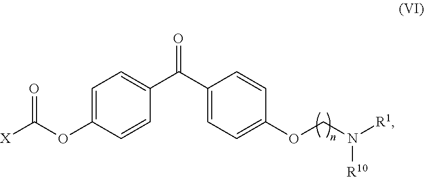

or a pharmaceutically acceptable salt, solvate, or tautomer thereof, wherein: R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; R¹⁰ is hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; X is C₁-C₆ alkyl, —OR¹², —SR¹² or —NR¹²R¹³; R¹² and R¹³ are each independently hydrogen or C₁-C₃ alkyl substituted with 0 or 1 instance of halogen; and n is an integer from 1 to 10.

In various aspects, the present disclosure provides a compound of Formula (VIa):

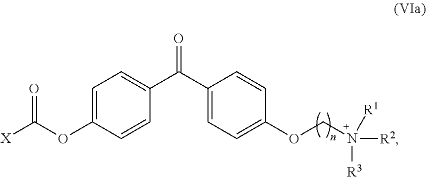

or a pharmaceutically acceptable salt, solvate, or tautomer thereof, wherein: R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; X is C₁-C₆ alkyl, —OR¹², —SR¹² or —NR¹²R¹³; R¹² and R¹³ are each independently hydrogen or C₁-C₃ alkyl substituted with 0 or 1 instance of halogen; and n is an integer from 1 to 10.

In some aspects, the compound of Formula (VI) is a compound of Formula (VIb):

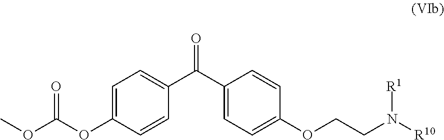

or a pharmaceutically acceptable salt, solvate, or tautomer thereof.

In some aspects, the compound of Formula (VIa) is a compound of Formula (VIc):

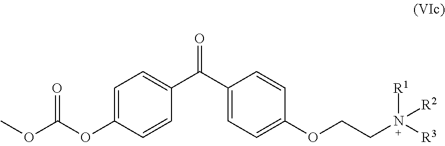

or a pharmaceutically acceptable salt, solvate, or tautomer thereof.

In some aspects, X is —OR¹² or —NR¹²R¹³. In some aspects, X is —OR¹² or —SR¹². In some aspects, X is —OR¹². In some aspects, X is —OCH₃.

In some aspects, R¹² and R¹³ are each independently C₁-C₃ alkyl. In some aspects, R¹² and R¹³ are each independently methyl. In some aspects, R¹² is methyl.

In various aspects, the present disclosure provides a compound of Formula (VII):

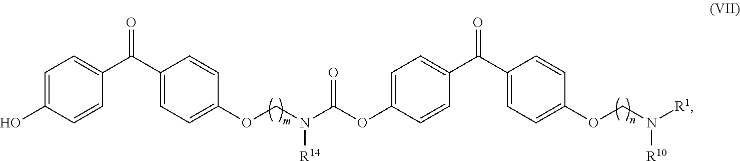

or a pharmaceutically acceptable salt, solvate, or tautomer thereof, wherein: R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and m and n are each independently integers from 1 to 10.

In various aspects, the present disclosure provides a compound of Formula (VIIa):

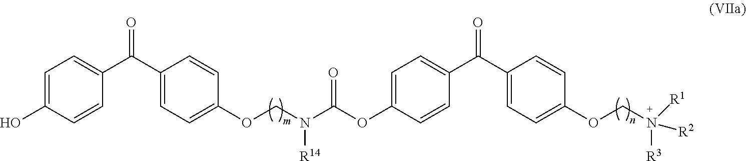

or a pharmaceutically acceptable salt, solvate, or tautomer thereof, wherein: R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and m and n are each independently integers from 1 to 10.

In some aspects, the compound of Formula (VII) is a compound of Formula (VIIb):

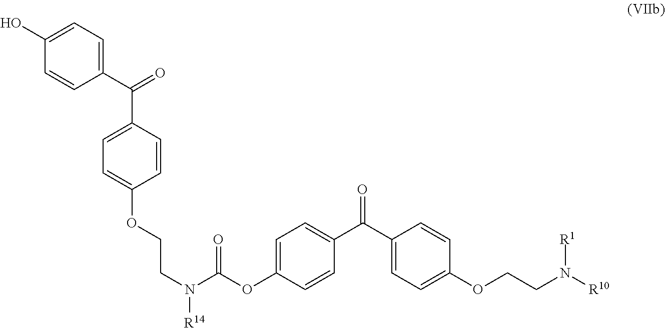

or a pharmaceutically acceptable salt, solvate, or tautomer thereof.

In some aspects, the compound of Formula (VIIa) is a compound of Formula (VIIc):

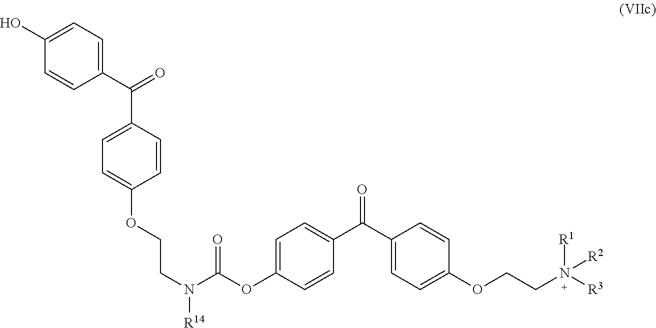

or a pharmaceutically acceptable salt, solvate, or tautomer thereof.

In various aspects, the present disclosure provides a compound of Formula (VIII):

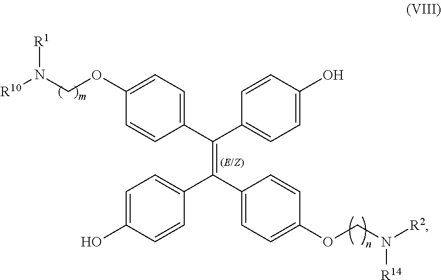

or a pharmaceutically acceptable salt, solvate, or tautomer thereof, wherein: R¹ and R² are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and m and n are each independently integers from 1 to 10.

In various aspects, the present disclosure provides a compound of Formula (VIIIa):

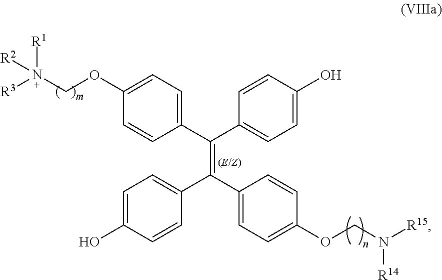

or a pharmaceutically acceptable salt, solvate, or tautomer thereof, wherein: R¹, R², R³, and R¹⁵ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and m and n are each independently integers from 1 to 10.

In various aspects, the present disclosure provides a compound of Formula (VIIIb):

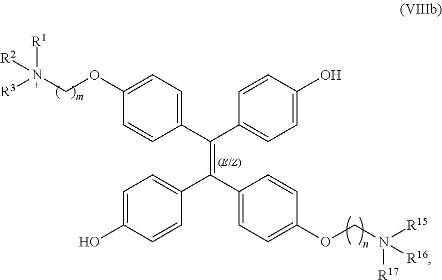

or a pharmaceutically acceptable salt, solvate, or tautomer thereof, wherein: R¹, R², R³, R¹⁵, R¹⁶, and R¹⁷ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶; each instance of R⁵ is independently halogen, hydroxyl, C₁-C₃ alkoxy, C₁-C₃ alkylthio, C₁-C₃ alkyl, —C(═O)OH, or —C(═O)OCH₃; each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and m and n are each independently integers from 1 to 10.

In some aspects, the compound of Formula (VIII) is a compound of Formula (VIIIc):

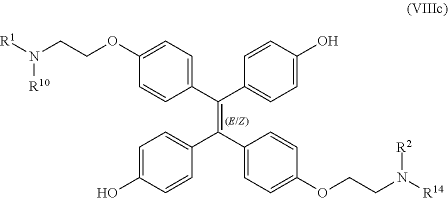

or a pharmaceutically acceptable salt, solvate, or tautomer thereof.

In some aspects, the compound of Formula (VIIIa) is a compound of Formula (VIIId):

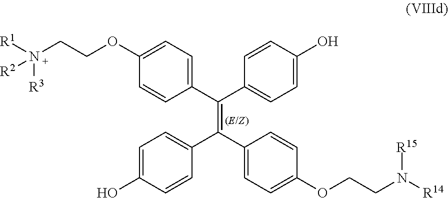

or a pharmaceutically acceptable salt, solvate, or tautomer thereof.

In some aspects, the compound of Formula (VIIIb) is a compound of Formula (VIIIe):

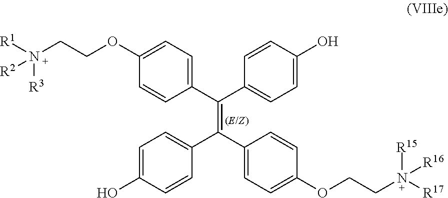

or a pharmaceutically acceptable salt, solvate, or tautomer thereof.

In some aspects, R¹, R², R³, R¹⁵, R¹⁶, and R¹⁷ are each independently C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some aspects, R¹, R², R³, R¹⁵, R¹⁶, and R¹⁷ are each independently C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some aspects, R¹, R², R³, R¹⁵, R¹⁶, and R¹⁷ are each independently C₁-C₆ alkyl substituted with 0 to 3 instances of R⁵. In some aspects, R¹, R², R³, R¹⁵, R¹⁶, and R¹⁷ are each independently C₁-C₆ alkyl substituted with 0 or 1 instance of R⁵. In some aspects, R¹, R², R³, R¹⁵, R¹⁶, and R¹⁷ are each independently C₁-C₃ alkyl substituted with 0 to 3 instances of R⁵. In some aspects, R¹, R², R³, R¹⁵, R¹⁶, and R¹⁷ are each independently C₁-C₃ alkyl substituted with 0 or 1 instance of R⁵. In some aspects, R¹, R², R³, R¹⁵, R¹⁶, and R¹⁷ are each independently C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some aspects, R¹, R², R³, R¹⁵, R¹⁶, and R¹⁷ are each independently C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some aspects, R¹, R², R³, R¹⁵, R¹⁶, and R¹⁷ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some aspects, R¹, R², R³, R¹⁵, R¹⁶, and R¹⁷ are each independently C₁-C₆ alkyl. In some aspects, R¹, R², R³, R¹⁵, R¹⁶, and R¹⁷ are each independently C₁-C₃ alkyl. In some aspects, R¹, R², R³, R¹⁵, R¹⁶, and R¹⁷ are each —CH₃. In some aspects, R¹, R², and R³ are each —CH₃. In some aspects, R¹⁵, R¹⁶, and R¹⁷ are each —CH₃. In some aspects, R¹ is —CH₃. In some aspects, R² is —CH₃. In some aspects, R¹⁵ is —CH₃.

In some aspects, R⁴ is C₁-C₆ alkyl substituted with 0 to 3 instances of R⁸. In some aspects, R⁴ is C₁-C₆ alkyl substituted with 0 or 1 instance of R⁸. In some aspects, R⁴ is C₁-C₃ alkyl substituted with 0 to 3 instances of R⁸. In some aspects, R⁴ is C₁-C₃ alkyl substituted with 0 or 1 instance of R⁸. In some aspects, R⁴ is C₁-C₃ alkyl. In some aspects, R⁴ is —CH₂CH₃.

In some aspects, each instance of R⁵ is independently halogen, hydroxyl, C₁-C₃ alkoxy, C₁-C₃ alkylthio, or C₁-C₃ alkyl. In some aspects, each instance of R⁵ is independently hydroxyl, C₁-C₃ alkoxy, or C₁-C₃ alkyl. In some aspects, each instance of R⁵ is independently hydroxyl or C₁-C₃ alkyl. In some aspects, each instance of R⁵ is hydroxyl. In some aspects, each instance of R⁵ is independently C₁-C₃ alkoxy. In some aspects, each instance of R⁵ is independently C₁-C₃ alkyl.

In some aspects, each instance of R⁶ and R⁷ is independently hydrogen or C₁-C₃ alkyl. In some aspects, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some aspects, each instance of R⁶ and R⁷ is independently hydrogen or methyl. In some aspects, each instance of R⁶ and R⁷ is independently C₁-C₃ alkyl. In some aspects, each instance of R⁶ and R⁷ is hydrogen.

In some aspects, each instance of R⁸ is independently hydroxyl, C₁-C₃ alkoxy, C₁-C₃ alkyl, or C₃-C₈ cycloalkyl. In some aspects, each instance of R⁸ is independently hydroxyl, C₁-C₃ alkoxy, or C₁-C₃ alkyl. In some aspects, each instance of R⁸ is independently hydroxyl or C₁-C₃ alkyl. In some aspects, each instance of R⁸ is independently C₁-C₃ alkyl.

In some aspects, R⁹ is hydrogen, hydroxyl, C₁-C₆ alkyl substituted with 0 or 1 instance of R¹¹, C₂-C₆ alkenyl substituted with 0 or 1 instance of R¹¹, or C₂-C₆ alkynyl substituted with 0 or 1 instance of R¹¹. In some aspects, R⁹ is hydrogen, hydroxyl, C₁-C₃ alkyl substituted with 0 to 3 instances of R¹¹, C₂-C₃ alkenyl substituted with 0 to 3 instances of R¹¹, or C₂-C₃ alkynyl substituted with 0 to 3 instances of R¹¹. In some aspects, R⁹ is hydrogen, hydroxyl, C₁-C₃ alkyl substituted with 0 or 1 instance of R¹¹, C₂-C₃ alkenyl substituted with 0 or 1 instance of R¹¹, or C₂-C₃ alkynyl substituted with 0 or 1 instance of R¹¹. In some aspects, R⁹ is hydrogen, hydroxyl, or C₁-C₆ alkyl substituted with 0 to 3 instances of R¹¹. In some aspects, R⁹ is C₁-C₆ alkyl substituted with 0 to 3 instances of R¹¹. In some aspects, R⁹ is hydrogen, hydroxyl, or C₁-C₆ alkyl substituted with 0 or 1 instance of R¹¹. In some aspects, R⁹ is C₁-C₆ alkyl substituted with 0 or 1 instance of R¹¹. In some aspects, R⁹ is hydrogen, hydroxyl, or C₁-C₆ alkyl. In some aspects, R⁹ is C₁-C₆ alkyl. In some aspects, R⁹ is hydrogen, hydroxyl, or C₁-C₃ alkyl substituted with 0 to 3 instances of R¹¹. In some aspects, R⁹ is C₁-C₃ alkyl substituted with 0 to 3 instances of R¹¹. In some aspects, R⁹ is hydrogen, hydroxyl, or C₁-C₃ alkyl substituted with 0 or 1 instance of R¹¹. In some aspects, R⁹ is C₁-C₃ alkyl substituted with 0 or 1 instance of R¹¹. In some aspects, R⁹ is hydrogen, hydroxyl, or C₁-C₃ alkyl. In some aspects, R⁹ is C₁-C₃ alkyl. In some aspects, R⁹ is hydrogen. In some aspects, R⁹ is methyl.

In some aspects, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OR⁶, —C(═O)NR⁶R⁷, or —C(═O)SR⁶. In some aspects, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OH, —C(═O)NH₂, or —C(═O)SH. In some aspects, R¹⁰ and R¹⁴ are each independently hydrogen, C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some aspects, R¹⁰ and R¹⁴ are each independently hydrogen or C₁-C₆ alkyl substituted with 0 to 3 instances of R⁵. In some aspects, R¹⁰ and R¹⁴ are each independently hydrogen or C₁-C₃ alkyl substituted with 0 to 3 instances of R⁵. In some aspects, R¹⁰ and R¹⁴ are each independently hydrogen or C₁-C₆ alkyl substituted with 0 or 1 instance of R⁵. In some aspects, R¹⁰ and R¹⁴ are each independently hydrogen or C₁-C₃ alkyl substituted with 0 or 1 instance of R⁵. In some aspects, R¹⁰ and R¹⁴ are each independently hydrogen or C₁-C₃ alkyl. In some aspects, R¹⁰ and R¹⁴ are each independently hydrogen or methyl. In some aspects, R¹⁰ and R¹⁴ are each independently hydrogen. In some aspects, R¹⁰ and R¹⁴ are each independently methyl. In some aspects, R¹⁰ is hydrogen. In some aspects, R¹⁰ is methyl. In some aspects, R¹⁴ is hydrogen. In some aspects, R¹⁴ is methyl.

In some aspects, each instance of R¹¹ is independently halogen, hydroxyl, C₁-C₃ alkoxy, C₁-C₃ alkylthio, or C₁-C₃ alkyl. In some aspects, each instance of R¹¹ is independently halogen, hydroxyl, C₁-C₃ alkylthio, or C₁-C₃ alkyl. In some aspects, each instance of R¹¹ is independently hydroxyl, C₁-C₃ alkoxy, or C₁-C₃ alkyl. In some aspects, each instance of R¹¹ is independently hydroxyl or C₁-C₃ alkyl. In some aspects, each instance of R¹¹ is hydroxyl. In some aspects, each instance of R¹¹ is independently C₁-C₃ alkyl.

In some aspects, n is 1 to 4. In some aspects, n is 2 to 4. In some aspects, n is 2 or 3. In some aspects, n is 2. In some aspects, m is 1 to 4. In some aspects, m is 2 to 4. In some aspects, m is 2 or 3. In some aspects, m is 2.

In various aspects, the present disclosure provides a compound of Formula (IX):

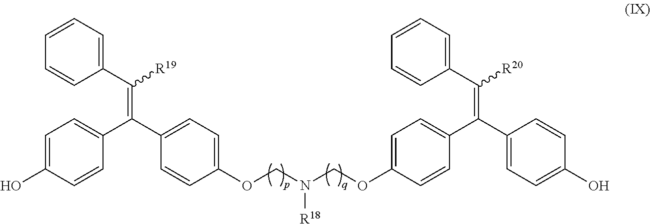

or a pharmaceutically acceptable salt, solvate, or tautomer thereof,
wherein:
   R¹⁸ is hydrogen, C₁-C₆ alkyl substituted with 0 to 3 instances of R²¹, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   R¹⁹ and R²⁰ are each independently C₁-C₆ alkyl substituted with 0 to 3 instances of R⁸, C₂-C₆ alkenyl substituted with 0 to 3 instances of R⁸, or C₂-C₆ alkynyl substituted with 0 to 3 instances of R²²;
   each instance of R²¹ is independently halogen, hydroxyl, C₁-C₃ alkoxy, C₁-C₃ alkylthio, C₁-C₃ alkyl, —C(═O)OH, or —C(═O)OCH₃;
each instance of R²² is independently halogen, hydroxyl, C₁-C₃ alkoxy, C₁-C₃ alkylthio, C₁-C₃ alkyl, or C₃-C₈ cycloalkyl; and
   p and q are each independently integers from 1 to 10.

In some aspects, the compound of Formula (IX) is a compound of Formula (IXa):

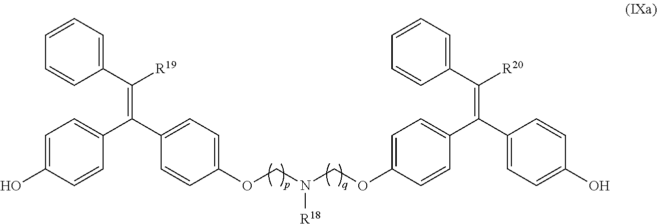

or a pharmaceutically acceptable salt, solvate, or tautomer thereof.

In some aspects, the compound of Formula (IX) is a compound of Formula (IXb):

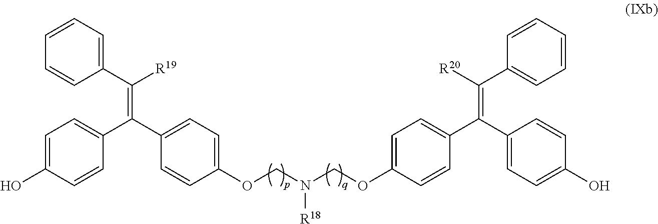

or a pharmaceutically acceptable salt, solvate, or tautomer thereof.

In some aspects, the compound of Formula (IX) is a compound of Formula (IXc):

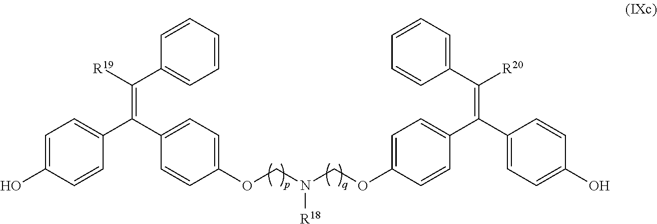

or a pharmaceutically acceptable salt, solvate, or tautomer thereof.

In some aspects, the compound of Formula (IX) is a compound of Formula (IXd):

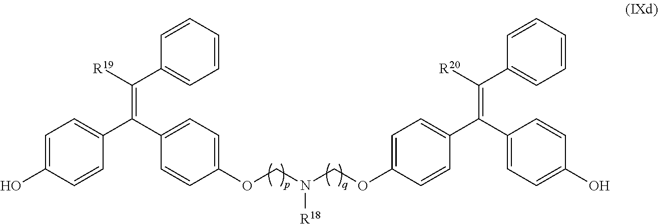

or a pharmaceutically acceptable salt, solvate, or tautomer thereof.

In some aspects, R¹⁹ is C₁-C₃ alkyl optionally substituted with one instance of R²². In other aspects, R¹⁹ is ethyl.

In further aspects, R²⁰ is C₁-C₃ alkyl optionally substituted with one instance of R²². In certain aspects, R²⁰ is ethyl.

In one aspect, R¹⁸ is hydrogen or C₁-C₆ alkyl. In a particular aspect, R¹⁸ is hydrogen. In another aspect, R¹⁸ is methyl.

In some aspects, p is 1 to 4. In additional aspects, p is 2 to 4. In particular aspects, p is 2 or 3. In a specific aspect, p is 2.

In some aspects, q is 1 to 4. In additional aspects, q is 2 to 4. In particular aspects, q is 2 or 3. In a specific aspect, q is 2.

In some aspects, the compound has any one of the following structures:

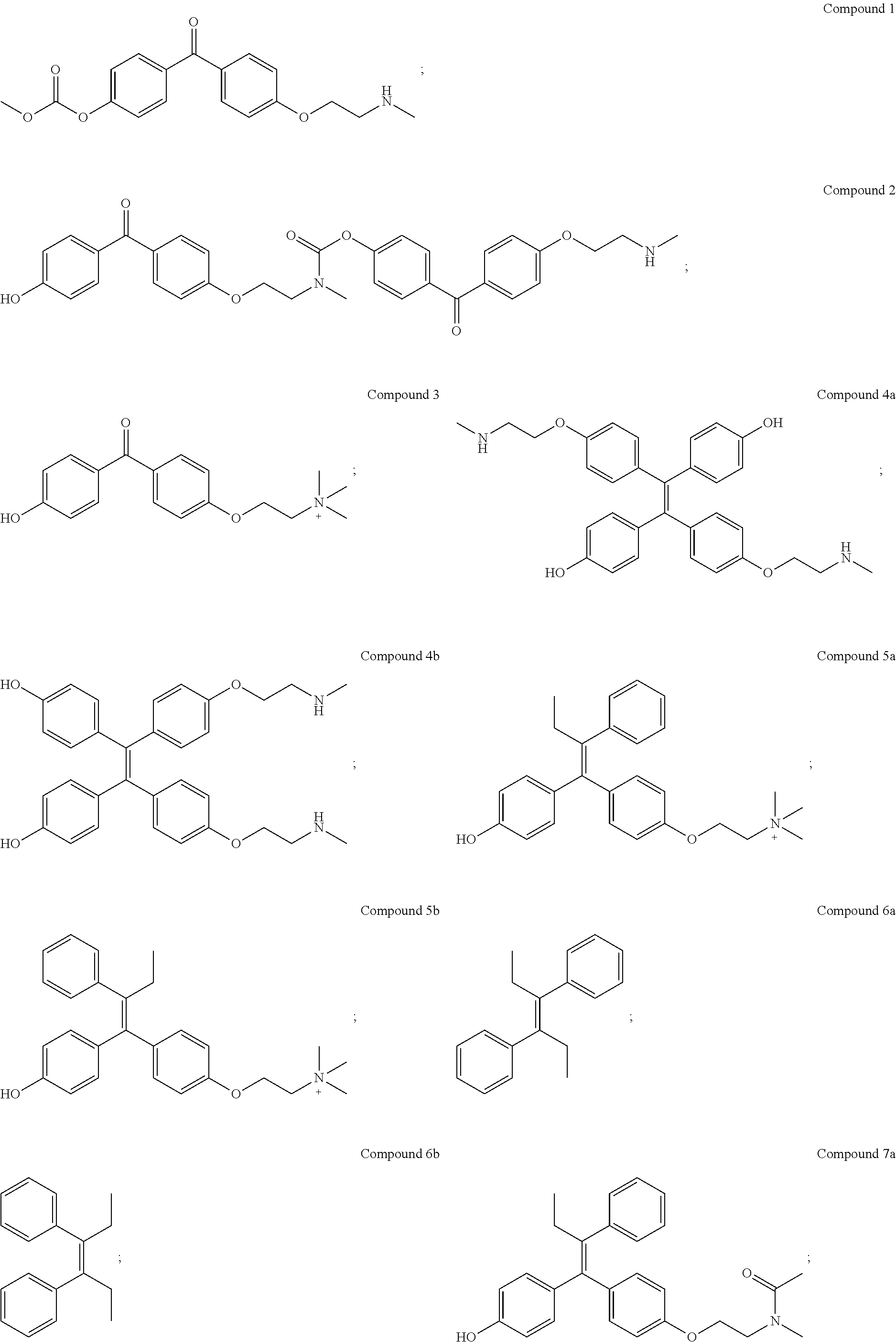

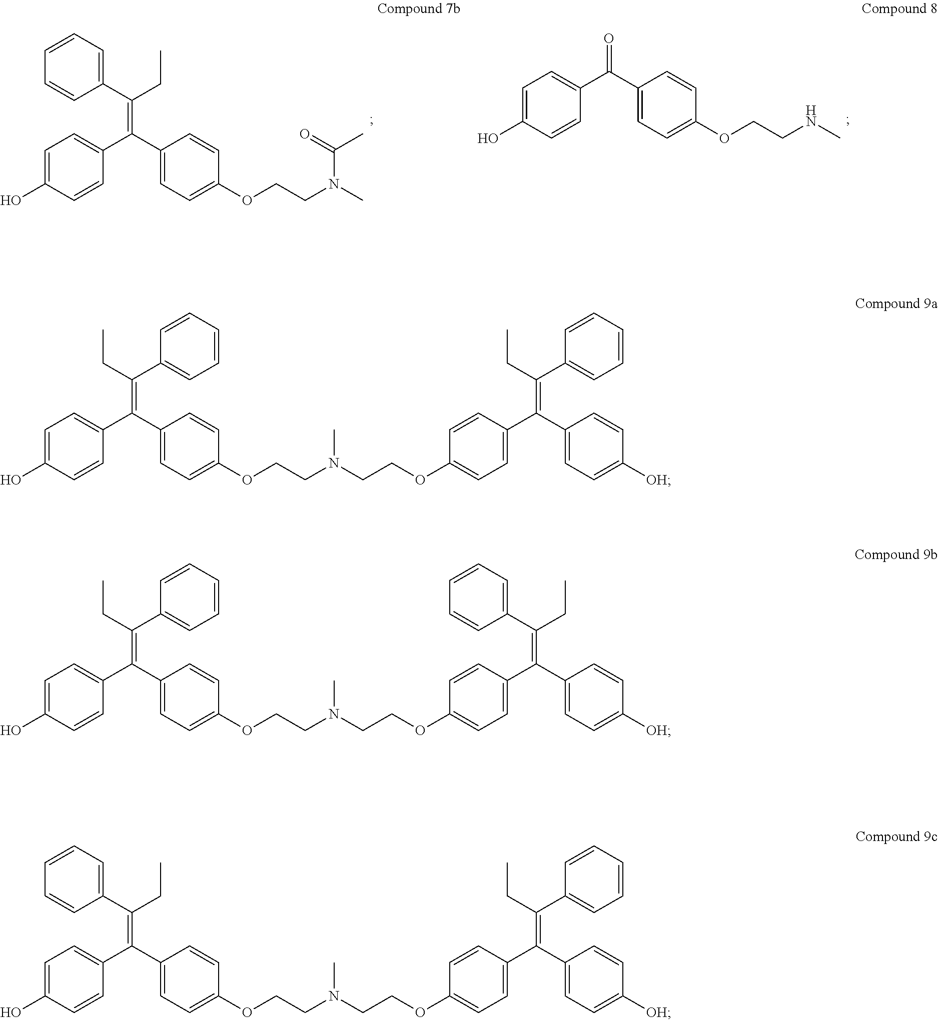 [SMILES: CC/C(=C(/c1ccc(O)cc1)c1ccc(OCCN(C)C(C)=O)cc1)c1ccccc1 ; CNCCOc1ccc(C(=O)c2ccc(O)cc2)cc1 ; CC/C(=C(\c1ccc(O)cc1)c1ccc(OCCN(C)CCOc2ccc(/C(=C(\CC)c3ccccc3)c3ccc(O)cc3)cc2)cc1)c1ccccc1 ; CC/C(=C(\c1ccc(O)cc1)c1ccc(OCCN(C)CCOc2ccc(/C(=C(/CC)c3ccccc3)c3ccc(O)cc3)cc2)cc1)c1ccccc1 ; CC/C(=C(/c1ccc(O)cc1)c1ccc(OCCN(C)CCOc2ccc(/C(=C(\CC)c3ccccc3)c3ccc(O)cc3)cc2)cc1)c1ccccc1]

a pharmaceutically acceptable salt, solvate, or tautomer thereof.

In some aspects, the compound has any one of the following structures:

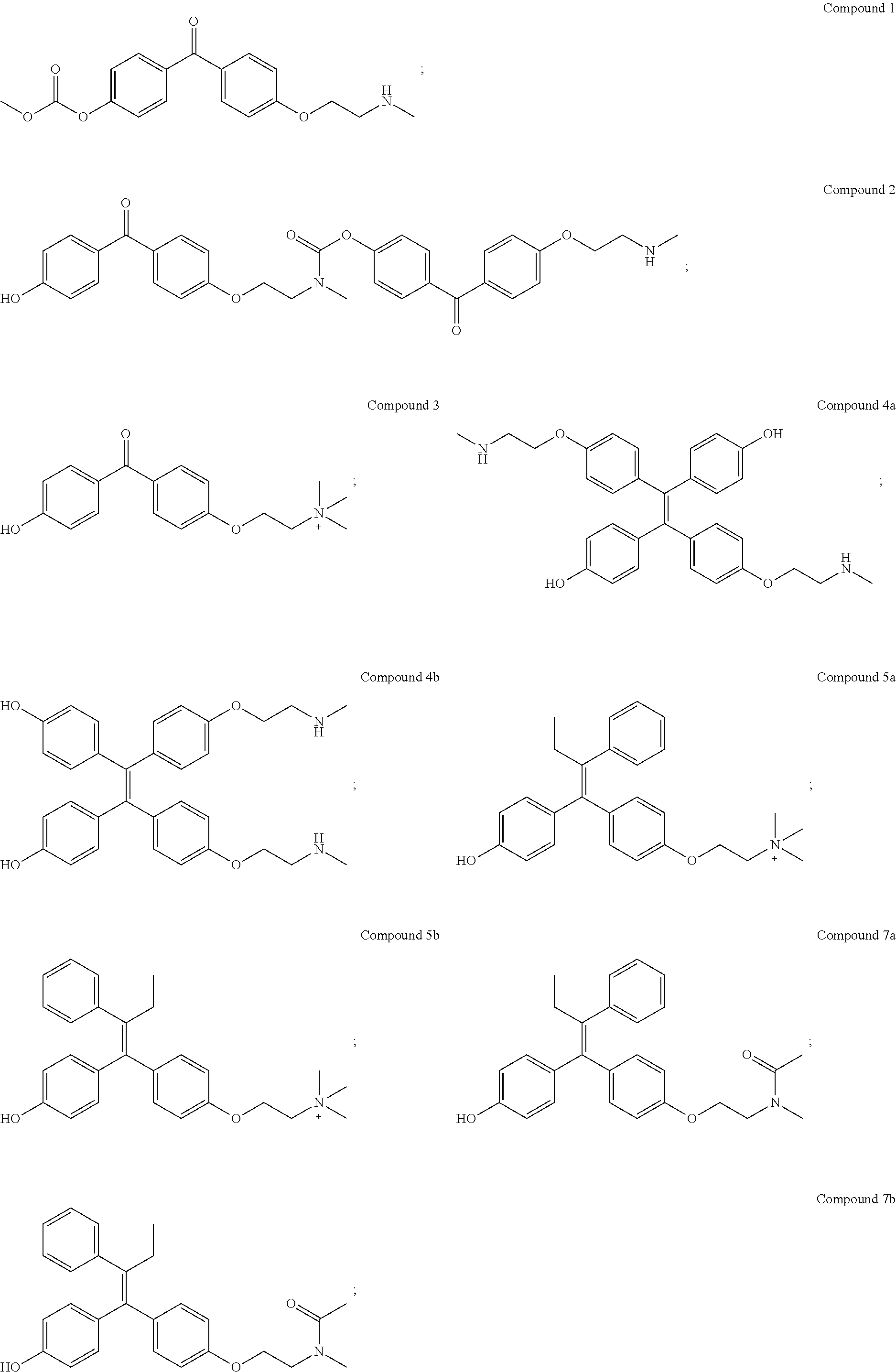

or a pharmaceutically acceptable salt, solvate, or tautomer thereof.

In various aspects, the present disclosure provides a pharmaceutical composition comprising a first compound as disclosed herein and a pharmaceutically acceptable carrier or diluent.

In some aspects, the pharmaceutical composition further comprises a second compound as disclosed herein. In some aspects, the first compound is present in between 40-60%, 50-70%, 60-80%, 70-90%, 80-90%, 85-95%, 90-95%, or 95-99% (w/w). In some aspects, the second compound is present between 1-5%, 5-10%, 5-15%, 10-20%, 10-30%, 20-40%, 30-50%, or 40-60% (w/w).

In some aspects, the first compound is:

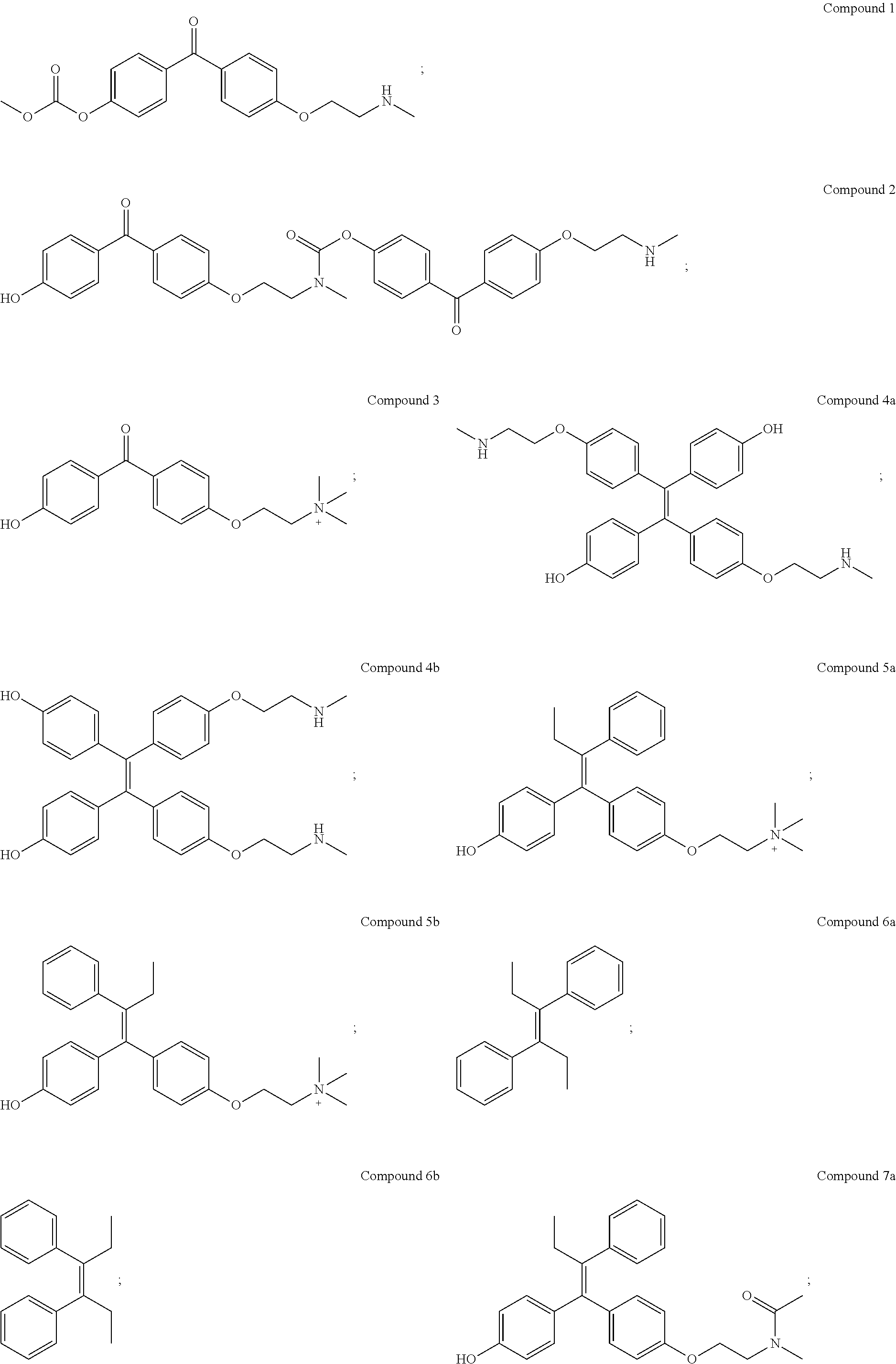

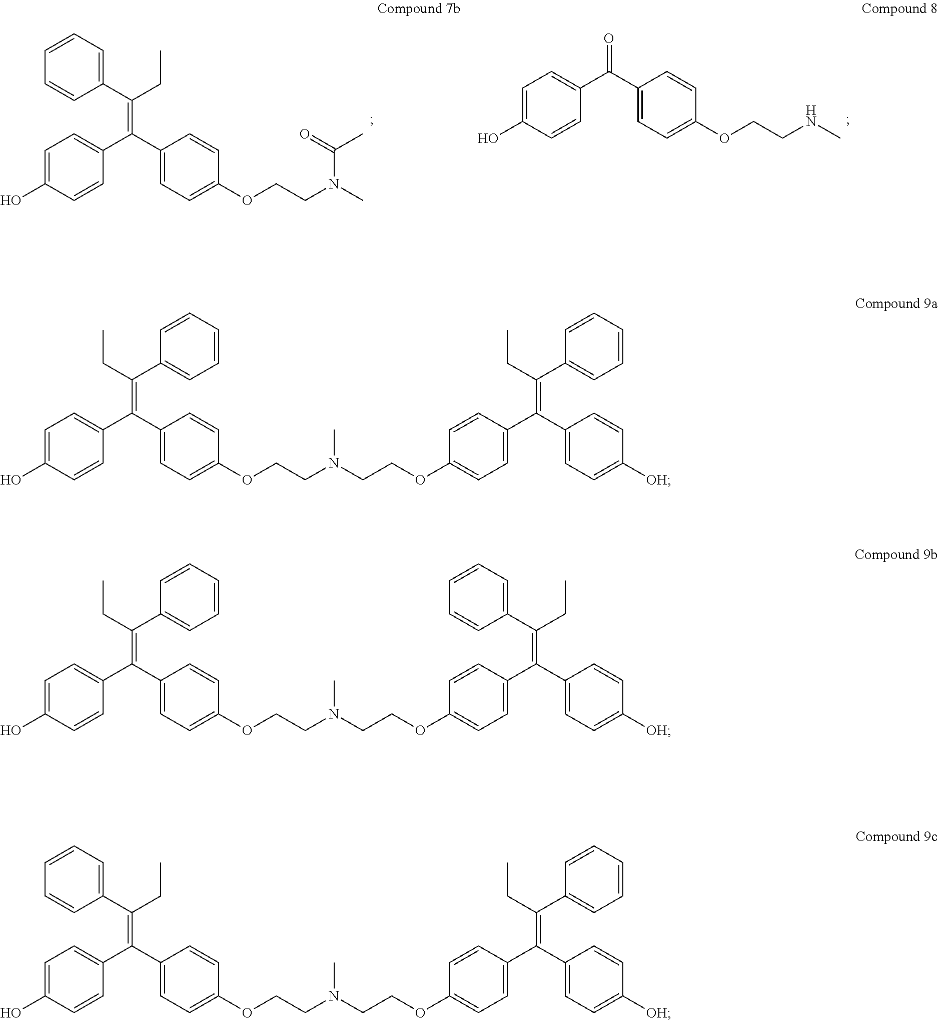 [SMILES: CC/C(=C(/c1ccc(O)cc1)c1ccc(OCCN(C)C(C)=O)cc1)c1ccccc1 ; CNCCOc1ccc(C(=O)c2ccc(O)cc2)cc1 ; CC/C(=C(\c1ccc(O)cc1)c1ccc(OCCN(C)CCOc2ccc(/C(=C(\CC)c3ccccc3)c3ccc(O)cc3)cc2)cc1)c1ccccc1 ; CC/C(=C(\c1ccc(O)cc1)c1ccc(OCCN(C)CCOc2ccc(/C(=C(/CC)c3ccccc3)c3ccc(O)cc3)cc2)cc1)c1ccccc1 ; CC/C(=C(/c1ccc(O)cc1)c1ccc(OCCN(C)CCOc2ccc(/C(=C(\CC)c3ccccc3)c3ccc(O)cc3)cc2)cc1)c1ccccc1]

or a pharmaceutically acceptable salt, solvate, or tautomer thereof.

In some aspects, the first compound is:

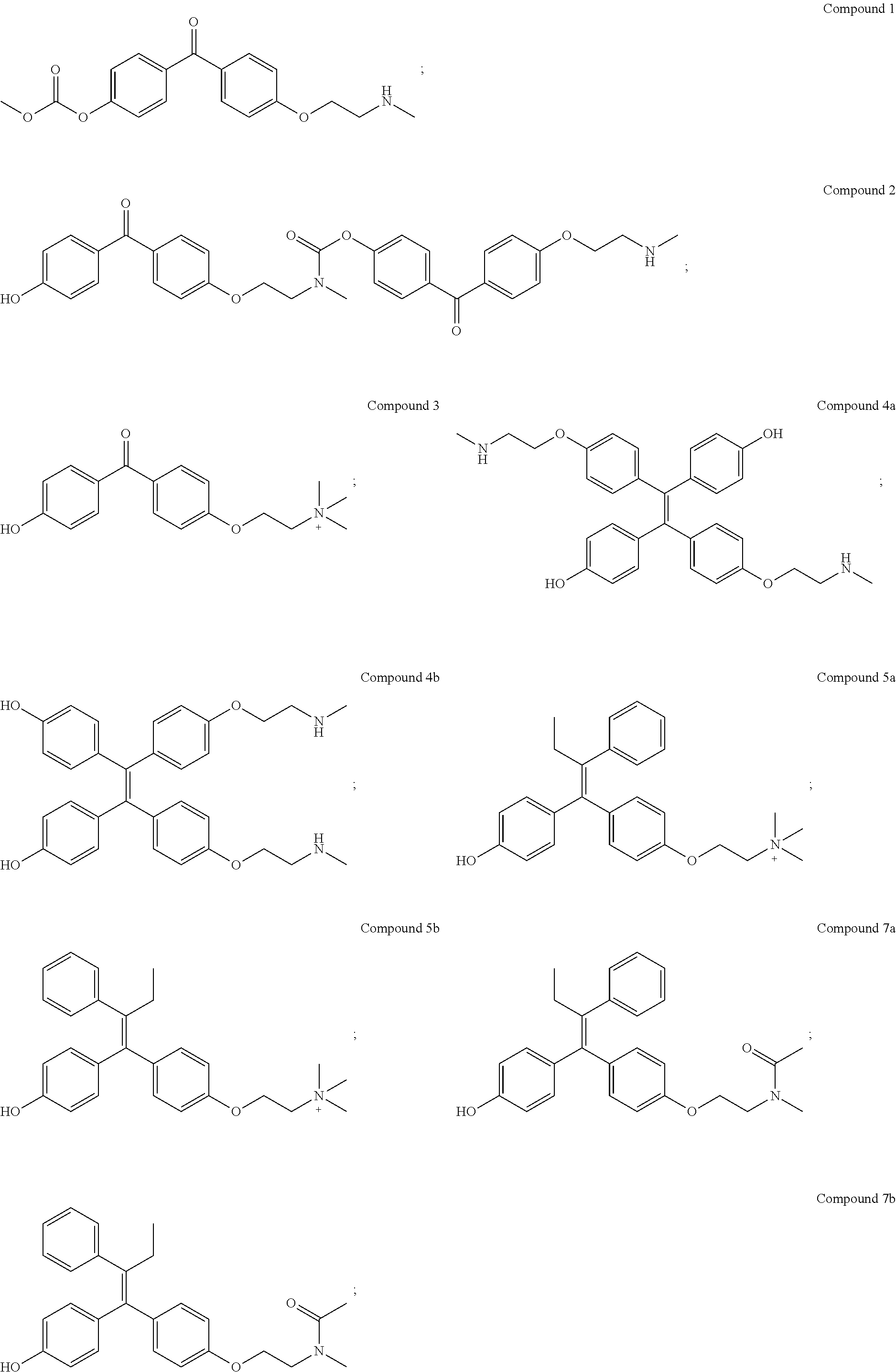

or a pharmaceutically acceptable salt, solvate, or tautomer thereof.

In some aspects, the second compound is:

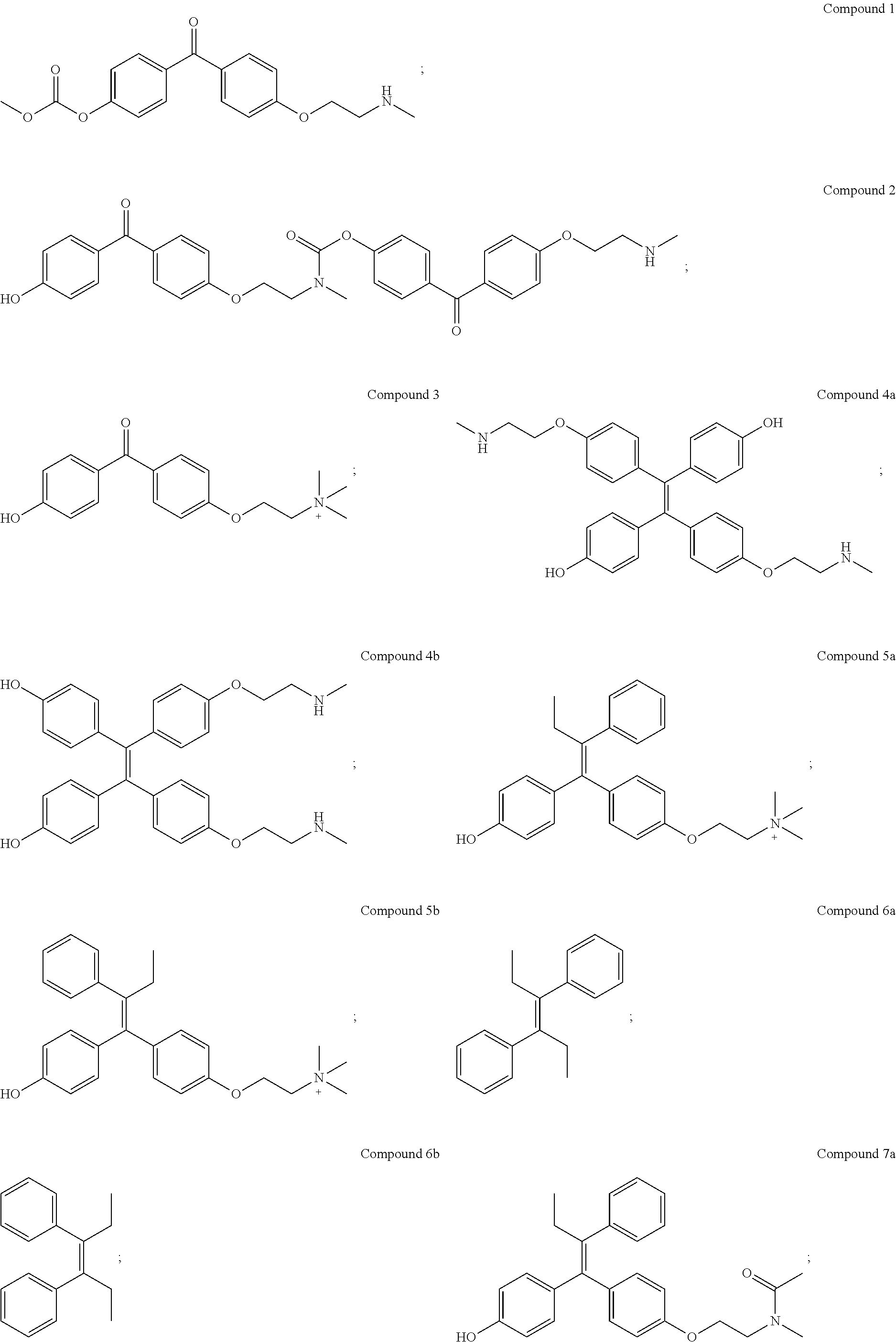

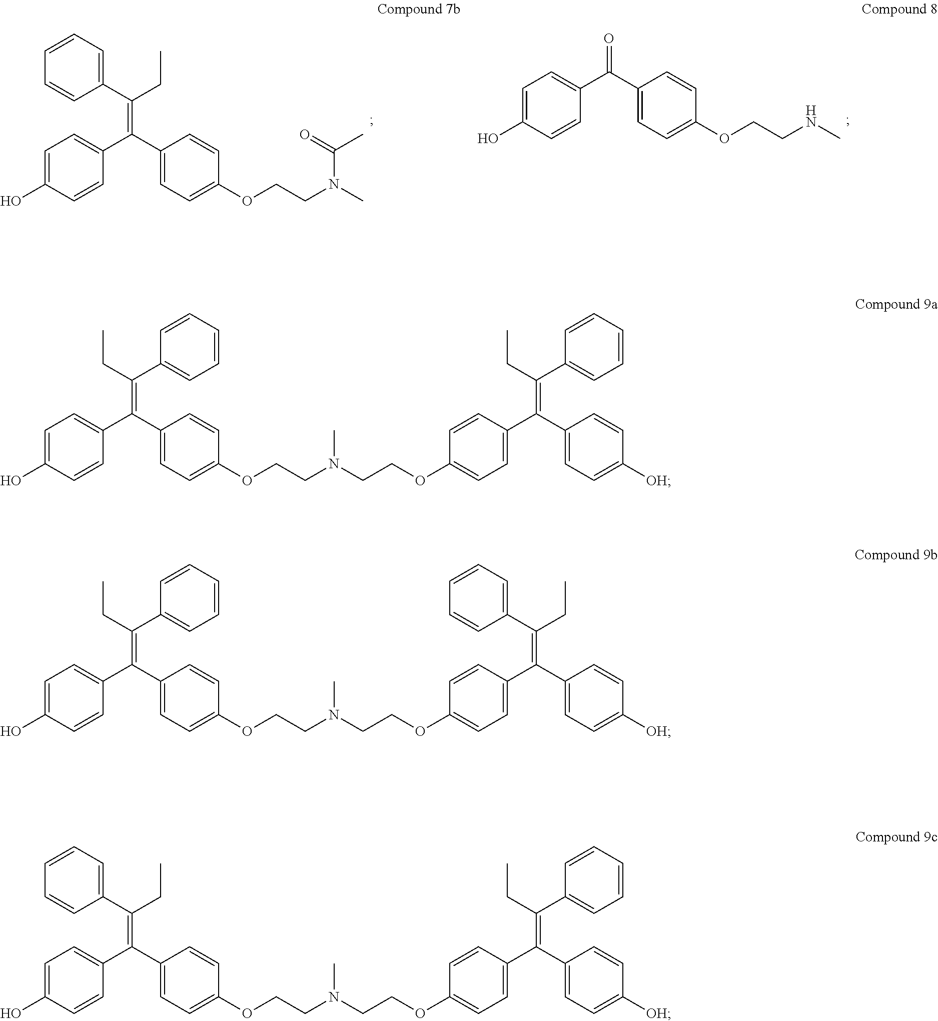 [SMILES: CC/C(=C(/c1ccc(O)cc1)c1ccc(OCCN(C)C(C)=O)cc1)c1ccccc1 ; CNCCOc1ccc(C(=O)c2ccc(O)cc2)cc1 ; CC/C(=C(\c1ccc(O)cc1)c1ccc(OCCN(C)CCOc2ccc(/C(=C(\CC)c3ccccc3)c3ccc(O)cc3)cc2)cc1)c1ccccc1 ; CC/C(=C(\c1ccc(O)cc1)c1ccc(OCCN(C)CCOc2ccc(/C(=C(/CC)c3ccccc3)c3ccc(O)cc3)cc2)cc1)c1ccccc1 ; CC/C(=C(/c1ccc(O)cc1)c1ccc(OCCN(C)CCOc2ccc(/C(=C(\CC)c3ccccc3)c3ccc(O)cc3)cc2)cc1)c1ccccc1]

or a pharmaceutically acceptable salt, solvate, or tautomer thereof.

In some aspects, the second compound is:

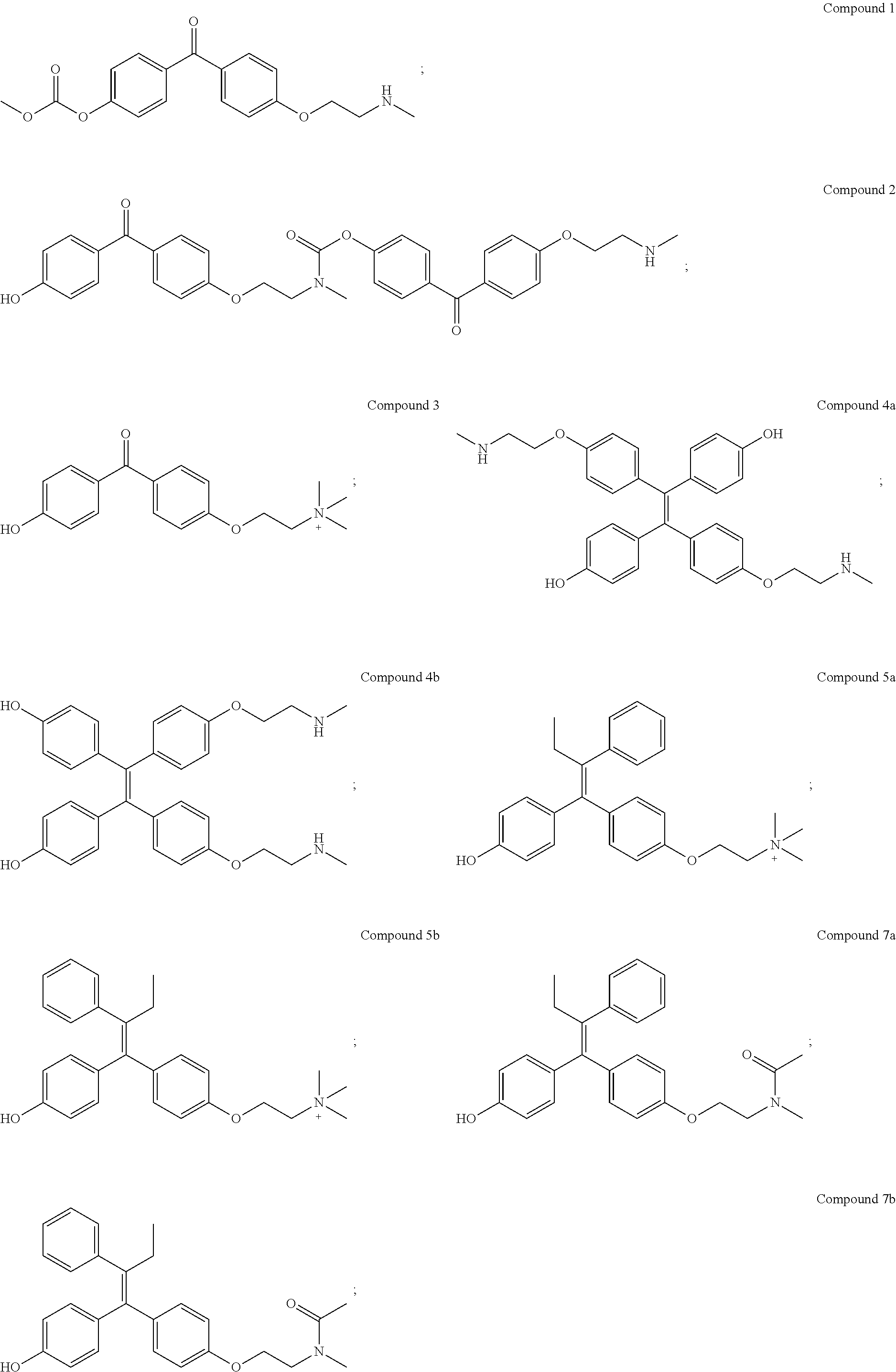

or a pharmaceutically acceptable salt, solvate, or tautomer thereof.

In some aspects, the pharmaceutical composition further comprises one or more additional compounds as disclosed herein. In some aspects, the pharmaceutical composition further comprises Compound 6A

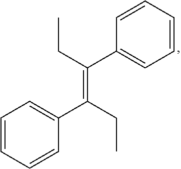 [SMILES: CC/C(=C(/CC)c1ccccc1)c1ccccc1]

### Compound 6B

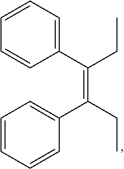 [SMILES: CC/C(=C(\CC)c1ccccc1)c1ccccc1]

a combination thereof, or a pharmaceutically acceptable salt, solvate, or tautomer thereof.

In some aspects, the pharmaceutical composition further comprises endoxifen, or a pharmaceutically acceptable salt, solvate, or tautomer thereof. In some aspects, the pharmaceutical composition is a solid. In some aspects, the pharmaceutical composition is a powder. In some aspects, the pharmaceutical composition further comprises a liquid. In some aspects, the first compound is suspended in the liquid.

In various aspects, the present disclosure provides a method comprising administering a compound as disclosed herein or the pharmaceutical composition as described herein to a subject.

In some aspects, the method further comprises treating a condition in a subject in need thereof. In some aspects, the condition is cancer. In some aspects, the compound as disclosed herein or the pharmaceutical composition as disclosed herein has aromatase inhibition activity. In some aspects, the compound as disclosed herein or the pharmaceutical composition as disclosed herein has affinity to an estrogen receptor.

In various aspects, the present disclosure provides a method of producing a compound as disclosed herein, the method comprising: coupling a compound of Formula (V):

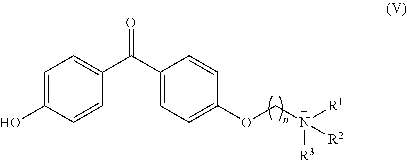

or a pharmaceutically acceptable salt, solvate, or tautomer thereof, with a compound of Formula (S2):

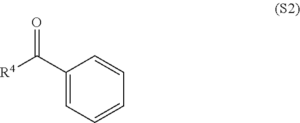

or a pharmaceutically acceptable salt, solvate, or tautomer thereof, thereby producing the compound as disclosed herein.

In some aspects, the coupling comprises an olefination reaction. In some aspects, the coupling comprises a McMurry reaction. In some aspects, the method comprises activating a compound of Formula (V) and a compound of Formula (S2) with a metal catalyst. In some aspects, the method comprises generating the metal catalyst by combining a metal salt with a reducing agent. In some aspects, the metal catalyst comprises titanium, zirconium, vanadium, niobium, molybdenum, tungsten, aluminum, or zinc. In some aspects, the metal catalyst comprises titanium. In some aspects, the metal salt comprises a titanium halide. In some aspects, the titanium halide comprises TiCl₃ or TiCl₄. In some aspects, the reducing agent comprises zinc, zirconium, vanadium, niobium, molybdenum, tungsten, aluminum, magnesium, potassium, zinc-copper couple, alkali and alkali earth metals, butylium, lithium, or lithium aluminum hydride. In some aspects, the zinc comprises zinc powder.

In some aspects, the compound of Formula (S2) is Compound S4:

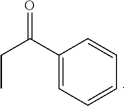

In some aspects, the method further comprises a first solution made by combining the reducing agent and an organic solvent under an inert atmosphere at a first temperature. In some aspects, the organic solvent is THF. In some aspects, the inert atmosphere is a N₂ atmosphere. In some aspects, the first temperature is between 15° to 30° C. In some aspects, the method further comprises cooling the first solution to a second temperature and adding the metal catalyst. In some aspects, the second temperature is between −10° to 5° C. In some aspects, the method further comprises maintaining the first solution at a third temperature during the addition of the metal catalyst. In some aspects, the third temperature is less than 20° C. In some aspects, the method further comprises heating the first solution to a fourth temperature for 1 hour, 1.5 hours, 2 hours, 2.5 hours, or 3 hours. In some aspects, the fourth temperature is between 50° to 70° C.

In some aspects, the method further comprises a second solution made by adding the compound of Formula (S2) and the compound of Formula (V) to an organic solvent. In some aspects, the organic solvent is THF. In some aspects, the method further comprises heating the second solution to between 50° to 70° C. In some aspects, the method further comprises a third solution made by adding the second solution to the first solution over 1 hour, 1.5 hours, or 2 hours. In some aspects, the method further comprises stirring the third solution at between 50° to 70° C. for 7 hours, 8 hours, 9 hours, or 10 hours.

In some aspects, the method further comprises distilling, washing, filtering, separating, extracting, precipitating, collecting, or a combination thereof, the compound of Formula (I) with an organic solvent, an acid, or an aqueous solution. In some aspects, the organic solvent is MeTHF. In some aspects, the washing comprises an organic solvent, an acid, a base, a solvent mixture, or a combination thereof. In some aspects, the organic solvent is MeTHF, acetonitrile, acetone, or a combination thereof. In some aspects, the acid is between 2N to 12 N HCl. In some aspects, the aqueous solution is 30-35% HCl, between 10% to 60% NaCl, or between 10% to 60% K₂CO₃.

In some aspects, the compound of Formula (V) is Compound 3:

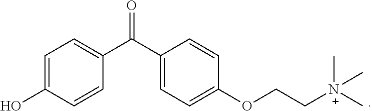 [SMILES: C[N+](C)(C)CCOc1ccc(C(=O)c2ccc(O)cc2)cc1]

In some aspects, the compound produced is Compounds 5a and 5b:

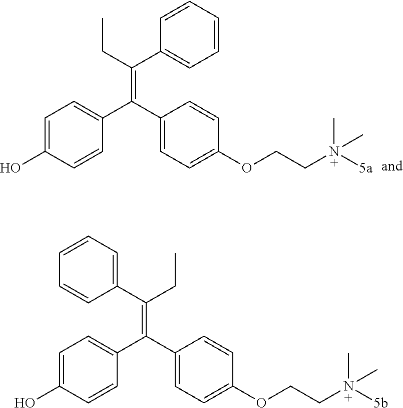

or a pharmaceutically acceptable salt, solvate, or tautomer thereof.

In some aspects, the method comprises producing a compound of Formula (V) by functionalizing a compound of Formula (S1):

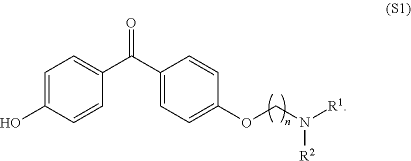

In some aspects, the method comprises producing a compound of Formula (V) by alkylating a compound of Formula (S1):

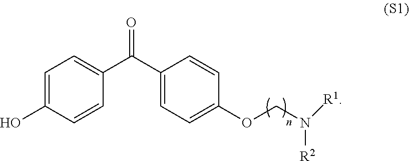

In some aspects, the method comprises producing a compound of Formula (V) by methylating a compound of Formula (S1):

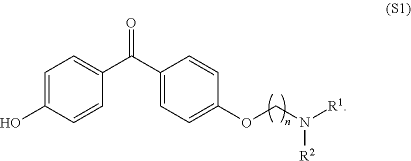

In some aspects, the method comprising coupling a compound of Formula (S3):

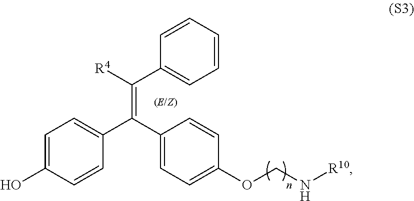

and a compound of Formula (S4):

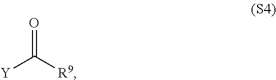

wherein Y is a leaving group, thereby producing a compound as disclosed herein.

In some aspects, Y is an alkoxy, a hydroxy, an activated hydroxyl, a halogen, or an ester. In some aspects, the compound of Formula (S4) is ethyl acetate. In some aspects, the compound of Formula (S3) is Compound S3a:

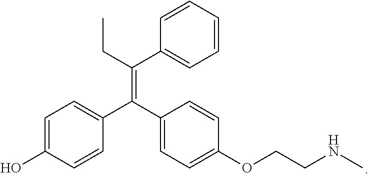 [SMILES: CC/C(=C(\c1ccc(O)cc1)c1ccc(OCCNC)cc1)c1ccccc1]

In some aspects, the compound of Formula (S3) is Compound S3b:

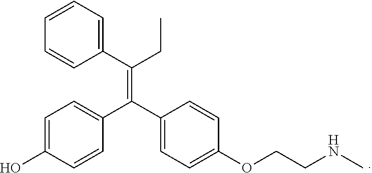 [SMILES: CC/C(=C(/c1ccc(O)cc1)c1ccc(OCCNC)cc1)c1ccccc1]

In some aspects, the method is performed in an organic solvent. In some aspects, the organic solvent comprises an aprotic solvent. In some aspects, the organic solvent comprises ethyl acetate. In some aspects, the method further comprises the addition of an acid. In some aspects, the acid comprises between 2 to 12 N of the acid. In some aspects, the acid comprises HCl. In some aspects, the method comprises maintaining the coupling at a temperature of between 0 to 20° C., between −5 to 10° C., or between −10 to 10° C. during the addition of the acid. In some aspects, the method further comprises heating the coupling to an elevated temperature. In some aspects, the elevated temperature is between 50 to 60° C., between 55 to 65° C., or between 60 to 70° C. In some aspects, the method further comprises stirring the solution for between 5 to 15 hours, between 8 to 12 hours, or between 9 to 11 hours.

In some aspects, the method further comprises washing, purifying, or separating the compound of Formula (III) using a base, an aqueous solution, an alcohol, or an organic solvent. In some aspects, the base is sodium hydroxide. In some aspects, the aqueous solution is a sodium chloride solution. In some aspects, the sodium chloride solution is between 5 to 25% NaCl, between 10 to 30% NaCl, or between 15 to 25% NaCl. In some aspects, the alcohol is ethanol, 2-propanol, or isopropyl alcohol. In some aspects, the organic solvent is ethyl acetate or acetone. In some aspects, the method further comprises adding a base to the coupling increasing the pH to a basic pH. In some aspects, the base comprises between 2N to 12N of the base. In some aspects, the base is sodium hydroxide. In some aspects, the basic pH is between 10 to 12.

In various aspects, the present disclosure provides a method for producing a compound as described herein, the method comprising coupling R³ to a compound of Formula (S4):

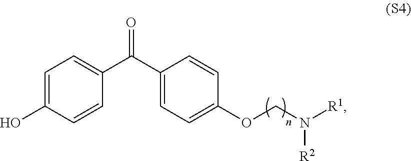

thereby producing the compound as described herein.

In some aspects, the coupling comprises alkylating the compound of Formula (S4):

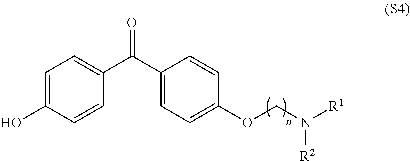

with R³.

In some aspects, the coupling comprises methylating the compound of Formula (S4):

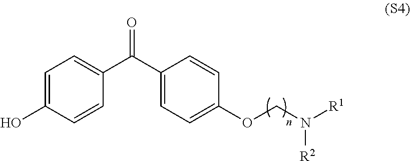

with a methylating agent.

In some aspects, the methylating agent is 1-chloroethyl chloroformate. In some aspects, the coupling comprises an organic solvent. In some aspects, the organic solvent comprises a solvent mixture. In some aspects, the solvent mixture comprises tetrahydrofuran and N-ethyldiisopropylamine. In some aspects, the method further comprises incubating the coupling under an inert atmosphere. In some aspects, the inert atmosphere is a N₂ atmosphere. In some aspects, the method further comprises heating the coupling to between 30 to 70° C., between 40 to 70° C., between 50 to 70° C., or between 55 to 65°. In some aspects, the method further comprises heating the coupling to an elevated temperature for between 8 to 16 hours, between 10 to 14 hours, or between 9 to 11 hours. In some aspects, the elevated temperature is a boiling point of the organic solvent. In some aspects, the method further comprising adding an alcohol to the coupling. In some aspects, the alcohol is methanol. In some aspects, the method further comprises heating the coupling to between 30 to 70° C., between 40 to 70° C., between 50 to 70° C., or between 55 to 65° C. and distilling the coupling to a reduced volume. In some aspects, the reduced volume to total volume ratio is 1:3, 1:5, or 1:10. In some aspects, the method further comprises adding an acid. In some aspects, the acid comprises 2N to 12N of the acid. In some aspects, the acid is hydrochloric acid. In some aspects, the method further comprises heating the coupling to between 30 to 70° C., between 40 to 70° C., between 50 to 70° C., or between 55 to 65° C. for between 2 to 20 hours, between 6 to 18 hours, or between 8 to 16 hours.

In some aspects, the method further comprises distilling, washing, purifying, or separating the compound of Formula (V) using an acid, a base, an aqueous solution, an alcohol, or an organic solvent. In some aspects, the aqueous solution comprises water. In some aspects, the base comprises 2N to 12N of the base. In some aspects, the base is sodium hydroxide. In some aspects, the organic solvent is ethyl acetate. In some aspects, the acid comprises 2N to 12N of the acid. In some aspects, the acid is hydrochloric acid.

In various aspects, the present disclosure provides a method for producing a compound as described herein, the method comprising coupling a compound of Formula (S5):

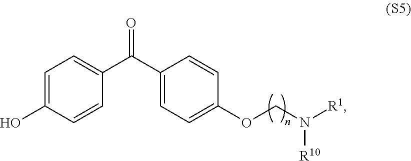

to a compound of Y²—C(═O)—Y³, wherein Y² and Y³ are each independently a leaving group, and a nucleophile, thereby producing the compound as described herein.

In some aspects, the leaving group is a halogen, —OR⁶, —SR⁶ or —NR⁶R⁷. In some aspects, the nucleophile is a C₁-C₆ alcohol, a C₁-C₆ thiol, a C₁-C₆ primary amine, or a C₁-C₆ secondary amine. In some aspects, the coupling comprises an organic solvent. In some aspects, the organic solvent comprises a solvent mixture. In some aspects, the solvent mixture comprises tetrahydrofuran and N-ethyldiisopropylamine. In some aspects, the method further comprises incubating the coupling under an inert atmosphere. In some aspects, the inert atmosphere is a N₂ atmosphere. In some aspects, the method comprises incubating the coupling at a temperature of at least 30° C. In some aspects, the method comprises incubating the coupling at a temperature of between 30 to 70° C., between 40 to 70° C., between 50 to 70° C., or between 55 to 65° C. In some aspects, the coupling comprises heating the compound of Formula (S5) for between 8 to 16 hours, between 10 to 14 hours, or between 9 to 11 hours. In some aspects, the method further comprising adding an alcohol to the coupling. In some aspects, the alcohol comprises methanol.

In some aspects, the method further comprises heating the coupling to between 30 to 70° C., between 40 to 70° C., between 50 to 70° C., or between 55 to 65° C. and distilling the coupling to a reduced volume. In some aspects, the reduced volume to total volume ratio is 1:3, 1:5, or 1:10. In some aspects, the method further comprises adding an acid. In some aspects, the acid comprises 2N to 12N of the acid. In some aspects, the acid comprises hydrochloric acid. In some aspects, the method further comprises heating the coupling to between 30 to 70° C., between 40 to 70° C., between 50 to 70° C., or between 55 to 65° C. for between 2 to 20 hours, between 6 to 18 hours, or between 8 to 16 hours.

In some aspects, the method further comprises distilling, washing, purifying, or separating the compound of Formula (VI) using an acid, a base, an aqueous solution, an alcohol, or an organic solvent. In some aspects, the aqueous solution comprises water. In some aspects, the base comprises 2N to 12N of the base. In some aspects, the base comprises sodium hydroxide. In some aspects, the organic solvent is ethyl acetate. In some aspects, the acid comprises 2N to 12N of the acid. In some aspects, the acid comprises hydrochloric acid.

In various aspects, the present disclosure provides a method for producing a compound as described herein, the method comprising coupling a compound of Formula (S6):

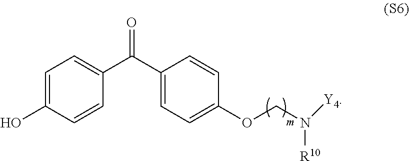

to a compound of Formula (VI):

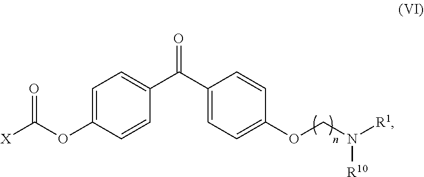

thereby producing a compound as described herein.

In some aspects, the coupling comprises an organic solvent. In some aspects, the organic solvent comprises a solvent mixture. In some aspects, the solvent mixture comprises tetrahydrofuran and N-ethyldiisopropylamine. In some aspects, the method further comprises incubating the coupling under an inert atmosphere. In some aspects, the inert atmosphere is a N₂ atmosphere. In some aspects, the method further comprises heating the coupling to between 30 to 70° C., between 40 to 70° C., between 50 to 70° C., or between 55 to 65° C. In some aspects, the method further comprises heating the coupling to an elevated temperature for between 8 to 16 hours, between 10 to 14 hours, or between 9 to 11 hours. In some aspects, the elevated temperature is a boiling point of the organic solvent. In some aspects, the method further comprises adding an alcohol to the coupling. In some aspects, the alcohol comprises methanol. In some aspects, the method further comprises heating the coupling to an elevated temperature and distilling the coupling to a reduced volume. In some aspects, the elevated temperature is between 30 to 70° C., between 40 to 70° C., between 50 to 70° C., or between 55 to 65° C. In some aspects, the reduced volume to total volume ratio is 1:3, 1:5, or 1:10. In some aspects, the method further comprises adding an acid. In some aspects, the acid comprises 2N to 12N of the acid. In some aspects, the acid comprises hydrochloric acid. In some aspects, the method further comprises heating the coupling to between 30 to 70° C., between 40 to 70° C., between 50 to 70° C., or between 55 to 65° C. for between 2 to 20 hours, between 6 to 18 hours, or between 8 to 16 hours.

In some aspects, the method further comprises distilling, washing, purifying, or separating the compound of Formula (VII) using an acid, a base, an aqueous solution, an alcohol, or an organic solvent. In some aspects, the aqueous solution comprises water. In some aspects, the base comprises 2N to 12N of the base. In some aspects, the base is sodium hydroxide. In some aspects, the organic solvent is ethyl acetate. In some aspects, the acid comprises 2N to 12N of the acid. In some aspects, the acid is hydrochloric acid.

In various aspects, the present disclosure provides a method for producing a compound as described herein, the method comprising coupling two compounds of Formula (S7):

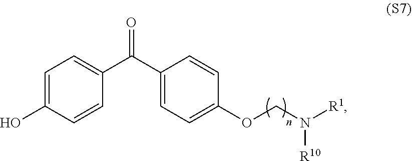

thereby producing the compound as described herein.

In some aspects, the coupling comprises an olefination. In some aspects, the coupling comprises a McMurry reaction. In some aspects, the method further comprises activating the compound of Formula (S7):

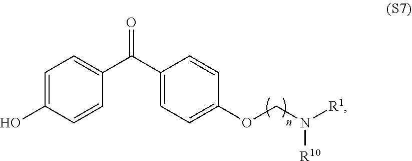

with a metal catalyst.

In some aspects, the method further comprises generating the metal catalyst by combining a metal salt with a reducing agent. In some aspects, the metal catalyst is titanium, zirconium, vanadium, niobium, molybdenum, tungsten, aluminum, or zinc. In some aspects, the metal catalyst is titanium. In some aspects, the metal catalyst is a titanium halide. In some aspects, the metal catalyst is TiCl₃ or TiCl₄. In some aspects, the reducing agent is zinc, zirconium, vanadium, niobium, molybdenum, tungsten, aluminum, magnesium, potassium, zinc-copper couple, alkali and alkali earth metals, butylium, lithium, or lithium aluminum hydride. In some aspects, the method further comprises a first solution made by combining the reducing agent and an organic solvent under an inert atmosphere at a first temperature. In some aspects, the organic solvent is THF. In some aspects, the inert atmosphere is a N₂ atmosphere. In some aspects, the first temperature is between 15° to 30° C. In some aspects, the method further comprises cooling the first solution to a second temperature and adding the metal catalyst. In some aspects, the second temperature is between −10° to 5° C. In some aspects, the method further comprises maintaining the first solution at a third temperature during the addition of the metal catalyst. In some aspects, the third temperature is less than 20° C. In some aspects, the method further comprises heating the first solution to a fourth temperature for 1 hour, 1.5 hours, 2 hours, 2.5 hours, or 3 hours. In some aspects, the fourth temperature is between 50° to 70° C.

In some aspects, the method further comprises a second solution made by adding the compound of Formula (S7) to an organic solvent. In some aspects, the organic solvent is THF. In some aspects, the method further comprises heating the second solution to between 50° to 70° C.

In some aspects, the method further comprises a third solution made by adding the second solution to the first solution over 1 hour, 1.5 hours, or 2 hours. In some aspects, the method further comprises stirring the third solution at between 50° to 70° C. for 7 hours, 8 hours, 9 hours, or 10 hours.

In some aspects, the method further comprises distilling, washing, filtering, separating, extracting, precipitating, collecting, or a combination thereof, the compound of Formula (VIII) with an organic solvent, an acid, or an aqueous solution. In some aspects, the distilling comprises an organic solvent. In some aspects, the organic solvent comprises MeTHF. In some aspects, the washing comprises an organic solvent, an acid, a base, a solvent mixture, or a combination thereof. In some aspects, the organic solvent is MeTHF, acetonitrile, acetone, or a combination thereof. In some aspects, the acid is between 2N to 12 N HCl. In some aspects, the aqueous solution is 30-35% HCl, between 10% to 60% NaCl, or between 10% to 60% K₂CO₃.

In various aspects, the present disclosure provides a method of generating an isomerically enriched mixture of a compound as described herein, the method comprising: providing a non-isomerically enriched mixture of the compound comprising a (Z) isomer and an (E) isomer of the compound, isomerizing the (E)-isomer of the compound into the (Z)-isomer of the compound, thereby generating an isomerically enriched mixture, and collecting the isomerically enriched mixture.

In some aspects, the isomerically enriched mixture comprises at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the (Z)-isomer of a compound as described herein. In some aspects, the collecting comprises precipitating the (Z)-isomer, distilling the (Z)-isomer, extracting the (Z)-isomer, chromatographically separating the (Z)-isomer, washing the (Z)-isomer, drying the (Z)-isomer, crystallizing the (Z)-isomer, or a combination thereof. In some aspects, the crystallizing is performed at a temperature of between −20° to 20° C. In some aspects, the non-isomerically enriched mixture is heated to a temperature of between 40° to 80° C. prior to the separating. In some aspects, the method comprises crystallizing the (Z)-isomer from an organic solvent. In some aspects, the organic solvent is an aprotic solvent. In some aspects, the organic solvent comprises ethyl acetate. In some aspects, the organic solvent comprises an acid. In some aspects, the organic solvent comprises between 2 to 12 N of the acid. In some aspects, the acid comprises HCl.

In various aspects, the present disclosure provides a method for producing a compound of Formula (IX), the method comprising coupling a compound of Formula (S8):

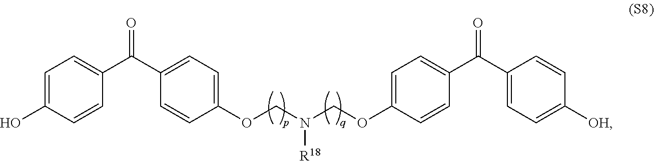

to compounds of Formulae (S9) and (S10):

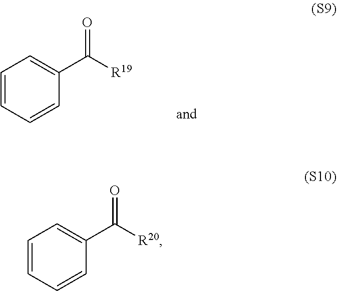

thereby producing the compound of Formula (IX).

In some aspects, the method further comprises activating the compounds of Formulae (S8), (S9), and (S10) with a metal catalyst. In some aspects, the method comprises generating the metal catalyst by combining a metal salt with a reducing agent. In some aspects, the metal catalyst comprises titanium, zirconium, vanadium, niobium, molybdenum, tungsten, aluminum, or zinc. In some aspects, the metal catalyst comprises titanium. In some aspects, the metal salt comprises a titanium halide. In some aspects, the titanium halide comprises TiCl₃ or TiCl₄. In some aspects, the reducing agent comprises zinc, zirconium, vanadium, niobium, molybdenum, tungsten, aluminum, magnesium, potassium, zinc-copper couple, alkali and alkali earth metals, butylium, lithium, or lithium aluminum hydride. In some aspects, the zinc comprises zinc powder.

In some aspects, the compounds of Formulae (S9) and (S10) are identical. In some aspects, the compound of Formula (S9) is Compound S4:

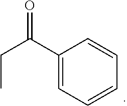

In some aspects, the compound of Formula (S10) is Compound S4:

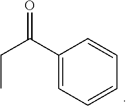

In some aspects, the coupling is performed in an organic solvent. In some aspects, the organic solvent is THF. In some aspects, the coupling is performed under reflux. In some aspects, the method further comprises adding an acid to the compounds of Formulae (S8), (S9), and S(10). In some aspects, the acid generates a biphasic mixture. In some aspects, the method further comprises heating the biphasic mixture to a temperature of between 3° and 70° C.

In some aspects, the coupling comprises an olefination reaction. In some aspects, the coupling comprises a McMurry reaction.

In some aspects, the method further comprises generating the compound of Formula (S8) by coupling compounds of Formulae (S11) and (S12):

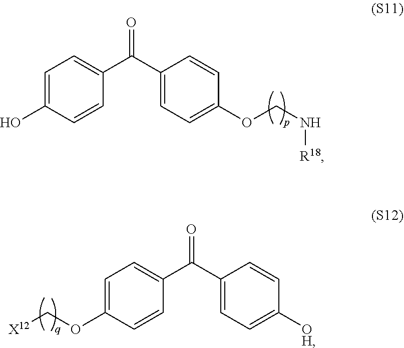

wherein X¹² is a halogen.

In some aspects, X¹² is Br. In some aspects, coupling the compound of Formulae (S11) and (S12) comprises an organic solvent. In some aspects, the organic solvent comprises a solvent mixture. In some aspects, the solvent mixture comprises ethanol and N-ethyldiisopropylamine. In some aspects, coupling the compound of Formulae (S11) and (S12) comprises reflux, vacuum distillation, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a UV chromatogram of Compound 1.

FIG. 2 illustrates a TIC chromatogram of Compound 1.

FIG. 3 illustrates a mass spectrum of Compound 1.

FIG. 4 illustrates a proton NMR spectrum of Compound 1.

FIG. 5 illustrates a carbon NMR spectrum of Compound 1.

FIG. 6 illustrates a proton NMR spectrum of Compound 2.

FIG. 7 illustrates a carbon NMR spectrum of Compound 2.

FIG. 8 illustrates a COSY NMR 2D spectrum of Compound 2.

FIG. 9 illustrates a UV chromatogram of Compound 2.

FIG. 10 illustrates a TIC chromatogram of Compound 2.

FIG. 11 illustrates a mass spectrum of Compound 2.

FIG. 12 illustrates a proton NMR spectrum of Compound 3.

FIG. 13 illustrates a UV chromatogram of Compound 3.

FIG. 14 illustrates a TIC chromatogram of Compound 3.

FIG. 15 illustrates a mass spectrum of Compound 3.

FIG. 16 illustrates a UV chromatogram of Compounds 4a and 4b.

FIG. 17 illustrates a TIC chromatogram of Compounds 4a and 4b.

FIG. 18 illustrates a mass spectrum of Compounds 4a and 4b.

FIG. 19 illustrates a UV chromatogram of Compound 7b.

FIG. 20 illustrates a TIC chromatogram of Compound 7b.

FIG. 21 illustrates a mass spectrum of Compounds 7a and 7b.

FIG. 22 illustrates a proton NMR spectrum of Compound 7b.

FIG. 23 illustrates a ROESY spectrum of Compound 7b.

FIG. 24 illustrates a proton NMR spectrum of Compound 7a.

FIG. 25 illustrates a ROESY spectrum of Compound 7a.

FIG. 26 illustrates a proton NMR spectrum of Compound 5b.

FIG. 27 illustrates a ROESY spectrum of Compound 5b.

FIG. 28 illustrates a proton NMR spectrum of Compounds 5a.

FIG. 29 illustrates a ROESY spectrum of Compounds 5a.

FIG. 30 illustrates a UV chromatogram of Compound 5b.

FIG. 31 illustrates a TIC chromatogram of Compound 5b.

FIG. 32 illustrates a mass spectrum of Compound 5b.

FIG. 33 illustrates an HPLC chromatogram of Compounds 6a and 6b and a reference standard of Compounds 6a and 6b.

FIG. 34 is a plot that summarizes crystal violet assay results on MCF7 cells with endoxifen, Compound 7b, Compound 7a, Compound 5b, Compound 5a, and Compound 3.

FIG. 35 is a plot that summarizes crystal violet assay results MCF7 cells with D538G ESR2 mutations with endoxifen, Compound 7b, Compound 7a, Compound 5b, Compound 5a, and Compound 3.

FIG. 36 is a plot that summarizes crystal violet assay results on MCF7 cells with Y537S ESR2 mutations with endoxifen, Compound 7b, Compound 7a, Compound 5b, Compound 5a, and Compound 3.

FIG. 37 is a plot that summarizes crystal violet assay results on T47D cells with endoxifen, Compound 7b, Compound 7a, Compound 5b, Compound 5a, and Compound 3.

FIG. 38 is a plot that summarizes crystal violet assay results on T47D cells with Y537S ESR2 mutations with endoxifen, Compound 7b, Compound 7a, Compound 5b, Compound 5a, and Compound 3.

FIG. 39 is a plot that summarizes crystal violet assay results on T47D cells with D538G ESR2 mutations with endoxifen, Compound 7b, Compound 7a, Compound 5b, Compound 5a, and Compound 3.

FIG. 40 is a plot of activities of Compound 5a to endoxifen on the cell line MCF7.

FIG. 41 is a plot of activities of Compound 5a to endoxifen on T47D cells.

FIG. 42 is a plot of activities of Compound 5a to endoxifen on MCF7 cells with a D538G ESR1 mutation.

FIG. 43 is a plot of activities of Compound 5a to endoxifen on MCF7 cells with a Y537S ESR1 mutation.

FIG. 44 is a plot of activities of endoxifen to Compound 7b in T47D cells with a Y537S mutation in ESR1.

FIG. 45 is a plot of activities of endoxifen to Compound 7b in T47D cells with a D538G mutation in ESR1.

FIG. 46 is a set of plots of crystal violet assay results with endoxifen, Compound 7b, Compound 7a, Compound 5b, Compound 5a, and Compound 3 on parental MCF7 cell lines.

FIG. 47 is a plot of HPLC results on a blank control.

FIG. 48 is a plot of HPLC results on a sample containing Compound 7b.

FIG. 49 is a plot of HPLC results on a sample containing Compound 5b.

FIG. 50 is a plot of HPLC results on a sample containing Compound 5a.

FIGS. 51A-E are chromatography and mass spectrometry results on a sample containing Compound 3. FIG. 51A provides chromatography results on the sample in variable wavelength detection mode. FIG. 51B provides chromatography results on the sample in total ion count mode. FIG. 51C provides chromatography/mass spectrometry results on the sample at a retention time of 5.983 minutes. FIG. 51D provides chromatography results on the sample. FIG. 51E provides chromatography/mass spectrometry results on the sample at a retention time of 5.668-5.751 minutes.

FIGS. 52A-C are plots and images from scratch-wound assays performed in the presence of 0-6 μM endoxifen. FIG. 52A is a plot of relative wound density as a function of endoxifen concentration and time. FIG. 52B is a plot of wound width as a function of endoxifen concentration and time. FIG. 52C is a series of wound images collected on samples treated with varying concentrations of endoxifen.

FIGS. 53A-C are plots and images from scratch-wound assays performed in the presence of 0-6 μM Compound 5b. FIG. 53A is a plot of relative wound density as a function of Compound 5b concentration and time. FIG. 53B is a plot of wound width as a function of Compound 5b concentration and time. FIG. 53C is a series of wound images collected on samples treated with varying concentrations of Compound 5b.

FIGS. 54A-C are plots and images from scratch-wound assays performed in the presence of 0-6 μM Compound 5a. FIG. 54A is a plot of relative wound density as a function of Compound 5a concentration and time. FIG. 54B is a plot of wound width as a function of Compound 5a concentration and time. FIG. 54C is a series of wound images collected on samples treated with varying concentrations of Compound 5a.

FIGS. 55A-C are plots and images from scratch-wound assays performed in the presence of 0-6 μM Compound 7a. FIG. 55A is a plot of relative wound density as a function of Compound 7a concentration and time. FIG. 55B is a plot of wound width as a function of Compound 7a concentration and time. FIG. 55C is a series of wound images collected on samples treated with varying concentrations of Compound 7a.

FIGS. 56A-C are plots and images from scratch-wound assays performed in the presence of 0-6 μM Compound 7b. FIG. 56A is a plot of relative wound density as a function of Compound 7b concentration and time. FIG. 56B is a plot of wound width as a function of Compound 7b concentration and time. FIG. 56C is a series of wound images collected on samples treated with varying concentrations of Compound 7b.

FIG. 57 is a plot of relative wound density over time (0 to 100 hours) for wounds treated with vehicle (DMSO), 5 μM endoxifen, 5 μM Compound 5b, or 5 μM Compound 5a.

FIG. 58 is a plot of relative wound density over time (0 to 100 hours) for wounds treated with vehicle (DMSO), 5 μM endoxifen, 5 μM Compound 7a, or 5 μM Compound 7b.

FIG. 59 is a plot of relative wound density over time (0 to 100 hours) for wounds treated with vehicle (DMSO), 2.5 μM endoxifen, 2.5 μM Compound 5b, or 2.5 μM Compound 5a.

FIG. 60 is a plot of relative wound density over time (0 to 100 hours) for wounds treated with vehicle (DMSO), 2.5 μM endoxifen, 2.5 μM Compound 7a, or 2.5 μM Compound 7b.

FIG. 61 is a plot of relative wound density over time (0 to 100 hours) for wounds treated with vehicle (DMSO), 1.25 μM endoxifen, 1.25 μM Compound 5b, and 1.25 μM Compound 5a.

FIG. 62 is a plot of relative wound density over time (0 to 100 hours) for wounds treated with vehicle (DMSO), 1.25 μM endoxifen, 1.25 μM Compound 7a, and 1.25 μM Compound 7b.

FIGS. 63A-G are plots of absolute cell counts for cells in different cell cycle phases (G1 through S; G1; and S, G2, and M) following treatment with vehicle (DMSO), endoxifen, or an endoxifen-related compound. FIG. 63A is a plot of absolute cell counts for cells in different cell cycle phases following treatment with a vehicle. FIG. 63B is a plot of absolute cell counts for cells in different cell cycle phases following treatment with a 1.25 μM endoxifen. FIG. 63C is a plot of absolute cell counts for cells in different cell cycle phases following treatment with 1.25 μM Compound 7b. FIG. 63D is a plot of absolute cell counts for cells in different cell cycle phases following treatment with 1.25 μM Compound 7a. FIG. 63E is a plot of absolute cell counts for cells in different cell cycle phases following treatment with 1.25 μM Compound 5b.

FIG. 63F is a plot of absolute cell counts for cells in different cell cycle phases following treatment with 1.25 μM Compound 5a. FIG. 63G is a plot of absolute cell counts for cells in different cell cycle phases following treatment with 1.25 μM Compound 3.

FIGS. 64A-G are plots of percent cell counts for cells in different cell cycle phases (G1 through S; G1; and S, G2, and M) following treatment with vehicle (DMSO), endoxifen, or an endoxifen-related compound. FIG. 64A is a plot of percent cell counts for cells in different cell cycle phases following treatment with a vehicle. FIG. 64B is a plot of percent cell counts for cells in different cell cycle phases following treatment with a 1.25 μM endoxifen. FIG. 64C is a plot of percent cell counts for cells in different cell cycle phases following treatment with 1.25 μM Compound 7b. FIG. 64D is a plot of percent cell counts for cells in different cell cycle phases following treatment with 1.25 μM Compound 7a. FIG. 64E is a plot of percent cell counts for cells in different cell cycle phases following treatment with 1.25 μM Compound 5b. FIG. 64F is a plot of percent cell counts for cells in different cell cycle phases following treatment with 1.25 μM Compound 5a. FIG. 64G is a plot of percent cell counts for cells in different cell cycle phases following treatment with 1.25 μM Compound 3.

FIGS. 65A-G are plots of absolute cell counts for cells in different cell cycle phases (G1 through S; G1; and S, G2, and M) following treatment with vehicle (DMSO), endoxifen, or an endoxifen-related compound. FIG. 65A is a plot of absolute cell counts for cells in different cell cycle phases following treatment with a vehicle. FIG. 65B is a plot of absolute cell counts for cells in different cell cycle phases following treatment with a 2.5 μM endoxifen. FIG. 65C is a plot of absolute cell counts for cells in different cell cycle phases following treatment with 2.5 μM Compound 7b. FIG. 65D is a plot of absolute cell counts for cells in different cell cycle phases following treatment with 2.5 μM Compound 7a. FIG. 65E is a plot of absolute cell counts for cells in different cell cycle phases following treatment with 2.5 μM Compound 5b. FIG. 65F is a plot of absolute cell counts for cells in different cell cycle phases following treatment with 2.5 μM Compound 5a. FIG. 65G is a plot of absolute cell counts for cells in different cell cycle phases following treatment with 2.5 μM Compound 3.

FIGS. 66A-G are plots of percent cell counts for cells in different cell cycle phases (G1 through S; G1; and S, G2, and M) following treatment with vehicle (DMSO), endoxifen, or an endoxifen-related compound. FIG. 66A is a plot of percent cell counts for cells in different cell cycle phases following treatment with a vehicle. FIG. 66B is a plot of percent cell counts for cells in different cell cycle phases following treatment with a 2.5 μM endoxifen. FIG. 66C is a plot of percent cell counts for cells in different cell cycle phases following treatment with 2.5 μM Compound 7b. FIG. 66D is a plot of percent cell counts for cells in different cell cycle phases following treatment with 2.5 μM Compound 7a. FIG. 66E is a plot of percent cell counts for cells in different cell cycle phases following treatment with 2.5 μM Compound 5b. FIG. 66F is a plot of percent cell counts for cells in different cell cycle phases following treatment with 2.5 μM Compound 5a. FIG. 66G is a plot of percent cell counts for cells in different cell cycle phases following treatment with 2.5 μM Compound 3.

FIGS. 67A-G are plots of absolute cell counts for cells in different cell cycle phases (G1 through S; G1; and S, G2, and M) following treatment with vehicle (DMSO), endoxifen, or an endoxifen-related compound. FIG. 67A is a plot of absolute cell counts for cells in different cell cycle phases following treatment with a vehicle. FIG. 67B is a plot of absolute cell counts for cells in different cell cycle phases following treatment with a 5 μM endoxifen. FIG. 67C is a plot of absolute cell counts for cells in different cell cycle phases following treatment with 5 μM Compound 7b. FIG. 67D is a plot of absolute cell counts for cells in different cell cycle phases following treatment with 5 μM Compound 7a. FIG. 67E is a plot of absolute cell counts for cells in different cell cycle phases following treatment with 5 μM Compound 5b. FIG. 67F is a plot of absolute cell counts for cells in different cell cycle phases following treatment with 5 μM Compound 5a. FIG. 67G is a plot of absolute cell counts for cells in different cell cycle phases following treatment with 5 μM Compound 3.

FIGS. 68A-G are plots of percent cell counts for cells in different cell cycle phases (G1 through S; G1; and S, G2, and M) following treatment with vehicle (DMSO), endoxifen, or an endoxifen-related compound. FIG. 68A is a plot of percent cell counts for cells in different cell cycle phases following treatment with a vehicle. FIG. 68B is a plot of percent cell counts for cells in different cell cycle phases following treatment with a 5 μM endoxifen. FIG. 68C is a plot of percent cell counts for cells in different cell cycle phases following treatment with 5 μM Compound 7b. FIG. 68D is a plot of percent cell counts for cells in different cell cycle phases following treatment with 5 μM Compound 7a. FIG. 68E is a plot of percent cell counts for cells in different cell cycle phases following treatment with 5 μM Compound 5b. FIG. 68F is a plot of percent cell counts for cells in different cell cycle phases following treatment with 5 μM Compound 5a. FIG. 68G is a plot of percent cell counts for cells in different cell cycle phases following treatment with 5 μM Compound 3.

FIGS. 68A-G are plots of percent cell counts for cells in different cell cycle phases (G1 through S; G1; and S, G2, and M) following treatment with vehicle (DMSO), endoxifen, or an endoxifen-related compound. FIG. 68A is a plot of percent cell counts for cells in different cell cycle phases following treatment with a vehicle. FIG. 68B is a plot of percent cell counts for cells in different cell cycle phases following treatment with a 5 μM endoxifen. FIG. 68C is a plot of percent cell counts for cells in different cell cycle phases following treatment with 5 μM Compound 7b. FIG. 68D is a plot of percent cell counts for cells in different cell cycle phases following treatment with 5 μM Compound 7a. FIG. 68E is a plot of percent cell counts for cells in different cell cycle phases following treatment with 5 μM Compound 5b. FIG. 68F is a plot of percent cell counts for cells in different cell cycle phases following treatment with 5 μM Compound 5a. FIG. 68G is a plot of percent cell counts for cells in different cell cycle phases following treatment with 5 μM Compound 3.

FIGS. 69A-G are plots of percent cell counts for cells in different cell cycle phases (G1 through S; G1; and S, G2, and M) following treatment with vehicle (DMSO), endoxifen, or an endoxifen-related compound. FIG. 69A is a plot of percent cell counts for cells in different cell cycle phases following treatment with a vehicle. FIG. 69B is a plot of percent cell counts for cells in different cell cycle phases following treatment with a 1.25 μM endoxifen. FIG. 69C is a plot of percent cell counts for cells in different cell cycle phases following treatment with 1.25 μM Compound 7b. FIG. 69D is a plot of percent cell counts for cells in different cell cycle phases following treatment with 1.25 μM Compound 7a. FIG. 69E is a plot of percent cell counts for cells in different cell cycle phases following treatment with 1.25 μM Compound 5b. FIG. 69F is a plot of percent cell counts for cells in different cell cycle phases following treatment with 1.25 μM Compound 5a. FIG. 69G is a plot of percent cell counts for cells in different cell cycle phases following treatment with 1.25 μM Compound 3.

FIGS. 70A-G are plots of percent cell counts for cells in different cell cycle phases (G1 through S; G1; and S, G2, and M) following treatment with vehicle (DMSO), endoxifen, or an endoxifen-related compound. FIG. 70A is a plot of percent cell counts for cells in different cell cycle phases following treatment with a vehicle. FIG. 70B is a plot of percent cell counts for cells in different cell cycle phases following treatment with a 2.5 μM endoxifen. FIG. 70C is a plot of percent cell counts for cells in different cell cycle phases following treatment with 2.5 μM Compound 7b. FIG. 70D is a plot of percent cell counts for cells in different cell cycle phases following treatment with 2.5 μM Compound 7a. FIG. 70E is a plot of percent cell counts for cells in different cell cycle phases following treatment with 2.5 μM Compound 5b. FIG. 70F is a plot of percent cell counts for cells in different cell cycle phases following treatment with 2.5 μM Compound 5a. FIG. 70G is a plot of percent cell counts for cells in different cell cycle phases following treatment with 2.5 μM Compound 3.

FIGS. 71A-G are plots of percent cell counts for cells in different cell cycle phases (G1 through S; G1; and S, G2, and M) following treatment with vehicle (DMSO), endoxifen, or an endoxifen-related compound. FIG. 71A is a plot of percent cell counts for cells in different cell cycle phases following treatment with a vehicle. FIG. 71B is a plot of percent cell counts for cells in different cell cycle phases following treatment with a 5 μM endoxifen. FIG. 71C is a plot of percent cell counts for cells in different cell cycle phases following treatment with 5 μM Compound 7b. FIG. 71D is a plot of percent cell counts for cells in different cell cycle phases following treatment with 5 μM Compound 7a. FIG. 71E is a plot of percent cell counts for cells in different cell cycle phases following treatment with 5 μM Compound 5b. FIG. 71F is a plot of percent cell counts for cells in different cell cycle phases following treatment with 5 μM Compound 5a. FIG. 71G is a plot of percent cell counts for cells in different cell cycle phases following treatment with 5 μM Compound 3.

FIGS. 72A-F are plots of results from cell viability assays on ER+ T47D cells. FIG. 72A is a plot of cell viability assay results on cells treated with endoxifen. FIG. 72B is a plot of cell viability assay results on cells treated with Compound 7b. FIG. 72C is a plot of cell viability assay results on cells treated with Compound 7a. FIG. 72D is a plot of cell viability assay results on cells treated with Compound 5b. FIG. 72E is a plot of cell viability assay results on cells treated with Compound 5a. FIG. 72F is a plot of cell viability assay results on cells treated with Compound 3.

FIGS. 73A-F are plots of results from cell viability assays on ER+ T47D cells. FIG. 73A is a plot of cell viability assay results on cells treated with endoxifen. FIG. 73B is a plot of cell viability assay results on cells treated with Compound 7b. FIG. 73C is a plot of cell viability assay results on cells treated with Compound 7a. FIG. 73D is a plot of cell viability assay results on cells treated with Compound 5b. FIG. 73E is a plot of cell viability assay results on cells treated with Compound 5a. FIG. 73F is a plot of cell viability assay results on cells treated with Compound 3.

FIGS. 74A-F are plots of results from cell viability assays on ER− T47D cells. FIG. 74A is a plot of cell viability assay results on cells treated with endoxifen. FIG. 74B is a plot of cell viability assay results on cells treated with Compound 7b. FIG. 74C is a plot of cell viability assay results on cells treated with Compound 7a. FIG. 74D is a plot of cell viability assay results on cells treated with Compound 5b. FIG. 74E is a plot of cell viability assay results on cells treated with Compound 5a. FIG. 74F is a plot of cell viability assay results on cells treated with Compound 3.

FIG. 75 is a series of images of masked and unmasked wounds from the cell migration assays, with the rightmost figure showing invadopodia in cells within wound sites.

FIGS. 76A-C are plots and images from scratch-wound assays performed on MCF7 samples with varying concentrations of endoxifen. FIG. 76A is a plot of relative wound density as a function of endoxifen concentration and time. FIG. 76B is a plot of wound width as a function of endoxifen concentration and time. FIG. 76C is a series of wound images collected on samples treated with varying concentrations of endoxifen.

FIGS. 77A-C are plots and images from scratch-wound assays performed on T47D samples with varying concentrations of endoxifen. FIG. 77A is a plot of relative wound density as a function of endoxifen concentration and time. FIG. 77B is a plot of wound width as a function of endoxifen concentration and time. FIG. 77C is a series of wound images collected on samples treated with varying concentrations of endoxifen.

FIGS. 78A-C are plots and images from scratch-wound assays performed on MCF7 samples with varying concentrations of Compound 5b. FIG. 78A is a plot of relative wound density as a function of Compound 5b concentration and time. FIG. 78B is a plot of wound width as a function of Compound 5b concentration and time. FIG. 78C is a series of wound images collected on samples treated with varying concentrations of Compound 5b.

FIGS. 79A-C are plots and images from scratch-wound assays performed on T47D samples with varying concentrations of Compound 5b. FIG. 79A is a plot of relative wound density as a function of Compound 5b concentration and time. FIG. 79B is a plot of wound width as a function of Compound 5b concentration and time. FIG. 79C is a series of wound images collected on samples treated with varying concentrations of Compound 5b.

FIGS. 80A-C are plots and images from scratch-wound assays performed on MCF7 samples with varying concentrations of Compound 5a. FIG. 80A is a plot of relative wound density as a function of Compound 5a concentration and time. FIG. 80B is a plot of wound width as a function of Compound 5a concentration and time. FIG. 80C is a series of wound images collected on samples treated with varying concentrations of Compound 5a.

FIGS. 81A-C are plots and images from scratch-wound assays performed on T47D samples with varying concentrations of Compound 5a. FIG. 81A is a plot of relative wound density as a function of Compound 5a concentration and time. FIG. 81B is a plot of wound width as a function of Compound 5a concentration and time. FIG. 81C is a series of wound images collected on samples treated with varying concentrations of Compound 5a.

FIGS. 82A-C are plots and images from scratch-wound assays performed on MCF7 samples with varying concentrations of Compound 7b. FIG. 82A is a plot of relative wound density as a function of Compound 7b concentration and time. FIG. 82B is a plot of wound width as a function of Compound 7b concentration and time. FIG. 82C is a series of wound images collected on samples treated with varying concentrations of Compound 7b.

FIGS. 83A-C are plots and images from scratch-wound assays performed on T47D samples with varying concentrations of Compound 7b. FIG. 83A is a plot of relative wound density as a function of Compound 7b concentration and time. FIG. 83B is a plot of wound width as a function of Compound 7b concentration and time. FIG. 83C is a series of wound images collected on samples treated with varying concentrations of Compound 7b.

FIGS. 84A-C are plots and images from scratch-wound assays performed on MCF7 samples with varying concentrations of Compound 7a. FIG. 84A is a plot of relative wound density as a function of Compound 7a concentration and time. FIG. 84B is a plot of wound width as a function of Compound 7a concentration and time. FIG. 84C is a series of wound images collected on samples treated with varying concentrations of Compound 7a.

FIGS. 85A-C are plots and images from scratch-wound assays performed on T47D samples with varying concentrations of Compound 7a. FIG. 85A is a plot of relative wound density as a function of Compound 7a concentration and time. FIG. 85B is a plot of wound width as a function of Compound 7a concentration and time. FIG. 85C is a series of wound images collected on samples treated with varying concentrations of Compound 7a.

FIG. 86 is a series of images from scratch wound assays following treatment with vehicle, endoxifen, and Compounds 5a and 5b.

FIGS. 87A-I are plots of wound density and wound width values from scratch wound treatment assays on MCF7 samples. FIG. 87A is a plot of wound densities of samples treated with vehicle, 1.25 μM endoxifen, 1.25 μM Compound 5a, or 1.25 μM Compound 5b. FIG. 87B is a plot of wound densities of samples treated with vehicle, 2.5 μM endoxifen, 2.5 μM Compound 5a, or 2.5 μM Compound 5b. FIG. 87C is a plot of wound densities of samples treated with vehicle, 5 μM endoxifen, 5 μM Compound 5a, or 5 μM Compound 5b. FIG. 87D is a plot of wound widths of samples treated with vehicle, 1.25 μM endoxifen, 1.25 μM Compound 5a, or 1.25 μM Compound 5b. FIG. 87E is a plot of wound widths of samples treated with vehicle, 2.5 μM endoxifen, 2.5 μM Compound 5a, or 2.5 μM Compound 5b. FIG. 87F is a plot of wound widths of samples treated with vehicle, 5 μM endoxifen, 5 μM Compound 5a, or 5 μM Compound 5b. FIG. 87G is a plot of wound width changes in samples treated with vehicle, 1.25 μM endoxifen, 1.25 μM Compound 5a, or 1.25 μM Compound 5b. FIG. 87H is a plot of wound width changes of samples treated with vehicle, 2.5 μM endoxifen, 2.5 μM Compound 5a, or 2.5 μM Compound 5b. FIG. 87I is a plot of wound width changes in samples treated with vehicle, 5 μM endoxifen, 5 μM Compound 5a, or 5 μM Compound 5b.

FIG. 88 is a series of images from scratch wound assays following treatment with vehicle, endoxifen, and Compounds 7a and 7b.

FIGS. 89A-I are plots of wound density and wound width values from scratch wound treatment assays on MCF7 samples. FIG. 89A is a plot of wound densities of samples treated with vehicle, 1.25 μM endoxifen, 1.25 μM Compound 7a, or 1.25 μM Compound 7b. FIG. 89B is a plot of wound densities of samples treated with vehicle, 2.5 μM endoxifen, 2.5 μM Compound 7a, or 2.5 μM Compound 7b. FIG. 89C is a plot of wound densities of samples treated with vehicle, 5 μM endoxifen, 5 μM Compound 7a, or 5 μM Compound 7b. FIG. 89D is a plot of wound widths of samples treated with vehicle, 1.25 μM endoxifen, 1.25 μM Compound 7a, or 1.25 μM Compound 7b. FIG. 89E is a plot of wound widths of samples treated with vehicle, 2.5 μM endoxifen, 2.5 μM Compound 7a, or 2.5 μM Compound 7b. FIG. 89F is a plot of wound widths of samples treated with vehicle, 5 μM endoxifen, 5 μM Compound 7a, or 5 μM Compound 7b. FIG. 89G is a plot of wound width changes in samples treated with vehicle, 1.25 μM endoxifen, 1.25 μM Compound 7a, or 1.25 μM Compound 7b. FIG. 89H is a plot of wound width changes of samples treated with vehicle, 2.5 μM endoxifen, 2.5 μM Compound 7a, or 2.5 μM Compound 7b. FIG. 89I is a plot of wound width changes in samples treated with vehicle, 5 μM endoxifen, 5 μM Compound 7a, or 5 μM Compound 7b.

FIGS. 90A-F are plots that summarize results of proliferation assays for multiple cancer cell lines in the presence of varying doses of endoxifen and multiple endoxifen-related compounds. FIG. 90A is a plot of relative proliferation values for MCF7 samples treated with endoxifen or an endoxifen-related compound. FIG. 90B is a plot of relative proliferation values for T47D samples treated with endoxifen or an endoxifen-related compound. FIG. 90C is a plot of relative proliferation values for E2_UCD4 samples treated with endoxifen or an endoxifen-related compound. FIG. 90D is a plot of relative proliferation values for E2_MCF7 samples treated with endoxifen or an endoxifen-related compound. FIG. 90E is a plot of proliferation values for E2_T47D samples treated with endoxifen or an endoxifen-related compound. FIG. 90F is a plot of E2_UCD12 samples treated with endoxifen or an endoxifen-related compound.

FIGS. 91A-C are plots that summarize results of wound confluence measurements from scratch wound assays on T47D samples. FIG. 91A is a plot of wound confluence measurements from scratch wound assays on T47D samples treated with 1.25 μM endoxifen or an endoxifen-related compound. FIG. 91B is a plot of wound confluence measurements from scratch wound assays on T47D samples treated with 2.5 μM endoxifen or an endoxifen-related compound. FIG. 91C is a plot of wound confluence measurements from scratch wound assays on T47D samples treated with 5 μM endoxifen or an endoxifen-related compound.

FIGS. 92A-C are plots that summarize results of wound density measurements from scratch wound assays on T47D samples. FIG. 92A is a plot of wound density measurements from scratch wound assays on T47D samples treated with 1.25 μM endoxifen or an endoxifen-related compound. FIG. 92B is a plot of wound density measurements from scratch wound assays on T47D samples treated with 2.5 μM endoxifen or an endoxifen-related compound. FIG. 92C is a plot of wound density measurements from scratch wound assays on T47D samples treated with 5 μM endoxifen or an endoxifen-related compound.

FIGS. 93A-C are plots that summarize results of wound confluence measurements from scratch wound assays on MCF7 samples. FIG. 93A is a plot of wound confluence measurements from scratch wound assays on MCF7 samples treated with 1.25 μM endoxifen or an endoxifen-related compound. FIG. 93B is a plot of wound confluence measurements from scratch wound assays on MCF7 samples treated with 2.5 μM endoxifen or an endoxifen-related compound. FIG. 93C is a plot of wound confluence measurements from scratch wound assays on MCF7 samples treated with 5 μM endoxifen or an endoxifen-related compound.

FIGS. 94A-C are plots that summarize results of wound density measurements from scratch wound assays on MCF7 samples. FIG. 94A is a plot of wound density measurements from scratch wound assays on MCF7 samples treated with 1.25 μM endoxifen or an endoxifen-related compound. FIG. 94B is a plot of wound density measurements from scratch wound assays on MCF7 samples treated with 2.5 μM endoxifen or an endoxifen-related compound. FIG. 94C is a plot of wound density measurements from scratch wound assays on MCF7 samples treated with 5 μM endoxifen or an endoxifen-related compound.

FIGS. 95A-F are plots that summarize results of caspase-3/7 apoptosis assays on MCF7 and T47D cells in the presence of varying concentrations of endoxifen and multiple endoxifen-related compounds as a function of time. FIG. 95A is a plot that summarizes results of caspase-3/7 apoptosis assays on T47D cells in the presence of vehicle or 1.25 μM endoxifen or an endoxifen-related compound. FIG. 95B is a plot that summarizes results of caspase-3/7 apoptosis assays on T47D cells in the presence of vehicle or 2.5 μM endoxifen or an endoxifen-related compound. FIG. 95C is a plot that summarizes results of caspase-3/7 apoptosis assays on T47D cells in the presence of vehicle or 5 μM endoxifen or an endoxifen-related compound. FIG. 95D is a plot that summarizes results of caspase-3/7 apoptosis assays on MCF7 cells in the presence of vehicle or 1.25 μM endoxifen or an endoxifen-related compound. FIG. 95E is a plot that summarizes results of caspase-3/7 apoptosis assays on MCF7 cells in the presence of vehicle or 2.5 μM endoxifen or an endoxifen-related compound. FIG. 95F is a plot that summarizes results of caspase-3/7 apoptosis assays on MCF7 cells in the presence of vehicle or 5 μM endoxifen or an endoxifen-related compound.

FIGS. 96A-F are plots that summarize averaged results of caspase-3/7 apoptosis assays on MCF7 and T47D cells in the presence of varying concentrations of endoxifen and multiple endoxifen-related compounds. FIG. 96A is a plot that summarizes averaged results of caspase-3/7 apoptosis assays on T47D cells in the presence of vehicle or 1.25 μM endoxifen or an endoxifen-related compound. FIG. 96B is a plot that summarizes averaged results of caspase-3/7 apoptosis assays on T47D cells in the presence of vehicle or 2.5 μM endoxifen or an endoxifen-related compound. FIG. 96C is a plot that summarizes averaged results of caspase-3/7 apoptosis assays on T47D cells in the presence of vehicle or 5 μM endoxifen or an endoxifen-related compound. FIG. 96D is a plot that summarizes averaged results of caspase-3/7 apoptosis assays on MCF7 cells in the presence of vehicle or 1.25 μM endoxifen or an endoxifen-related compound. FIG. 96E is a plot that summarizes averaged results of caspase-3/7 apoptosis assays on MCF7 cells in the presence of vehicle or 2.5 μM endoxifen or an endoxifen-related compound. FIG. 96F is a plot that summarizes averaged results of caspase-3/7 apoptosis assays on MCF7 cells in the presence of vehicle or 5 μM endoxifen or an endoxifen-related compound.

FIGS. 97A-D are chromatograms of Compound S15. FIGS. 97A-B are HPLC and LC-MS chromatograms of a first Compound S15 sample. FIGS. 97C-D are HPLC and LC-MS chromatograms of a second Compound S15 sample.

FIGS. 98A-B are HPLC and LC-MS chromatograms of a Compounds 9a, 9b, and 9c, sample.

FIGS. 99A-B are HPLC and LC-MS chromatograms of a Compound S15 sample.

FIG. 100 is a chromatogram of a sample with multiple Compounds 9a, 9b, and 9c isomers.

### DETAILED DESCRIPTION

Described herein are active pharmaceutical compounds and compositions thereof that may have anti-tumor activity and provide treatments for diseases, such as cancer. Also described are synthetic methods of making the pharmaceutical compounds described herein and methods of making the pharmaceutical compositions as described herein. The compounds or compositions described herein may exhibit aromatase inhibition activity or estrogen receptor activity. The compounds and compositions described herein may be useful in the treatment of a condition in a subject (e.g., the treatment of cancer in a subject).

### Chemical Formulae

The chemical compounds of the present disclosure may be represented as generic chemical structures and formulas (e.g., Formula (I)-Formula (XIII)).

### Compounds of Formula (I)

Aspects of the present disclosure provide a compound of Formula (I):

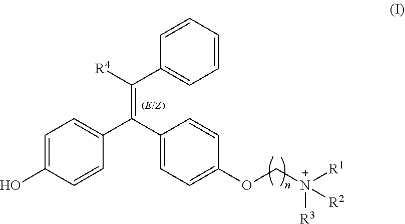

or a pharmaceutically acceptable salt, solvate, or tautomer thereof, wherein:
R¹, R², R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
R⁴ is C₁-C₆ alkyl substituted with 0 to 3 instances of R⁸, C₂-C₆ alkenyl substituted with 0 to 3 instances of R⁸, or C₂-C₆ alkynyl substituted with 0 to 3 instances of R⁸;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl;
each instance of R⁸ is independently halogen, hydroxyl, C₁-C₃ alkoxy, C₁-C₃ alkylthio, C₁-C₃ alkyl, or C₃-C₈ cycloalkyl; and
   n is an integer from 1 to 10.

In some embodiments, the compound of Formula (I) is a compound of Formula (Ia). Formula (Ia):

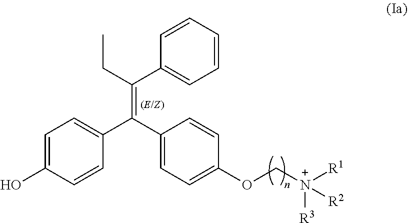

or a pharmaceutically acceptable salt, solvate, or tautomer thereof.

In some embodiments, the compound of Formula (I) is a compound of Formula (Ib):

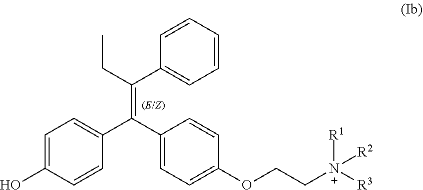

or a pharmaceutically acceptable salt, solvate, or tautomer thereof.

In some embodiments, the compound of Formula (I) is a compound of Formula (II):

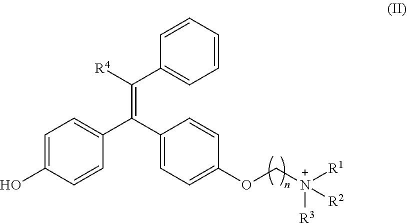

or a pharmaceutically acceptable salt, solvate, or tautomer thereof.

In some embodiments, the compound of Formula (I) is a compound of Formula (IIa):

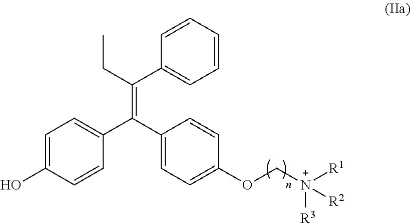

or a pharmaceutically acceptable salt, solvate, or tautomer thereof.

In some embodiments, the compound of Formula (I) is a compound of Formula (IIb):

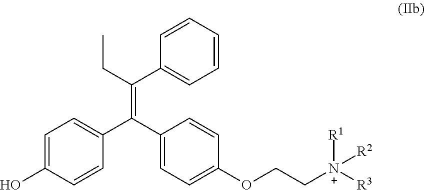

or a pharmaceutically acceptable salt, solvate, or tautomer thereof.

In some embodiments, the compound is a (Z)-isomer of the compound of Formula (I). In some embodiments, the compound is a (E)-isomer of the compound of Formula (I). In some embodiments, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (I) is a compound of Formula (II):

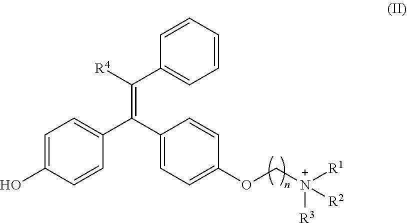

or a pharmaceutically acceptable salt, solvate, or tautomer thereof.

In some embodiments, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (I) is a compound of Formula (IIa):

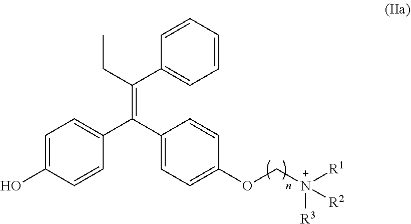

or a pharmaceutically acceptable salt, solvate, or tautomer thereof.

In some embodiments, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (I) is a compound of Formula (IIb):

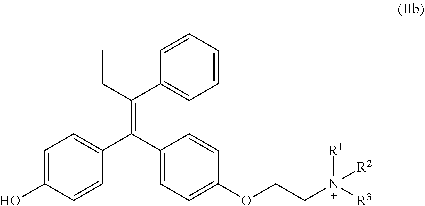

or a pharmaceutically acceptable salt, solvate, or tautomer thereof.

A compound of Formula (I) may have a variety of substituents as defined by the R groups and the value of “n” to define the length of a carbon chain. In some embodiments, R¹, R², and R³ are each independently C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some embodiments, R¹, R², and R³ are each independently C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹, R², and R³ are each independently C₁-C₆ alkyl substituted with 0 to 3 instances of R⁵. In some embodiments, R¹, R², and R³ are each independently C₁-C₆ alkyl substituted with 0 or 1 instance of R⁵. In some embodiments, R¹, R², and R³ are each independently C₁-C₃ alkyl substituted with 0 to 3 instances of R⁵. In some embodiments, R¹, R², and R³ are each independently C₁-C₃ alkyl substituted with 0 or 1 instance of R⁵. In some embodiments, R¹, R², and R³ are each independently C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹, R², and R³ are each independently C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some embodiments, R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹, R², and R³ are each independently C₁-C₆ alkyl. In some embodiments, R¹, R², and R³ are each independently C₁-C₃ alkyl. In some embodiments, R¹, R², and R³ are each —CH₃. In some embodiments, R¹, R², and R³ are each —CH₃. In some embodiments, R¹ is —CH₃. In some embodiments, R² is —CH₃. In some embodiments, R³ is —CH₃. In some embodiments, R⁴ is C₁-C₆ alkyl substituted with 0 to 3 instances of R⁸. In some embodiments, R⁴ is C₁-C₆ alkyl substituted with 0 or 1 instance of R⁸. In some embodiments, R⁴ is C₁-C₃ alkyl substituted with 0 to 3 instances of R⁸. In some embodiments, R⁴ is C₁-C₃ alkyl substituted with 0 or 1 instance of R⁸. In some embodiments, R⁴ is C₁-C₃ alkyl. In some embodiments, R⁴ is —CH₂CH₃. In some embodiments, each instance of R⁸ is independently hydroxyl, C₁-C₃ alkoxy, C₁-C₃ alkyl, or C₃-C₈ cycloalkyl. In some embodiments, each instance of R⁸ is independently hydroxyl, C₁-C₃ alkoxy, or C₁-C₃ alkyl. In some embodiments, each instance of R⁸ is independently hydroxyl or C₁-C₃ alkyl. In some embodiments, each instance of R⁸ is independently C₁-C₃ alkyl. In some embodiments, each instance of R⁵ is independently halogen, hydroxyl, C₁-C₃ alkoxy, C₁-C₃ alkylthio, or C₁-C₃ alkyl. In some embodiments, each instance of R⁵ is independently hydroxyl, C₁-C₃ alkoxy, or C₁-C₃ alkyl. In some embodiments, each instance of R⁵ is independently hydroxyl or C₁-C₃ alkyl. In some embodiments, each instance of R⁵ is hydroxyl. In some embodiments, each instance of R⁵ is independently C₁-C₃ alkoxy. In some embodiments, each instance of R⁵ is independently C₁-C₃ alkyl. In some embodiments, each instance of R⁶ and R⁷ is independently hydrogen or C₁-C₃ alkyl. In some embodiments, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some embodiments, each instance of R⁶ and R⁷ is independently hydrogen or methyl. In some embodiments, each instance of R⁶ and R⁷ is independently C₁-C₃ alkyl. In some embodiments, each instance of R⁶ and R⁷ is hydrogen. In some embodiments n is 1 to 2. In some embodiments, n is 2 to 4. In some embodiments, n is 2 or 3. In some embodiments, n is 2.

A compound of Formula (I) may be a cation or a polycation. In some embodiments, the compound of Formula (I) is a cation due to a positive charge on a nitrogen. A compound of Formula (I) may be an anion or a polyanion. In some embodiments, the compound of Formula (I) is an anion due to a negative charge on an oxygen (e.g., a hydroxyl). A compound of Formula (I) may be a zwitterion. In some embodiments, the compound of Formula (I) is a zwitterion due to a positive charge on a nitrogen and a negative charge on an oxygen.

A “salt” of a compound of Formula (I) may be derived from inorganic or organic acids, bases, or a combination thereof. In some cases, a salt includes a cationic form of Formula (I) and an anion. Suitable anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. In some cases, a salt includes an anionic form of Formula (I) and a cation. Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

### Compounds of Formula (III)

Aspects of the present disclosure provide a compound of Formula (III)

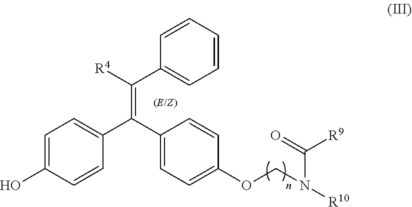

or a pharmaceutically acceptable salt, solvate, or tautomer thereof, wherein:
   R⁹ is hydrogen, hydroxyl, C₁-C₆ alkyl substituted with 0 to 3 instances of R¹¹, C₂-C₆ alkenyl substituted with 0 to 3 instances of R¹¹, or C₂-C₆ alkynyl substituted with 0 to 3 instances of R¹¹;
   R¹⁰ is hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   R⁴ is C₁-C₆ alkyl substituted with 0 to 3 instances of R¹, C₂-C₆ alkenyl substituted with 0 to 3 instances of R⁸, or C₂-C₆ alkynyl substituted with 0 to 3 instances of R⁸;
   each instance of R¹¹ is independently halogen, hydroxyl, C₁-C₃ alkoxy, C₁-C₃ alkylthio, C₁-C₃ alkyl, or C₃-C₈ cycloalkyl, aryl, or —C(═O)OCH₃;
   each instance of R⁵ is independently halogen, hydroxyl, C₁-C₃ alkoxy, C₁-C₃ alkylthio, C₁-C₃ alkyl, —C(═O)OH, or —C(═O)OCH₃;
   each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl;
   each instance of R⁸ is independently halogen, hydroxyl, C₁-C₃ alkoxy, C₁-C₃ alkylthio, C₁-C₃ alkyl, or C₃-C₈ cycloalkyl; and
   n is an integer from 1 to 10.

In some embodiments, the compound of Formula (III) is a compound of Formula (IIIa):

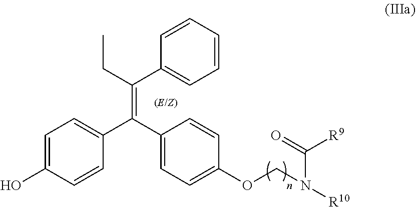

or a pharmaceutically acceptable salt, solvate, or tautomer thereof.

In some embodiments, the compound of Formula (III) is a compound of Formula (IUIb):

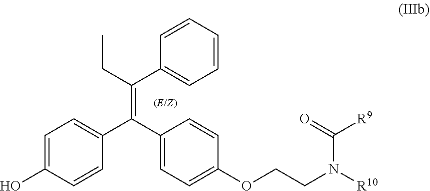

or a pharmaceutically acceptable salt, solvate, or tautomer thereof.

In some embodiments, the compound of Formula (III) is a compound of Formula (IV):

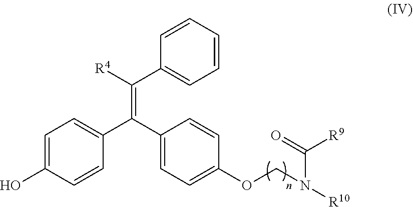

or a pharmaceutically acceptable salt, solvate, or tautomer thereof.

In some embodiments, the compound of Formula (III) is a compound of Formula (IVa):

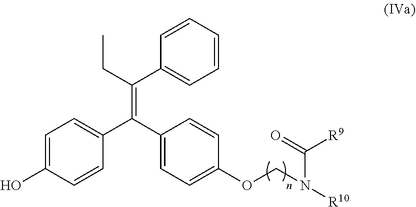

or a pharmaceutically acceptable salt, solvate, or tautomer thereof.

In some embodiments, the compound of Formula (III) is a compound of Formula (IVb):

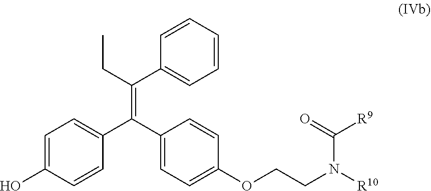

or a pharmaceutically acceptable salt, solvate, or tautomer thereof.

In some embodiments, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (III) is a compound of Formula (IV):

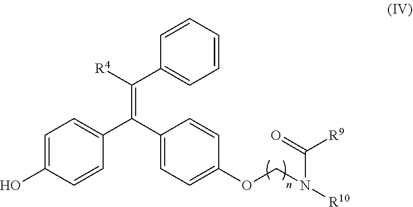

or a pharmaceutically acceptable salt, solvate, or tautomer thereof.

In some embodiments, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (IIIa) is a compound of Formula (IVa):

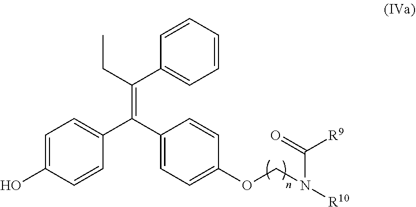

or a pharmaceutically acceptable salt, solvate, or tautomer thereof.

In some embodiments, at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the compound of Formula (IIIb) is a compound of Formula (IVb):

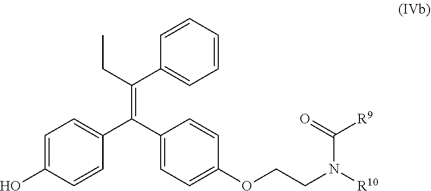

or a pharmaceutically acceptable salt, solvate, or tautomer thereof.

A compound of Formula (III) may have a variety of substituents as defined by the R groups and the value of “n” to define the length of a carbon chain. In some embodiments, R⁴ is C₁-C₆ alkyl substituted with 0 to 3 instances of R⁸. In some embodiments, R⁴ is C₁-C₆ alkyl substituted with 0 or 1 instance of R⁸. In some embodiments, R⁴ is C₁-C₃ alkyl substituted with 0 to 3 instances of R⁸. In some embodiments, R⁴ is C₁-C₃ alkyl substituted with 0 or 1 instance of R¹. In some embodiments, R⁴ is C₁-C₃ alkyl. In some embodiments, R⁴ is —CH₂CH₃. In some embodiments, each instance of R⁸ is independently hydroxyl, C₁-C₃ alkoxy, C₁-C₃ alkyl, or C₃-C₈ cycloalkyl. In some embodiments, each instance of R⁸ is independently hydroxyl, C₁-C₃ alkoxy, or C₁-C₃ alkyl. In some embodiments, each instance of R⁸ is independently hydroxyl or C₁-C₃ alkyl. In some embodiments, each instance of R⁸ is independently C₁-C₃ alkyl. In some embodiments, R⁹ is hydrogen, hydroxyl, C₁-C₆ alkyl substituted with 0 or 1 instance of R¹¹, C₂-C₆alkenyl substituted with 0 or 1 instance of R¹¹, or C₂-C₆ alkynyl substituted with 0 or 1 instance of R¹¹. In some embodiments, R⁹ is hydrogen, hydroxyl, C₁-C₃ alkyl substituted with 0 to 3 instances of R¹¹, C₂-C₃ alkenyl substituted with 0 to 3 instances of R¹¹, or C₂-C₃ alkynyl substituted with 0 to 3 instances of R¹¹. In some embodiments, R⁹ is hydrogen, hydroxyl, C₁-C₃ alkyl substituted with 0 or 1 instance of R¹¹, C₂-C₃ alkenyl substituted with 0 or 1 instance of R¹¹, or C₂-C₃ alkynyl substituted with 0 or 1 instance of R¹¹. In some embodiments, R⁹ is hydrogen, hydroxyl, or C₁-C₆ alkyl substituted with 0 to 3 instances of R¹¹. In some embodiments, R⁹ is C₁-C₆ alkyl substituted with 0 to 3 instances of R¹¹. In some embodiments, R⁹ is hydrogen, hydroxyl, or C₁-C₆ alkyl substituted with 0 or 1 instance of R¹¹. In some embodiments, R⁹ is C₁-C₆ alkyl substituted with 0 or 1 instance of R¹¹. In some embodiments, R⁹ is hydrogen, hydroxyl, or C₁-C₆ alkyl. In some embodiments, R⁹ is C₁-C₆ alkyl. In some embodiments, R⁹ is hydrogen, hydroxyl, or C₁-C₃ alkyl substituted with 0 to 3 instances of R¹¹. In some embodiments, R⁹ is C₁-C₃ alkyl substituted with 0 to 3 instances of R¹¹. In some embodiments, R⁹ is hydrogen, hydroxyl, or C₁-C₃ alkyl substituted with 0 or 1 instance of R¹¹. In some embodiments, R⁹ is C₁-C₃ alkyl substituted with 0 or 1 instance of R¹¹. In some embodiments, R⁹ is hydrogen, hydroxyl, or C₁-C₃ alkyl. In some embodiments, R⁹ is C₁-C₃ alkyl. In some embodiments, R⁹ is C₁-C₃ alkyl. In some embodiments, R⁹ is methyl. In some embodiments, each instance of R¹¹ is independently halogen, hydroxyl, C₁-C₃ alkoxy, C₁-C₃ alkylthio, or C₁-C₃ alkyl. In some embodiments, each instance of R¹¹ is independently halogen, hydroxyl, C₁-C₃ alkylthio, or C₁-C₃ alkyl. In some embodiments, each instance of R¹¹ is independently hydroxyl, C₁-C₃ alkoxy, or C₁-C₃ alkyl. In some embodiments, each instance of R¹¹ is independently hydroxyl or C₁-C₃ alkyl. In some embodiments, each instance of R¹¹ is hydroxyl. In some embodiments, each instance of R¹¹ is independently C₁-C₃ alkyl. In some embodiments, R¹⁰ is hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OR⁶, —C(═O)NR⁶R⁷, or —C(═O)SR⁶. In some embodiments, R¹⁰ is hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OH, —C(═O)NH₂, or —C(═O)SH. In some embodiments, R¹⁰ is hydrogen, C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹⁰ is hydrogen or C₁-C₆ alkyl substituted with 0 to 3 instances of R⁵. In some embodiments, R¹⁰ is hydrogen or C₁-C₃ alkyl substituted with 0 to 3 instances of R⁵. In some embodiments, R¹⁰ is hydrogen or C₁-C₆ alkyl substituted with 0 or 1 instance of R⁵. In some embodiments, R¹⁰ is hydrogen or C₁-C₃ alkyl substituted with 0 or 1 instance of R⁵. In some embodiments, R¹⁰ is hydrogen or C₁-C₃ alkyl. In some embodiments, R¹⁰ is hydrogen or methyl. In some embodiments, R¹⁰ is hydrogen. In some embodiments, R¹⁰ is methyl. In some embodiments, each instance of R⁵ is independently halogen, hydroxyl, C₁-C₃ alkoxy, C₁-C₃ alkylthio, or C₁-C₃ alkyl. In some embodiments, each instance of R⁵ is independently hydroxyl, C₁-C₃ alkoxy, or C₁-C₃ alkyl. In some embodiments, each instance of R⁵ is independently hydroxyl or C₁-C₃ alkyl. In some embodiments, each instance of R⁵ is hydroxyl. In some embodiments, each instance of R⁵ is independently C₁-C₃ alkoxy. In some embodiments, each instance of R⁵ is independently C₁-C₃ alkyl. In some embodiments, each instance of R⁶ and R⁷ is independently hydrogen or C₁-C₃ alkyl. In some embodiments, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some embodiments, each instance of R⁶ and R⁷ is independently hydrogen or methyl. In some embodiments, each instance of R⁶ and R⁷ is independently C₁-C₃ alkyl. In some embodiments, each instance of R⁶ and R⁷ is hydrogen. In some embodiments n is 1 to 2. In some embodiments, n is 2 to 4. In some embodiments, n is 2 or 3. In some embodiments, n is 2.

A compound of Formula (III) may be a cation or a polycation. In some embodiments, the compound of Formula (III) is a cation due to a positive charge on a nitrogen. A compound of Formula (III) may be an anion or a polyanion. In some embodiments, the compound of Formula (III) is an anion due to a negative charge on an oxygen. A compound of Formula (III) may be a zwitterion. In some embodiments, the compound of Formula (III) is a zwitterion due to a positive charge on a nitrogen and a negative charge on an oxygen.

A “salt” of a compound of Formula (III) may be derived from inorganic or organic acids, bases, or a combination thereof. In some cases, a salt includes a cationic form of Formula (III) and an anion. Suitable anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. In some cases, a salt includes an anionic form of Formula (III) and a cation. Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

### Compounds of Formula (V)

Aspects of the present disclosure provide a compound of Formula (V)

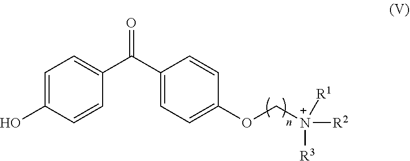

or a pharmaceutically acceptable salt, solvate, or tautomer thereof,
wherein:
R¹, R², R³ are each independently C₁-C₆ alkyl substituted with 0 to 3 instances of R⁵, C₂-C₆alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
n is an integer from 1 to 10.

In some embodiments, the compound of Formula (V) is a compound of Formula (Va):

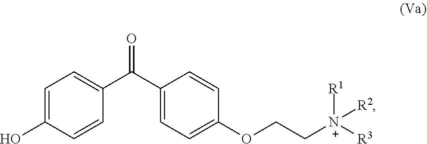

or a pharmaceutically acceptable salt, solvate, or tautomer thereof.

In some embodiments, the compound of Formula (V) is a compound of Formula (Vb):

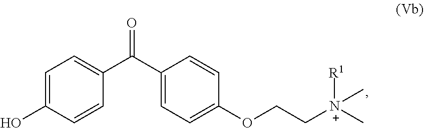

or a pharmaceutically acceptable salt, solvate, or tautomer thereof.

A compound of Formula (V) may have a variety of substituents as defined by the R groups and the value of “n” to define the length of a carbon chain. In some embodiments, R¹, R², and R³ are each independently C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some embodiments, R¹, R², and R³ are each independently C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹, R², and R³ are each independently C₁-C₆ alkyl substituted with 0 to 3 instances of R⁵. In some embodiments, R¹, R², and R³ are each independently C₁-C₆ alkyl substituted with 0 or 1 instance of R⁵. In some embodiments, R¹, R², and R³ are each independently C₁-C₃ alkyl substituted with 0 to 3 instances of R⁵. In some embodiments, R¹, R², and R³ are each independently C₁-C₃ alkyl substituted with 0 or 1 instance of R⁵. In some embodiments, R¹, R², and R³ are each independently C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹, R², and R³ are each independently C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some embodiments, R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹, R², and R³ are each independently C₁-C₆ alkyl. In some embodiments, R¹, R², and R³ are each independently C₁-C₃ alkyl. In some embodiments, R¹, R², and R³ are each —CH₃. In some embodiments, R¹, R², and R³ are each —CH₃. In some embodiments, R¹ is —CH₃. In some embodiments, R² is —CH₃. In some embodiments, R³ is —CH₃. In some embodiments, each instance of R⁵ is independently halogen, hydroxyl, C₁-C₃ alkoxy, C₁-C₃ alkylthio, or C₁-C₃ alkyl. In some embodiments, each instance of R⁵ is independently hydroxyl, C₁-C₃ alkoxy, or C₁-C₃ alkyl. In some embodiments, each instance of R⁵ is independently hydroxyl or C₁-C₃ alkyl. In some embodiments, each instance of R⁵ is hydroxyl. In some embodiments, each instance of R⁵ is independently C₁-C₃ alkoxy. In some embodiments, each instance of R⁵ is independently C₁-C₃ alkyl. In some embodiments, each instance of R⁶ and R⁷ is independently hydrogen or C₁-C₃ alkyl. In some embodiments, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some embodiments, each instance of R⁶ and R⁷ is independently hydrogen or methyl. In some embodiments, each instance of R⁶ and R⁷ is independently C₁-C₃ alkyl. In some embodiments, each instance of R⁶ and R⁷ is hydrogen. In some embodiments n is 1 to 2. In some embodiments, n is 2 to 4. In some embodiments, n is 2 or 3. In some embodiments, n is 2.

A compound of Formula (V) may be a cation or a polycation. In some embodiments, the compound of Formula (V) is a cation due to a positive charge on a nitrogen. A compound of Formula (V) may be an anion or a polyanion. In some embodiments, the compound of Formula (V) is an anion due to a negative charge on an oxygen. A compound of Formula (V) may be a zwitterion. In some embodiments, the compound of Formula (V) is a zwitterion due to a positive charge on a nitrogen and a negative charge on an oxygen.

A “salt” of a compound of Formula (V) may be derived from inorganic or organic acids, bases, or a combination thereof. In some cases, a salt includes a cationic form of Formula (V) and an anion. Suitable anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. In some cases, a salt includes an anionic form of Formula (V) and a cation. Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

### Compounds of Formula (VI)

Aspects of the present disclosure provide a compound of Formula (VI):

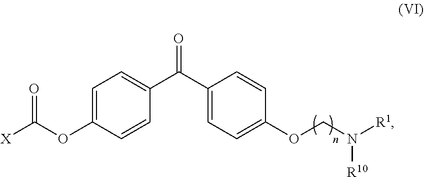

or a pharmaceutically acceptable salt, solvate, or tautomer thereof, wherein:
R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵,
—C(═O)OR⁶, —C(═O)NR⁶R⁷, or —C(═O)SR⁶;
R¹⁰ is hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl;
X is C₁-C₆ alkyl, —OR¹², —SR¹² or —NR¹²R¹³;
R¹² and R¹³ are each independently hydrogen or C₁-C₃ alkyl substituted with 0 or 1 instance of halogen; and
n is an integer from 1 to 10.

Aspects of the present disclosure provide a compound of Formula (VIa):

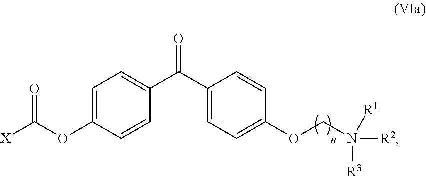

or a pharmaceutically acceptable salt, solvate, or tautomer thereof, wherein:
R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R¹ are each independently hydrogen or C₁-C₃ alkyl, or an instance of R⁶ and an instance of R⁷ on the same nitrogen are taken together with the nitrogen to form C₂-C₇ heterocycloalkyl;
X is C₁-C₆ alkyl, —OR¹², —SR¹² or —NR¹²R¹³;
R¹² and R¹³ are each independently hydrogen or C₁-C₃ alkyl substituted with 0 or 1 instance of halogen; and
n is an integer from 1 to 10.

In some embodiments, the compound is a compound of Formula (VI). In some embodiments, the compound is a compound of Formula (VIa).

In some embodiments, the compound of Formula (VI) is a compound of Formula (VIb):

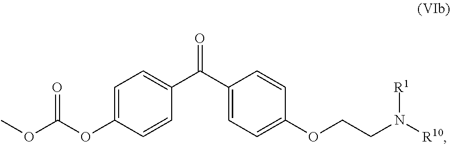

or a pharmaceutically acceptable salt, solvate, or tautomer thereof.

In some embodiments, the compound of Formula (VIa) is a compound of Formula (VIc):

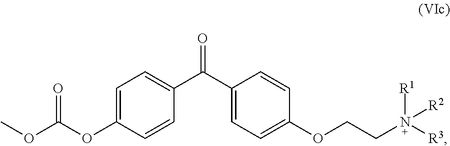

or a pharmaceutically acceptable salt, solvate, or tautomer thereof.

A compound of Formula (VI) may have a variety of substituents as defined by the R groups and the value of “n” to define the length of a carbon chain. In some embodiments, Riis C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some embodiments, R¹ is C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹ is C₁-C₆ alkyl substituted with 0 to 3 instances of R⁵. In some embodiments, R¹ is C₁-C₆ alkyl substituted with 0 or 1 instance of R⁵. In some embodiments, R¹ is C₁-C₃ alkyl substituted with 0 to 3 instances of R⁵. In some embodiments, R¹ is C₁-C₃ alkyl substituted with 0 or 1 instance of R⁵. In some embodiments, R¹ is C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹ is C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some embodiments, R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹ is C₁-C₆ alkyl. In some embodiments, R¹ is C₁-C₃ alkyl. In some embodiments, R¹ is —CH₃. In some embodiments, R¹, R², and R³ are each independently C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some embodiments, R¹, R², and R³ are each independently C₁-C₆alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹, R², and R³ are each independently C₁-C₆ alkyl substituted with 0 to 3 instances of R⁵. In some embodiments, R¹, R², and R³ are each independently C₁-C₆ alkyl substituted with 0 or 1 instance of R⁵. In some embodiments, R¹, R², and R³ are each independently C₁-C₃ alkyl substituted with 0 to 3 instances of R⁵. In some embodiments, R¹, R², and R³ are each independently C₁-C₃ alkyl substituted with 0 or 1 instance of R⁵. In some embodiments, R¹, R², and R³ are each independently C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹, R², and R³ are each independently C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some embodiments, R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹, R², and R³ are each independently C₁-C₆ alkyl. In some embodiments, R¹, R², and R³ are each independently C₁-C₃ alkyl. In some embodiments, R¹, R², and R³ are each —CH₃. In some embodiments, R¹, R², and R³ are each —CH₃. In some embodiments, R¹ is —CH₃. In some embodiments, R² is —CH₃. In some embodiments, R³ is —CH₃. In some embodiments, R¹⁰ is hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OR⁶, —C(═O)NR⁶R⁷, or —C(═O)SR⁶. In some embodiments, R¹⁰ is hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OH, —C(═O)NH₂, or —C(═O)SH. In some embodiments, R¹⁰ is hydrogen, C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹⁰ is hydrogen or C₁-C₆ alkyl substituted with 0 to 3 instances of R⁵. In some embodiments, R¹⁰ is hydrogen or C₁-C₃ alkyl substituted with 0 to 3 instances of R⁵. In some embodiments, R¹⁰ is hydrogen or C₁-C₆ alkyl substituted with 0 or 1 instance of R⁵. In some embodiments, R¹⁰ is hydrogen or C₁-C₃ alkyl substituted with 0 or 1 instance of R⁵. In some embodiments, R¹⁰ is hydrogen or C₁-C₃ alkyl. In some embodiments, R¹⁰ is hydrogen or methyl. In some embodiments, R¹⁰ is hydrogen. In some embodiments, R¹⁰ is methyl. In some embodiments, each instance of R⁵ is independently halogen, hydroxyl, C₁-C₃ alkoxy, C₁-C₃ alkylthio, or C₁-C₃ alkyl. In some embodiments, each instance of R⁵ is independently hydroxyl, C₁-C₃ alkoxy, or C₁-C₃ alkyl. In some embodiments, each instance of R⁵ is independently hydroxyl or C₁-C₃ alkyl. In some embodiments, each instance of R⁵ is hydroxyl. In some embodiments, each instance of R⁵ is independently C₁-C₃ alkoxy. In some embodiments, each instance of R⁵ is independently C₁-C₃ alkyl. In some embodiments, each instance of R⁶ and R⁷ is independently hydrogen or C₁-C₃ alkyl. In some embodiments, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some embodiments, each instance of R⁶ and R⁷ is independently hydrogen or methyl. In some embodiments, each instance of R⁶ and R⁷ is independently C₁-C₃ alkyl. In some embodiments, each instance of R⁶ and R⁷ is hydrogen. In some embodiments n is 1 to 2. In some embodiments, n is 2 to 4. In some embodiments, n is 2 or 3. In some embodiments, n is 2.

Compounds of Formula (VI) or (VIa) may be a cation or a polycation. In some embodiments, the compound of Formula (VI) is a cation due to a positive charge on a nitrogen. A compound of Formula (VI) may be an anion or a polyanion. In some embodiments, the compound of Formula (VI) is an anion due to a negative charge on an oxygen. A compound of Formula (VI) may be a zwitterion. In some embodiments, the compound of Formula (VI) is a zwitterion due to a positive charge on a nitrogen and a negative charge on an oxygen.

A “salt” of compounds of Formula (VI) or (VIa) may be derived from inorganic or organic acids, bases, or a combination thereof. In some cases, a salt includes a cationic form of Formula (VI) or (VIa) and an anion. Suitable anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. In some cases, a salt includes an anionic form of Formula (VI) or (VIa) and a cation. Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

### Compounds of Formula (VII)

Aspects of the present disclosure provide a compound of Formula (VII)

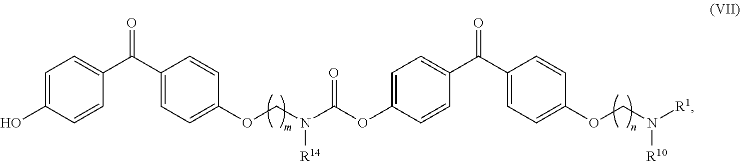

or a pharmaceutically acceptable salt, solvate, or tautomer thereof, wherein:
R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵,
—C(═O)OR⁶, —C(═O)NR⁶R⁷, or —C(═O)SR⁶;
R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
m and n are each independently integers from 1 to 10.

Aspects of the present disclosure provide a compound of Formula (VIIa):

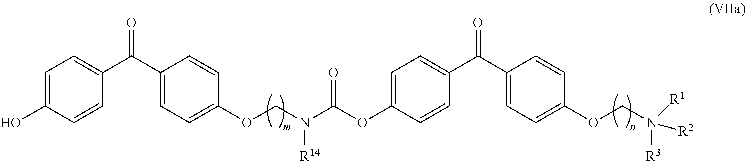

or a pharmaceutically acceptable salt, solvate, or tautomer thereof, wherein:
R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
m and n are each independently integers from 1 to 10.

In some embodiments, the compound is a compound of Formula (VII). In some embodiments, the compound is a compound of Formula (VIIa).

In some embodiments, the compound of Formula (VII) is a compound of Formula (VIIb):

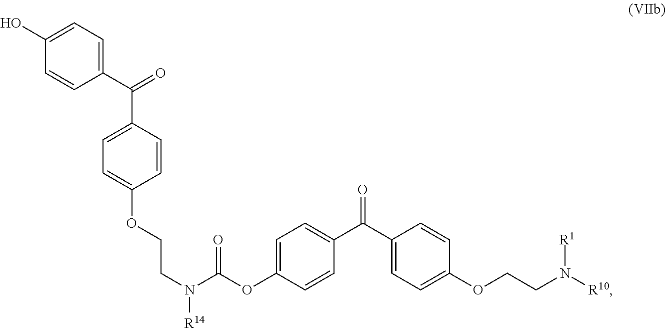

or a pharmaceutically acceptable salt, solvate, or tautomer thereof.

In some embodiments, the compound of Formula (VIIa) is a compound of Formula (VIIc):

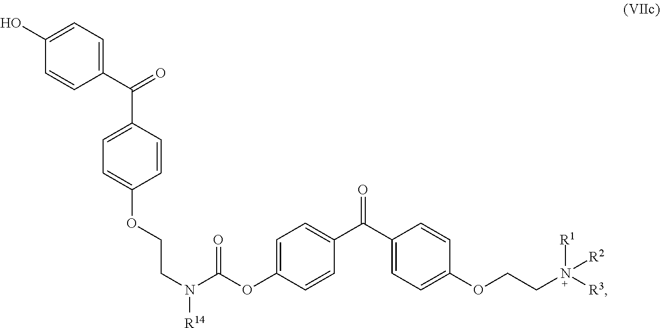

or a pharmaceutically acceptable salt, solvate, or tautomer thereof.

A compound of Formula (VII) may have a variety of substituents as defined by the R groups and the value of “n” or “m” to define the length of a carbon chain. In some embodiments, Riis C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some embodiments, R¹ is C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹ is C₁-C₆ alkyl substituted with 0 to 3 instances of R⁵. In some embodiments, R¹ is C₁-C₆ alkyl substituted with 0 or 1 instance of R⁵. In some embodiments, R¹ is C₁-C₃ alkyl substituted with 0 to 3 instances of R⁵. In some embodiments, R¹ is C₁-C₃ alkyl substituted with 0 or 1 instance of R⁵. In some embodiments, R¹ is C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹ is C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some embodiments, R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹ is C₁-C₆ alkyl. In some embodiments, R¹ is C₁-C₃ alkyl. In some embodiments, R¹ is —CH₃. In some embodiments, R¹, R², and R³ are each independently C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some embodiments, R¹, R², and R³ are each independently C₁-C₆alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹, R², and R³ are each independently C₁-C₆ alkyl substituted with 0 to 3 instances of R⁵. In some embodiments, R¹, R², and R³ are each independently C₁-C₆ alkyl substituted with 0 or 1 instance of R⁵. In some embodiments, R¹, R², and R³ are each independently C₁-C₃ alkyl substituted with 0 to 3 instances of R⁵. In some embodiments, R¹, R², and R³ are each independently C₁-C₃ alkyl substituted with 0 or 1 instance of R⁵. In some embodiments, R¹, R², and R³ are each independently C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹, R², and R³ are each independently C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some embodiments, R¹, R², and R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹, R², and R³ are each independently C₁-C₆ alkyl. In some embodiments, R¹, R², and R³ are each independently C₁-C₃ alkyl. In some embodiments, R¹, R², and R³ are each —CH₃. In some embodiments, R¹, R², and R³ are each —CH₃. In some embodiments, R¹ is —CH₃. In some embodiments, R² is —CH₃. In some embodiments, R³ is —CH₃. In some embodiments, R¹⁰ is hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OR⁶, —C(═O)NR⁶R⁷, or —C(═O)SR⁶. In some embodiments, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OR⁶, —C(═O)NR⁶R⁷, or —C(═O)SR⁶. In some embodiments, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OH, —C(═O)NH₂, or —C(═O)SH. In some embodiments, R¹⁰ and R¹⁴ are each independently hydrogen, C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹⁰ and R¹⁴ are each independently hydrogen or C₁-C₆ alkyl substituted with 0 to 3 instances of R⁵. In some embodiments, R¹⁰ and R¹⁴ are each independently hydrogen or C₁-C₃ alkyl substituted with 0 to 3 instances of R⁵. In some embodiments, R¹⁰ and R¹⁴ are each independently hydrogen or C₁-C₆ alkyl substituted with 0 or 1 instance of R⁵. In some embodiments, R¹⁰ and R¹⁴ are each independently hydrogen or C₁-C₃ alkyl substituted with 0 or 1 instance of R⁵. In some embodiments, R¹⁰ and R¹⁴ are each independently hydrogen or C₁-C₃ alkyl. In some embodiments, R¹⁰ and R¹⁴ are each independently hydrogen or methyl. In some embodiments, R¹⁰ and R¹⁴ are each independently hydrogen. In some embodiments, R¹⁰ and R¹⁴ are each independently methyl. In some embodiments, R¹⁰ is hydrogen. In some embodiments, R¹⁰ is methyl. In some embodiments, R¹⁴ is hydrogen. In some embodiments, R¹⁴ is methyl. In some embodiments, R¹⁴ is hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OH, —C(═O)NH₂, or —C(═O)SH. In some embodiments, R¹⁴ is hydrogen, C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹⁴ is hydrogen or C₁-C₆ alkyl substituted with 0 to 3 instances of R⁵. In some embodiments, R¹⁴ is hydrogen or C₁-C₃ alkyl substituted with 0 to 3 instances of R⁵. In some embodiments, R¹⁴ is hydrogen or C₁-C₆ alkyl substituted with 0 or 1 instance of R⁵. In some embodiments, R¹⁴ is hydrogen or C₁-C₃ alkyl substituted with 0 or 1 instance of R⁵. In some embodiments, R¹⁴ is hydrogen or C₁-C₃ alkyl. In some embodiments, R¹⁴ is hydrogen or methyl. In some embodiments, R¹⁴ is hydrogen. In some embodiments, R¹⁴ is methyl. In some embodiments, each instance of R⁵ is independently halogen, hydroxyl, C₁-C₃ alkoxy, C₁-C₃ alkylthio, or C₁-C₃ alkyl. In some embodiments, each instance of R⁵ is independently hydroxyl, C₁-C₃ alkoxy, or C₁-C₃ alkyl. In some embodiments, each instance of R⁵ is independently hydroxyl or C₁-C₃ alkyl. In some embodiments, each instance of R⁵ is hydroxyl. In some embodiments, each instance of R⁵ is independently C₁-C₃ alkoxy. In some embodiments, each instance of R⁵ is independently C₁-C₃ alkyl. In some embodiments, each instance of R⁶ and R⁷ is independently hydrogen or C₁-C₃ alkyl. In some embodiments, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some embodiments, each instance of R⁶ and R⁷ is independently hydrogen or methyl. In some embodiments, each instance of R⁶ and R⁷ is independently C₁-C₃ alkyl. In some embodiments, each instance of R⁶ and R⁷ is hydrogen. In some embodiments n is 1 to 2. In some embodiments, n is 2 to 4. In some embodiments, n is 2 or 3. In some embodiments, n is 2. In some embodiments m is 1 to 2. In some embodiments, m is 2 to 4. In some embodiments, m is 2 or 3. In some embodiments, m is 2.

Compounds of Formula (VII) or (VIIa) may be a cation or a polycation. In some embodiments, the compounds of Formula (VII) or (VIIa) are cations due to a positive charge on a nitrogen. Compounds of Formula (VII) or (VIIa) may be anions or polyanions. In some embodiments, the compounds of Formula (VII) or (VIIa) are anions due to a negative charge on an oxygen. Compounds of Formula (VII) or (VIIa) may be zwitterions. In some embodiments, the compounds of Formula (VII) or (VIIa) are zwitterions due to a positive charge on a nitrogen and a negative charge on an oxygen.

A “salt” of compounds of Formula (VII) or (VIIa) may be derived from inorganic or organic acids, bases, or a combination thereof. In some cases, a salt includes a cationic form of Formula (VII) or (VIIa) and an anion. Suitable anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. In some cases, a salt includes an anionic form of Formula (VII) or (VIIa) and a cation. Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

### Compounds of Formula (VIII)

Aspects of the present disclosure provide a compound of Formula (VIII):

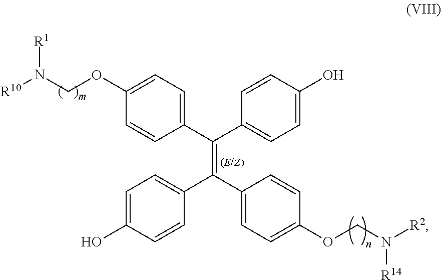

or a pharmaceutically acceptable salt, solvate, or tautomer thereof, wherein:
R¹ and R² are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
m and n are each independently integers from 1 to 10.

Aspects of the present disclosure provide a compound of Formula (VIIIa):

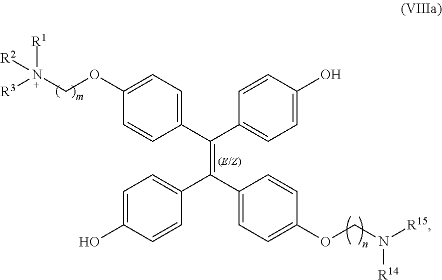

or a pharmaceutically acceptable salt, solvate, or tautomer thereof, wherein:
R¹, R², R³, and R¹⁵ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
m and n are each independently integers from 1 to 10.

Aspects of the present disclosure provide a compound of Formula (VIIIb):

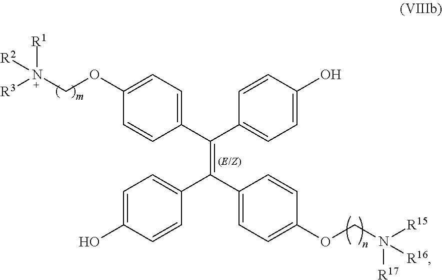

or a pharmaceutically acceptable salt, solvate, or tautomer thereof, wherein:
R¹, R², R³, R¹⁵, R¹⁶, and R¹⁷ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or
—C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
m and n are each independently integers from 1 to 10.

In some embodiments, the compound is a compound of Formula (VIII). In some embodiments, the compound is a compound of Formula (VIIIa). In some embodiments, the compound is a compound of Formula (VIIIb).

In some embodiments, the compound of Formula (VIII) is a compound of Formula (VIIIc):

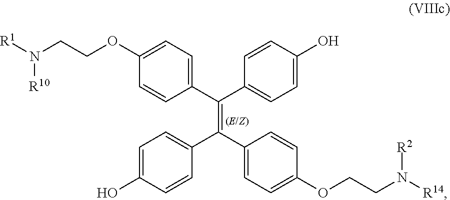

or a pharmaceutically acceptable salt, solvate, or tautomer thereof.

In some embodiments, the compound of Formula (VIIIa) is a compound of Formula (VIIId):

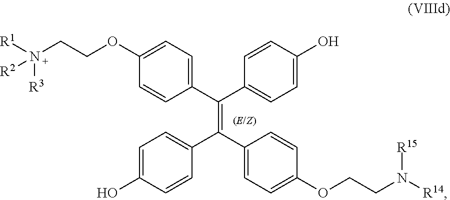

or a pharmaceutically acceptable salt, solvate, or tautomer thereof.

In some embodiments, the compound of Formula (VIIIb) is a compound of Formula (VIIIe):

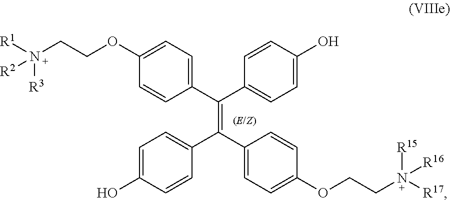

or a pharmaceutically acceptable salt, solvate, or tautomer thereof.

A compound of Formula (VIII) may have a variety of substituents as defined by the R groups and the value of “n” or “m” to define the length of a carbon chain. In some embodiments, R¹, R², R³, R¹⁵, R¹⁶, and R¹⁷ are each independently C₁-C₆ alkyl substituted with 0 to 1 instances of R⁵, C₂-C₆ alkenyl substituted with 0 to 1 instances of R⁵, C₂-C₆ alkynyl substituted with 0 to 1 instances of R⁵, —C(═O)OR⁶, —C(═O)NR⁶R⁷, or —C(═O)SR⁶. In some embodiments, R¹, R², R³, R¹⁵, R¹⁶, and R¹⁷ are each independently C₁-C₆ alkyl substituted with 0 to 3 instances of R⁵, C₂-C₆alkenyl substituted with 0 to 3 instances of R⁵, or C₂-C₆ alkynyl substituted with 0 to 3 instances of R⁵. In some embodiments, R¹, R², R³, R¹⁵, R¹⁶, and R¹⁷ are each independently C₁-C₆alkyl substituted with 0 to 3 instances of R⁵. In some embodiments, R¹, R², R³, R¹⁵, R¹⁶, and R¹⁷ are each independently C₁-C₆ alkyl substituted with 0 or 1 instance of R⁵. In some embodiments, R¹, R², R³, R¹⁵, R¹⁶, and R¹⁷ are each independently C₁-C₃ alkyl substituted with 0 to 3 instances of R⁵. In some embodiments, R¹, R², R³, R¹⁵, R¹⁶, and R¹⁷ are each independently C₁-C₃ alkyl substituted with 0 or 1 instance of R⁵. In some embodiments, R¹, R², R³, R¹⁵, R¹⁶, and R¹⁷ are each independently C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹, R², R³, R¹⁵, R¹⁶, and R¹⁷ are each independently C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R. In some embodiments, R¹, R², R³, R¹⁵, R¹⁶, and R¹⁷ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹, R², R³, R¹⁵, R¹⁶, and R¹⁷ are each independently C₁-C₆ alkyl. In some embodiments, R¹, R², R³, R¹⁵, R¹⁶, and R¹⁷ are each independently C₁-C₃ alkyl. In some embodiments, R¹, R², R³, R¹⁵, R¹⁶, and R¹⁷ are each —CH₃. In some embodiments, R¹, R², and R³ are each —CH₃. In some embodiments, R¹⁵, R¹⁶, and R¹⁷ are each —CH₃. In some embodiments, R¹ is —CH₃. In some embodiments, R² is —CH₃. In some embodiments, R¹⁵ is —CH₃. In some embodiments, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OR⁶, —C(═O)NR⁶R⁷, or —C(═O)SR⁶. In some embodiments, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OH, —C(═O)NH₂, or —C(═O)SH. In some embodiments, R¹⁰ and R¹⁴ are each independently hydrogen, C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹⁰ and R¹⁴ are each independently hydrogen or C₁-C₆ alkyl substituted with 0 to 3 instances of R⁵. In some embodiments, R¹⁰ and R¹⁴ are each independently hydrogen or C₁-C₃ alkyl substituted with 0 to 3 instances of R⁵. In some embodiments, R¹⁰ and R¹⁴ are each independently hydrogen or C₁-C₆ alkyl substituted with 0 or 1 instance of R⁵. In some embodiments, R¹⁰ and R¹⁴ are each independently hydrogen or C₁-C₃ alkyl substituted with 0 or 1 instance of R⁵. In some embodiments, R¹⁰ and R¹⁴ are each independently hydrogen or C₁-C₃ alkyl. In some embodiments, R¹⁰ and R¹⁴ are each independently hydrogen or methyl. In some embodiments, R¹⁰ and R¹⁴ are each independently hydrogen. In some embodiments, R¹⁰ and R¹⁴ are each independently methyl. In some embodiments, R¹⁰ is hydrogen. In some embodiments, R¹⁰ is methyl. In some embodiments, R¹⁴ is hydrogen. In some embodiments, R¹⁴ is methyl. In some embodiments, each instance of R⁵ is independently halogen, hydroxyl, C₁-C₃ alkoxy, C₁-C₃ alkylthio, or C₁-C₃ alkyl. In some embodiments, each instance of R⁵ is independently hydroxyl, C₁-C₃ alkoxy, or C₁-C₃ alkyl. In some embodiments, each instance of R⁵ is independently hydroxyl or C₁-C₃ alkyl. In some embodiments, each instance of R⁵ is hydroxyl. In some embodiments, each instance of R⁵ is independently C₁-C₃ alkoxy. In some embodiments, each instance of R⁵ is independently C₁-C₃ alkyl. In some embodiments, each instance of R⁶ and R⁷ is independently hydrogen or C₁-C₃ alkyl. In some embodiments, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some embodiments, each instance of R⁶ and R⁷ is independently hydrogen or methyl. In some embodiments, each instance of R⁶ and R⁷ is independently C₁-C₃ alkyl. In some embodiments, each instance of R⁶ and R⁷ is hydrogen. In some embodiments n is 1 to 2. In some embodiments, n is 2 to 4. In some embodiments, n is 2 or 3. In some embodiments, n is 2. In some embodiments m is 1 to 2. In some embodiments, m is 2 to 4. In some embodiments, m is 2 or 3. In some embodiments, m is 2.

Compounds of Formula (VIII), Formula (VIIIa), and Formula (VIIIb) may be cations or polycations. In some embodiments, the compounds of Formula (VIII), Formula (VIIIa), and Formula (VIIIb) are cations due to a positive charge on a nitrogen. Compounds of Formula (VIII), Formula (VIIIa), and Formula (VIIIb) may be anions or polyanions. In some embodiments, the compounds of Formula (VIII), Formula (VIIIa), and Formula (VIIIb) are anions due to a negative charge on an oxygen. Compounds of Formula (VIII), Formula (VIIIa), and Formula (VIIIb) may be zwitterions. In some embodiments, the compounds of Formula (VIII), Formula (VIIIa), and Formula (VIIIb) are zwitterions due to a positive charge on a nitrogen and a negative charge on an oxygen.

A “salt” of compounds of Formula (VIII), Formula (VIIIa), and Formula (VIIIb) may be derived from inorganic or organic acids, bases, or a combination thereof. In some cases, a salt includes a cationic form of Formula (VIII), Formula (VIIIa), or Formula (VIIIb) and an anion. Suitable anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. In some cases, a salt includes an anionic form of Formula (VIII), Formula (VIIIa), or Formula (VIIIb) and a cation. Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

### Substituents of Chemical Formulas

Compounds of the current disclosure may be represented by the generic structures and chemical formulas described herein and have variable substituents as defined by their R group definition.

R¹, R², R³, R¹⁵, R¹⁶, and R¹⁷ Substituents

In some embodiments, R¹, R², R³, R¹⁵, R¹⁶, and R¹⁷ are each independently C₁-C₆ alkyl substituted with 0 to 1 instances of R⁵, C₂-C₆ alkenyl substituted with 0 to 1 instances of R⁵, C₂-C₆alkynyl substituted with 0 to 1 instances of R⁵, —C(═O)OR⁶, —C(═O)NR⁶R⁷, or —C(═O)SR⁶. In some embodiments, R¹, R², R³, R¹⁵, R¹⁶, and R¹⁷ are each independently C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹, R², R³, R¹⁵, R¹⁶, and R¹⁷ are each independently C₁-C₆ alkyl substituted with 0 to 3 instances of R⁵. In some embodiments, R¹, R², R³, R¹⁵, R¹⁶, and R¹⁷ are each independently C₁-C₆ alkyl substituted with 0 or 1 instance of R⁵. In some embodiments, R¹, R², R³, R¹⁵, R¹⁶, and R¹⁷ are each independently C₁-C₃ alkyl substituted with 0 to 3 instances of R⁵. In some embodiments, R¹, R², R³, R¹⁵, R¹⁶, and R¹⁷ are each independently C₁-C₃ alkyl substituted with 0 or 1 instance of R⁵. In some embodiments, R¹, R², R³, R¹⁵, R¹⁶, and R¹⁷ are each independently C₁-C₃ alkyl substituted with 0 to 3 instances of R⁵, C₂-C₃ alkenyl substituted with 0 to 3 instances of R⁵, or C₂-C₃ alkynyl substituted with 0 to 3 instances of R⁵. In some embodiments, R¹, R², R³, R¹⁵, R¹⁶, and R¹⁷ are each independently C₁-C₃ alkyl substituted with 0 or 1 instance of R⁵, C₂-C₃ alkenyl substituted with 0 or 1 instance of R⁵, or C₂-C₃ alkynyl substituted with 0 or 1 instance of R⁵. In some embodiments, R¹, R², R³, R¹⁵, R¹⁶, and R¹⁷ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OH, —C(═O)NH₂, or —C(═O)SH. In some embodiments, R¹, R², R³, R¹⁵, R¹⁶, and R¹⁷ are each independently C₁-C₆ alkyl. In some embodiments, R¹, R², R³, R¹⁵, R¹⁶, and R¹⁷ are each independently C₁-C₃ alkyl. In some embodiments, R¹, R², R³, R¹⁵, R¹⁶, and R¹⁷ are each —CH₃. In some embodiments, R¹, R², and R³ are each —CH₃. In some embodiments, R¹⁵, R¹⁶, and R¹⁷ are each —CH₃. In some embodiments, R¹ is —CH₃. In some embodiments, R² is —CH₃. In some embodiments, R¹⁵ is —CH₃.

### R⁴ Substituents

In some embodiments, R⁴ is C₁-C₆ alkyl substituted with 0 to 3 instances of R⁸. In some embodiments, R⁴ is C₁-C₆ alkyl substituted with 0 or 1 instance of R⁸. In some embodiments, R⁴ is C₁-C₃ alkyl substituted with 0 to 3 instances of R⁸. In some embodiments, R⁴ is C₁-C₃ alkyl substituted with 0 or 1 instance of R⁸. In some embodiments, R⁴ is C₁-C₃ alkyl. In some embodiments, R⁴ is —CH₂CH₃.

### R⁵ Substituents

In some embodiments, each instance of R⁵ is independently halogen, hydroxyl, C₁-C₃ alkoxy, C₁-C₃ alkylthio, or C₁-C₃ alkyl. In some embodiments, each instance of R⁵ is independently hydroxyl, C₁-C₃ alkoxy, or C₁-C₃ alkyl. In some embodiments, each instance of R⁵ is independently hydroxyl or C₁-C₃ alkyl. In some embodiments, each instance of R⁵ is hydroxyl. In some embodiments, each instance of R⁵ is independently C₁-C₃ alkoxy. In some embodiments, each instance of R⁵ is independently C₁-C₃ alkyl.

R⁶ and R⁷ Substituents

In some embodiments, each instance of R⁶ and R⁷ is independently hydrogen or C₁-C₃ alkyl. In some embodiments, each instance of R⁶ and R⁷ is independently C₁-C₃ alkyl, or an instance of R⁶ and an instance of R⁷ on the same nitrogen are taken together with the nitrogen to form C₂-C₇ heterocycloalkyl. In some embodiments, each instance of R⁶ and R⁷ is independently hydrogen or methyl. In some embodiments, each instance of R⁶ and R⁷ is independently C₁-C₃ alkyl. In some embodiments, each instance of R⁶ and R⁷ is hydrogen.

### R⁸ Substituents

In some embodiments, each instance of R⁸ is independently hydroxyl, C₁-C₃ alkoxy, C₁-C₃ alkyl, or C₃-C₈ cycloalkyl. In some embodiments, each instance of R⁸ is independently hydroxyl, C₁-C₃ alkoxy, or C₁-C₃ alkyl. In some embodiments, each instance of R⁸ is independently hydroxyl or C₁-C₃ alkyl. In some embodiments, each instance of R⁸ is independently C₁-C₃ alkyl.

### R⁹ Substituents

In some embodiments, R⁹ is hydrogen, hydroxyl, C₁-C₆ alkyl substituted with 0 or 1 instance of R¹¹, C₂-C₆ alkenyl substituted with 0 or 1 instance of R¹¹, or C₂-C₆ alkynyl substituted with 0 or 1 instance of R¹¹. In some embodiments, R⁹ is hydrogen, hydroxyl, C₁-C₃ alkyl substituted with 0 to 3 instances of R¹¹, C₂-C₃ alkenyl substituted with 0 to 3 instances of R¹¹, or C₂-C₃ alkynyl substituted with 0 to 3 instances of R¹¹. In some embodiments, R⁹ is hydrogen, hydroxyl, C₁-C₃ alkyl substituted with 0 or 1 instance of R¹¹, C₂-C₃ alkenyl substituted with 0 or 1 instance of R¹¹, or C₂-C₃ alkynyl substituted with 0 or 1 instance of R¹¹. In some embodiments, R⁹ is hydrogen, hydroxyl, or C₁-C₆ alkyl substituted with 0 to 3 instances of R¹¹. In some embodiments, R⁹ is C₁-C₆ alkyl substituted with 0 to 3 instances of R¹¹. In some embodiments, R⁹ is hydrogen, hydroxyl, or C₁-C₆ alkyl substituted with 0 or 1 instance of R¹¹. In some embodiments, R⁹ is C₁-C₆ alkyl substituted with 0 or 1 instance of R¹¹. In some embodiments, R⁹ is hydrogen, hydroxyl, or C₁-C₆ alkyl. In some embodiments, R⁹ is C₁-C₆ alkyl. In some embodiments, R⁹ is hydrogen, hydroxyl, or C₁-C₃ alkyl substituted with 0 to 3 instances of R¹¹. In some embodiments, R⁹ is C₁-C₃ alkyl substituted with 0 to 3 instances of R¹¹. In some embodiments, R⁹ is hydrogen, hydroxyl, or C₁-C₃ alkyl substituted with 0 or 1 instance of R¹¹. In some embodiments, R⁹ is C₁-C₃ alkyl substituted with 0 or 1 instance of R¹¹. In some embodiments, R⁹ is hydrogen, hydroxyl, or C₁-C₃ alkyl. In some embodiments, R⁹ is C₁-C₃ alkyl. In some embodiments, R⁹ is C₁-C₃ alkyl. In some embodiments, R⁹ is methyl.

R¹⁰ and R¹⁴ Substituents

In some embodiments, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆alkynyl substituted with 0 or 1 instances of R⁵, —C(═O)OR⁶, —C(═O)NR⁶R⁷, or —C(═O)SR⁶. In some embodiments, R¹⁰ and R¹⁴ are each independently hydrogen, C₁-C₆ alkyl substituted with 0 or 1 instances of R⁵, C₂-C₆ alkenyl substituted with 0 or 1 instances of R⁵, C₂-C₆ alkynyl substituted with 0 or 1 instances of R⁵, —C(═O)OH, —C(═O)NH₂, or —C(═O)SH. In some embodiments, R¹⁰ and R¹⁴ are each independently hydrogen, C₁-C₆ alkyl substituted with 0 to 3 instances of R⁵, C₂-C₆ alkenyl substituted with 0 to 3 instances of R⁵, or C₂-C₆ alkynyl substituted with 0 to 3 instances of R⁵. In some embodiments, R¹⁰ and R¹⁴ are each independently hydrogen or C₁-C₆ alkyl substituted with 0 to 3 instances of R⁵. In some embodiments, R¹⁰ and R¹⁴ are each independently hydrogen or C₁-C₃ alkyl substituted with 0 to 3 instances of R⁵. In some embodiments, R¹⁰ and R¹⁴ are each independently hydrogen or C₁-C₆ alkyl substituted with 0 or 1 instance of R⁵. In some embodiments, R¹⁰ and R¹⁴ are each independently hydrogen or C₁-C₃ alkyl substituted with 0 or 1 instance of R⁵. In some embodiments, R¹⁰ and R¹⁴ are each independently hydrogen or C₁-C₃ alkyl. In some embodiments, R¹⁰ and R¹⁴ are each independently hydrogen or methyl. In some embodiments, R¹⁰ and R¹⁴ are each independently hydrogen. In some embodiments, R¹⁰ and R¹⁴ are each independently methyl. In some embodiments, R¹⁰ is hydrogen. In some embodiments, R¹⁰ is methyl. In some embodiments, R¹⁴ is hydrogen. In some embodiments, R¹⁴ is methyl.

### R¹¹ Substituents

In some embodiments, each instance of R¹¹ is independently halogen, hydroxyl, C₁-C₃ alkoxy, C₁-C₃ alkylthio, or C₁-C₃ alkyl. In some embodiments, each instance of R¹¹ is independently halogen, hydroxyl, C₁-C₃ alkylthio, or C₁-C₃ alkyl. In some embodiments, each instance of R¹¹ is independently hydroxyl, C₁-C₃ alkoxy, or C₁-C₃ alkyl. In some embodiments, each instance of R¹¹ is independently hydroxyl or C₁-C₃ alkyl. In some embodiments, each instance of R¹¹ is hydroxyl. In some embodiments, each instance of R¹¹ is independently C₁-C₃ alkyl.

### Variable Carbon Chain Length

Compounds of the current disclosure may have variable carbon lengths represented in the generic structures as “n”. In some embodiments n is 1 to 2. In some embodiments, n is 2 to 4. In some embodiments, n is 2 or 3. In some embodiments, n is 2.

### Variable Carbon Chain Length

Compounds of the current disclosure may have variable carbon lengths represented in the generic structures as “m”. In some embodiments m is 1 to 2. In some embodiments, m is 2 to 4. In some embodiments, m is 2 or 3. In some embodiments, m is 2.

X, R¹², and R¹³ Substituents

Compounds of the current disclosure may comprise a carbonate or carbamate functional group. A carbonate or carbamate functional group may be represented by an ester connected to a generic “X” group as a substituent. The carbonate or carbamate functional group may comprise a variety of substituents which is represented by “X” in generic structures of the present disclosure. In some embodiments X is —OR¹² or —NR¹²R¹³. In some embodiments, X is —OR¹² or —SR¹². In some embodiments, X is —OR¹². In some embodiments, X is —OCH₃. In some embodiments, R¹² and R¹³ are each independently C₁-C₃ alkyl. In some embodiments, R¹² and R³ are each independently methyl. In some embodiments, R¹² is methyl.

### Chemical Compounds

### Compounds of Formula (IX)

Aspects of the present disclosure provides a compound of Formula (IX):

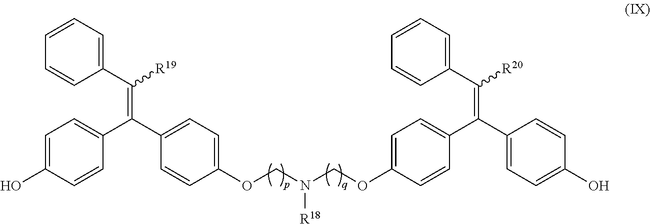

or a pharmaceutically acceptable salt, solvate, or tautomer thereof,
wherein:
   R¹⁸ is hydrogen, C₁-C₆ alkyl substituted with 0 to 3 instances of R²¹, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   R¹⁹ and R²⁰ are each independently C₁-C₆ alkyl substituted with 0 to 3 instances of R⁸, C₂-C₆ alkenyl substituted with 0 to 3 instances of R⁸, or C₂-C₆ alkynyl substituted with 0 to 3 instances of R²²;
   each instance of R²¹ is independently halogen, hydroxyl, C₁-C₃ alkoxy, C₁-C₃ alkylthio, C₁-C₃ alkyl, —C(═O)OH, or —C(═O)OCH₃;
each instance of R²² is independently halogen, hydroxyl, C₁-C₃ alkoxy, C₁-C₃ alkylthio, C₁-C₃ alkyl, or C₃-C₈ cycloalkyl; and
   p and q are each independently integers from 1 to 10.

In some aspects, the compound of Formula (IX) is a compound of Formula (IXa):

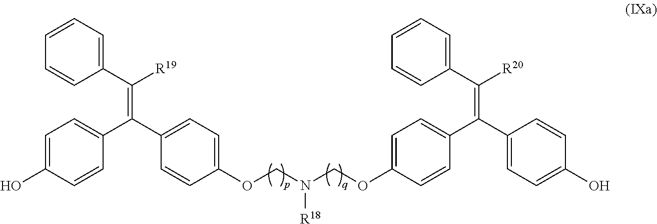

or a pharmaceutically acceptable salt, solvate, or tautomer thereof.

In some aspects, the compound of Formula (IX) is a compound of Formula (IXb):

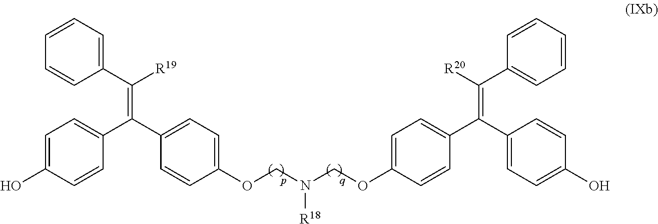

or a pharmaceutically acceptable salt, solvate, or tautomer thereof.

In some aspects, the compound of Formula (IX) is a compound of Formula (IXc):

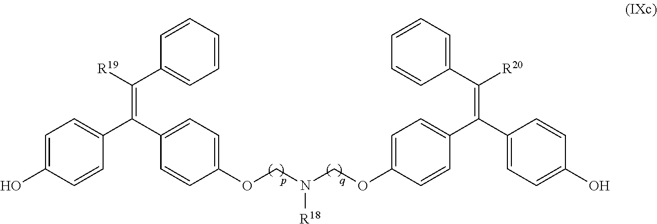

or a pharmaceutically acceptable salt, solvate, or tautomer thereof.

In some aspects, the compound of Formula (IX) is a compound of Formula (IXd):

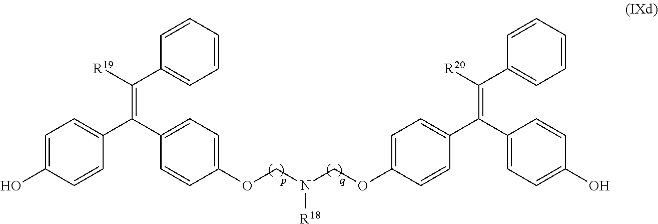

or a pharmaceutically acceptable salt, solvate, or tautomer thereof.

In some aspects, the compound of Formula (IX) comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% Formula (IXa). In some aspects, the compound of Formula (IX) comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% Formula (IXb). In some aspects, the compound of Formula (IX) comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% Formula (IXc). In some aspects, the compound of Formula (IX) comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% Formula (IXd).

In some aspects, the compound of Formula (IX) comprises less than 0.5%, less than 1%, less than 2%, less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 98%, less than 99%, or less than 99.5% Formula (IXa). In some aspects, the compound of Formula (IX) comprises less than 0.5%, less than 1%, less than 2%, less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 98%, less than 99%, or less than 99.5% Formula (IXb). In some aspects, the compound of Formula (IX) comprises less than 0.5%, less than 1%, less than 2%, less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 98%, less than 99%, or less than 99.5% Formula (IXc). In some aspects, the compound of Formula (IX) comprises less than 0.5%, less than 1%, less than 2%, less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 98%, less than 99%, or less than 99.5% Formula (IXd).

In some aspects, R¹⁹ is C₁-C₃ alkyl optionally substituted with 0 to 3 instances of R²². In some aspects, R¹⁹ is C₁-C₃ alkyl optionally substituted with 0 to 2 instances of R²². In some aspects, R¹⁹ is C₁-C₃ alkyl optionally substituted with one instance of R²². In some aspects, R¹⁹ is C₁-C₃ alkyl. In other aspects, R¹⁹ is ethyl.

In some aspects, R²⁰ is C₁-C₃ alkyl optionally substituted with 0 to 3 instances of R²². In some aspects, R²⁰ is C₁-C₃ alkyl optionally substituted with 0 to 2 instances of R²². In some aspects, R²⁰ is C₁-C₃ alkyl optionally substituted with one instance of R²². In some aspects, R²⁰ is C₁-C₃ alkyl. In other aspects, R²⁰ is ethyl.

In one aspect, R¹⁸ is hydrogen or C₁-C₆ alkyl. In one aspect, R¹⁸ is hydrogen or C₁-C₃ alkyl. In a particular aspect, R¹⁸ is hydrogen. In another aspect, R¹⁸ is methyl.

In some aspects, p is 1 to 4. In additional aspects, p is 2 to 4. In particular aspects, p is 2 or 3. In a specific aspect, p is 2. In some aspects, q is 1 to 4. In additional aspects, q is 2 to 4. In particular aspects, q is 2 or 3. In a specific aspect, q is 2.

Compounds of the current disclosure may be represented by the chemical formulas as described herein or represented by compound structures. TABLE 1 provides compounds of the present disclosure.

TABLE 1
Chemical Compounds of the Present Disclosure or Pharmaceutically
Acceptable Salts, Solvates, or Tautomers Thereof

| Compound | Structure |
|---|---|
| Compound 1 | 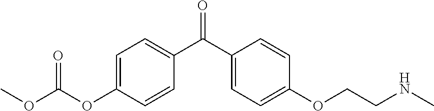 |
| Compound 2 | 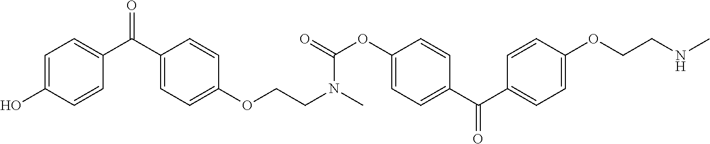 [SMILES: CNCCOc1ccc(C(=O)c2ccc(OC(=O)N(C)CCOc3ccc(C(=O)c4ccc(O)cc4)cc3)cc2)cc1] |
| Compound 3 | 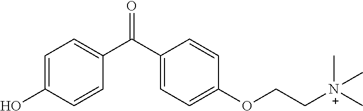 [SMILES: C[N+](C)(C)CCOc1ccc(C(=O)c2ccc(O)cc2)cc1] |
| Compound 4a | 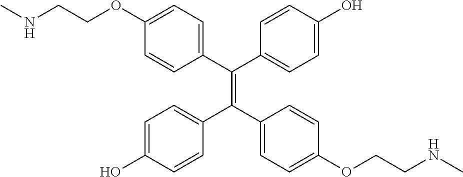 |
| Compound 4b | 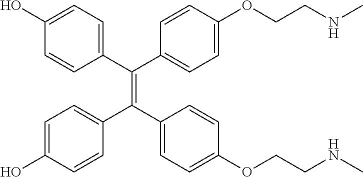 [SMILES: CNCCOc1ccc(/C(=C(/c2ccc(O)cc2)c2ccc(OCCNC)cc2)c2ccc(O)cc2)cc1] |
| Compound 5a | 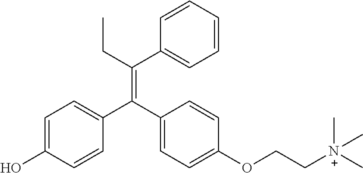 [SMILES: CC/C(=C(\c1ccc(O)cc1)c1ccc(OCC[N+](C)(C)C)cc1)c1ccccc1] |
| Compound 5b | 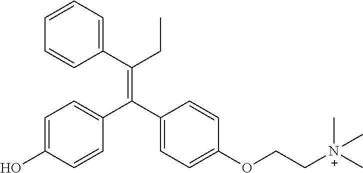 [SMILES: CC/C(=C(/c1ccc(O)cc1)c1ccc(OCC[N+](C)(C)C)cc1)c1ccccc1] |
| Compound 6a | 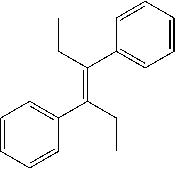 [SMILES: CC/C(=C(/CC)c1ccccc1)c1ccccc1] |
| Compound 6b | 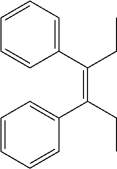 [SMILES: CC/C(=C(\CC)c1ccccc1)c1ccccc1] |
| Compound 7a | 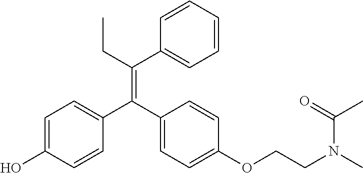 [SMILES: CC/C(=C(\c1ccc(O)cc1)c1ccc(OCCN(C)C(C)=O)cc1)c1ccccc1] |
| Compound 7b | 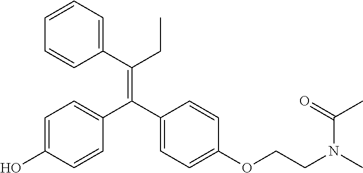 [SMILES: CC/C(=C(/c1ccc(O)cc1)c1ccc(OCCN(C)C(C)=O)cc1)c1ccccc1] |
| Compound 8 | 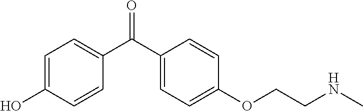 [SMILES: CNCCOc1ccc(C(=O)c2ccc(O)cc2)cc1] |
| Compound 9a | 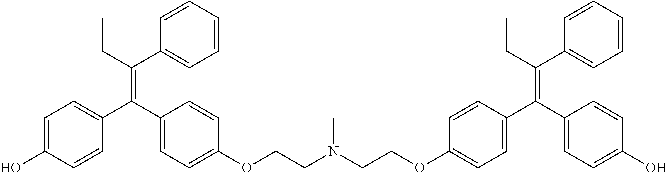 [SMILES: CC/C(=C(\c1ccc(O)cc1)c1ccc(OCCN(C)CCOc2ccc(/C(=C(\CC)c3ccccc3)c3ccc(O)cc3)cc2)cc1)c1ccccc1] |
| Compound 9b | 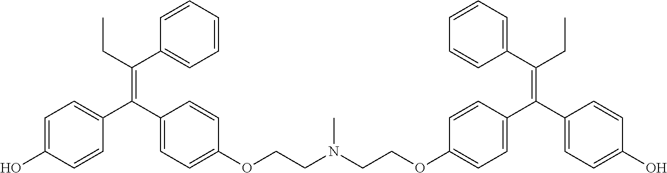 [SMILES: CC/C(=C(\c1ccc(O)cc1)c1ccc(OCCN(C)CCOc2ccc(/C(=C(/CC)c3ccccc3)c3ccc(O)cc3)cc2)cc1)c1ccccc1] |
| Compound 9c | 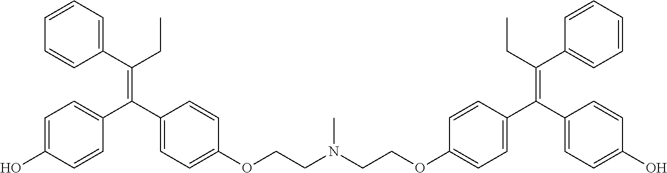 [SMILES: CC/C(=C(/c1ccc(O)cc1)c1ccc(OCCN(C)CCOc2ccc(/C(=C(\CC)c3ccccc3)c3ccc(O)cc3)cc2)cc1)c1ccccc1] |

The compounds of the present disclosure may have isomers (e.g., structural isomers, tautomers, stereoisomers, enantiomers, rotamers, topoisomers, or isotopomers). The compounds of the present disclosure may have (E) and (Z) isomers.

Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof may be in a cationic or anionic form. For example, compounds may comprise a deprotonated hydroxyl or a quaternary amine resulting in an overall negative or positive charge, respectively. A “salt” of a compound described herein may be derived from inorganic or organic acids and bases. Suitable counter-anions include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide. Suitable counter-cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

### Pharmaceutical Compositions

Compositions of the present disclosure may comprise one or more compounds as described herein and a pharmaceutically acceptable carrier or diluent. In some embodiments, a pharmaceutical composition may comprise a first compound of any compound described herein and a pharmaceutically acceptable carrier or diluent. In some embodiments, a pharmaceutical composition may further comprise a second compound of any compound described herein. In some embodiments, the first compound may be a compound of Formulas (I), (II), (III), (IV), (V), (VI), (VII), (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof. In some embodiments, a pharmaceutical composition may further comprise a second compound of any compound described herein. In some embodiments, the second compound may be a compound of Formulas (I), (II), (III), (IV), (V), (VI), (VII), (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof.

In some embodiments, the first compound is present in between 40-60%, 50-70%, 60-80%, 70-90%, 80-90%, 85-95%, 90-95%, or 95-99% (w/w). In some embodiments, the first compound is present in between 20-30%, 30-40%, 40-50%, 50-60%, 60-70%, 70-80%, 80-85%, 85-90%, 90-92.5%, 92.5-95%, 95-96%, 96-97%, 97.0-97.5%, 97.5-98%, 98.0-98.5%, 98.5-99.0%, 99.0-99.1%, 99.1-99.2%, 99.2-99.3%, 99.3-99.4%, 99.4-99.5%, 99.5-99.6%, 99.6-99.7%, 99.70-99.75%, 99.75-99.80%, 99.80-99.85%, 99.85-99.90%, 99.90-99.91%, 99.91-99.92%, 99.92-99.93%, 99.93-99.94%, 99.94-99.95%, 99.95-99.96%, 99.96-99.97%, 99.97-99.98%, or 99.98-99.99% (w/w).

In some embodiments, the second compound is present between 1-5%, 5-10%, 5-15%, 10-20%, 10-30%, 20-40%, 30-50%, or 40-60% (w/w). In some embodiments, the second compound is present between 0.01-0.02%, 0.02-0.03%, 0.03-0.04%, 0.04-0.05%, 0.05-0.06%, 0.06-0.07%, 0.07-0.08%, 0.08-0.09%, 0.09-0.10%, 0.10-0.15%, 0.15-0.20%, 0.20-0.25%, 0.25-0.30%, 0.3-0.4%, 0.4-0.5%, 0.5-0.6%, 0.6-0.7%, 0.7-0.8%, 0.8-0.9%, 0.9-1.0%, 1.0-1.5%, 1.5-2.0%, 2.0-2.5%, 2.5-3.0%, 3-4%, 4-5%, 5-7.5%, 7.5-10%, 10-15%, 15-20%, 20-30%, 30-40%, 40-50%, 50-60%, 60-70%, or 70-80% (w/w).

In some embodiments, the first compound is Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof.

In some embodiments, the second compound is Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof.

In some embodiments, the pharmaceutical composition further comprises endoxifen. In some embodiments, the pharmaceutical composition further comprises (Z)-endoxifen. In some embodiments, the pharmaceutical composition further comprises (E)-endoxifen. In some embodiments, the pharmaceutical composition further comprises:

### Compound S3a:

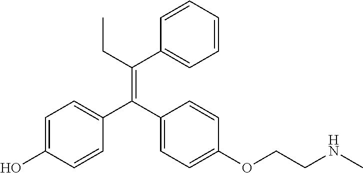 [SMILES: CC/C(=C(\c1ccc(O)cc1)c1ccc(OCCNC)cc1)c1ccccc1]

### and Compound S3b

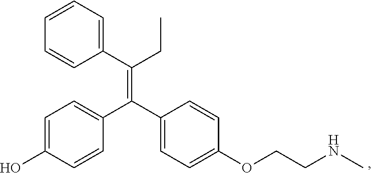 [SMILES: CC/C(=C(/c1ccc(O)cc1)c1ccc(OCCNC)cc1)c1ccccc1]

or a pharmaceutically acceptable salt, solvate, or tautomer thereof. In some embodiments, the pharmaceutical composition further comprises:

### Compound S3a:

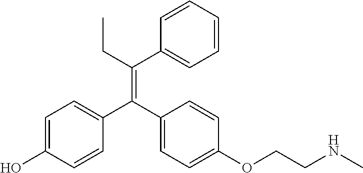 [SMILES: CC/C(=C(\c1ccc(O)cc1)c1ccc(OCCNC)cc1)c1ccccc1]

### or Compound S3b

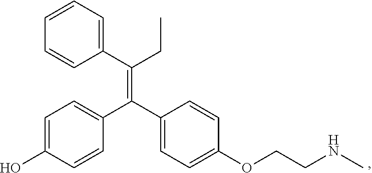 [SMILES: CC/C(=C(/c1ccc(O)cc1)c1ccc(OCCNC)cc1)c1ccccc1]

or a pharmaceutically acceptable salt, solvate, or tautomer thereof. In some embodiments, the pharmaceutical composition further comprises:

### Compound S3a:

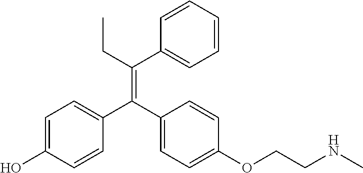 [SMILES: CC/C(=C(\c1ccc(O)cc1)c1ccc(OCCNC)cc1)c1ccccc1]

or a pharmaceutically acceptable salt, solvate, or tautomer thereof. In some embodiments, the pharmaceutical composition further comprises:

### Compound S3b

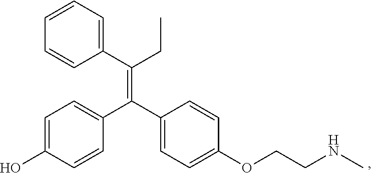 [SMILES: CC/C(=C(/c1ccc(O)cc1)c1ccc(OCCNC)cc1)c1ccccc1]

or a pharmaceutically acceptable salt, solvate, or tautomer thereof.

Endoxifen compositions of the current disclosure may comprise at least 90% (Z)-endoxifen. In some embodiments, an endoxifen composition of the current disclosure may comprise at least 90%, at least 90.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, at least 99%, at least 99.1%, at least 99.2%, at least 99.3%, at least 99.4%, at least 99.5%, at least 99.6%, at least 99.7%, at least 99.75%, at least 99.8%, at least 99.85%, at least 99.9%, at least 99.91%, at least 99.92%, at least 99.93%, at least 99.94%, at least 99.95%, at least 99.96%, at least 99.97%, at least 99.98%, or at least 99.99% (Z)-endoxifen. In some embodiments, an endoxifen composition may comprise between 50% and 60%, between 50% and 70%, between 50% and 75%, between 50% and 80%, between 50% and 85%, between 50% and 90%, between 50% and 95%, between 50% and 97.5%, between 50% and 99%, between 50% and 99.5%, between 60% and 70%, between 60% and 75%, between 60% and 80%, between 60% and 85%, between 60% and 90%, between 60% and 95%, between 60% and 97.5%, between 60% and 99%, between 60% and 99.5%, between 70% and 75%, between 70% and 80%, between 70% and 85%, between 70% and 90%, between 70% and 95%, between 70% and 97.5%, between 70% and 99%, between 70% and 99.5%, between 75% and 80%, between 75% and 85%, between 75% and 90%, between 75% and 95%, between 75% and 97.5%, between 75% and 99%, between 75% and 99.5%, between 80% and 85%, between 80% and 90%, between 80% and 95%, between 80% and 97.5%, between 80% and 99%, between 80% and 99.5%, between 85% and 90%, between 85% and 95%, between 85% and 97.5%, between 85% and 99%, between 85% and 99.5%, between 90% and 95%, between 90% and 97.5%, between 90% and 99%, between 90% and 99.5%, between 95% and 97.5%, between 95% and 99%, between 95% and 99.5%, between 97.5% and 99.5%, not less than 95%, not less than 96%, not less than 97%, not less than 98%, not less than 99%, or not less than 99.5% (Z)-endoxifen. In some embodiments, an endoxifen composition of the current disclosure may comprise not less than 90% and not more than 92.5%, not less than 92.5% and not more than 95%, not less than 95% and not more than 96%, not less than 96% and not more than 97%, not less than 97.0% and not more than 97.5%, not less than 97.5% and not more than 98%, not less than 98.0% and not more than 98.5%, not less than 98.5% and not more than 99.0%, not less than 99.0% and not more than 99.1%, not less than 99.1% and not more than 99.2%, not less than 99.2% and not more than 99.3%, not less than 99.3% and not more than 99.4%, not less than 99.4% and not more than 99.5%, not less than 99.5% and not more than 99.6%, not less than 99.6% and not more than 99.7%, not less than 99.70% and not more than 99.75%, not less than 99.75% and not more than 99.80%, not less than 99.80% and not more than 99.85%, not less than 99.85% and not more than 99.90%, not less than 99.90% and not more than 99.91%, not less than 99.91% and not more than 99.92%, not less than 99.92% and not more than 99.93%, not less than 99.93% and not more than 99.94%, not less than 99.94% and not more than 99.95%, not less than 99.95% and not more than 99.96%, not less than 99.96% and not more than 99.97%, not less than 99.97% and not more than 99.98%, or not less than 99.98% and not more than 99.99% (Z)-endoxifen.

Endoxifen compositions of the current disclosure may comprise at least 90% (E)-endoxifen. In some embodiments, an endoxifen composition of the current disclosure may comprise at least 90%, at least 90.5%, at least 91%, at least 91.5%, at least 92%, at least 92.5%, at least 93%, at least 93.5%, at least 94%, at least 94.5%, at least 95%, at least 95.25%, at least 95.5%, at least 95.75%, at least 96%, at least 96.25%, at least 96.5%, at least 96.75%, at least 97%, at least 97.25%, at least 97.5%, at least 97.75%, at least 98%, at least 98.25%, at least 98.5%, at least 98.75%, at least 99%, at least 99.1%, at least 99.2%, at least 99.3%, at least 99.4%, at least 99.5%, at least 99.6%, at least 99.7%, at least 99.75%, at least 99.8%, at least 99.85%, at least 99.9%, at least 99.91%, at least 99.92%, at least 99.93%, at least 99.94%, at least 99.95%, at least 99.96%, at least 99.97%, at least 99.98%, or at least 99.99% (E)-endoxifen. In some embodiments, an endoxifen composition may comprise between 50% and 60%, between 50% and 70%, between 50% and 75%, between 50% and 80%, between 50% and 85%, between 50% and 90%, between 50% and 95%, between 50% and 97.5%, between 50% and 99%, between 50% and 99.5%, between 60% and 70%, between 60% and 75%, between 60% and 80%, between 60% and 85%, between 60% and 90%, between 60% and 95%, between 60% and 97.5%, between 60% and 99%, between 60% and 99.5%, between 70% and 75%, between 70% and 80%, between 70% and 85%, between 70% and 90%, between 70% and 95%, between 70% and 97.5%, between 70% and 99%, between 70% and 99.5%, between 75% and 80%, between 75% and 85%, between 75% and 90%, between 75% and 95%, between 75% and 97.5%, between 75% and 99%, between 75% and 99.5%, between 80% and 85%, between 80% and 90%, between 80% and 95%, between 80% and 97.5%, between 80% and 99%, between 80% and 99.5%, between 85% and 90%, between 85% and 95%, between 85% and 97.5%, between 85% and 99%, between 85% and 99.5%, between 90% and 95%, between 90% and 97.5%, between 90% and 99%, between 90% and 99.5%, between 95% and 97.5%, between 95% and 99%, between 95% and 99.5%, between 97.5% and 99.5%, not less than 95%, not less than 96%, not less than 97%, not less than 98%, not less than 99%, or not less than 99.5% (E)-endoxifen. In some embodiments, an endoxifen composition of the current disclosure may comprise not less than 90% and not more than 92.5%, not less than 92.5% and not more than 95%, not less than 95% and not more than 96%, not less than 96% and not more than 97%, not less than 97.0% and not more than 97.5%, not less than 97.5% and not more than 98%, not less than 98.0% and not more than 98.5%, not less than 98.5% and not more than 99.0%, not less than 99.0% and not more than 99.1%, not less than 99.1% and not more than 99.2%, not less than 99.2% and not more than 99.3%, not less than 99.3% and not more than 99.4%, not less than 99.4% and not more than 99.5%, not less than 99.5% and not more than 99.6%, not less than 99.6% and not more than 99.7%, not less than 99.70% and not more than 99.75%, not less than 99.75% and not more than 99.80%, not less than 99.80% and not more than 99.85%, not less than 99.85% and not more than 99.90%, not less than 99.90% and not more than 99.91%, not less than 99.91% and not more than 99.92%, not less than 99.92% and not more than 99.93%, not less than 99.93% and not more than 99.94%, not less than 99.94% and not more than 99.95%, not less than 99.95% and not more than 99.96%, not less than 99.96% and not more than 99.97%, not less than 99.97% and not more than 99.98%, or not less than 99.98% and not more than 99.99% (E)-endoxifen.

### Oral Compositions

In some embodiments, the present disclosure relates to a composition formulated for oral administration comprising: 1 mg to 200 mg of one or more compounds selected from TABLE 1 (e.g., Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) per unit dose; and wherein the composition is stable for at least 6 months. In some aspects, the composition formulated for oral administration comprises from 1 mg to 80 mg of one or more compounds selected from TABLE 1 per unit dose, from 2 mg to 80 mg per unit dose, from 4 mg to 80 mg per unit dose, or from 4 mg to 40 mg per unit dose. In some aspects, the composition formulated for oral administration comprises about 1 mg, about 2 mg, about 4 mg, about 6 mg, about 8 mg, about 10 mg, about 20 mg, about 40 mg, or about 80 mg per unit dose.

In a still further aspect, the present disclosure relates to compositions comprising one or more compounds selected from TABLE 1 wherein the composition comprises 0.1% to 10% (wt/wt) of one or more compounds selected from TABLE 1. In some cases, a composition comprising one or more compounds selected from TABLE 1 is a solid, a powder, a liquid, a suspension, or an emulsion. A solid of one or more compounds selected from TABLE 1 of a composition disclosed herein can be amorphous, polycrystalline, or of a single crystalline form. In some embodiments, a pharmaceutical composition comprising one or more compounds selected from TABLE 1, may further comprise a liquid. In some embodiments, a composition comprising one or more compounds selected from TABLE 1 may be suspended in the liquid. Liquids for suspension of a composition comprising one or more compounds selected from TABLE 1 may comprise water, an alcohol, an organic solvent, a pharmaceutically acceptable diluent, a pharmaceutically acceptable liquid or solvent, or an aqueous solution.

In another aspect, the present disclosure relates to a composition comprising one or more compounds selected from TABLE 1 further comprising one or more excipients.

In one aspect, the present disclosure relates to a composition comprising one or more compounds selected from TABLE 1, wherein the composition is formulated for oral, parenteral, topical, and intraductal delivery.

In another aspect, the present disclosure relates to a composition comprising one or more compounds selected from TABLE 1, wherein the composition formulated for oral delivery is a tablet, a caplet, a capsule, a pill, a powder, a troche, an elixir, a suspension, a syrup, a wafer, a chewing gum, a dragee, and a lozenge.

### Synthetic Methods

Compounds of Formula (I)-Formula (VIII), or pharmaceutically acceptable salts, solvates, or tautomers thereof, or any one of Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof, may be made by synthetic methods as described in the following section.

### Methods of Making Formula (I)

Methods of making compounds of Formula (I) may include following a synthetic reaction scheme as illustrated in SCHEME 1. SCHEME 1 may also be used for the synthesis of Compound 1, Compound 2, Compound 3, or a combination thereof.

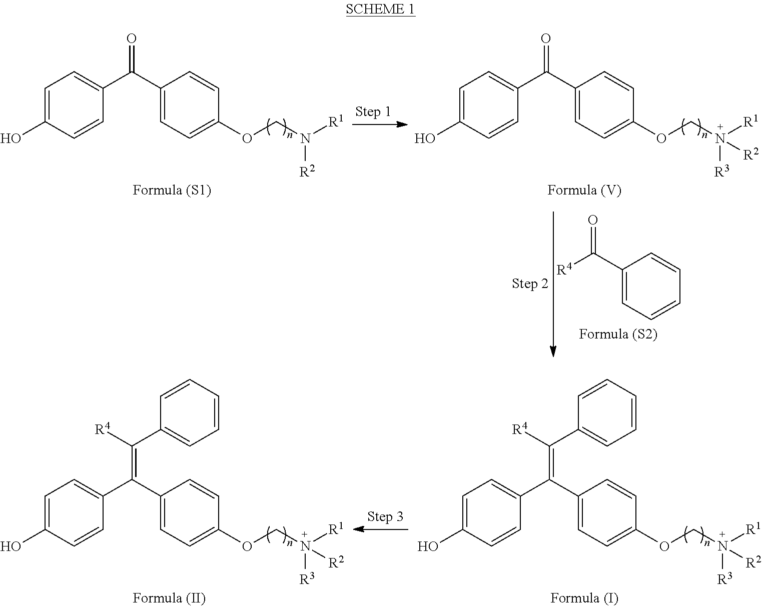

The present disclosure provides a method of producing a compound as described herein, or a pharmaceutically acceptable salt, solvate, or tautomer thereof, the method comprising: coupling a compound of Formula (V):

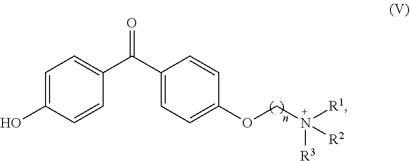

or a pharmaceutically acceptable salt, solvate, or tautomer thereof with a compound of Formula (S2):

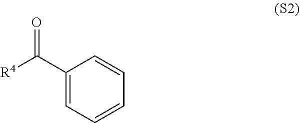

or a pharmaceutically acceptable salt, solvate, or tautomer thereof, thereby producing the compound of Formula (I):

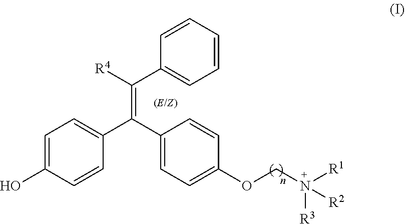

or a pharmaceutically acceptable salt, solvate, or tautomer thereof,
wherein:
   R¹, R², R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   R⁴ is C₁-C₆ alkyl substituted with 0 to 3 instances of R⁸, C₂-C₆ alkenyl substituted with 0 to 3 instances of R¹, or C₂-C₆ alkynyl substituted with 0 to 3 instances of R¹;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl;
each instance of R¹ is independently halogen, hydroxyl, C₁-C₃ alkoxy, C₁-C₃ alkylthio, C₁-C₃ alkyl, or C₃-C₈ cycloalkyl; and
n is an integer from 1 to 10.

In some embodiments, the coupling comprises an olefination reaction. In some embodiments, the coupling comprises a McMurry reaction. In some embodiments, the method comprises activating a compound of Formula (V) and a compound of Formula (S2) with a metal catalyst. In some embodiments, the method comprises generating the metal catalyst by combining a metal salt with a reducing agent. In some embodiments, the metal catalyst is titanium, zirconium, vanadium, niobium, molybdenum, tungsten, aluminum, or zinc. In some embodiments, the metal catalyst comprises titanium. In some embodiments, the metal salt is a titanium halide. In some embodiments, the titanium halide is TiCl₃ or TiCl₄.

In some embodiments, the reducing agent is zinc, zirconium, vanadium, niobium, molybdenum, tungsten, aluminum, magnesium, potassium, zinc-copper couple, alkali and alkali earth metals, butylium, lithium, or lithium aluminum hydride. In some embodiments, the zinc is zinc powder.

In some embodiments, the compound of Formula (S2) is Compound S4:

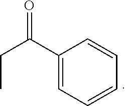

In some embodiments, the method further comprises a first solution made by combining the reducing agent and an organic solvent under an inert atmosphere at a first temperature. In some embodiments, the organic solvent is THF. In some embodiments, the inert atmosphere is a N₂ atmosphere. In some embodiments, the first temperature is between 15° to 30° C. In some embodiments, the method further comprises cooling the first solution to a second temperature and adding the metal catalyst. In some embodiments, the second temperature is between −10° to 5° C. In some embodiments, the method further comprises maintaining the first solution at a third temperature during the addition of the metal catalyst. In some embodiments, the third temperature is less than 20° C. In some embodiments, the method further comprises heating the first solution to a fourth temperature for 1 hour, 1.5 hours, 2 hours, 2.5 hours, or 3 hours. In some embodiments, the fourth temperature is between 50° to 70° C. In some embodiments, the method further comprises a second solution made by adding the compound of Formula (S2) and the compound of Formula (V) to an organic solvent. In some embodiments, the organic solvent is THF. In some embodiments, the method further comprises heating the second solution to between 50° to 70° C. In some embodiments, the method further comprises a third solution made by adding the second solution to the first solution over 1 hour, 1.5 hours, or 2 hours. In some embodiments, the method further comprises stirring the third solution at between 50° to 70° C. for 7 hours, 8 hours, 9 hours, or 10 hours. In some embodiments, the method further comprises distilling, washing, filtering, separating, extracting, precipitating, collecting, or a combination thereof, the compound of Formula (I) with an organic solvent, an acid, or an aqueous solution. In some embodiments, the organic solvent is MeTHF. In some embodiments, the washing comprises an organic solvent, an acid, a base, a solvent mixture, or a combination thereof. In some embodiments, the organic solvent is MeTHF, acetonitrile, acetone, or a combination thereof. In some embodiments, the acid is between 2N to 12 N HCl. In some embodiments, the aqueous solution is 30-35% HCl, between 10% to 60% NaCl, or between 10% to 60% K₂CO₃.

In some embodiments, the compound of Formula (V) is Compound 3:

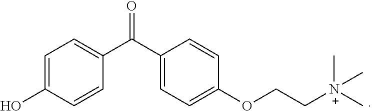 [SMILES: C[N+](C)(C)CCOc1ccc(C(=O)c2ccc(O)cc2)cc1]

In some embodiments, the compound of Formula (I) is Compounds 5a and 5b:

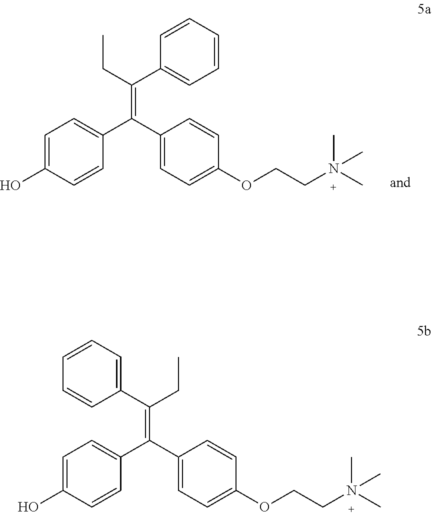

In some embodiments, the method comprises producing a compound of Formula (V) by functionalizing a compound of Formula (S1):

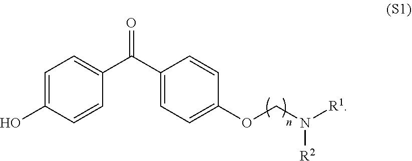

In some embodiments, the method comprises producing a compound of Formula (V) by alkylating a compound of Formula (S1):

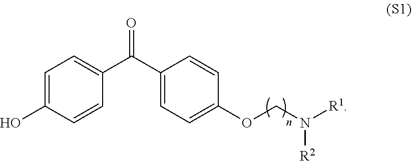

In some embodiments, producing a compound of Formula (V) by methylating a compound of Formula (S1):

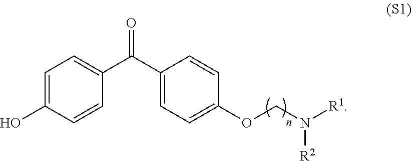

In some embodiments, the nitrogen of Formula (S1) is methylated by the methylating agent.

### Methods of Making Formula (III)

Methods of making compounds of Formula (III) may include following a synthetic reaction scheme as illustrated in SCHEME 2. SCHEME 2 may also be used for the synthesis of Compound 4a, Compound 4b, Compound 5a, Compound 5b, Compound 6A, Compound 6B, or a combination thereof.

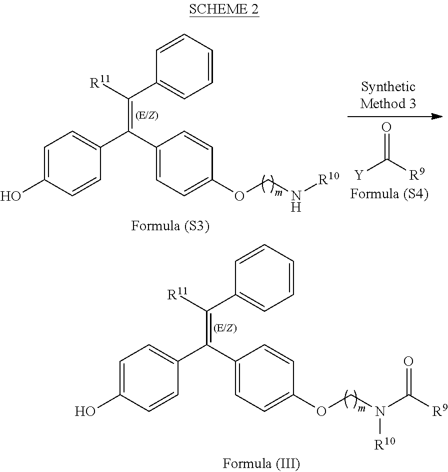

The present disclosure provides a method comprising coupling a compound of Formula (S3):

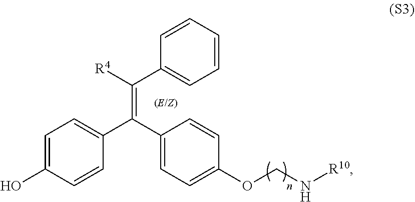

and a compound of Formula (S4):

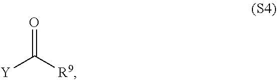

wherein Y is a leaving group,
thereby producing a compound of Formula (III):

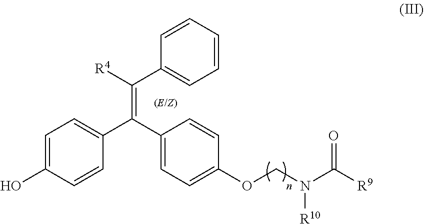

or a pharmaceutically acceptable salt, solvate, or tautomer thereof,
wherein:
   R⁹ is hydrogen, hydroxyl, C₁-C₆ alkyl substituted with 0 to 3 instances of R¹¹, C₂-C₆ alkenyl substituted with 0 to 3 instances of R¹¹, or C₂-C₆ alkynyl substituted with 0 to 3 instances of R¹¹;
   R¹⁰ is hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   R⁴ is C₁-C₆ alkyl substituted with 0 to 3 instances of R⁸, C₂-C₆ alkenyl substituted with 0 to 3 instances of R⁸, or C₂-C₆ alkynyl substituted with 0 to 3 instances of R⁸;
   each instance of R¹¹ is independently halogen, hydroxyl, C₁-C₃ alkoxy, C₁-C₃ alkylthio, C₁-C₃ alkyl, or C₃-C₈ cycloalkyl, aryl, or —C(═O)OCH₃;
   each instance of R⁵ is independently halogen, hydroxyl, C₁-C₃ alkoxy, C₁-C₃ alkylthio, C₁-C₃ alkyl, —C(═O)OH, or —C(═O)OCH₃;
   each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl;
   each instance of R⁸ is independently halogen, hydroxyl, C₁-C₃ alkoxy, C₁-C₃ alkylthio, C₁-C₃ alkyl, or C₃-C₅ cycloalkyl; and
n is an integer from 1 to 10.

In some embodiments, Y is an alkoxy, a hydroxy, an activated hydroxyl, a halogen, or an ester. In some embodiments, the compound of Formula (S4) is ethyl acetate. In some embodiments, the compound of Formula (S3) is Compound S3a:

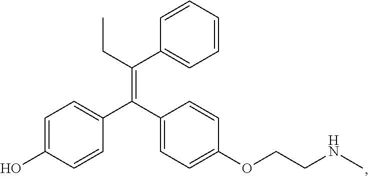 [SMILES: CC/C(=C(\c1ccc(O)cc1)c1ccc(OCCNC)cc1)c1ccccc1]

or a pharmaceutically acceptable salt, solvate, or tautomer thereof. In some embodiments, the compound of Formula (S3) is Compound S3b:

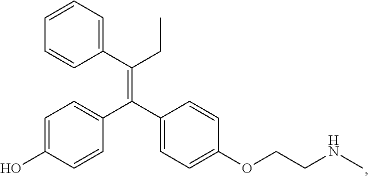 [SMILES: CC/C(=C(/c1ccc(O)cc1)c1ccc(OCCNC)cc1)c1ccccc1]

or a pharmaceutically acceptable salt, solvate, or tautomer thereof.

In some embodiments, the method is performed in an organic solvent. In some embodiments, the organic solvent is an aprotic solvent. In some embodiments, the organic solvent comprises ethyl acetate. In some embodiments, the method further comprises the addition of an acid. In some embodiments, the acid comprises between 2 to 12 N of the acid. In some embodiments, the acid comprises HCl. In some embodiments, the method comprises maintaining the coupling at a temperature of between 0 to 20° C., between −5 to 10° C., or between −10 to 10° C. during the addition of the acid. In some embodiments, the method further comprises heating the coupling to an elevated temperature. In some embodiments, the elevated temperature is between 50 to 60° C., between 55 to 65° C., or between 60 to 70° C. In some embodiments, the method further comprises stirring the solution for between 5 to 15 hours, between 8 to 12 hours, or between 9 to 11 hours.

In some embodiments, the method further comprises washing, purifying, or separating the compound of Formula (VI) using a base, an aqueous solution, an alcohol, or an organic solvent. In some embodiments, the base is sodium hydroxide. In some embodiments, the aqueous solution is a sodium chloride solution. In some embodiments, the sodium chloride solution is between 5 to 25% NaCl, between 10 to 30% NaCl, or between 15 to 25% NaCl. In some embodiments, the alcohol is ethanol, 2-propanol, or isopropyl alcohol. In some embodiments, the organic solvent is ethyl acetate or acetone. In some embodiments, adding a base to the coupling increasing the pH to a basic pH. In some embodiments, the base comprises between 2N to 12N of the base. In some embodiments, the base is sodium hydroxide. In some embodiments, the basic pH is between 10 to 12.

### Methods of Making Formula (V)

Methods of making compounds of Formula (V) may include following a synthetic reaction scheme as illustrated in SCHEME 3. SCHEME 3 may also be used for the synthesis of Compounds 7a and 7b.

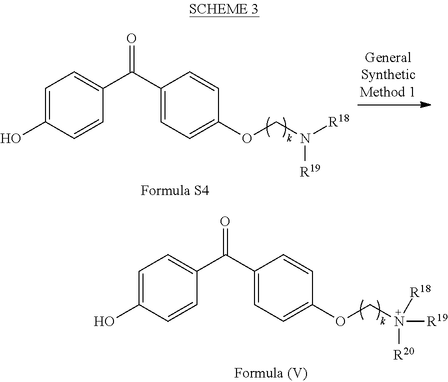

The present disclosure provides a method for synthesizing compounds described herein, the method comprising coupling R³ to a compound of Formula (S4):

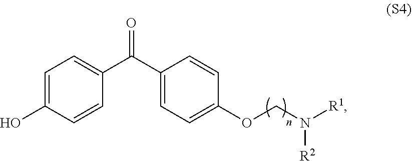

thereby producing the compound of Formula (V):

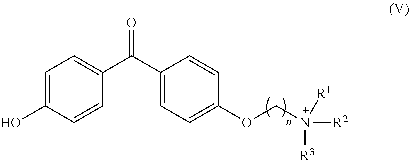

or a pharmaceutically acceptable salt, solvate, or tautomer thereof,
wherein:
   R¹, R², R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R⁵ is independently halogen, hydroxyl, C₁-C₃ alkoxy, C₁-C₃ alkylthio, C₁-C₃ alkyl, —C(═O)OH, or —C(═O)OCH₃;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
n is an integer from 1 to 10.

In some embodiments, the coupling comprises alkylating the compound of Formula (S4):

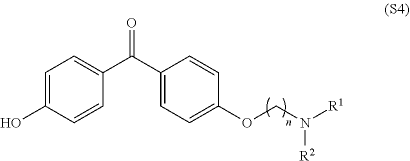

with R³.

In some embodiments, the coupling comprises methylating the compound of Formula (S4):

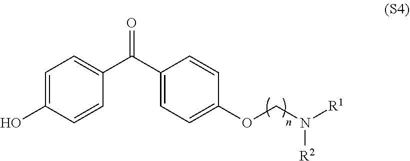

with a methylating agent.

In some embodiments, the nitrogen of Formula (S4) is methylated by the methylating agent.

In some embodiments, the methylating agent is 1-chloroethyl chloroformate. In some embodiments, the coupling comprises an organic solvent. In some embodiments, the organic solvent is a solvent mixture. In some embodiments, the solvent mixture comprises tetrahydrofuran and N-ethyldiisopropylamine. In some embodiments, the method further comprises incubating the coupling under an inert atmosphere. In some embodiments, the inert atmosphere is a N₂ atmosphere. In some embodiments, the method further comprises heating the coupling to between 30 to 70° C., between 40 to 70° C., between 50 to 70° C., or between 55 to 65°. In some embodiments, the method further comprises heating the coupling to an elevated temperature for between 8 to 16 hours, between 10 to 14 hours, or between 9 to 11 hours. In some embodiments, the elevated temperature is a boiling point of the organic solvent. In some embodiments, the method further comprises adding an alcohol to the coupling. In some embodiments, the alcohol is methanol. In some embodiments, the method further comprises heating the coupling to between 30 to 70° C., between 40 to 70° C., between 50 to 70° C., or between 55 to 65° C. and distilling the coupling to a reduced volume. In some embodiments, the reduced volume to total volume ratio is 1:3, 1:5, or 1:10. In some embodiments, the method further comprises adding an acid. In some embodiments, the acid comprises 2N to 12N of the acid. In some embodiments, the acid is hydrochloric acid. In some embodiments, the method further comprises heating the coupling to between 30 to 70° C., between 40 to 70° C., between 50 to 70° C., or between 55 to 65° C. for between 2 to 20 hours, between 6 to 18 hours, or between 8 to 16 hours.

In some embodiments, the method further comprises distilling, washing, purifying, or separating the compound of Formula (V) using an acid, a base, an aqueous solution, an alcohol, or an organic solvent. In some embodiments, the aqueous solution comprises water. In some embodiments, the base comprises 2N to 12N of the base. In some embodiments, the base is sodium hydroxide. In some embodiments, the organic solvent is ethyl acetate. In some embodiments, the acid comprises 2N to 12N of the acid. In some embodiments, the acid is hydrochloric acid.

### Methods of Making Formula (VI)

Methods of making compounds of Formula (VI) may include following a synthetic reaction scheme as illustrated in SCHEME 4.

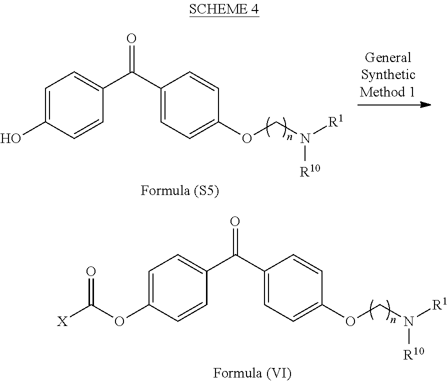

The present disclosure provides a method for synthesizing compounds described herein, the method comprising coupling a compound of Formula (S5):

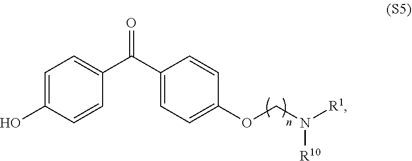

to a compound of Y²—C(═O)—Y³, wherein Y² and Y³ are each independently a leaving group, and a nucleophile, thereby producing the compound of Formula (VI):

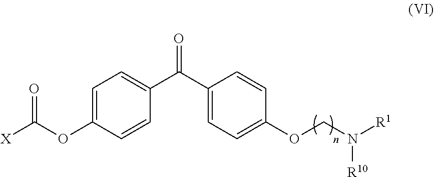

or a pharmaceutically acceptable salt, solvate, or tautomer thereof,
wherein:
   R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵,
   —C(═O)OR⁶, —C(═O)NR⁶R⁷, or —C(═O)SR⁶;
   R¹⁰ is hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   each instance of R⁵ is independently halogen, hydroxyl, C₁-C₃ alkoxy, C₁-C₃ alkylthio, C₁-C₃ alkyl, —C(═O)OH, or —C(═O)OCH₃;
   each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl;
   X is C₁-C₆ alkyl, —OR¹², —SR¹² or —NR¹²R¹³;
   R¹² and R¹³ are each independently hydrogen or C₁-C₃ alkyl substituted with 0 or 1 instance of halogen; and
   n is an integer from 1 to 10.

In some embodiments, the leaving group is a halogen, —OR⁶, —SR⁶ or —NR⁶R⁷. In some embodiments, the nucleophile is a C₁-C₆ alcohol, a C₁-C₆ thiol, a C₁-C₆ primary amine, or a C₁-C₆ secondary amine. In some embodiments, the coupling comprises an organic solvent. In some embodiments, the organic solvent is a solvent mixture. In some embodiments, the solvent mixture comprises tetrahydrofuran and N-ethyldiisopropylamine. In some embodiments, the method further comprises incubating the coupling under an inert atmosphere. In some embodiments, the inert atmosphere is a N₂ atmosphere. In some embodiments, the coupling comprises a temperature of at least 30° C. In some embodiments, the coupling comprises a temperature of between 30 to 70° C., between 40 to 70° C., between 50 to 70° C., or between 55 to 65° C. In some embodiments, the coupling comprises heating the compound of Formula (S5) for between 8 to 16 hours, between 10 to 14 hours, or between 9 to 11 hours. In some embodiments, the method further comprises adding an alcohol to the coupling. In some embodiments, the alcohol is methanol. In some embodiments, the method further comprises heating the coupling to between 30 to 70° C., between 40 to 70° C., between 50 to 70° C., or between 55 to 65° C. and distilling the coupling to a reduced volume. In some embodiments, the reduced volume to total volume ratio is 1:3, 1:5, or 1:10. In some embodiments, the method further comprises comprising adding an acid. In some embodiments, the acid comprises 2N to 12N of the acid. In some embodiments, the acid is hydrochloric acid. In some embodiments, the method further comprises heating the coupling to between 30 to 70° C., between 40 to 70° C., between 50 to 70° C., or between 55 to 65° C. for between 2 to 20 hours, between 6 to 18 hours, or between 8 to 16 hours. In some embodiments, the method further comprises distilling, washing, purifying, or separating the compound of Formula (VI) using an acid, a base, an aqueous solution, an alcohol, or an organic solvent. In some embodiments, the aqueous solution comprises water. In some embodiments, the base comprises 2N to 12N of the base. In some embodiments, the base is sodium hydroxide. In some embodiments, the organic solvent is ethyl acetate. In some embodiments, the acid comprises 2N to 12N of the acid. In some embodiments, the acid is hydrochloric acid.

### Methods of Making Formula (VII)

Methods of making compounds of Formula (VII) may include following a synthetic reaction scheme as illustrated in SCHEME 5.

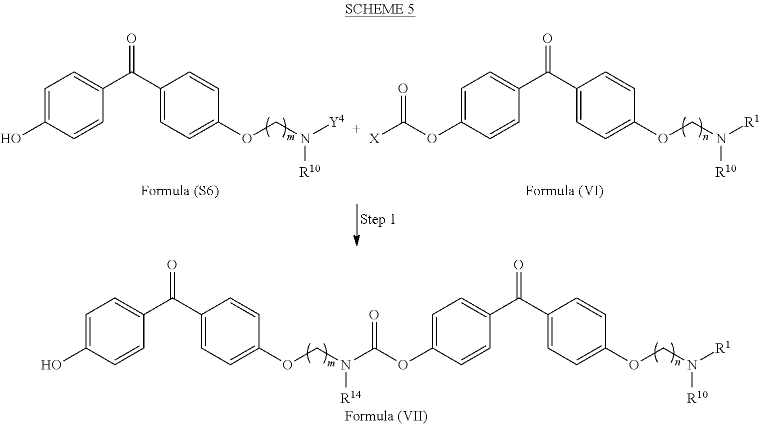

The present disclosure provides a method for synthesizing compounds described herein, the method comprising coupling a compound of Formula (S6):

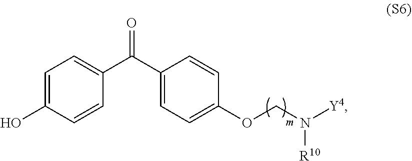

to a compound of Formula (VI):

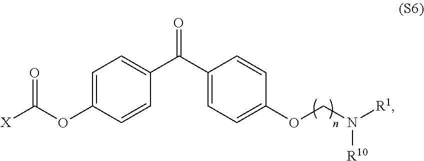

thereby producing a compound of Formula (VII):

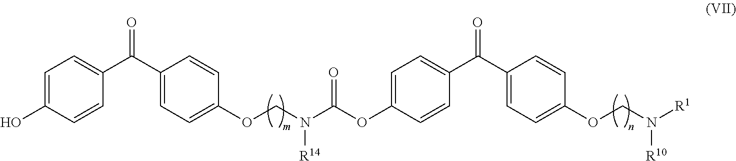

or a pharmaceutically acceptable salt, solvate, or tautomer thereof,
wherein:
   R¹ is C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵,
   —C(═O)OR⁶, —C(═O)NR⁶R⁷, or —C(═O)SR⁶;
   R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   each instance of R⁵ is independently halogen, hydroxyl, C₁-C₃ alkoxy, C₁-C₃ alkylthio, C₁-C₃ alkyl, —C(═O)OH, or —C(═O)OCH₃;
   each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
   m and n are each independently integers from 1 to 10.

In some embodiments, the coupling comprises an organic solvent. In some embodiments, the organic solvent is a solvent mixture. In some embodiments, the solvent mixture comprises tetrahydrofuran and N-ethyldiisopropylamine. In some embodiments, the method further comprises incubating the coupling under an inert atmosphere. In some embodiments, the inert atmosphere is a N₂ atmosphere. In some embodiments, the method further comprises heating the coupling to between 30 to 70° C., between 40 to 70° C., between 50 to 70° C., or between 55 to 65° C. In some embodiments, the method further comprises heating the coupling to an elevated temperature for between 8 to 16 hours, between 10 to 14 hours, or between 9 to 11 hours. In some embodiments, the elevated temperature is a boiling point of the organic solvent. In some embodiments, the method further comprises adding an alcohol to the coupling. In some embodiments, the alcohol is methanol. In some embodiments, the method further comprises heating the coupling to an elevated temperature and distilling the coupling to a reduced volume. In some embodiments, the elevated temperature is between 30 to 70° C., between 40 to 70° C., between 50 to 70° C., or between 55 to 65° C. In some embodiments, the reduced volume to total volume ratio is 1:3, 1:5, or 1:10. In some embodiments, the method further comprises adding an acid. In some embodiments, the acid comprises 2N to 12N of the acid. In some embodiments, the acid is hydrochloric acid. In some embodiments, the method further comprises heating the coupling to between 30 to 70° C., between 40 to 70° C., between 50 to 70° C., or between 55 to 65° C. for between 2 to 20 hours, between 6 to 18 hours, or between 8 to 16 hours.

In some embodiments, the method further comprises distilling, washing, purifying, or separating the compound of Formula (VII) using an acid, a base, an aqueous solution, an alcohol, or an organic solvent. In some embodiments, the aqueous solution comprises water. In some embodiments, the base comprises 2N to 12N of the base. In some embodiments, the base is sodium hydroxide. In some embodiments, the organic solvent is ethyl acetate. In some embodiments, the acid comprises 2N to 12N of the acid. In some embodiments, the acid is hydrochloric acid.

### Methods of Making Formula (VIII)

Methods of making compounds of Formula (VIII) may include following a synthetic reaction scheme as illustrated in SCHEME

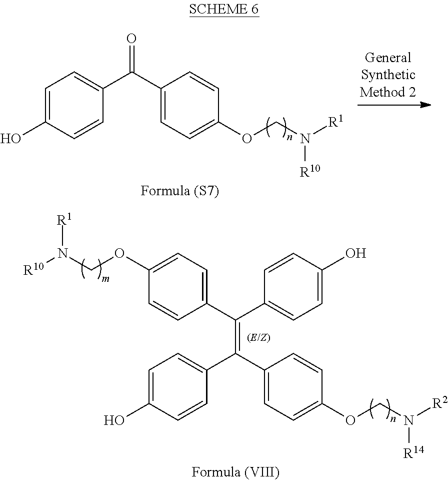

The present disclosure provides a method for synthesizing compounds described herein, the method comprising coupling two compounds of Formula (S7):

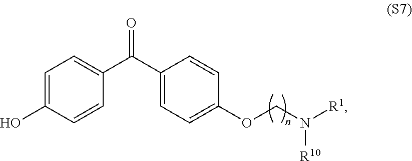

thereby producing the compound of Formula (VIII):

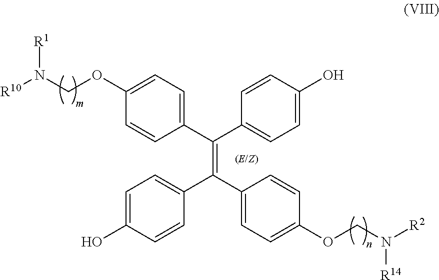

or a pharmaceutically acceptable salt, solvate, or tautomer thereof,
wherein:
   R¹ and R² are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   R¹⁰ and R¹⁴ are each independently hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
   each instance of R⁵ is independently halogen, hydroxyl, C₁-C₃ alkoxy, C₁-C₃ alkylthio, C₁-C₃ alkyl, —C(═O)OH, or —C(═O)OCH₃;
   each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl; and
   m and n are each independently integers from 1 to 10.

In some embodiments, the coupling comprises an olefination. In some embodiments, the coupling comprises a McMurry reaction. In some embodiments, the method further comprises activating the compound of Formula (S7):

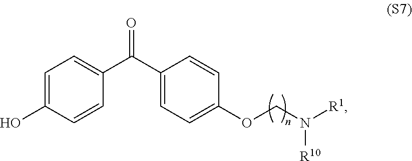

with a metal catalyst.

In some embodiments, generating the metal catalyst by combining a metal salt with a reducing agent. In some embodiments, the metal catalyst is titanium, zirconium, vanadium, niobium, molybdenum, tungsten, aluminum, or zinc. In some embodiments, the metal catalyst is titanium. In some embodiments, the metal catalyst is a titanium halide. In some embodiments, the metal catalyst is TiCl₃ or TiCl₄. In some embodiments, the reducing agent is zinc, zirconium, vanadium, niobium, molybdenum, tungsten, aluminum, magnesium, potassium, zinc-copper couple, alkali and alkali earth metals, butylium, lithium, or lithium aluminum hydride.

In some embodiments, the method further comprises a first solution made by combining the reducing agent and an organic solvent under an inert atmosphere at a first temperature. In some embodiments, the organic solvent is THF. In some embodiments, the inert atmosphere is a N₂ atmosphere. In some embodiments, the first temperature is between 15° to 30° C. In some embodiments, the method further comprises cooling the first solution to a second temperature and adding the metal catalyst. In some embodiments, the second temperature is between −10° to 5° C. In some embodiments, the method further comprises maintaining the first solution at a third temperature during the addition of the metal catalyst. In some embodiments, the third temperature is less than 20° C. In some embodiments, the method further comprises heating the first solution to a fourth temperature for 1 hour, 1.5 hours, 2 hours, 2.5 hours, or 3 hours. In some embodiments, the fourth temperature is between 50° to 70° C. In some embodiments, the method further comprises a second solution made by adding the compound of Formula (S7) to an organic solvent. In some embodiments, the organic solvent is THF. In some embodiments, the method further comprises heating the second solution to between 50° to 70° C. In some embodiments, the method further comprises a third solution made by adding the second solution to the first solution over 1 hour, 1.5 hours, or 2 hours. In some embodiments, the method further comprises stirring the third solution at between 50° to 70° C. for 7 hours, 8 hours, 9 hours, or 10 hours.

In some embodiments, the method further comprises distilling, washing, filtering, separating, extracting, precipitating, collecting, or a combination thereof, the compound of Formula (VIII) with an organic solvent, an acid, or an aqueous solution. In some embodiments, the distilling comprises an organic solvent. In some embodiments, the organic solvent is MeTHF. In some embodiments, the washing comprises an organic solvent, an acid, a base, a solvent mixture, or a combination thereof. In some embodiments, the organic solvent is MeTHF, acetonitrile, acetone, or a combination thereof. In some embodiments, the acid is between 2N to 12 N HCl. In some embodiments, the aqueous solution is 30-35% HCl, between 10% to 60% NaCl, or between 10% to 60% K₂CO₃.

### Methods of Making Formula (IX)

In various aspects, the present disclosure provides a method for producing a compound of Formula (IX), the method comprising coupling a compound of Formula (S8):

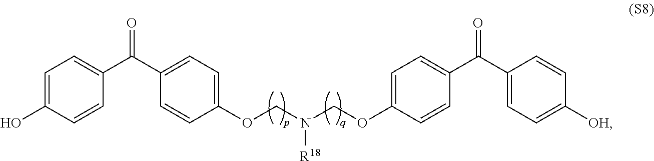

to compounds of Formulae (S9) and (S10):

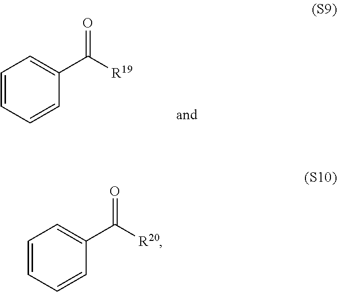

thereby producing the compound of Formula (IX).

In some aspects, the method further comprises activating the compounds of Formulae (S8), (S9), and (S10) with a metal catalyst. In some aspects, the method comprises generating the metal catalyst by combining a metal salt with a reducing agent. In some aspects, the metal catalyst comprises titanium, zirconium, vanadium, niobium, molybdenum, tungsten, aluminum, or zinc. In some aspects, the metal catalyst comprises titanium. In some aspects, the metal salt comprises a titanium halide. In some aspects, the titanium halide comprises TiCl₃ or TiCl₄. In some aspects, the reducing agent comprises zinc, zirconium, vanadium, niobium, molybdenum, tungsten, aluminum, magnesium, potassium, zinc-copper couple, alkali and alkali earth metals, butylium, lithium, or lithium aluminum hydride. In some aspects, the zinc comprises zinc powder.

In some aspects, the compounds of Formulae (S9) and (S10) are identical. In some aspects, the compound of Formula (S9) is Compound S4:

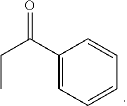

In some aspects, the compound of Formula (S10) is Compound S4:

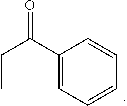

In some aspects, the coupling is performed in an organic solvent. In some aspects, the organic solvent is THF. In some aspects, the coupling is performed under reflux. In some aspects, the method further comprises adding an acid to the compounds of Formulae (S8), (S9), and S(10). In some aspects, the acid generates a biphasic mixture. In some aspects, the method further comprises heating the biphasic mixture to a temperature of between 3° and 70° C.

In some aspects, the coupling comprises an olefination reaction. In some aspects, the coupling comprises a McMurry reaction.

In some aspects, the method further comprises generating the compound of Formula (S8) by coupling compounds of Formulae (S11) and (S12):

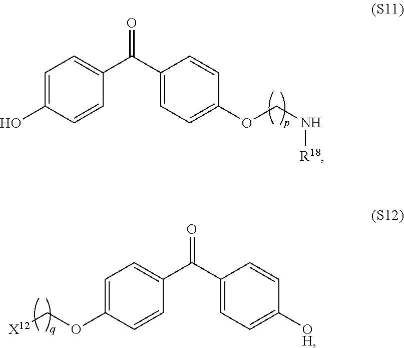

wherein X¹² is a halogen.

In some aspects, X¹² is Br. In some aspects, coupling the compound of Formulae (S11) and (S12) comprises an organic solvent. In some aspects, the organic solvent comprises a solvent mixture. In some aspects, the solvent mixture comprises ethanol and N-ethyldiisopropylamine. In some aspects, coupling the compound of Formulae (S11) and (S12) comprises reflux, vacuum distillation, or a combination thereof.

### Methods for Isomeric Enrichment

Compounds of the current disclosure may exist in an isomeric mixture. An isomeric mixture may contain both the (E) and (Z) isomers of a compound disclosed herein. Synthetic and purification methods may be used to increase the isomeric ratio. In some embodiments, the mixture of isomers may be enriched in the (Z)-isomeric form. In some embodiments, the mixture of isomers may be enriched in the (E)-isomeric form.

The methods of the present disclosure may be used for generating an isomerically enriched mixture of a compound disclosed herein, the method comprising:
providing a non-isomerically enriched mixture of the compound comprising a (Z) isomer and an (E) isomer of the compound,
isomerizing the (E)-isomer of the compound into the (Z)-isomer of the compound, thereby generating an isomerically enriched mixture, and
collecting the isomerically enriched mixture.

Isomerizing a compound of the present disclosure refers to the process in which a compound is transformed into an isomer with a different chemical structure. For example, isomerizing a compound of the present disclosure may involve transforming the compound from an (E)-isomer of the compound to a (Z)-isomer of the compound.

In some embodiments, the isomerically enriched mixture comprises at least 50%, at least 60%, at least 65%, at least 70%, at least 72.5%, at least 75%, at least 77.5%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.75%, or at least 99.9% of the (Z)-isomer of a compound described herein.

In some embodiments, the collecting comprises precipitating the (Z)-isomer, distilling the (Z)-isomer, extracting the (Z)-isomer, chromatographically separating the (Z)-isomer, washing the (Z)-isomer, drying the (Z)-isomer, crystallizing the (Z)-isomer, or a combination thereof. In some embodiments, the crystallizing is performed at a temperature of between −20° to 20° C. In some embodiments, the non-isomerically enriched mixture is heated to a temperature of between 40° to 80° C. prior to the separating. In some embodiments, the method comprises crystallizing the (Z)-isomer from an organic solvent. In some embodiments, the organic solvent is an aprotic solvent. In some embodiments, the organic solvent comprises ethyl acetate. In some embodiments, the organic solvent comprises an acid. In some embodiments, the organic solvent comprises between 2 to 12 N of the acid. In some embodiments, the acid comprises HCl.

### Methods of Use

The present disclosure may provide a method for delivering a compound as disclosed herein or a pharmaceutical composition as disclosed herein to a subject, the method comprising administering the compound or the pharmaceutical composition to a subject. In some embodiments, the method further comprises treating a condition in the subject. In some embodiments, the condition is cancer. Compounds of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof disclosed herein, and compositions comprising them may be used in the manufacture of medicaments for use in the treatment of a subject in need thereof, for example, subjects having or at risk of having a hormone-dependent breast disorder, a hormone-dependent reproductive tract disorder, or both. In some embodiments, a hormone-dependent breast disorder, a hormone-dependent reproductive tract disorder, or both is treated by a compound with aromatase inhibition activity. In some embodiments, compounds disclosed herein have aromatase inhibition activity. In some embodiments, compounds disclosed herein have affinity to an estrogen receptor. In some embodiments, an active pharmaceutical ingredient (API) disclosed herein (e.g., a compound of any one of Formula (I)-Formula (VIII) or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) has aromatase inhibition activity. In some embodiments, a hormone-dependent breast disorder, a hormone-dependent reproductive tract disorder, or both is treated by a compound with affinity to an estrogen receptor. In some embodiments, an active pharmaceutical ingredient (API) disclosed herein (e.g., a compound of any one of Formula (I)-Formula (VIII) or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) may have affinity to an estrogen receptor.

The compositions of the present disclosure may be used as a primary therapy, as a part of a neo-adjuvant therapy (to primary therapy), or as part of adjuvant therapy regimen, where the intention is to ameliorate or cure a subject having or at risk of having a hormone-dependent breast disorder, hormone-dependent reproductive tract disorder, or both.

In certain embodiments, the disorder is a hormone-dependent breast disorder. In other embodiments, the disorder is hormone-dependent reproductive tract disorder. In still other embodiments, the subject has both a hormone-dependent breast disorder and a hormone-dependent reproductive tract disorder. In some embodiments, the hormone dependent disorder is a benign breast disorder, hyperplasia, atypia, atypical ductal hyperplasia, atypical lobular hyperplasia, increased breast density, gynecomastia, DCIS, LCIS, breast cancer, endometrial cancer, ovarian cancer, uterine cancer, cervical cancer, vaginal cancer or vulvar cancer.

In some embodiments, the breast disorder is increased breast density. For example, the breast disorder is a class B (formerly Class II), class C (formerly class III) or class D (formerly class IV) breast density.

In some embodiments, the hormone-dependent breast disorder or hormone-dependent reproductive tract disorder is precocious puberty. In other embodiments, the hormone-dependent breast disorder or hormone-dependent reproductive tract disorder is McCune-Albright Syndrome.

In some embodiments, the breast disorder is gynecomastia. In some embodiments, gynecomastia is presented secondarily to an underlying disease. Accordingly, in some embodiments, the subject also has underlying disease selected from the group consisting of prostate cancer, cirrhosis and liver disease, male hypogonadism, hyperthyroidism, renal failure and in patients undergoing hemodialysis, or type I diabetes mellitus. In certain embodiments, the subject has prostate cancer as the underlying disease, wherein the subject has or is at risk of having gynecomastia.

In certain embodiments, the breast cancer is DCIS, LCIS, ILC, IDC, MIC, inflammatory breast cancer, ER-positive (ER+) breast cancer, HER2+ breast cancer, adenoid cystic (adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. In at least one embodiment, a single breast cancer tumor may be a combination of the foregoing or be a mixture of invasive and in situ cancer.

The present disclosure contemplates the use of the compounds and compositions disclosed herein at various stages in tumor development and progression, including the treatment of advanced and/or aggressive neoplasms, i.e., overt disease in a subject that is not amenable to cure by local modalities of treatment such as surgery or radiotherapy, metastatic disease, or locally advanced disease. Accordingly, in some embodiments, the breast cancer is a pre-cancer, an early-stage cancer, a non-metastatic cancer, a pre-metastatic cancer, or a locally advanced cancer. In at least one embodiment, the breast disorder is metastatic cancer. In some embodiments, the subject further has prostate cancer.

Provided herein in certain embodiments are patient populations for whom the pharmaceutical compositions are particularly useful. The compositions of the present disclosure are also particularly important in the treatment of tamoxifen-refractory subjects with hormone-dependent breast disorders or hormone-dependent reproductive tract disorders. Accordingly, in some embodiments, the compositions disclosed herein are useful for the treatment of tamoxifen refractory or tamoxifen resistant subjects having or at risk of having hormone-dependent breast disorders, hormone-dependent reproductive tract disorders, or both.

Drug interactions between tamoxifen and selective serotonin reuptake inhibitors (SSRI) drugs like Prozac and Paxil (paroxetine) may occur and may be detrimental to breast cancer subjects. The SSRI drugs reduce or stop liver metabolism of tamoxifen to endoxifen in subjects on SSRI drugs. Thus, provided herein in certain embodiments are patient populations being treated or to be treated with SSRI drugs that would be benefitted by treatment with compositions of the present disclosure.

It also advantageous for subjects being treated or to be treated with antidepressant drugs such as SSRI drugs such as citalopram (Celexa), escitalopram (Lexapro), fluoxetine (Prozac), paroxetine (Paxil, Pexeva), sertraline (Zoloft), vilazodone (Viibryd) and the like, for example, a subject having or likely to have depression.

In an aspect, the present disclosure contemplates a method of treating a subject having or at risk of having a hormone-dependent breast disorder, a hormone-dependent reproductive tract disorder, or both, the method comprising resection of breast tissue of the subject or administering radiotherapy to the subject and administering an oral composition comprising one or more compounds disclosed herein (e.g., a compound of any one of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof). In another aspect, the present disclosure contemplates a method of treating a subject having or at risk of having a hormone-dependent breast disorder, a hormone-dependent reproductive tract disorder, or both, the method comprising administering an oral composition disclosed herein prior to resection of breast tissue of the subject or administering radiotherapy to the subject.

Dosage to be administered to a subject will be usually in a unit dosage form. Examples of ranges for comprising one or more compounds disclosed herein (e.g., a compound of any one of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) in each dosage unit form are from 0.01 mg to 200 mg. Dosage shall generally be an effective amount and equivalent, on a molar basis, of the pharmacologically of a composition comprising one or more compounds disclosed herein (e.g., a compound of any one of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) produced by a dosage formulation upon metabolic release of the composition to achieve its desired pharmacological and physiological effects. In some embodiments, the compositions comprising one or more compounds disclosed herein (e.g., a compound of any one of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) are administered to the subject at a dose of 0.01 mg to 200.0 mg. In other embodiments, the oral compositions comprising one or more compounds disclosed herein (e.g., a compound of any one of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) are administered to the subject at a dose of 1 mg to 200.0 mg. In some embodiments, the oral compositions comprising one or more compounds disclosed herein (e.g., a compound of any one of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) are administered to the subject at a dose of 0.01 mg, 0.05 mg, 0.1 mg, 0.25 mg, 0.5 mg, 0.75 mg, 1.0 mg, 1.5 mg, 2.0 mg, 4.0 mg, 5 mg, 6 mg, 7 mg, 8 mg, 9 mg, 10 mg, 12.5 mg, 15 mg, 17.5 mg, 20 mg, 40 mg, 50 mg, 100 mg or 200 mg per unit dose. In some embodiments, the oral compositions comprising one or more compounds disclosed herein (e.g., a compound of any one of Formula (I)-Formula (VIII), or a pharmaceutically acceptable salt, solvate, or tautomer thereof, or Compound 1, Compound 2, Compound 3, Compound 4a, Compound 4b, Compound 5a, Compound 5b, Compound 6a, Compound 6b, Compound 7a, Compound 7b, Compound 8, Compound 9a, Compound 9b, Compound 9c, or a pharmaceutically acceptable salt, solvate, or tautomer thereof) are administered to the subject at a dose ranging from 0.01 mg-0.05 mg, from 0.01 mg-0.1 mg, from 0.01 mg-0.2 mg, from 0.01 mg-0.3 mg, from 0.01 mg-0.4 mg, from 0.01 mg-0.5 mg, from 0.01 mg-1 mg, from 0.01 mg-5 mg, from 0.01 mg-10 mg, from 0.1 mg-0.2 mg, from 0.1 mg-0.3 mg, from 0.1 mg-0.4 mg, from 0.1 mg-0.5 mg, from 0.1 mg-1 mg, from 0.1 mg-5 mg, from 0.1 mg-10 mg, from 0.5 mg-1 mg, from 0.5 mg-1.5 mg from 0.5 mg-2 mg per unit dose, from 0.5 mg-2.5 mg, from 0.5 mg-3 mg, from 0.5 mg-4 mg, from 0.5 mg-5 mg, from 0.5 mg-10 mg, from 0.5 mg-20 mg, from 1 mg-5 mg, from 1 mg-7.5 mg, from 1 mg-10 mg, from 1 mg-15 mg, from 1 mg-20 mg, from 5 mg-20 mg, from 5 mg-30 mg, from 5 mg-40 mg, from 5 mg-50 mg, from 5 mg-100 mg, from 5 mg-200 mg, from 10 mg-50 mg, from 10 mg-100 mg, or from 10 mg-200 mg per unit dose.

Breast cancer growth rate studies have shown, using mammographic screening of subjects with breast cancer, that the breast cancer growth rate in the 25^{th} percentiles of women ages 50 to 59 indicate an unmet need for fast exposure of the subject to therapeutics (Weeden-Fekjaer et al. Breast Cancer Research 200810:R41). Rapid absorption and bioavailability of the anti-cancer therapeutics that can further reduce the cancer growth rate is highly desirable.

A healthcare professional, such as an attending physician, may adjust the dosing regimen based on the pharmacokinetic profile of the composition in the subject.

In one aspect, the compositions of the disclosure can be used alone or in a combination therapy. For example, compositions disclosed herein may be used in combination with one or more therapeutic agents as part of primary therapy, neoadjuvant therapy, or an adjuvant therapy. It is an aspect of the present disclosure that the compositions of the disclosure can be used in combination with other therapies such as surgery and radiation as neo-adjuvant or adjuvant therapy. Combinations of the compositions may act to improve the efficacy of the therapeutic agents, and therefore can be used to improve standard cancer therapies. For example, when a subject has prostate cancer and is on bicalutamide or enzalutamide therapy for the treatment of prostate cancer, the subject is likely to develop gynecomastia a result of the therapy. The compositions disclosed herein can be administered as a combination therapy to the subject having prostate cancer in order to prevent and/or treat gynecomastia. As another example, a subject with ER+/Her2+ positive breast cancer would be on a combination therapy with trastuzumab or other oncology drugs such as anti-neoplastics or immunotherapy, and a composition disclosed herein can be used to treat such a subject with ER+/Her2+ positive breast cancer. Accordingly, in some embodiments, the compositions further comprise bicalutamide, enzalutamide or anticancer drugs such as trastuzumab, antineoplastics such as capecitabine (Xeloda), carboplatin (Paraplatin), cisplatin (Platinol), cyclophosphamide (Neosar), docetaxel (Docefrez, Taxotere), doxorubicin (Adriamycin), pegylated liposomal doxorubicin (Doxil), epirubicin (Ellence), fluorouracil (5-FU, Adrucil), gemcitabine (Gemzar), methotrexate (multiple brand names), paclitaxel (Taxol), protein-bound paclitaxel (Abraxane), vinorelbine (Navelbine), eribulin (Halaven), ixabepilone (Ixempra), and ATP-cassette binding protein inhibitors.

In another aspect, a composition disclosed herein may comprise therapeutic agents that increase bioavailability of a compound of the current disclosure in a subject. P-glycoprotein (P-gp, ABCB1) is a highly efficient drug efflux pump expressed in brain, liver, and small intestine, but also in cancer cells, that affects pharmacokinetics and confers therapy resistance for many anticancer drugs. Accordingly, in some embodiments, the compositions further comprise inhibitors of ATP-binding cassette (ABC family) transporters, such as inhibitors of breast cancer resistance protein (BCRP protein) and P-gp. Several inhibitors of BCRP protein and P-Gp are known in the art. For example, inhibitors of BCRP protein include cyclosporine, omeprazole, pantoprazole, saquinavir, and tacrolimus.

Non-limiting examples of P-gp inhibitors include first generation inhibitors such as Verapamil, cyclosporin A, reserpine, quinidine, yohimbine, tamoxifen and toremifene, second generation inhibitors such as Dexverapamil, dexniguldipine, valspodar (PSC 833), and Dofequidar fumarate (MS-209), third generation P-gp inhibitors such as Cyclopropyldibenzosuberane zosuquidar (LY335979), laniquidar (R101933), mitotane (NSC-38721), biricodar (VX-710), elacridar (GF120918/GG918), ONT-093, tariquidar (XR9576), and HM30181 and anti-P-gp monoclonal antibodies such as MRK-16).

The present disclosure additionally provides for therapeutic kits containing one or more of the compositions for use in the treatment of a subject having or at risk of having a hormone-dependent breast disorder, a hormone-dependent reproductive tract disorder, or both. The kits of the present disclosure may include an oral composition disclosed herein, a sealed container for housing the composition, and instructions for use of the orally administered composition. In an aspect, the kits of the present disclosure can include a second therapeutic agent. Such a second therapeutic agent may be bicalutamide, enzalutamide or an anticancer drug such as trastuzumab, antineoplasitcs such as capecitabine (Xeloda), carboplatin (Paraplatin), cisplatin (Platinol), cyclophosphamide (Neosar), docetaxel (Docefrez, Taxotere), doxorubicin (Adriamycin), pegylated liposomal doxorubicin (Doxil), epirubicin (Ellence), fluorouracil (5-FU, Adrucil), gemcitabine (Gemzar), methotrexate (multiple brand names), paclitaxel (Taxol), protein-bound paclitaxel (Abraxane), vinorelbine (Navelbine), eribulin (Halaven), ixabepilone (Ixempra), and ATP-binding cassette (ABC transporter) inhibitors such as P-gp inhibitors.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21th Ed., Mack Publ. Co., Easton, Pa. (2005), incorporated herein by reference in its entirety.

The term “inhibit” or “inhibition of” means to reduce by a measurable amount, or to prevent entirely (e.g., 100% inhibition).

All terms, chemical names, expressions, and designations have their usual meanings which are well-known to those skilled in the art. When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individually or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

As used herein, the term “group” may refer to a reactive functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, the term “substituted” refers to a compound (e.g., an alkyl chain) wherein a hydrogen is replaced by another reactive functional group or atom, as described herein.

The following definitions are used, unless otherwise described: halogen is fluorine, chlorine, bromine, or iodine. Alkyl, alkoxy, alkenyl, alkynyl, etc. denote both straight and branched groups; but reference to an individual radical such as propyl embraces only the straight chain radical, a branched chain isomer such as isopropyl being specifically referred to.

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

Specific values listed below for radicals, substituents, and ranges, are for illustration only; they do not exclude other defined values or other values within defined ranges for the radicals and substituents.

Specifically, C₁-C₆ alkyl can be methyl, ethyl, propyl, isopropyl, butyl, iso-butyl, sec-butyl, pentyl, 3-pentyl, or hexyl; C₃-C₈ cycloalkyl can be cyclopropyl, cyclobutyl, cyclopentyl, or cyclohexyl; C₁-C₃ alkoxy can be methoxy, ethoxy, propoxy, or isopropoxy; C₂-C₆ alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; C₂-C₆ alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl;

The term “alkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkenyl” refers to an unsubstituted straight chain or branched, hydrocarbon having at least one carbon-carbon double bond and the indicated number of carbon atoms (e.g., “C₂-C₈ alkenyl” or “C₂-C₁₀” alkenyl have from 2 to 8 or 2 to 10 carbon atoms, respectively). When the number of carbon atoms is not indicated, the alkenyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “alkylthio” refers to an alkyl group, as defined herein, which is attached to a molecule via a sulfur atom. For example, alkythio groups include, but are not limited to thiomethyl, thioethyl, thio-n-propyl, thio-iso-propyl, and the like.

The term “cycloalkyl” refers to a cyclic, saturated, or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C₃₋₆ cycloalkyl” groups include, cyclopropyl, cyclobutyl, cyclopentyl, and cyclohexyl.

The term “heterocycle” and “heterocycloalkyl” refer to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl. The term “hydroxyl” refers to an —OH group.

All compounds disclosed herein are further understood to include all possible isotopes of atoms occurring in the compounds. Isotopes include those atoms having the same atomic number but different mass numbers. By way of example, and without limitation, isotopes of hydrogen include tritium and deuterium and isotopes of carbon include ¹¹C, ¹³C and ¹⁴C.

As used herein and in the claims, the terms “comprising,” “containing,” and “including” are inclusive, open-ended and do not exclude additional unrecited elements, compositional components or method steps. Accordingly, the terms “comprising” and “including” encompass the more restrictive terms “consisting of” and “consisting essentially of”

As used herein, the term “combination therapy” refers to the use of a composition described herein in combination with one or more additional treatment. Treatment in combination therapy can be any treatment such as any prophylactic agent, therapeutic agent (such as chemotherapy), radiotherapy, surgery and the like. The combination can refer to inclusion of a therapeutic or prophylactic agent in a same composition as a composition disclosed herein (for example, in the same capsule, tablet, ointment, etc.) or in separate compositions (for example, in 2 separate capsules). The separate compositions may be in a different dosage form. The use of the terms “combination therapy” and “in combination with” does not restrict the order in which a composition described herein and prophylactic and/or therapeutic agent and/or treatment are administered to a subject in need thereof. Compositions of the present disclosure can be administered prior to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year before), concomitant with, or subsequent to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year after) administration of one or more prophylactic and/or therapeutic agent and/or treatment to a subject in thereof. Combination therapy as used herein can also refer to treatment of a subject having a single disease or multiple diseases, for example, prostate cancer in men and gynecomastia.

As used herein, the term “test sample” means sample of blood obtained from a subject. It is to be understood that when blood sample is obtained from a subject, subject's blood is used for determining the subject's drug levels and/or other biomarkers that may be measured or tested. As used herein “plasma drug level” is used to refer to drug levels in the subject's test sample, whether the test is conducted on whole blood, plasma, or serum.

As used herein, the term “dosage form” means the form in which the compounds or compositions of the present disclosure are delivered to a patient.

As used herein, the term “pharmaceutically acceptable” or “pharmacologically acceptable” means materials, compositions, or vehicles that are compatible with other ingredients of the formulation and that they do not substantially produce adverse reactions, e.g., toxic, allergic, or immunological reactions, when administered to a subject. They may be approved by a regulatory agency, e.g., of the U.S. Federal or state government or listed in the U.S. pharmacopeia or other generally recognized pharmacopeia for use in animals, and more particularly in humans.

As used herein, the term “pharmaceutically acceptable carrier” or “carrier” means a pharmaceutically acceptable material, composition, or vehicle, such as a liquid or solid filler, diluent, excipient, solvent, or encapsulating material, involved in carrying or transporting one or more of the compounds of the present disclosure from one tissue, organ, or portion of the body or across the skin.

As used herein, the term “pharmaceutically acceptable salt” refers to any salt (e.g., obtained by reaction with an acid or a base) of a compound of the present disclosure that is physiologically tolerated in a subject (e.g., a mammal, and/or in vivo, ex-vivo, in vitro cells, tissues, or organs). A “salt” of a compound of the present disclosure may be derived from inorganic or organic acids and bases. Suitable anion salts include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide.

Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

For the purposes of this application, the salts of the compounds of the present disclosure are contemplated to be pharmaceutically acceptable for therapeutic uses. However, salts of acids and bases that are non-pharmaceutically acceptable may also be useful, for example, in the preparation or purification of a pharmaceutically acceptable compound.

As used herein, the term “pharmaceutical composition” means a combination of the active agent (e.g., an active pharmaceutical compound or ingredient, API) with a carrier, inert or active (e.g., a phospholipid), making the compositions especially suitable for diagnostic or therapeutic uses in vitro, in vivo, or ex vivo.

As used herein “primary therapy” refers to a first line of treatment upon initial diagnosis of a hormone-dependent breast disorder, a hormone-dependent reproductive tract disorder, or both in a subject. Exemplary primary therapies may involve surgery, a wide range of chemotherapies, and radiotherapy.

As used herein, the terms “subject,” “patient,” “participant,” and “individual,” may be used interchangeably herein and refer to a mammal such as a human. Mammals also include pet animals such as dogs, cats, laboratory animals, such as rats, mice, and farm animals such as cows and horses. Unless otherwise specified, a mammal may be of any gender or sex.

As used herein, the term “tamoxifen refractory” refers to a subject that has been dosed daily with tamoxifen for at least 2 days and has a level of plasma endoxifen of less than 30 nM (e.g., less than 20 nM, less than 25 nM, or less than 30 nM). As used herein, the term “tamoxifen resistance” refers to two classes of resistance: (a) de novo resistance, i.e., non-responsiveness to tamoxifen therapy from the beginning of the treatment, or (b) acquired resistance, i.e., non-responsiveness to tamoxifen therapy after initial responsiveness or tamoxifen-dependent growth/stimulated growth while continuing to express estrogen receptors (Minsun Chang. Biomol. Ther. 20(3), 256-267 (2012)). The acquired resistance to tamoxifen may develop as early as 3 m to 1 year to as late as 5 to 10 years. As used herein, the term “reference plasma endoxifen level” refers to a value of 30 nM.

As used herein, the term “unit dosage form” refers to physically discrete units suitable for unitary dosages for subjects, each unit containing a predetermined quantity of active material calculated to produce the desired therapeutic effect, in association with a suitable pharmaceutical excipient.

It is specifically understood that any numerical value cited herein includes all values from the lower value to the upper value, i.e., all possible combination of numerical values between the lowest value and the highest value enumerated are to be considered to be expressly stated in this application and the endpoint of all ranges are included within the range and independently combinable. For example, if a concentration range or beneficial range is stated as 1% to 50%, it is intended that values such as 2% to 40%, 10% to 30%, or 1% to 3% etc., are expressly enumerated in this specification. It is also to be understood that if a concentration or dose is stated as a specific value such as 1 mg or 10 mg, it is intended that it is intended to include 10% variation. As another example, a stated concentration of 20% is intended to include values ±10%. Yet another example, if a ratio of 1:10 to 10:1 is stated, then it is intended that ratios such as 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1 or from 2:5 to 3:5 etc. are specifically intended. There are only some examples of what is specifically intended. Unless specified otherwise, the values of the constituents or components of the compositions are expressed in weight percent of each ingredient in the component.

All methods described herein can be performed in a suitable order unless otherwise indicated or otherwise clearly contradicted by context. The use of any and all examples, or exemplary language (e.g., “such as” and “the like”) is intended merely to illustrate the invention and does not pose a limitation on the scope of the invention unless otherwise claimed. No language in the specification should be construed as any indicating any non-claimed element as essential to practice of the invention as used herein.

As used herein, the terms “hormone-dependent breast disorder,” “hormone-dependent reproductive tract disorder,” “hormone-dependent breast and reproductive tract disorder” each and collectively include, without limitation, any breast or reproductive tract (gynecologic) disorder that is related to or is sensitive to high estrogen or normal estrogen levels that need to be reduced, disorders with estrogen-receptor positive (ER+) and/or progesterone-receptor positive (PR+) disorders, for example, breast disorders, endometriosis, uterine fibroids (also called leiomyomas) etc. Reproductive tract disorders include endometrial, ovarian, cervical, uterus, vaginal, and vulvar cancers. The terms “estrogen-related disorder” and “estrogen-receptor related disorder” may be used interchangeably to refer to the foregoing hormone dependent disorders. The disorders may be presented primarily or secondarily to an underlying disease, for example, prostate cancer or other disorders such as liver diseases. Hormone-dependent breast and reproductive tract disorder include, for example, McCune-Albright syndrome, which is a disorder caused by a mutation in the GNAS gene affecting bones, skin, and several hormone-producing (endocrine) tissues, often resulting in abnormal scar-like (fibrous) tissue in their bones, a condition called polyostotic fibrous dysplasia, hyperthyroidism in individuals carrying such mutations, and in girls often resulting in precocious puberty.

As used herein, “breast disorder” means any aberration or a constellation of aberrations in the breast. Such aberration may be proliferative, non-proliferative, benign or malignant. Breast disorders include benign lesions of the breast (e.g., hyperplasia), increased breast density, gynecomastia, mastalgia, and breast cancer. Benign breast lesions include, but are not limited to, hyperplasia, atypia, ductal hyperplasia, lobular hyperplasia, atypical ductal hyperplasia (ADH), and atypical lobular hyperplasia (ALH). While not cancerous, ADH and ALH may be indicative of a predisposition for breast cancer.

As used herein, “breast cancer” means any malignant tumor of breast cells. Breast cancer may be at any stage of breast cancer, including stages of a pre-cancer, an early-stage cancer, a non-metastatic cancer, a pre-metastatic cancer, a locally advanced cancer, and a metastatic cancer. There are several types of breast cancer. Exemplary breast cancers include, but are not limited to, ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, ER-negative (ER−) breast cancer, HER2+ breast cancer, triple negative breast cancer (TNBC), adenoid cystic (adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. A single breast cancer tumor can be a combination of these types or be a mixture of invasive and in situ cancer.

### EXAMPLES

The invention is further illustrated by the following non-limiting examples.

### Example 1

### General Synthetic Method 1

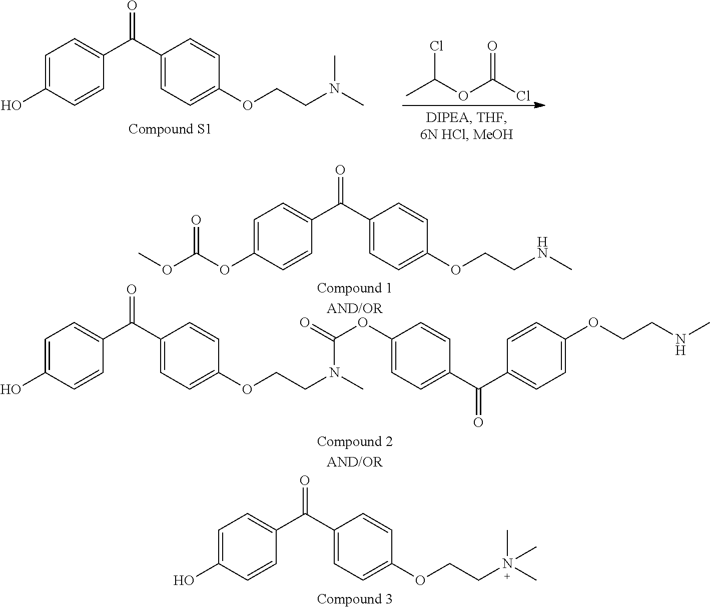

This example describes the general synthetic method for generating Compound 1, Compound 2, and Compound 3. Compound S1 (1.0 equiv.)

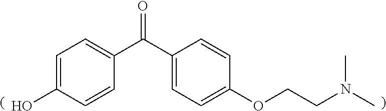

was added to tetrahydrofuran (THF) (9 vol., 7.9 wt.) in a suitable reactor. N-diisoethylpropylamine (DIEPA) (4.0 equiv., 1.8 wt.) was then added to the mixture over 30 minutes under an N₂ atmosphere. The mixture was then heated to 60° C. and 1-chloroethyl chloroformate (4.0 equiv., 2.0 wt.) was added to the mixture over 90 minutes. The mixture was then heated to reflux under stirring for 12 hours. A sample of the mixture was then tested by HPLC for reaction progress. If the HPLC result showed more than 1% of Compound S1, indicating residual starting material, an additional 0.23 wt. DIPEA and 0.25 wt. 1-chloroethyl chloroformate was added to the mixture and the mixture was held at reflux under stirring for an additional 2 hours. After at least 12 hours of reflux (or 14 hours reflux if there was residual starting material), methanol (5 vol., 4 wt.) was then added to the mixture over 2 hours at reflux and the mixture was held at reflux under stirring for an additional 30 minutes. After reflux, the mixture was distilled under partial vacuum to achieve a 5 volume residue. 6N HCl (4 wt.) was then added slowly over 2 hours to maintain an internal reaction temperature of 60° C. and the mixture was heated to reflux under stirring for 12 hours. A sample of the mixture was the tested by HPLC for impurities. If the HPLC showed more than 1% of a Compound 1, the mixture was further held at reflux under stirring for an additional 3 hours. After the second reflux finished, the mixture was concentrated by applying a partial vacuum to achieve a 4 volume residue. Water (1 vol.) was then added to the mixture and the mixture was distilled to achieve a 4 volume residue under full vacuum. The mixture was then cooled and 8N NaOH (at least 5.0 vol., 5.0 wt.) was added slowly to the mixture until the mixture turned from a suspension to a dark brown solution with a pH greater or equal to 13. A sample of the solution was then tested by HPLC for impurities. If the HPLC showed more than 1.5% of Compound 2, the mixture was heated to 60° C. and stirred for 2 hours and re-tested for Compound 2. Once the concentration of Compound 2 is below 1.5%, the mixture was then cooled, and ethyl acetate (4.0 vol., 3.6 wt.) was added for extraction. The mixture was stirred for about 30 minutes, and then allowed to settle under non-stirring conditions. The mixture was then separated into the organic layer and aqueous layer. If an emulsion was observed at the interface, it was collected with the organic layer (e.g., ethyl acetate). The aqueous layer was kept under full vacuum for 30 minutes to remove any residual organic solvent. 6N HCl (0.6 vol., 0.6 wt.) was then added to the aqueous layer until its pH was between 8-10. The aqueous mixture was then cooled for one hour at 0±5° C. and stirred for 2 hours. The aqueous mixture was then filtered and washed with purified water (3.0 vol., 3.0 wt.) to yield a wet solid cake. A solvent mixture of water:isopropyl alcohol (3:1 v/v) was then added to the wet solid cake and the mixture was stirred for 30 minutes at 40° C. and filtered again yielding a wet solid cake. The wet solid cake filtrate was then washed with ethyl acetate (2 vol., 1.8 wt.). The wet solid cake was then dried at 60° C. under vacuum to yield the product.

### Example 2

### General Synthetic Method 2

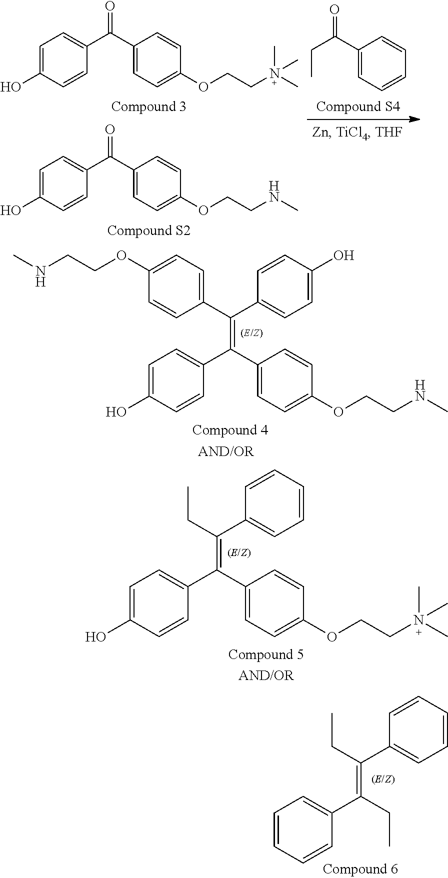

This example describes the general synthetic method for generating Compound 4a, Compound 4b, Compound 5a, Compound 5b, Compound 6A and Compound 6B. In a dry vessel containing THF (10 vol., 8.9 wt., KF at most about 0.05%), zinc powder (0.96 wt.) was added under stirring at room temperature and N₂ flow. The vessel was then cooled to 0° C. and TiCl₄ (1.4 wt.) was added slowly to maintain an internal reaction temperature below 20° C. The solution was then heated to 60° C. and stirred for 2 hours. In a separate vessel, carbonyl containing compounds (e.g., Compound 3 or Compound S2) (1 equiv. wt.) and propiophenone (Compound S4, 0.74 wt.) were added to THF (8 vol., 7.1 wt.) and heated to 60° C. The suspension of carbonyl containing compounds were then added to the first solution of THF, zinc powder, and TiCL₄ over 1 hour and was stirred for 8 hours at 60° C. A sample of the solution of was then tested by HPLC for impurities. If the HPLC showed more than 1% of the starting material (e.g., Compound S2), TiCl4 (0.07 wt.) was added to the reaction and the reaction was further stirred for 2 hours at reflux temperature.

The reaction mixture was then distilled under vacuum until only 10 vol. remained. Then, MeTHF (10 vol.) was added to the distillate and distillation was re-performed until only 10 vol. remained. Another solvent addition of MeTHF (10 vol.) and the distillation was performed again until 10 vol. remained. During the distillation the temperature did not exceed 40° C. 3N HCl (10 vol.) was added to the 10-vol. distillate solution under stirring at 30° C. until gas release was observed. When gas is no longer being released, observed by the absence of bubbling, the solution was heated to 50° C. and stirred for 30 minutes. The solution was then filtered with a biphasic system and washed with MeTHF (1:1 w/w). The solution was then stirred for 15 minutes at 50° and left to undergo phase separation for 30 minutes at 50° C. The aqueous phase was then discharged. The remaining organic phase was washed with 3N HCl (3 vol.) and stirred for 30 minutes at 50° C. and the aqueous phase was again discharged. This wash step was repeated once more. The organic phase was then cooled to 20-30° C. and 40% K₂CO₃ was added, and the solution was stirred for 1 hour after which a solid precipitate was observed. 30-35% HCl was added until the pH of the aqueous phase was 0-1 and the solid was completely dissolved. The solution was then heated to 50° C. and stirred for 30 minutes and then left to settle for an additional 30 minutes. The aqueous phase was discharged, and the organic phase was washed with 1N NaOH (10 vol.) at 50° C. and stirred for 1 hour after which NaCl (1:1 w/w) was added to the solution and it was stirred for an additional 30 minutes at 50° C. and a solid precipitate was observed. The solution was then filtered, and the filtered precipitate (filter cake) was washed with MeTHF (1:1 w/w). The filter cake in MeTHF solution was then heated to 50° C. and stirred for 15 minutes and left to settle for 30 minutes. The aqueous phase was then discharged from the flask. 1N NaOH (10 vol.) was added to the remaining solution, which was then stirred at 50° C. for 15 minutes. NaCl (1:1 w/w) was added, and the solution was stirred for an additional 30 minutes and left to settle under non-stirring conditions for 30 minutes. The aqueous phase was discharged, and the organic phase was subjected to a second wash. After the second wash, 20% NaCl was added to the organic phase and the solution was stirred for 30 minutes at 50° C. and left to settle under non-stirring conditions for 30 minutes at 50° C. The aqueous phase was discharged from the bottom of the flask. The organic phase was then distilled at 50° C. to 2 vol. and ACN (10 vol.) was added, and the solution was again distilled back to 2 vol. ACN (10 vol.) was again added to the distillate and the solution was distilled once more to 3 vol.

A sample of the distillate was then tested by GC for impurities. If the GC analysis showed more than 2% of the MeTHF, another distillation was performed. After GC analysis, acetone (3 vol.) was added to the distillate and the solution was heated at 50° C. under stirring for 1 hour. The suspension was then cooled down to 5° C. over a time period of 4 hours (cooling rate of 15° C. per hour). The suspension was then stirred at 5° C. for 8 hours. A solid precipitate was observed and the solution was filtered. The filtered precipitate (filter cake) was then washed with a pre-cooled solvent mixture of ACN:acetone (1:1 v/v, total 2 vol.). The wet filter cake was then dried for 4 hours at 70° C. under vacuum to yield the product.

### Example 3

### General Synthetic Method 3

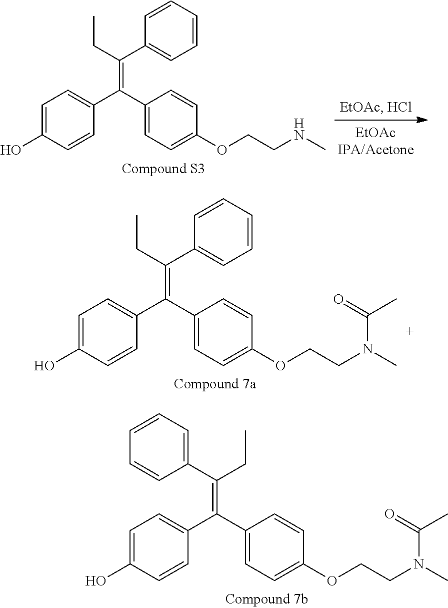

This example describes a method for generating Compounds 7a and 7b. While this method utilizes E/Z endoxifen (Compounds S3a and S3b) and ethyl acetate to generate Compounds 7a and 7b, this method can be applied to other amines and acylating agents. In a vessel containing ethyl acetate (10.0 L, 9.0 kg, 9.0 wt.), a mixture of E/Z endoxifen (Compounds S3a and S3b) 6N HCl (4.0 L, 4.0 kg, 4.0 wt.) was slowly added to the solution while maintaining an internal reaction temperature of 10° C. The mixture was then heated to 60±5° C. and stirred for 10 hours. A portion of the solution was then subjected to HPLC analysis to determine isomeric ratios, and to ensure that the Z-isomer was present at no less than 46%. After HPLC analysis, 8N NaOH (8.0 L, 8.0 kg, 8.0 kg) was slowly added to the solution until the solution reached a pH of 12 while also maintaining an internal reaction temperature of 10° C. Once the pH was equal or greater than 12, ethyl acetate (5 vol.) was added to the solution under stirring and then the solution was left to settle under non-stirring conditions for 10 minutes at a temperature of 10° C. or less. The aqueous phase was then discharged, and if there was an emulsion phase present, it was kept with the organic phase. The organic phase was then heated to 20-30° C. A 20% NaCl solution (3.0 L, 3.0 kg, 3.0 vol, 3.0 wt.) was then added to the organic phase and the mixture was heated to 50-60° C. The mixture was stirred and then left to settle under non-stirring conditions for 10 minutes. The aqueous phase was then discharged, and the organic phase was filtered through activated carbon cartridges and washed with ethyl acetate (5.0 vol., 4.5 kg, 5 vol., 4.5 wt.). The solution was then filtered to remove any residue from the activated carbon cartridges and any residue was rinsed at 50-60° C. to avoid product precipitation. The filtrate was then concentrated under vacuum until the volume reached 3 vol. Then ethyl acetate (9 vol.) was added to the solution (12 vol. total).

Ethanol (EtOH) was then added to the solution according to the formula: EtOH (Kg)=[(6.25−GC value)/93.75]*(Solution Volume)*0.902 g/mL. A sample of the solution was then used for GC analysis to ensure any residual EtOH was between 5-7.5%. Once the solution was confirmed to have an EtOH concentration between 5-7.5%, the solution was cooled to −2° C. over a time period of 2 hours and maintained at −2° C. for 2 hours. The solution was then heated to 50° C. in 1 hour and stirred for 20 minutes and cooled down to 0° C. over a time period of 4 hours. After 3 hours of cooling, a sample of the mixture was taken for analysis of the E/Z isomerization ratio by HPLC. Once the E/Z ratio was determined to be no more than 0.25 by HPLC analysis, the mixture was then filtered and washed with pre-cooled ethyl acetate (1.5 L, 1.5 kg, 1.5 vol., 1.3 wt.). The filtered wet cake was then dried at 90° C. The filtrate was then concentrated and IPA (10.0 L, 3 vol.) was added and the solution was further concentrated. IPA (10.0 L, 3 vol.) was added again and the mixture was concentrated until a volume of 5 vol. A sample of the solution was then taken for GC analysis to ensure the ethyl acetate concentration was below 1%. If the ethyl acetate concentration was above 1%, IPA (5 vol.) was added and the mixture was again re-concentrated until a volume of 5 vol. and re-tested. Once the ethyl acetate concentration was confirmed to be lower than 1%, the solution was further distilled to 2 vol. The distillate was the cooled to 25° C. and acetone (1.5 L) was added for a total solution volume of 3.5 vol. The solution was then heated for 1 hour at 50° C. and stirred for 2 hours. The solution was then cooled to 0° C. in a time period of 4 hours (cooling rate less than 15° C. per hour). The solution was then stirred at 0±5° C. for 6 hours. The mixture was then filtered and washed with pre-cooled 2-propanol (2.0 L, 1.6 kg, 2.0 vol., 1.6 wt.). The filtered wet cake was then dried at 80° C. to yield the product. The product (1 wt.) was then dissolved in ethyl acetate (10 vol., 9 wt.). The mixture was agitated at 25° C. for 24 hours. The mixture was then filtered and washed with pre-cooled ethyl acetate (2 vol., 1.8 wt.). The filtered wet cake was then dried at 80° C. The wet filter cake was then dried for 4 hours at 70° C. under vacuum to yield the product.

### Example 4

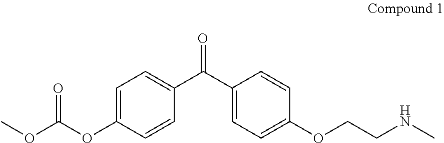

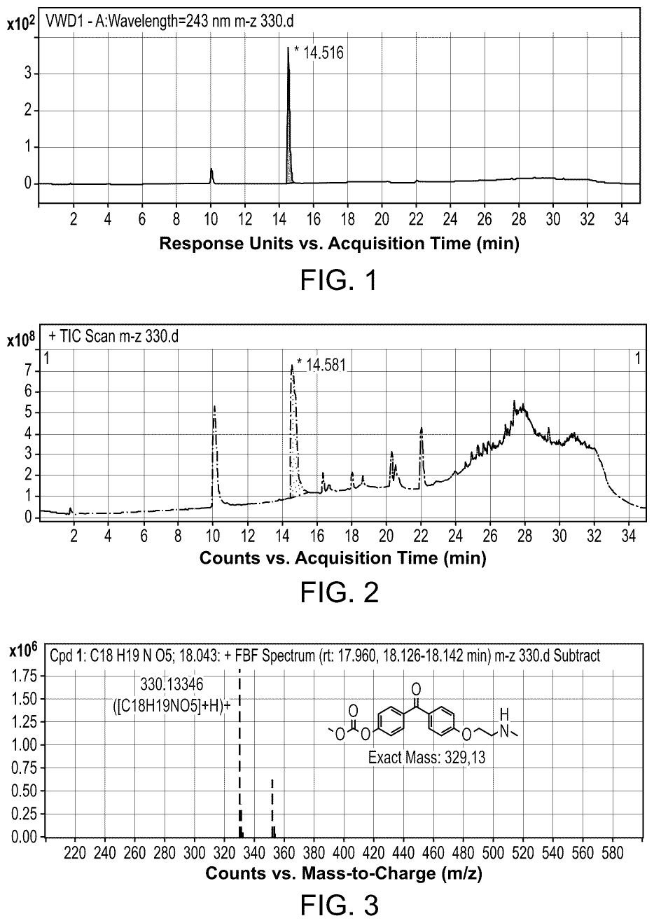

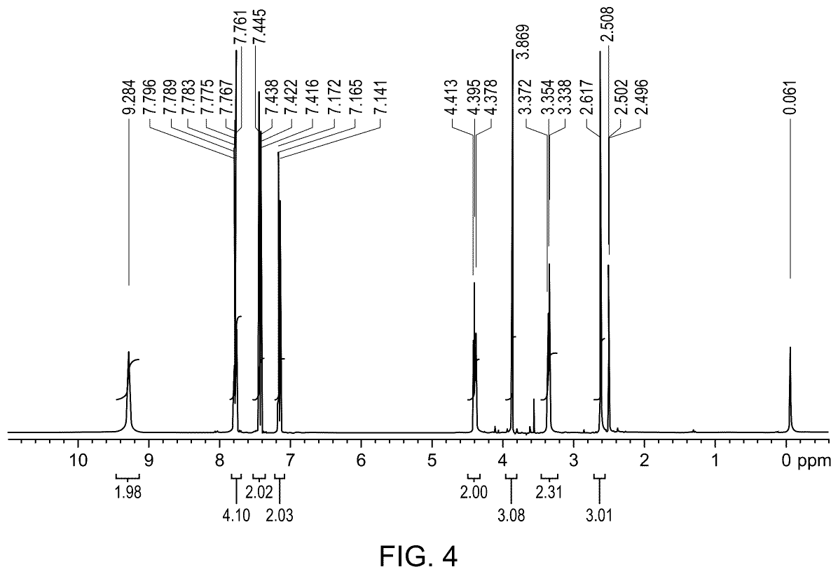

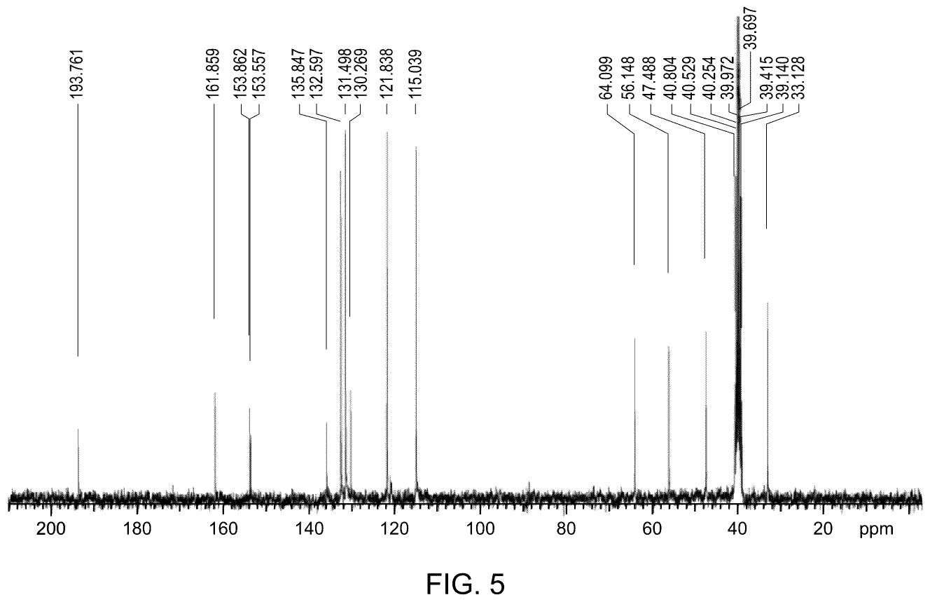

This example describes the synthesis and characterization of Compound 1. Compound 1 was generated with General Synthetic Method 1 (as described in EXAMPLE 1), and was further characterized by UV, TIC, mass spectrometry, and NMR. The UV chromatogram of Compound 1 is shown in FIG. 1 and displays a peak of absorbance at 243 nm around 14 minutes. The TIC chromatogram of Compound 1 is shown in FIG. 2 and displays a peak at 14 minutes. The mass spectrum of Compound 1 is displayed in FIG. 3 and shows a peak at 330 m/z consistent with Compound 1. The proton NMR spectrum of Compound 1 is shown in FIG. 4 and the carbon NMR spectrum of Compound 1 is shown in FIG. 5.

### Example 5

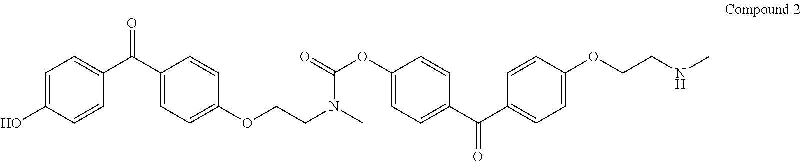 [SMILES: CNCCOc1ccc(C(=O)c2ccc(OC(=O)N(C)CCOc3ccc(C(=O)c4ccc(O)cc4)cc3)cc2)cc1]

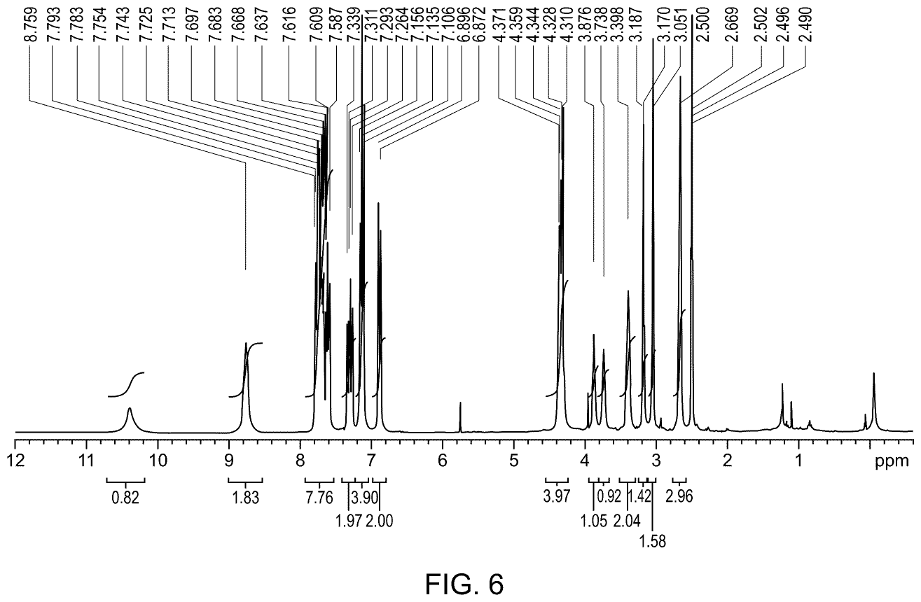

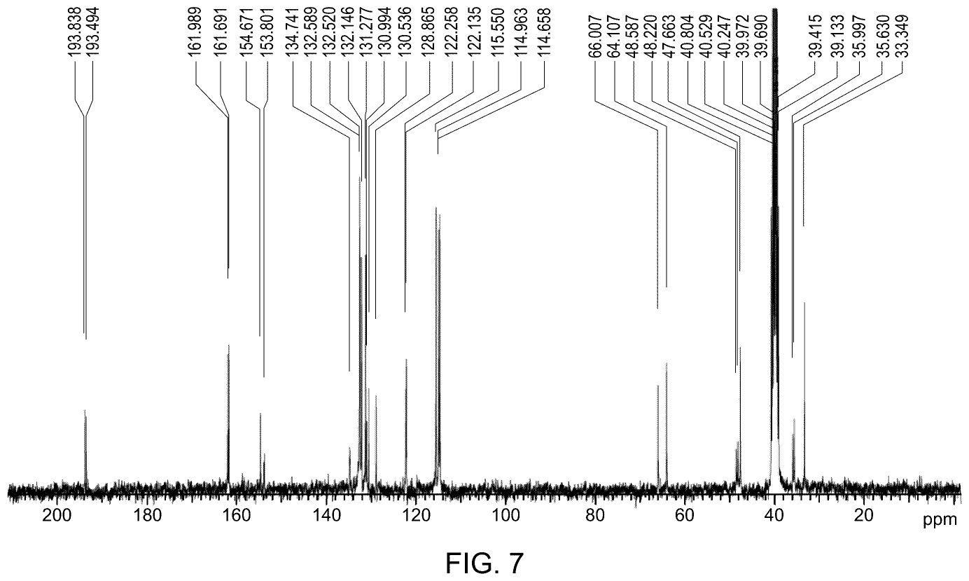

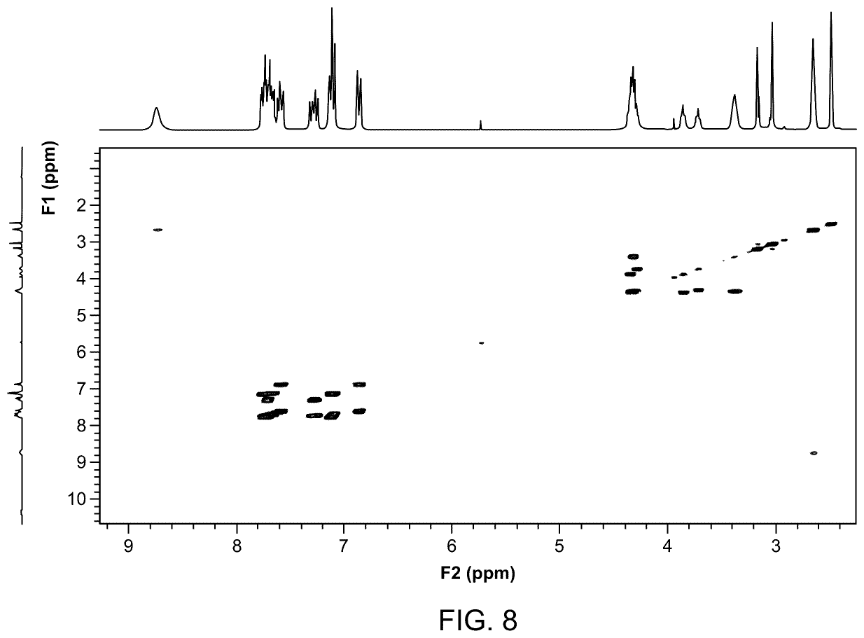

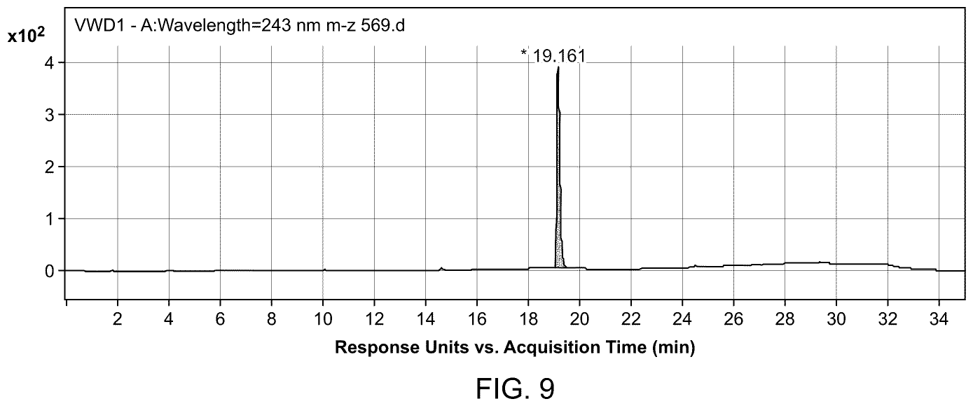

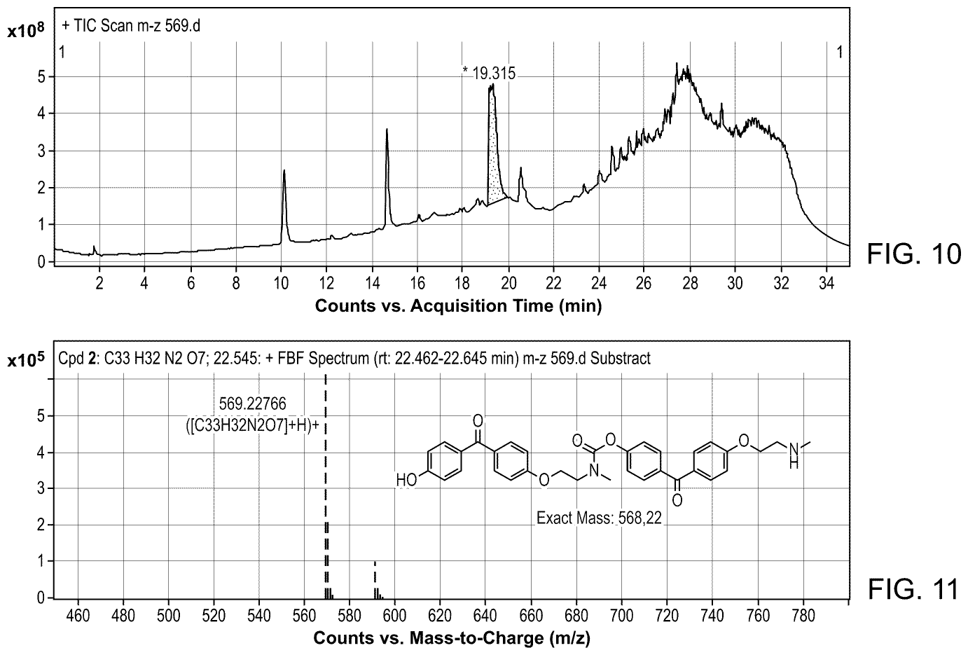

This example describes the synthesis and characterization of Compound 2. Compound 2 was generated with General Synthetic Method 1 (as described in EXAMPLE 1), and was further characterized by NMR, correlated spectroscopy NMR (COSY NMR), UV, TIC, and mass spectrometry. The proton NMR spectrum of Compound 2 is displayed in FIG. 6, the carbon NMR spectrum of Compound 2 is shown in FIG. 7, and the COSY NMR 2D spectrum is shown in FIG. 8. The UV chromatogram of Compound 2 is shown in FIG. 9 and displays a peak of absorbance at 243 nm around 19 minutes. The TIC chromatogram of Compound 2 is shown in FIG. 10 and displays a peak of at 19 minutes. The mass spectrum of Compound 2 is shown in FIG. 11 and displays a peak at 569 m/z consistent with Compound 2.

### Example 6

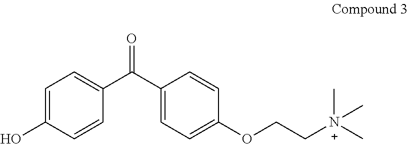 [SMILES: C[N+](C)(C)CCOc1ccc(C(=O)c2ccc(O)cc2)cc1]

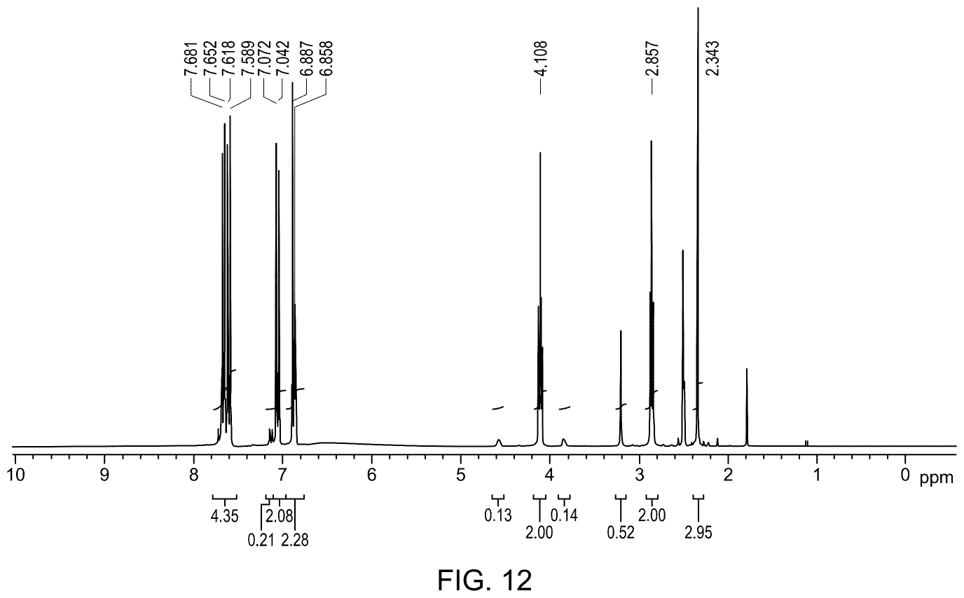

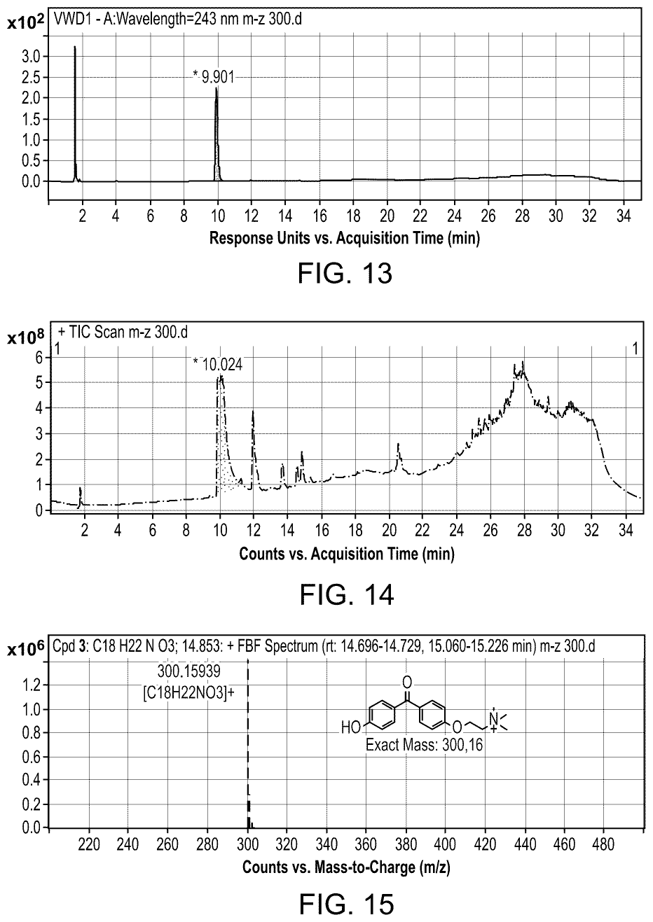

This example describes the synthesis and characterization of Compound 3. Compound 3 was generated with General Synthetic Method 1 (as described in EXAMPLE 1), resulting from the methylation of unreacted starting material (Compound S1) and was further characterized by NMR, UV, TIC, and mass spectrometry. The proton NMR spectrum of Compound 3 is displayed in FIG. 12. The UV chromatogram of Compound 3 is displayed in FIG. 13 and shows a peak of absorbance at 243 nm around 10 minutes. The TIC chromatogram of Compound 3 is shown in FIG. 14 and shows a peak at 10 minutes. The mass spectrum of Compound 3 is shown in FIG. 15 and shows a peak at 300 m/z consistent with Compound 3.

### Example 7

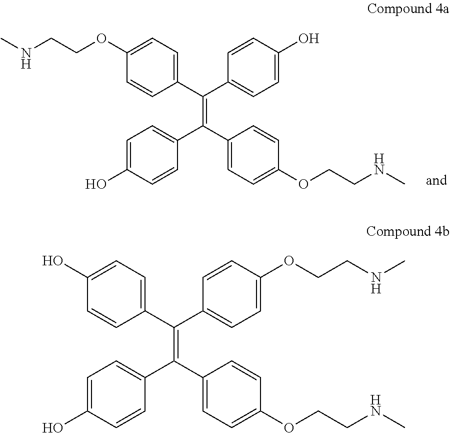

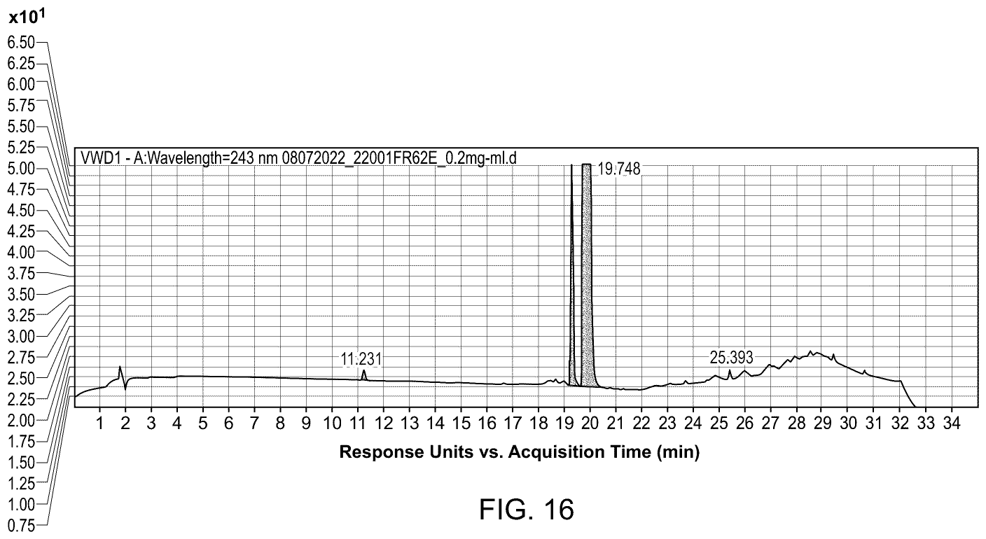

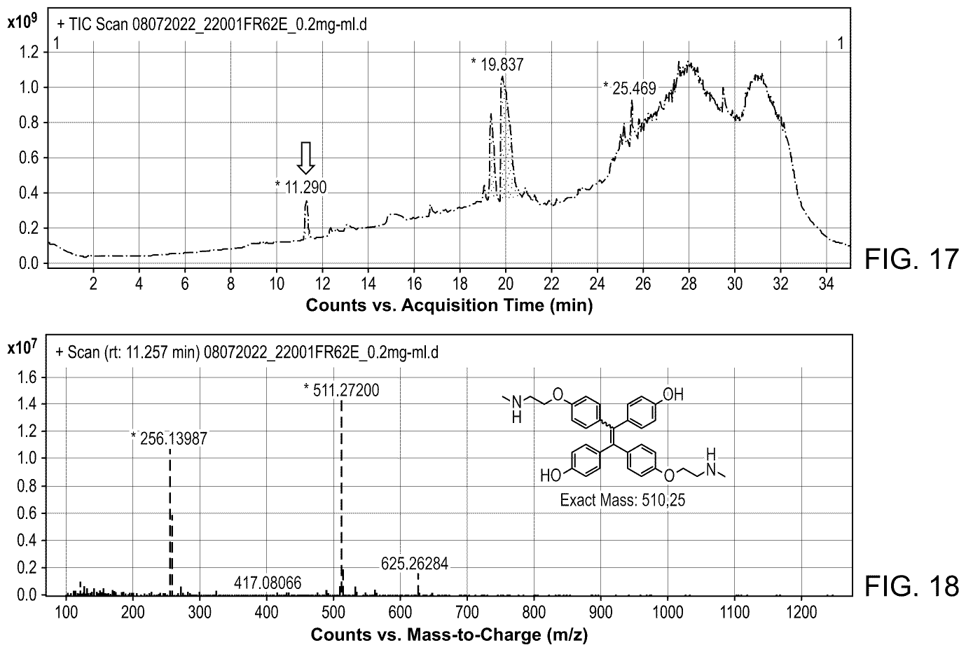

This example describes the synthesis and characterization of Compound 4a and Compound 4b. Compound 4a and Compound 4b were generated with General Synthetic Method 2 (as described in EXAMPLE 2), by a homocoupling of Compound S2. Compound 4a and Compound 4b were characterized by UV, TIC, and mass spectrometry. The UV chromatogram of Compound 4a and Compound 4b is shown in FIG. 16 and displays a peak of absorbance at 243 nm at around 11 minutes. The TIC chromatogram of Compound 4a and Compound 4b is shown in FIG. 17 and displays a peak at 11 minutes. The mass spectrum of Compound 4a and Compound 4b is shown in FIG. 18 and has a peak at 511 m/z consistent with Compound 4a and Compound 4b.

### Example 8

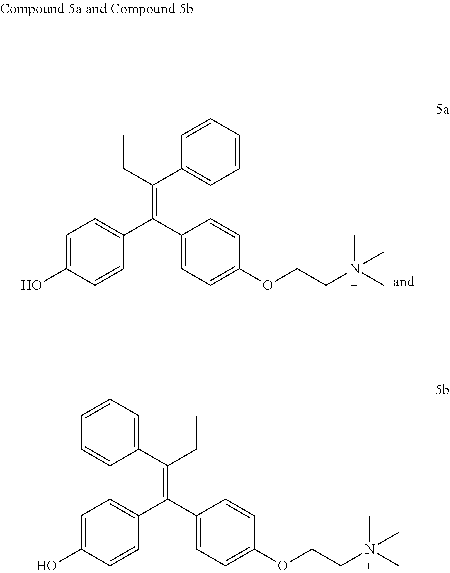

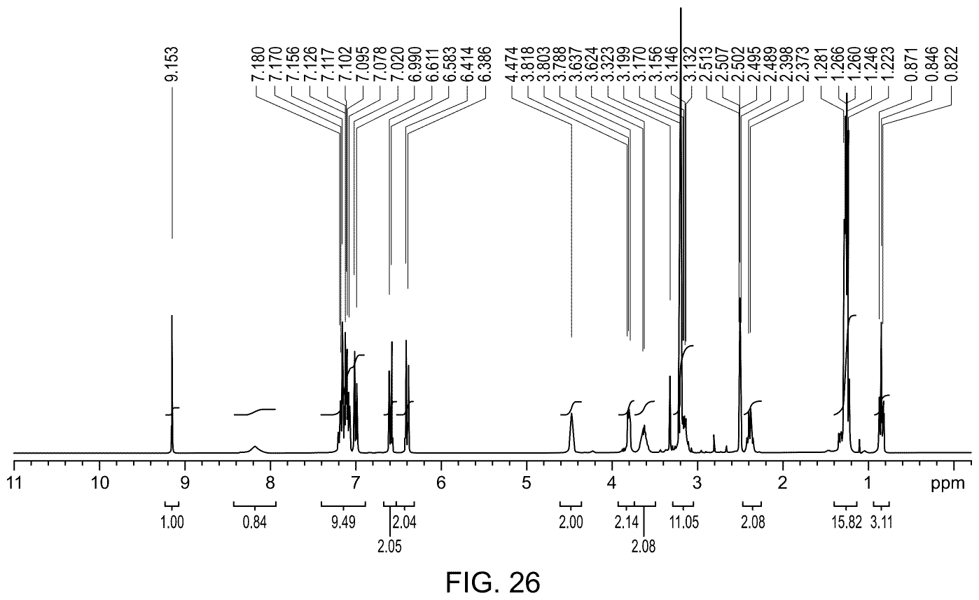

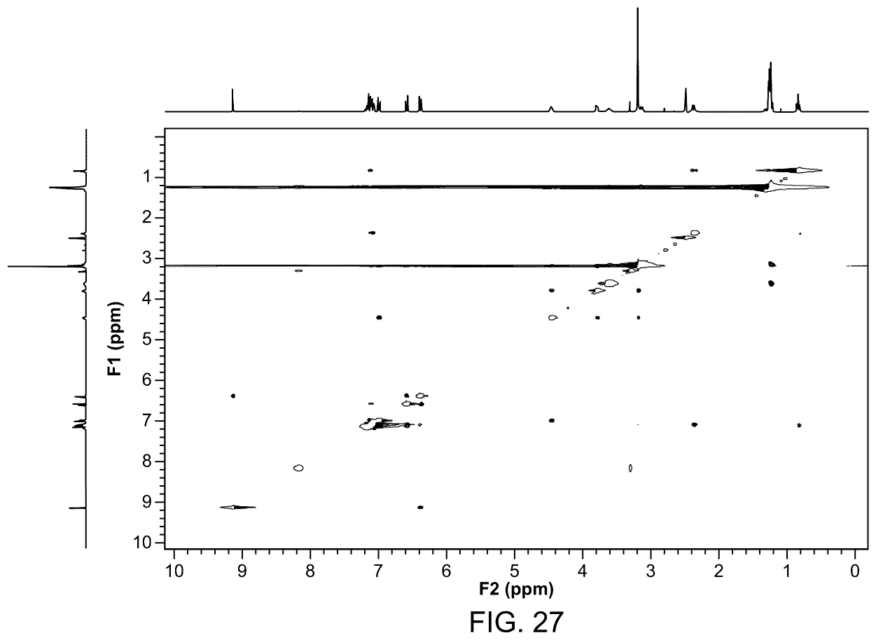

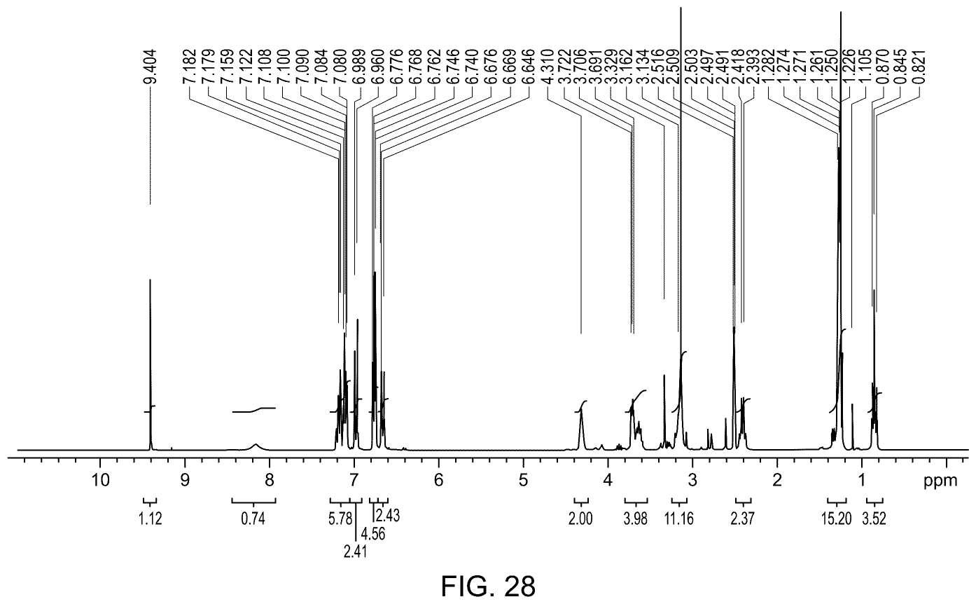

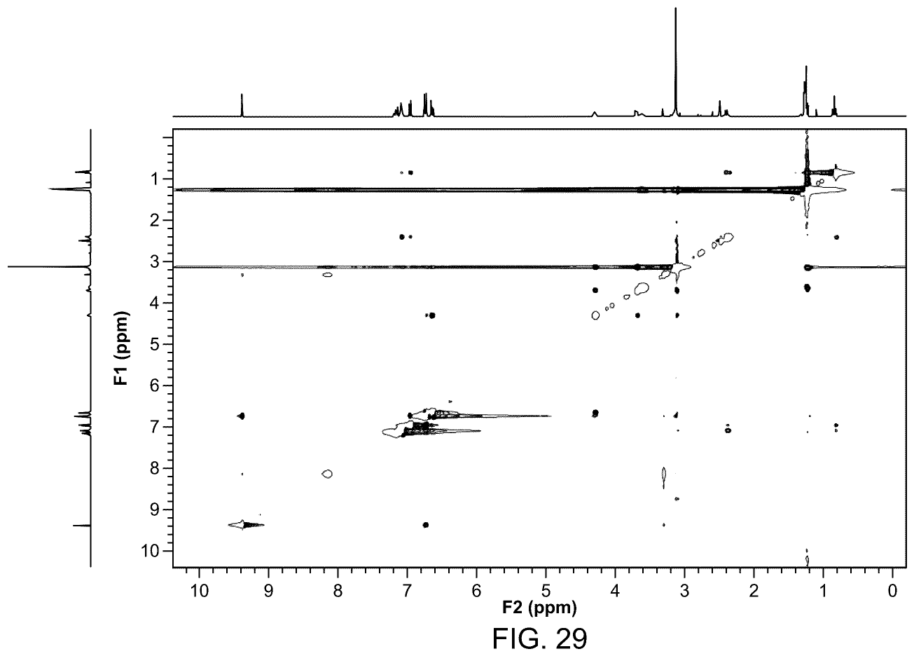

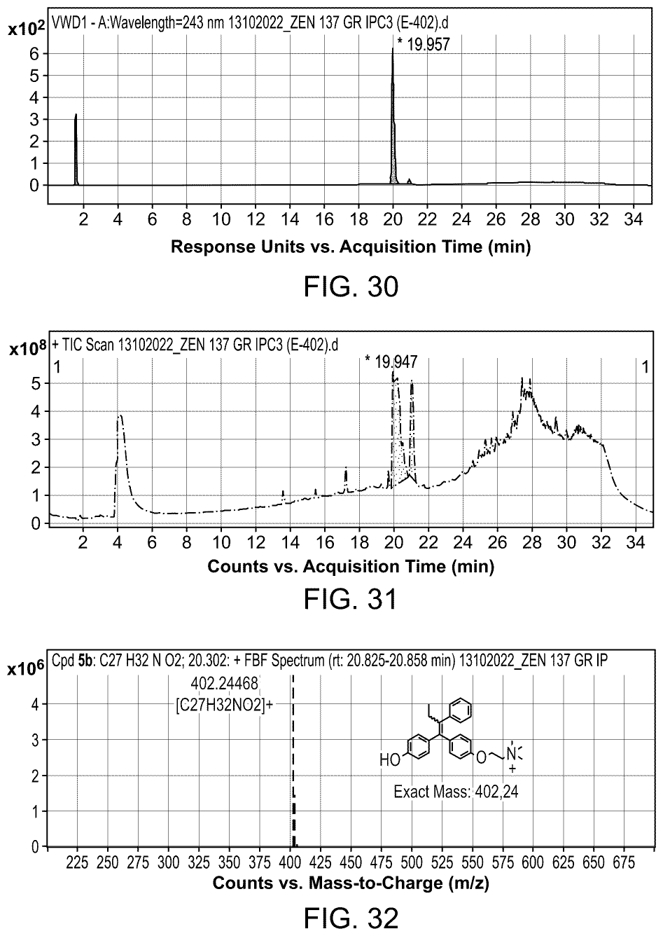

This example describes the synthesis and characterization of Compounds 5a and 5b. Compounds 5a and 5b were generated with General Synthetic Method 2 (as described in EXAMPLE 2), by the coupling of Compound 3 and propiophenone. Compounds 5a and 5b were characterized by UV, TIC, mass spectrometry, NMR, and Rotating Frame Overhauser Enhancement Spectroscopy (ROESY). Compounds 5a and 5b were further purified to yield both the (E)-isomer, Compound 5b, and the (Z)-isomer, Compound 5a, that were both characterized by NMR, and Rotating Frame Overhauser Enhancement Spectroscopy (ROESY). The proton NMR of the (E)-isomer (Compound 5b) is shown in FIG. 26, the ROESY spectrum of the (E) isomer (Compound 5b) is shown in FIG. 27, the proton NMR of the (Z) isomer (Compound 5a) is shown in FIG. 28, and the ROESY spectrum of the (Z) isomer (Compound 5a) is shown in FIG. 29. The UV chromatogram of Compound 5b is shown in FIG. 30 and displays a peak of absorbance at 243 nm around 20 minutes. The TIC chromatogram of Compound 5b is shown in FIG. 31 and displays a peak at 20 minutes. The mass spectrum of Compound 5b is shown in FIG. 32 and has a peak at 402 m/z consistent with Compound 5b.

### Example 9

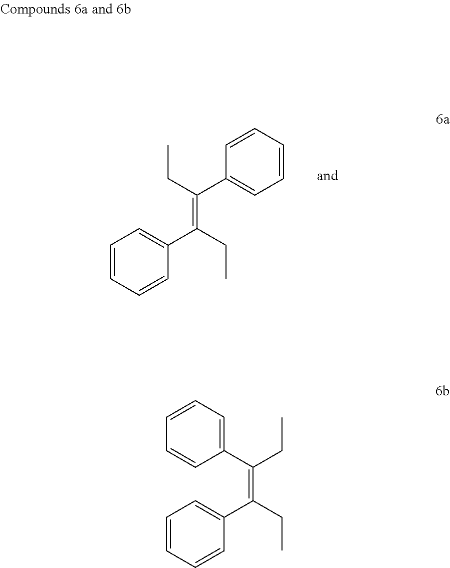

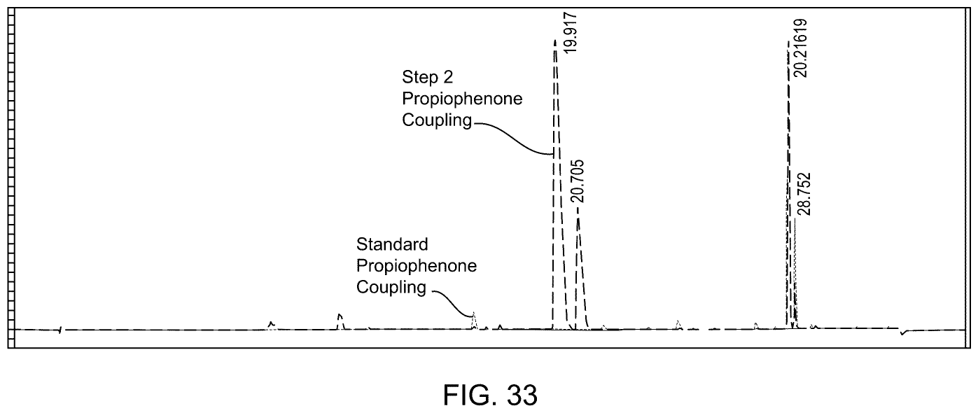

This example describes the synthesis and characterization of Compounds 6a and 6b. Compounds 6a and 6b were generated with General Synthetic Method 2 (as described in EXAMPLE 2), from a homocoupling of 2 propiophenone molecules. The product of the propiophenone coupling lacks any ionizable groups and could not be characterized by mass spectrometry. Compounds 6a and 6b were analyzed by HPLC. Compounds 6a and 6b were compared against a reference chromatogram of a standard of Compounds 6a and 6b. As seen in FIG. 33, Compounds 6a and 6b have both (E) and (Z) isomers and overlayed well with the standard of Compounds 6a and 6b (Standard Propiophenone Coupling).

### Example 10

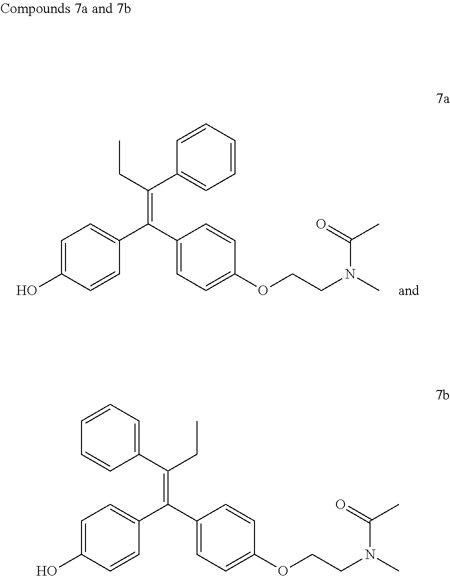 [SMILES: CC/C(=C(\c1ccc(O)cc1)c1ccc(OCCN(C)C(C)=O)cc1)c1ccccc1 ; CC/C(=C(/c1ccc(O)cc1)c1ccc(OCCN(C)C(C)=O)cc1)c1ccccc1]

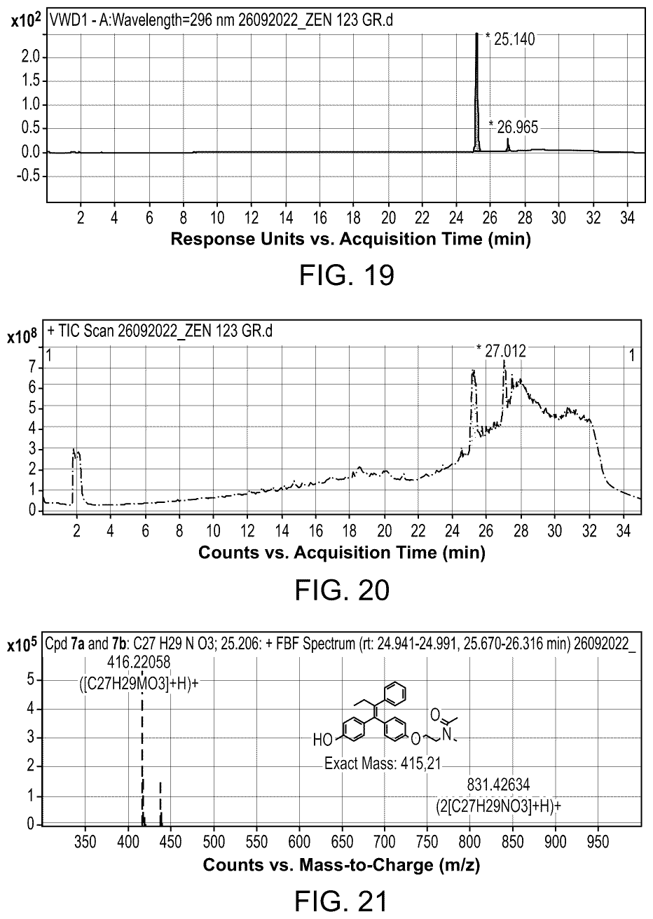

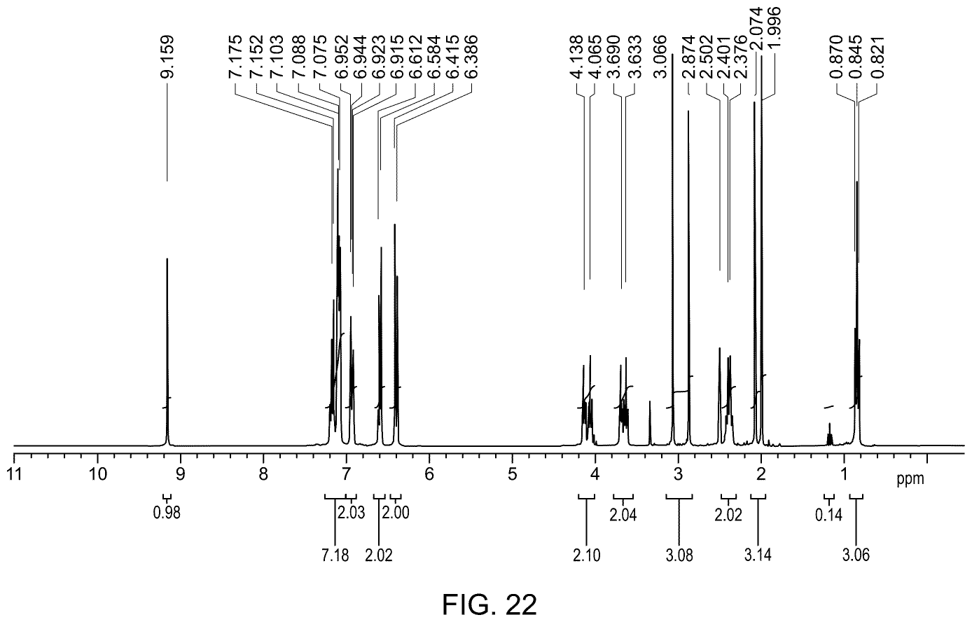

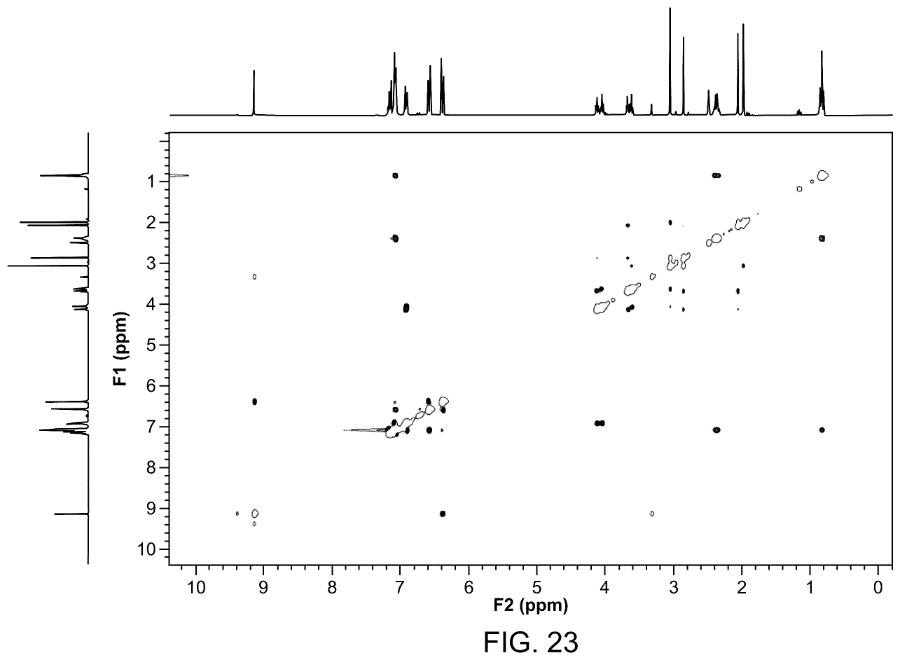

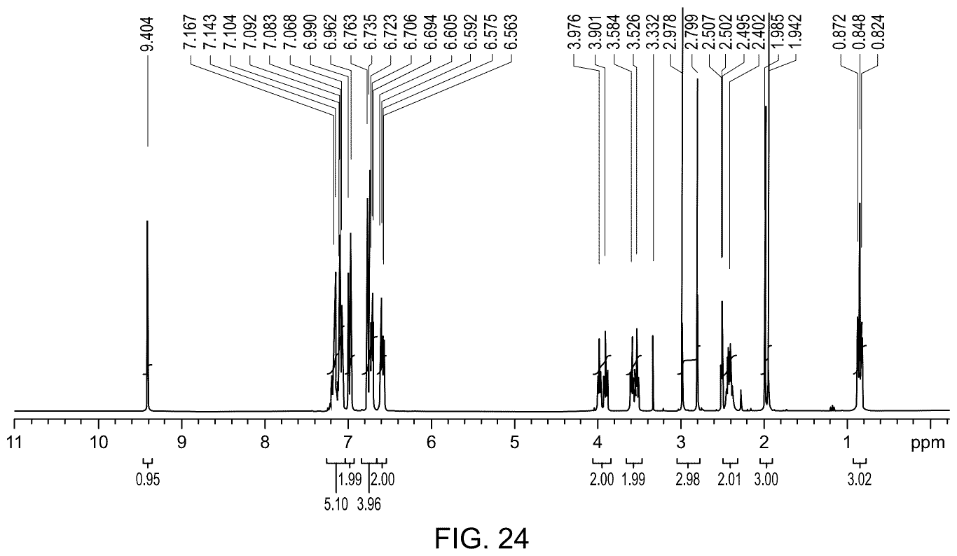

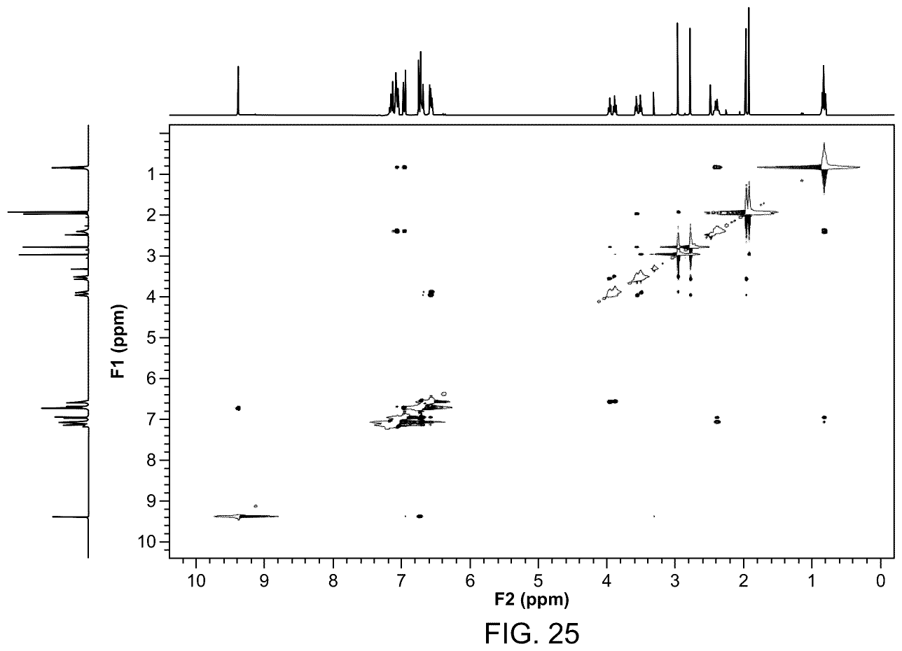

This example describes the synthesis and characterization of Compounds 7a and 7b. Compounds 7a and 7b were generated by acetylation of Compounds S3a and S3b in the presence of ethyl acetate during General Synthetic Method 3 (as described in EXAMPLE 3). Compounds 7a and 7b were formed and characterized by UV, TIC, and mass spectrometry. The UV chromatograph of Compound 7b is shown in FIG. 19 and displays a peak of absorbance at 296 nm around 25 minutes. The TIC chromatogram of Compound 7b is shown in FIG. 20 and displays a peak at 25 minutes, and 27 minutes. The mass spectrum of Compounds 7a and 7b is shown in FIG. 21 and shows a peak at 416 m/z consistent with Compounds 7a and 7b. Compounds 7a and 7b were further purified to yield both the (E)-isomer, Compound 7b, and the (Z)-isomer, Compound 7a, that were both characterized by NMR, and Rotating Frame Overhauser Enhancement Spectroscopy (ROESY). The proton NMR of the (E) isomer (Compound 7b) is shown in FIG. 22, the ROESY spectrum of the (E) isomer (Compound 7b) is shown in FIG. 23, the proton NMR of the (Z) isomer (Compound 7a) is shown in FIG. 24, and the ROESY spectrum of the (Z) isomer (Compound 7a) is shown in FIG. 25.

### Example 11

### Estrogen Receptor Binding Activities of Compounds 5a and 5b

This example describes the estrogen receptor binding activities of Compounds 5a and 5b. Compounds 5a and 5b are tested for estrogen receptor binding activity in a competitive assay binding experiment. The binding assays measures the competitive inhibition of radiolabeled estradiol ([³H-estradiol]) binding to an estrogen receptor isolated from a tissue including ERα and ERβ. The measurement is performed in triplicate with three biological replicates for each concentration. To a solution containing both ERα and ERβ, ³H-estradiol is added at saturation concentration to ensure all ERα and ERP binding sites are bound. The unbound ³H-estradiol is rinsed from the assay. The radiation (radioactive counts or disintegrations per minute) is measured to determine the radiation value at which all ERα and ERP sites are occupied by ³H-estradiol. Then, increasing concentrations of Compounds 5a and 5b are added to the assay, incubated for a period of time and rinsed. The radiation is re-measured after each addition and rinse step. The loss of radiation is then plotted against the added concentration of Compounds 5a and 5b to determine an affinity constant (kₐ) for Compounds 5a and 5b.

### Example 12

### Estrogen Receptor Binding Activities of Compounds 7a and 7b

This example describes the estrogen receptor binding activities of Compounds 7a and 7b. Compounds 7a and 7b were tested for estrogen receptor binding activity in a competitive assay binding experiment. The binding assays measures the competitive inhibition of radiolabeled estradiol ([³H-estradiol]) binding to an estrogen receptor isolated from a tissue including ERα and ERβ. The measurement is performed in triplicate with three biological replicates for each concentration. To a solution containing both ERα and ERβ, ³H-estradiol is added at saturation concentration to ensure all ERα and ERP binding sites are bound. The unbound ³H-estradiol is rinsed from the assay. The radiation (radioactive counts or disintegrations per minute) is measured to determine the radiation value at which all ERα and ERP sites are occupied by ³H-estradiol. Then, increasing concentrations of Compounds 7a and 7b were added to the assay, incubated for a period of time and rinsed. The radiation is re-measured after each addition and rinse step. The loss of radiation is then plotted against the added concentration of Compounds 7a and 7b to determine an affinity constant (kₐ) for Compounds 7a and 7b.

### Example 13

### Aromatase Inhibition Activities of Compounds 5a and 5b

This example describes the aromatase inhibition activities of Compounds 5a and 5b. An aromatase inhibition assay is performed by combing aromatase (CYP19A) and a fluorogenic substrate of aromatase that is converted into a highly fluorescent product by aromatase. An appropriate concentration of the fluorogenic substrate is added to obtain an optimal fluorescence signal measured by a detector. Increasing concentrations of Compounds 5a and 5b were then added. The concentration of Compounds 5a and 5b that causes a 50% reduction in fluorescence signal compared to no aromatase inhibition is then calculated to determine an IC₅₀ of Compounds 5a and 5b.

### Example 14

### Aromatase Inhibition Activities of Compounds 7a and 7b

This example describes the aromatase inhibition activities of Compounds 7a and 7b. An aromatase inhibition assay is performed by combing aromatase (CYP19A) and a fluorogenic substrate of aromatase that is converted into a highly fluorescent product by aromatase. An appropriate concentration of the fluorogenic substrate is added to obtain an optimal fluorescence signal measured by a detector. Increasing concentrations of Compounds 7a and 7b were then added. The concentration of Compounds 7a and 7b that causes a 50% reduction in fluorescence signal compared to no aromatase inhibition is then calculated to determine an IC₅₀ of Compounds 7a and 7b.

### Example 15

Treatment of a Patient with Breast Cancer with Compounds 5a and 5b

This example describes treatment of breast cancer using a composition of Compounds 5a and 5b. A patient having breast cancer is orally administered a composition of Compounds 5a and 5b every day for up to 6 months, or until the breast cancer is treated. Oral administration of a composition of Compounds 5a and 5b eliminates, slows the spread of, shrinks, or reduces symptoms of the breast cancer, thereby treating the breast cancer.

### Example 16

Treatment of a Patient with Breast Cancer with Compounds 7a and 7b

This example describes treatment of breast cancer using a composition of Compounds 7a and 7b. A patient having breast cancer is orally administered a composition of Compounds 7a and 7b every day for up to 6 months, or until the breast cancer is treated. Oral administration of a composition of Compounds 7a and 7b eliminates, slows the spread of, shrinks, or reduces symptoms of the breast cancer, thereby treating the breast cancer.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 17

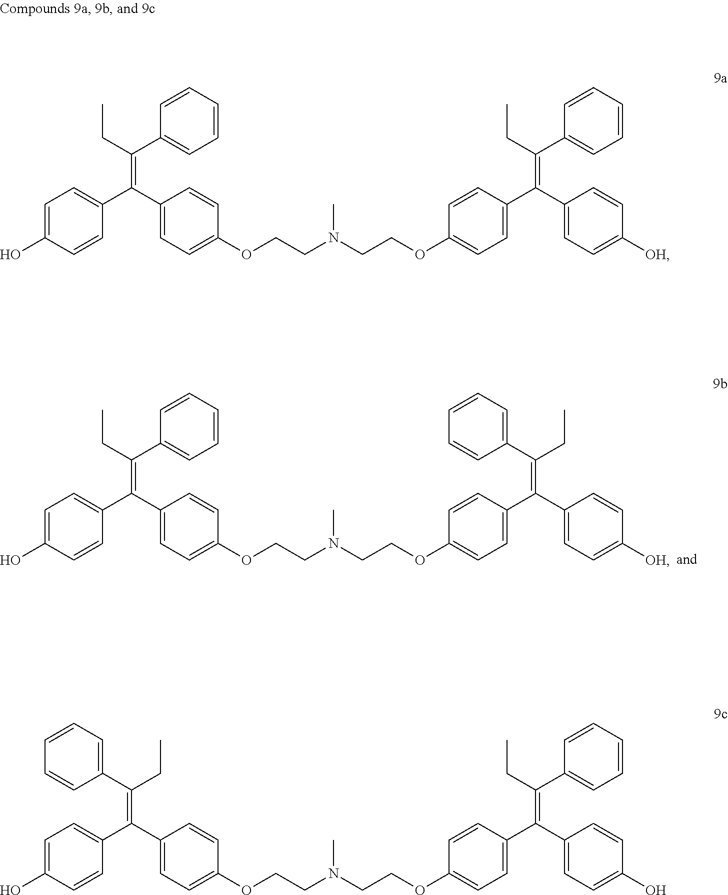 [SMILES: CC/C(=C(\c1ccc(O)cc1)c1ccc(OCCN(C)CCOc2ccc(/C(=C(\CC)c3ccccc3)c3ccc(O)cc3)cc2)cc1)c1ccccc1 ; CC/C(=C(\c1ccc(O)cc1)c1ccc(OCCN(C)CCOc2ccc(/C(=C(/CC)c3ccccc3)c3ccc(O)cc3)cc2)cc1)c1ccccc1 ; CC/C(=C(/c1ccc(O)cc1)c1ccc(OCCN(C)CCOc2ccc(/C(=C(\CC)c3ccccc3)c3ccc(O)cc3)cc2)cc1)c1ccccc1]

This example covers the production and characterization of Compounds 9a, 9b, and 9c. This compound was detected during Z-endoxifen production in amounts as high as 0.42%, and was identified based on its mass spectrometry parent peak at 715.37.

### Synthesis

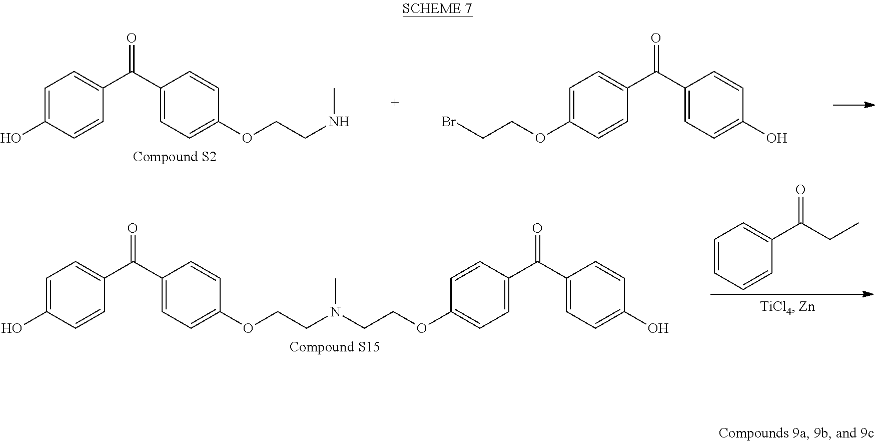

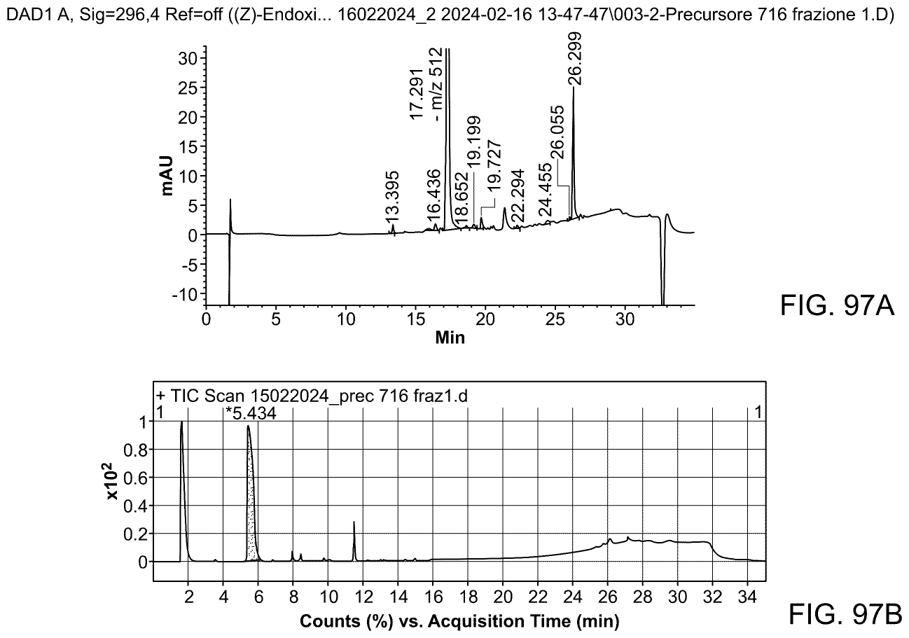

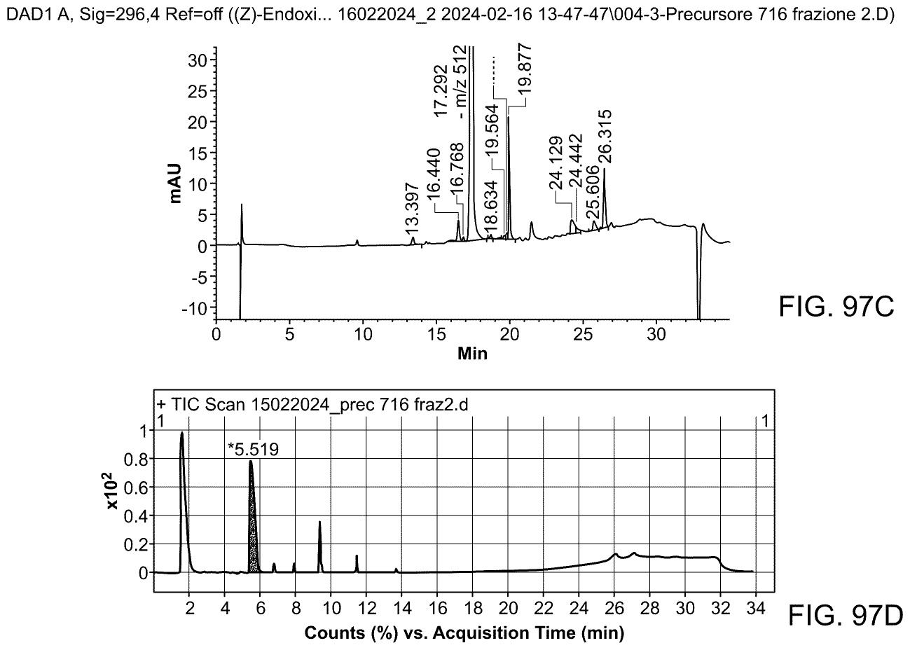

The proposed strategy consists of two steps: synthesis of Compound S15, which had a parent mass spectrometric peak at m/z 512, followed by (2) a McMurry reaction with propiophenone leading to a mixture of Compounds 9a, 9b, and 9c. [4-(2-Bromoethoxy)phenyl](4-hydroxyphenyl)methanone (1 eq.), Compound S2 (1.4 eq.) and DIPEA (1.1 eq.) in ethanol were stirred under reflux for 32 h. Then the ethanol was removed by distillation under vacuum. The resulting residue was purified on silica gel column, collecting two fractions of Compound S15 with HPLC purities of 96.6% and 95.8%, respectively. HPLC chromatograms of these two fractions are shown in FIGS. 97A and 97C, respectively. LC-MS chromatograms of these two fractions are shown in FIGS. 97B and 97D, respectively.

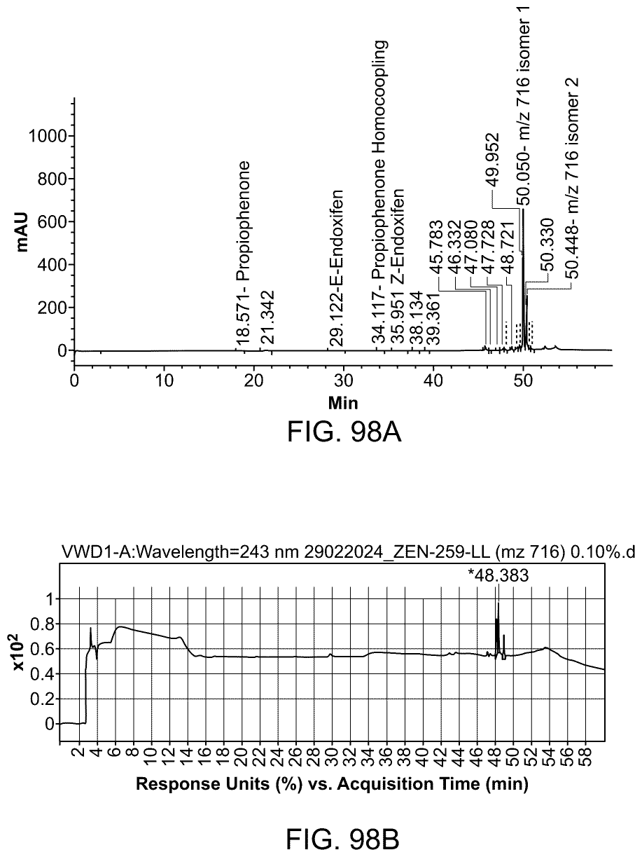

Compound S15 (1 eq.), propiophenone (3 eq.) in THF were added to TiCl₄/Zn mixture in THF and stirred 16 h under reflux. Then THF/MeTHF solvent switch was performed. The reaction was cooled down and HCl 3M was added. After the HCl addition, the biphasic mixture was heated at Tⱼ=50° C. and stirred for 30 minutes. The solid was filtered off and the cake was rinsed with 1 w/w of MeTHF. The biphasic mixture was settled and separated. The organic layer was then washed twice at Tⱼ=50° C. with 3 vol of HCl 3M and the solvent was removed. The HPLC purity of obtained crude m/z 716 was about 84% as a sum of three peaks, that were found by LC-MS to be the three possible isomers of the molecule. An HPLC chromatogram and an LC-MS chromatogram of this sample are shown in FIGS. 98A-B. The maximum reached HPLC purity was around 86%.

An observed impurity of the propiophenone homocoupling was 3,4-diphenylhex-3-one.

### Synthesis Replicates

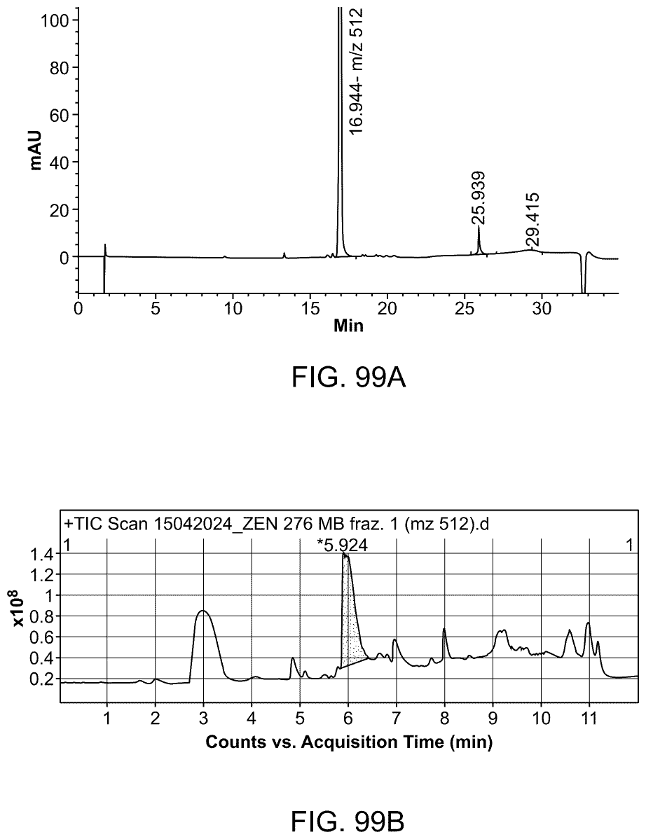

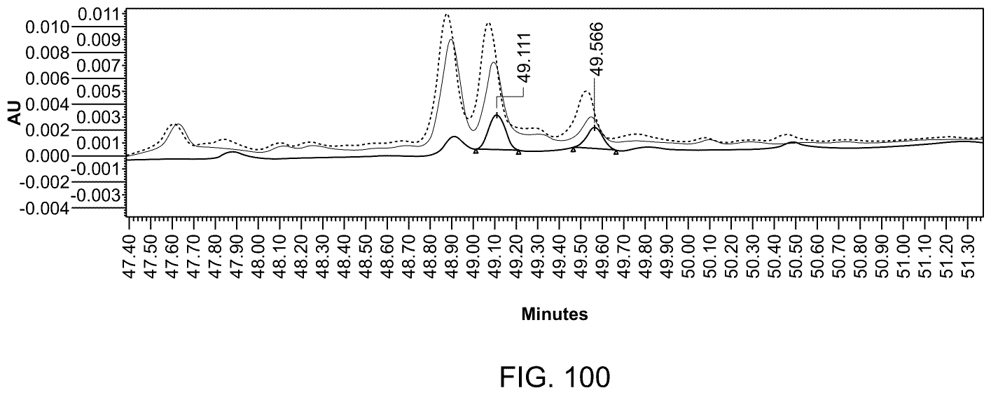

Compounds 9a, 9b, and 9c synthesis was repeated according to the above method. Three batches of Compound S15 were generated, this time with purities of 98.8%, 94.6%, and 99.0%. HPLC and LC-MS chromatograms of the batch with 98.8% purity are shown in FIGS. 99A-B. This batch was used to synthesize Compounds 9a, 9b, and 9c. As shown in FIG. 100, spiking raw Compounds 9a, 9b, and 9c demonstrated the peak match of both m/z 716 isomer peaks, as confirming the proposed structure.

### Example 18

### HPLC Quantification Methods

This example covers methods for quantifying endoxifen and endoxifen-related compounds using high-pressure liquid chromatography (HPLC). A first method utilized an HPLC Agilent 1260 instrument with quaternary pumps (or an equivalent HPLC system); Luna Phenyl-Hexyl 150×4.6 mm, 3.0 μm columns; ambient of 25° C. autosampler temperatures; 40° C. column temperatures; and 1.0 mL/minute flow rates. 10 mM ammonium formate in H₂O plus 0.3 mL formic acid (“Mobile Phase A”) and 10 mM ammonium formate in MeOH (“Mobile Phase B”) were utilized as mobile phases in this method. Mobile phase gradients for this method are summarized in TABLE 2.

TABLE 2

| Time (min) | Phase A (% v/v) | Phase B (% v/v) |
|---|---|---|
| 0 | 80 | 20 |
| 2 | 80 | 20 |
| 17 | 30 | 70 |
| 20 | 30 | 70 |
| 27 | 5 | 95 |
| 30 | 5 | 95 |
| 30.1 | 80 | 20 |
| 35 | 80 | 20 |

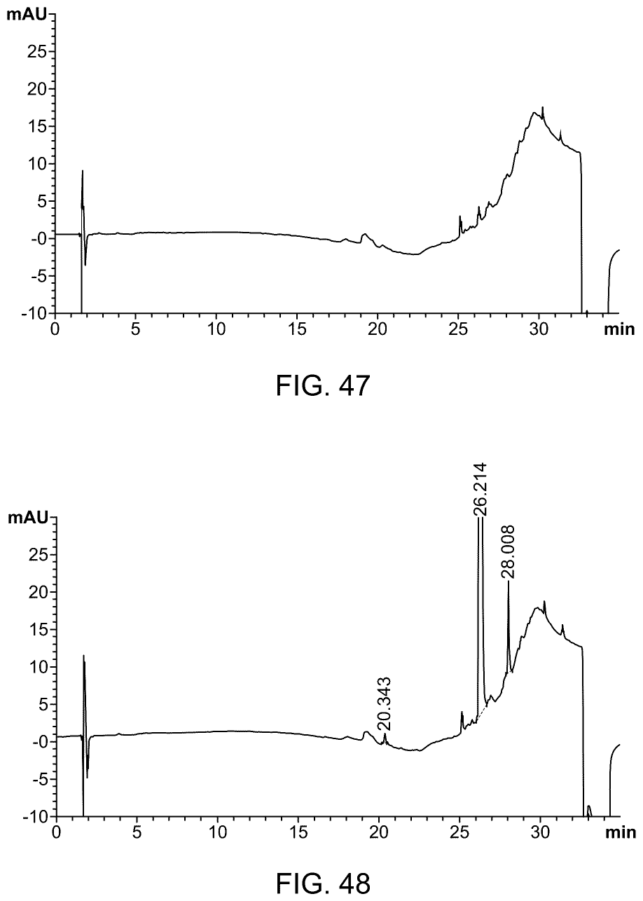

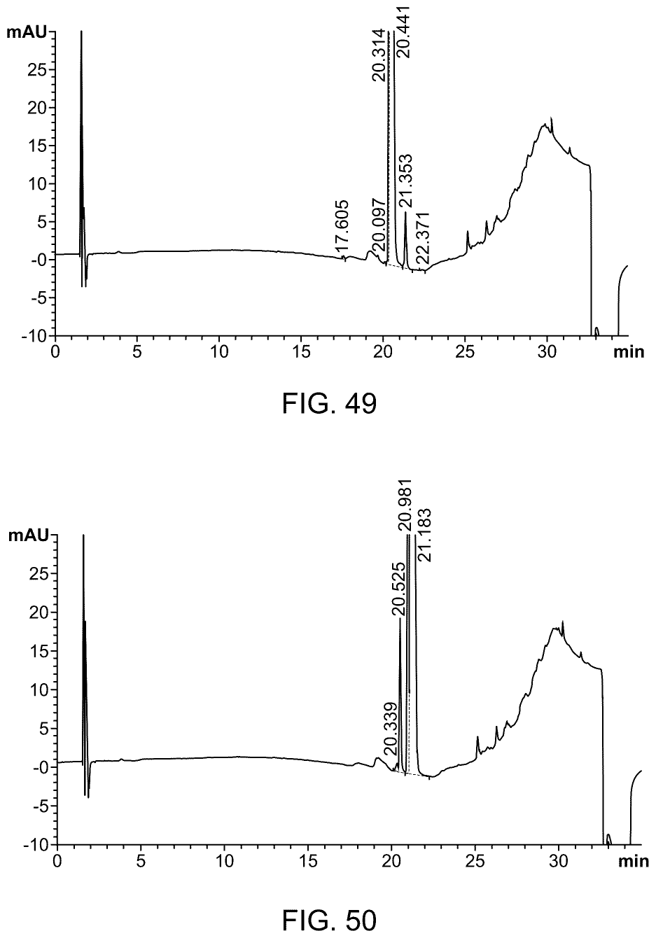

HPLC results with this method are shown on a blank control (FIG. 47), Compound 7b (FIG. 48), Compound 5b (FIG. 49) and Compound 5a (FIG. 50).

A second method utilized an HPLC Agilent 1290 instrument with binary bumps (or an equivalent HPLC system); Poroshell 120 EC-C18 50×4.6 mm, 2.7 μm columns; ambient of 25° C. autosampler temperatures; 40° C. column temperatures; and 0.4 mL/minute flow rates. 0.05% TFA in H₂O (“Mobile Phase C”) and acetonitrile (“Mobile Phase D”) were utilized as mobile phases in this method. Mobile phase gradients for this method are summarized in TABLE 3.

TABLE 3

| Time (min) | Phase C (% v/v) | Phase D (% v/v) |
|---|---|---|
| 0 | 98 | 2 |
| 1 | 98 | 2 |
| 8 | 5 | 95 |
| 13 | 5 | 95 |
| 13.01 | 98 | 2 |
| 14 | 98 | 2 |

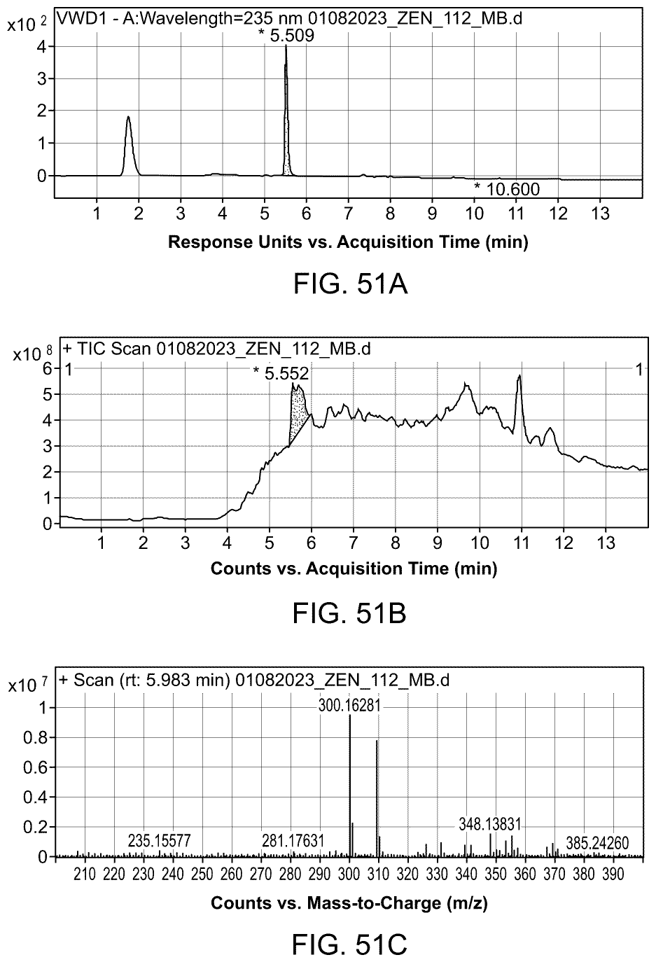

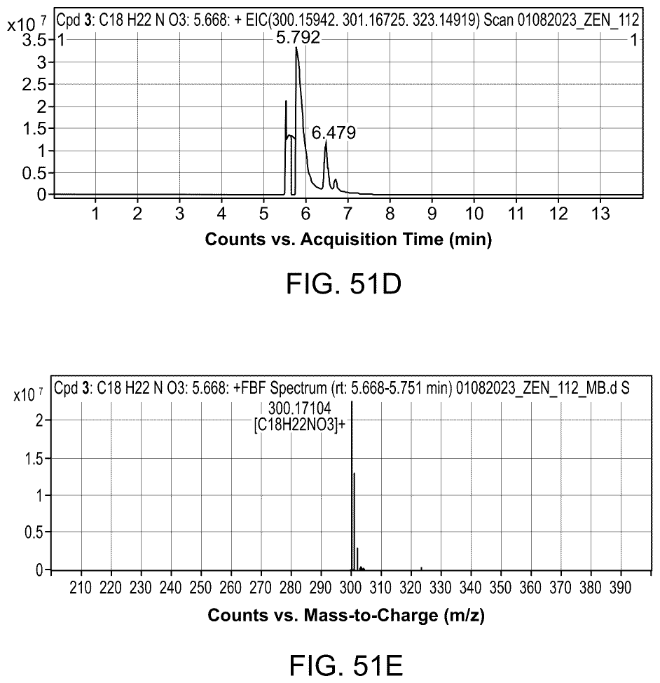

Purification results for Compound 3 using this method are shown in FIGS. 51A-E. FIG. 51A provides chromatography results on the sample in variable wavelength detection mode. FIG. 51B provides chromatography results on the sample in total ion count mode. FIG. 51C provides chromatography/mass spectrometry results on the sample at a retention time of 5.983 minutes. FIG. 51D provides chromatography results on the sample. FIG. 51E provides chromatography/mass spectrometry results on the sample at a retention time of 5.668-5.751 minutes.

### Example 19

### Toxicity Assays

This example covers the cell toxicities of endoxifen and various endoxifen-related compounds. The toxicities of these compounds were measured using crystal violet cell viabilities assays per the protocols outlined in Feoktistova et al., Cold Spring Harb Protoc., 2016; 4 (PMID: 27037069). This assay utilizes crystal violet, which is a triarylmethane dye that binds to ribose type molecules such as DNA in nuclei. The amount of crystal violet staining is proportional to the cell biomass that is attached to the plate and thus this cell biomass can be used to infer levels of cell viability and cytotoxicity in control/experimental conditions. The cell viability assays were performed on the ERα+ cancer cell lines MCF7 and T47D, as well as the MCF7 and T47D cell line derivatives MCF7_ESR1_D538G, MCF7_ESR1_Y537S, T47D, ESR1_Y537S, and T47D_ESR1_D538G, which include ERα point mutations that render this receptor constrictively active. These point mutations (D538G and Y537S) are frequently observed in patients with recurrence following aromatase inhibitor therapy. Upwards of 40% of all patients that have recurrent metastatic disease have activating mutations in ESR1, of which most are one of these two forms. Identifying compounds that can inhibit cells harboring these mutations is thus a major clinical need.

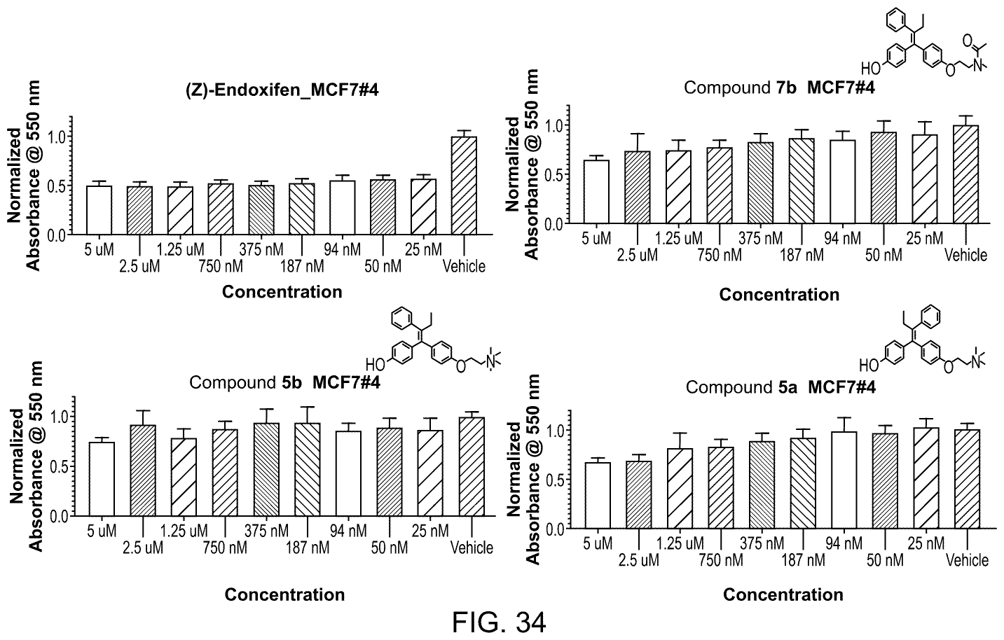

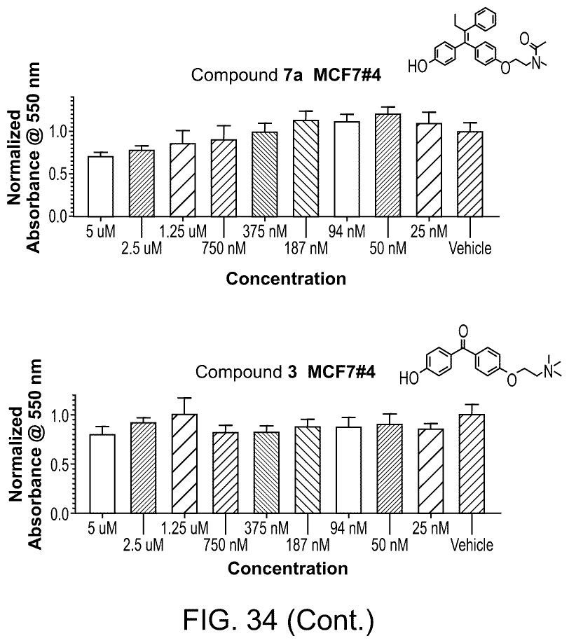

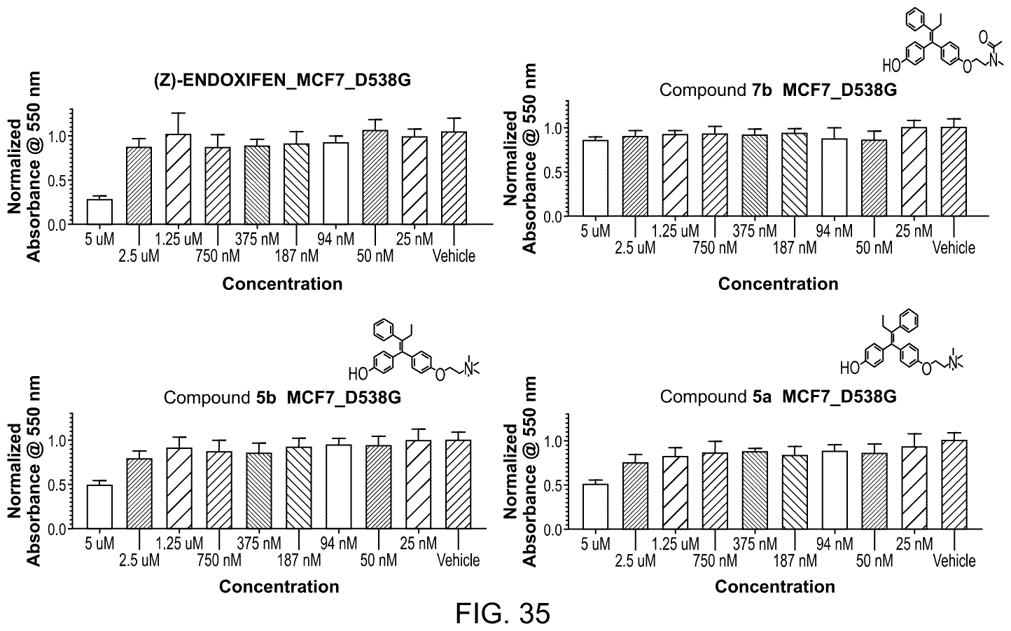

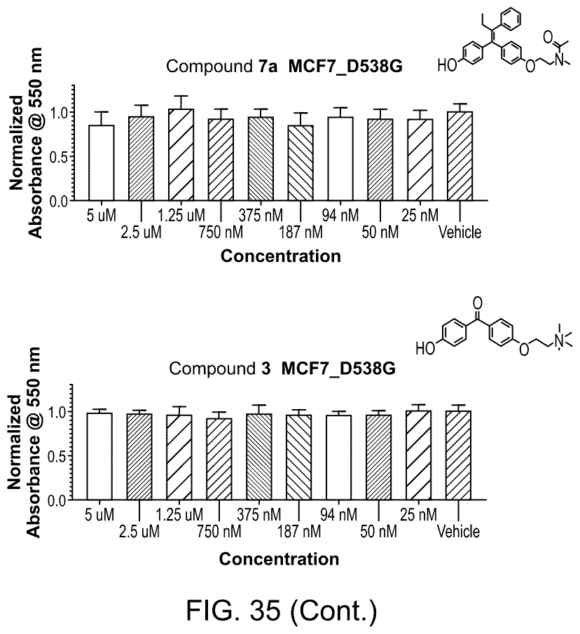

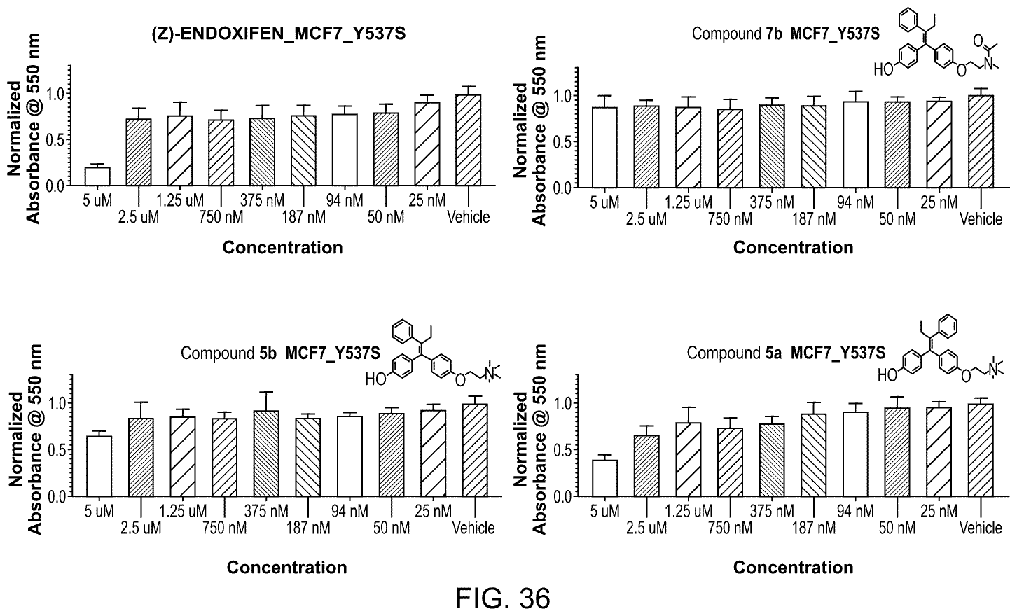

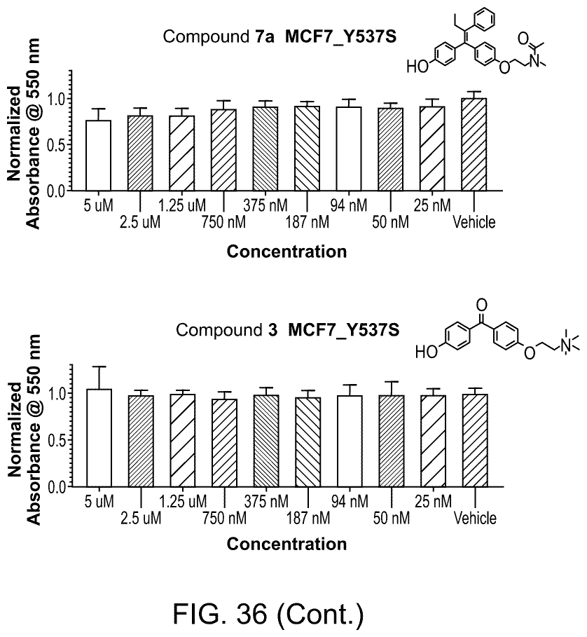

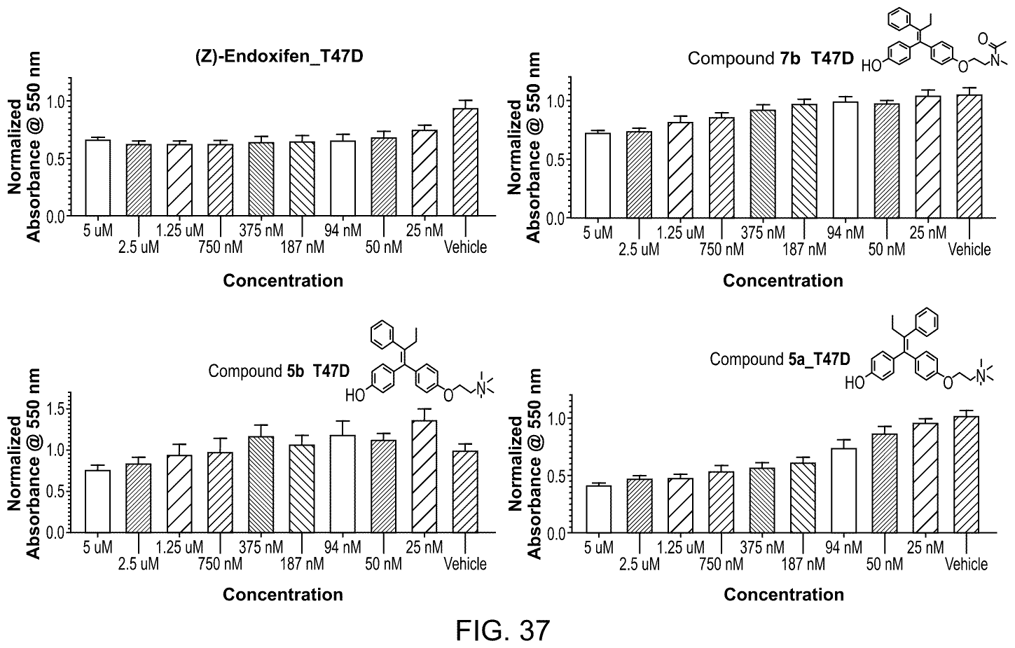

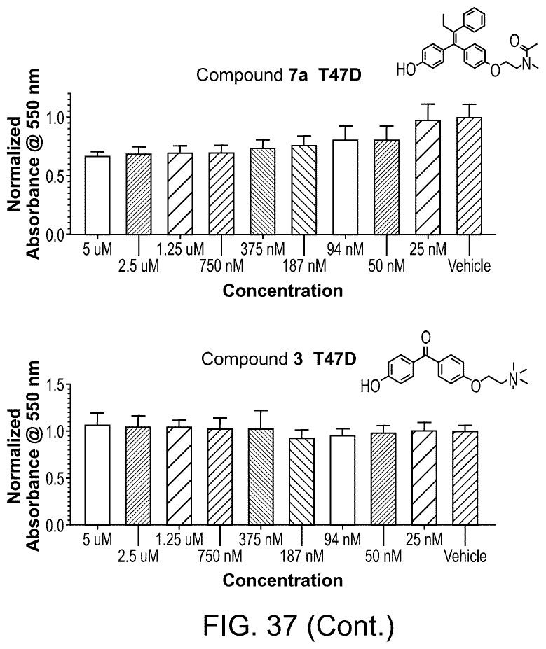

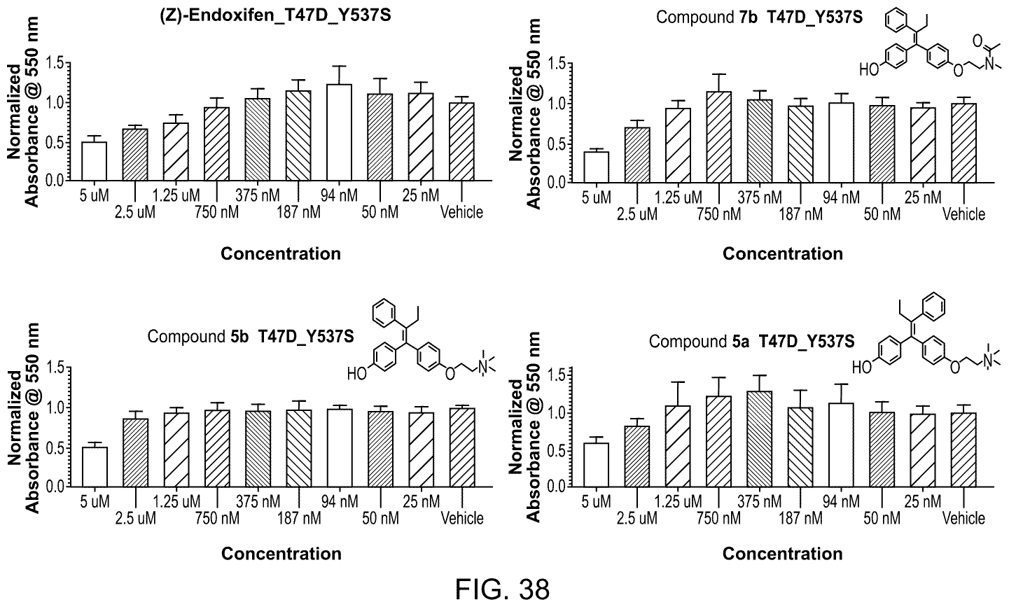

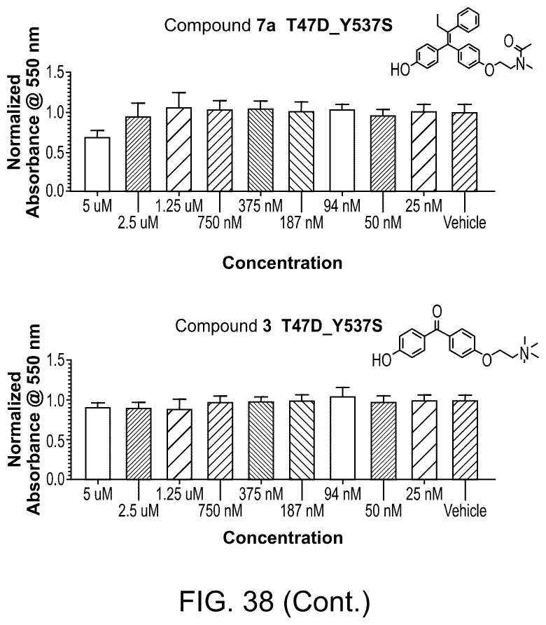

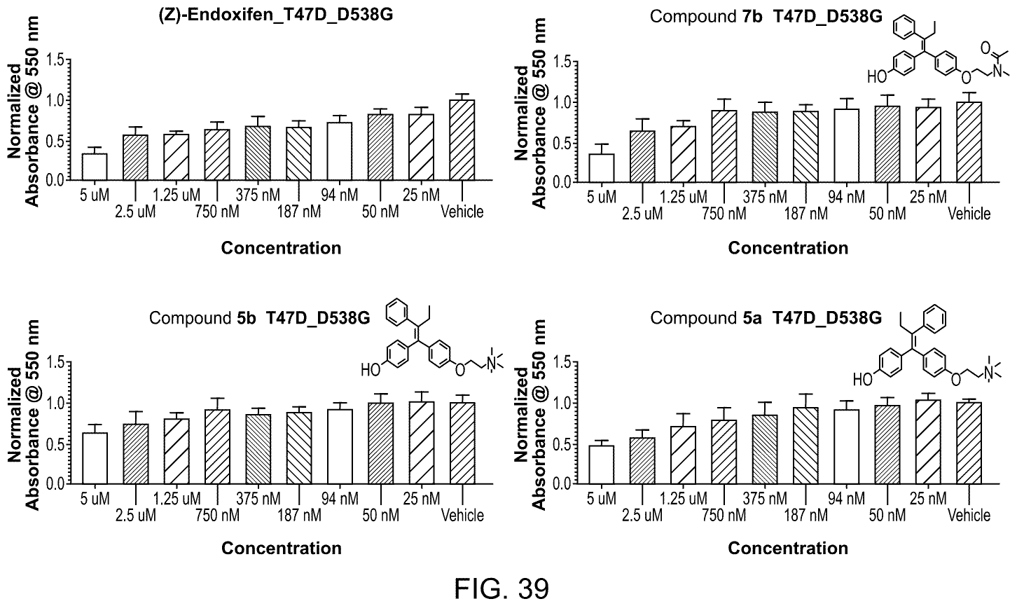

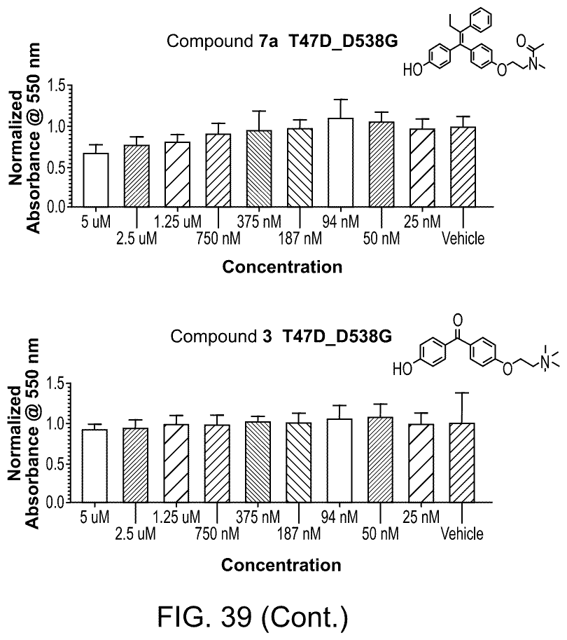

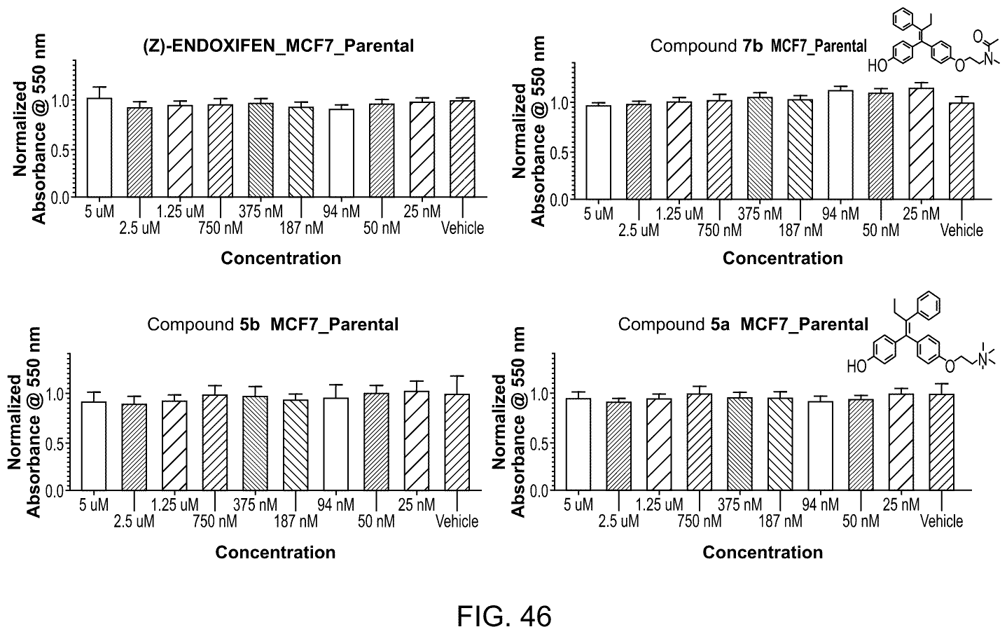

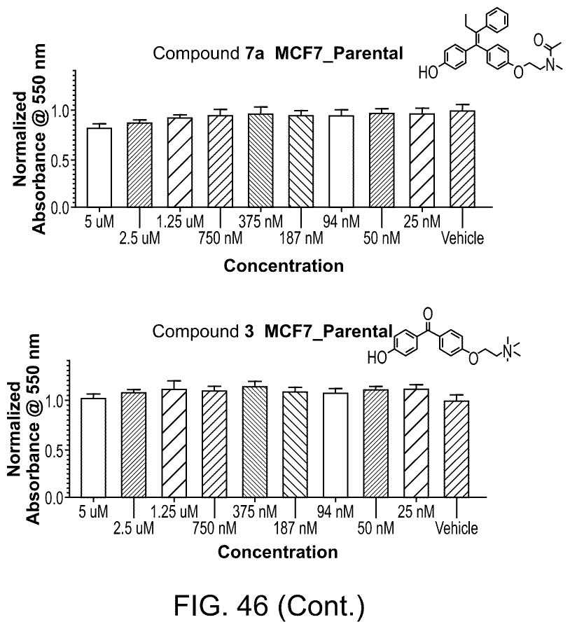

FIGS. 34-39 summarize crystal violet assays with endoxifen (top-left plot in each figure), Compound 7b (top-middle plot in each figure), Compound 7a (top-right plot in each figure), Compound 5b (bottom-left plot in each figure), Compound 5a (bottom-middle plot in each figure), and Compound 3 (bottom-right plot in each figure). Each individual plot provides normalized absorbance (corresponding to cell concentration) over 25 nM to 5 μM concentration ranges, with the rightmost entry in each plot corresponding to vehicle treatment. FIGS. 34-39 correspond to MCF7, MCF7_ESR1_D538G, MCF7_ESR1_Y537S, T47D, T47D_ESR1_Y537S, and T47D_ESR1_D538G, respectively. FIG. 46 is a collection of plots of crystal violet assay results with endoxifen (top-left plot), Compound 7b (top-middle plot), Compound 7a (top-right plot), Compound 5b (bottom-left plot), Compound 5a (bottom-middle plot), and Compound 3 (bottom-right plot) on parental MCF7 cell lines.

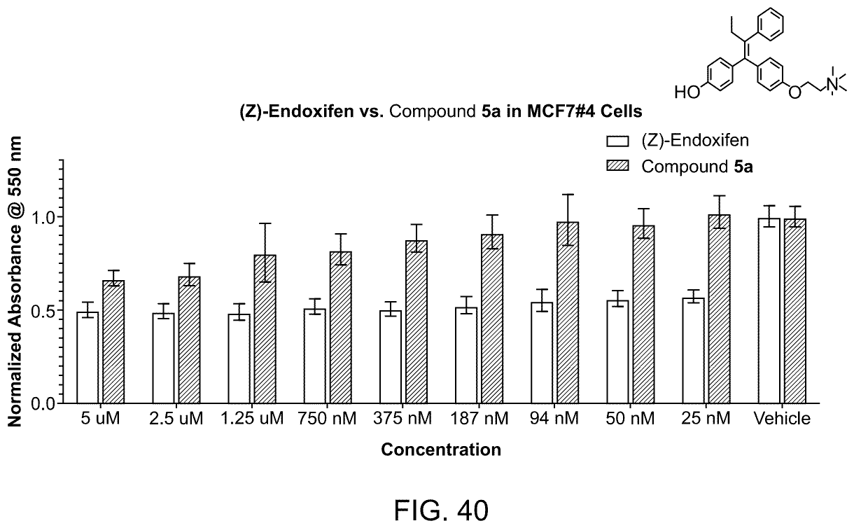

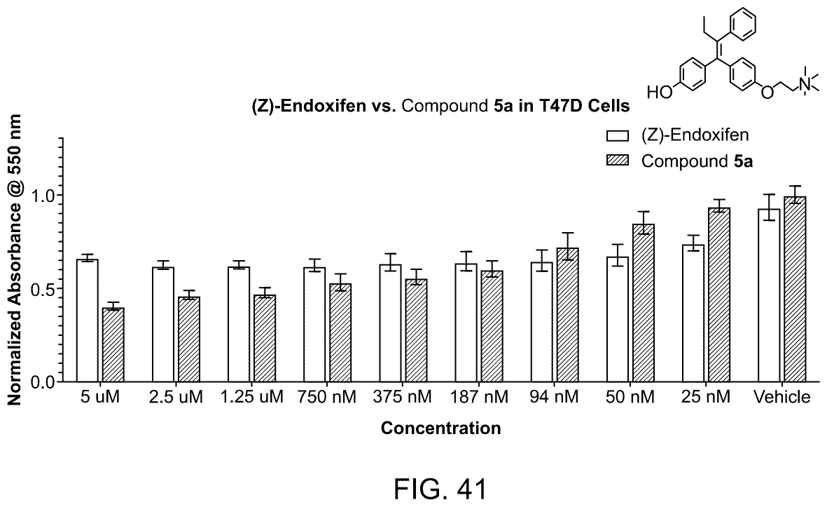

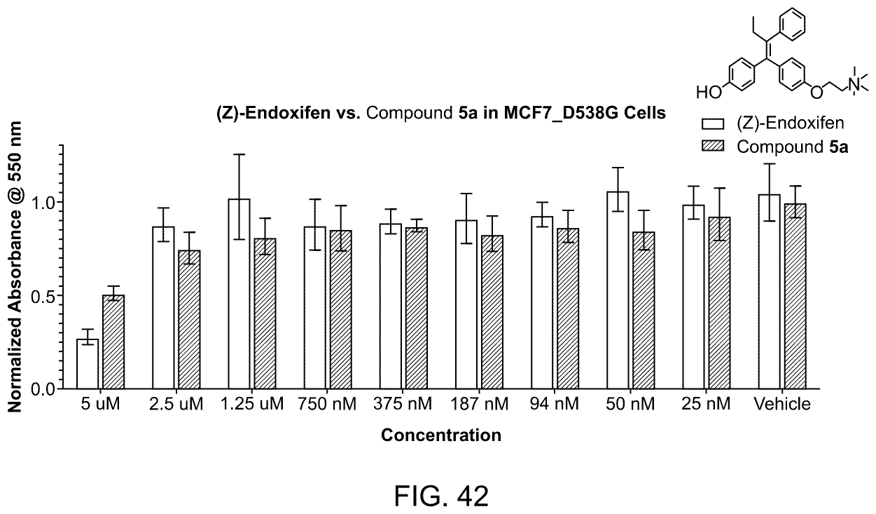

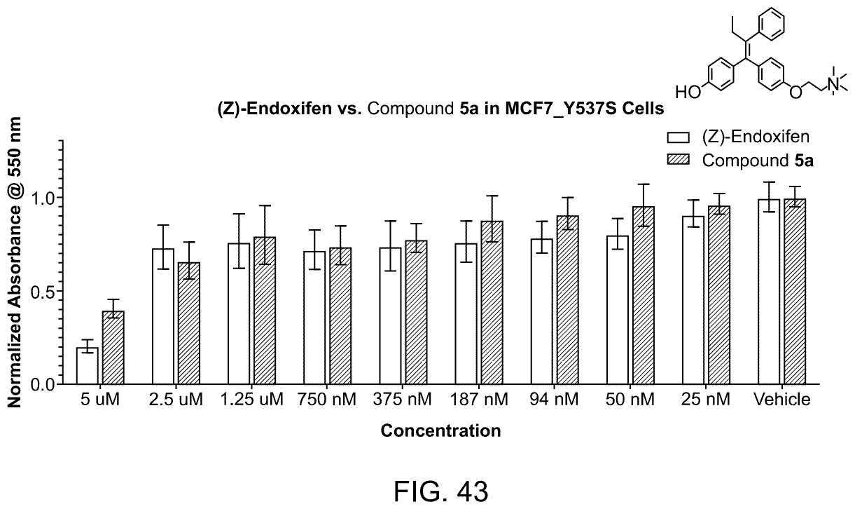

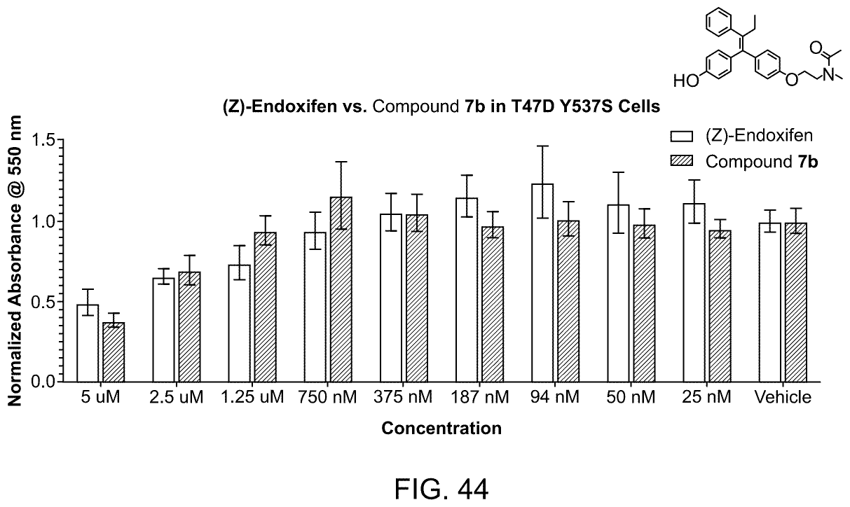

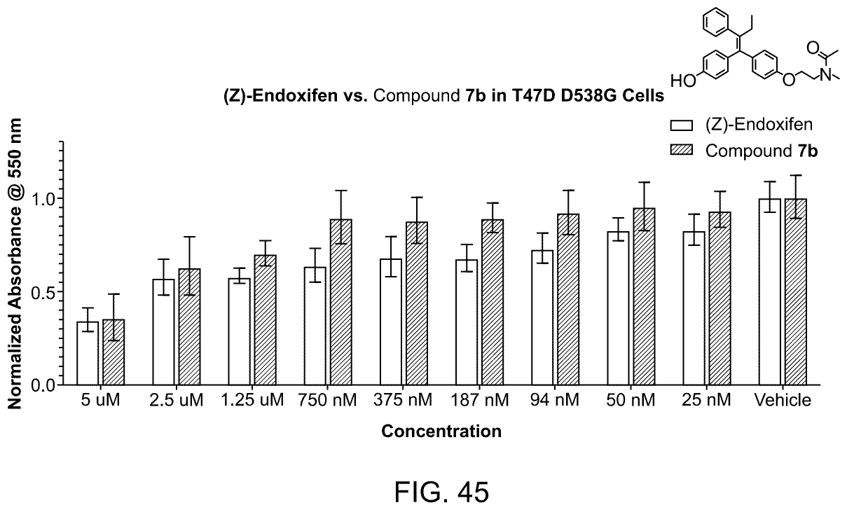

FIGS. 40-43 compare the activities of Compound 5a to endoxifen on the cell lines MCF7, T47D, MCF7_ESR1_D538G, and MCF7_ESR1_Y537S, respectively. FIGS. 44 and 45 compare the activities of endoxifen to Compound 7b in T47D_ESR1_Y537S and T47D_ESR1_D538G cell lines, respectively. In FIGS. 40-45, each compound was separately tested at concentrations ranging from 25 nM to 5 μM. Vehicle treatments are shown at the right of each plot for comparison. As can be seen from these plots, Compounds 5a and 7b exhibit similar activities as endoxifen against the tested cancer cell lines. However, these compounds also exhibit different concentration dependencies than Endoxifen. For example, as shown in FIG. 41, Compound 5a exhibits lower activity against T47D cells than endoxifen at sub-100 nanomolar concentrations and greater activity than endoxifen at micromolar concentrations.

### Example 20

### Cell Migration Assays

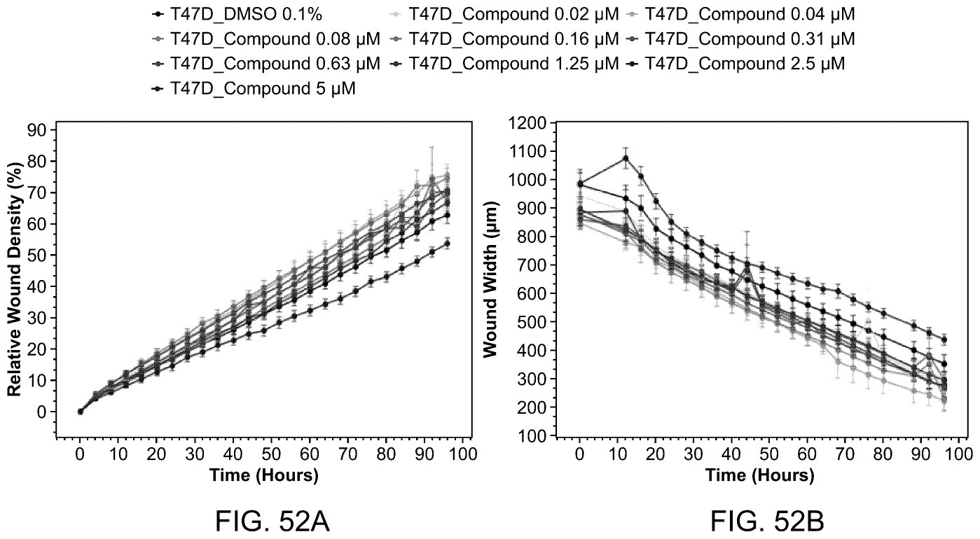

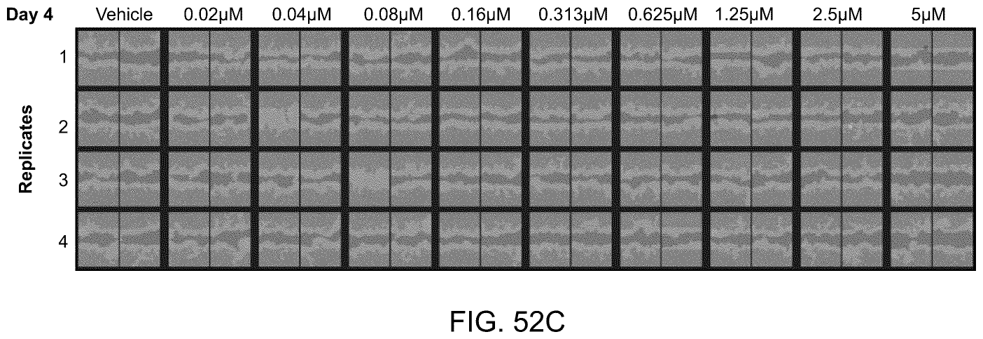

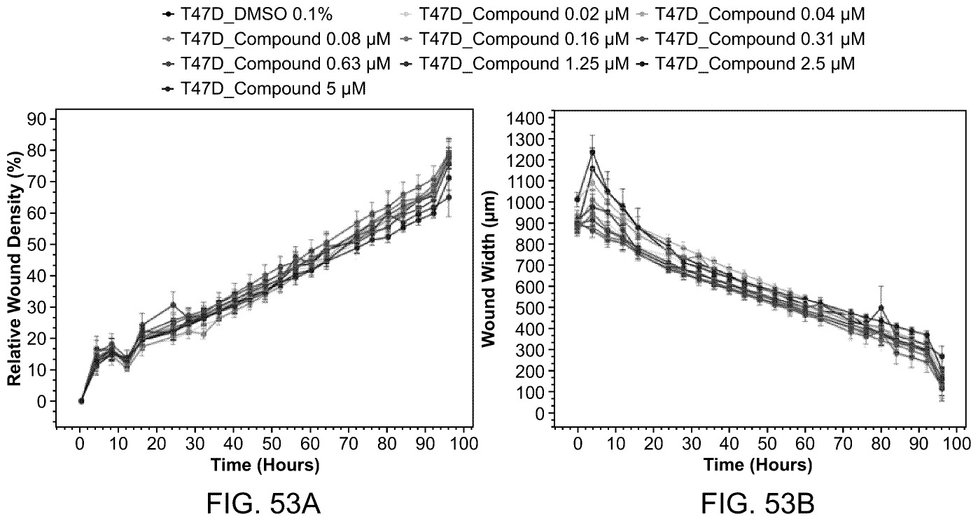

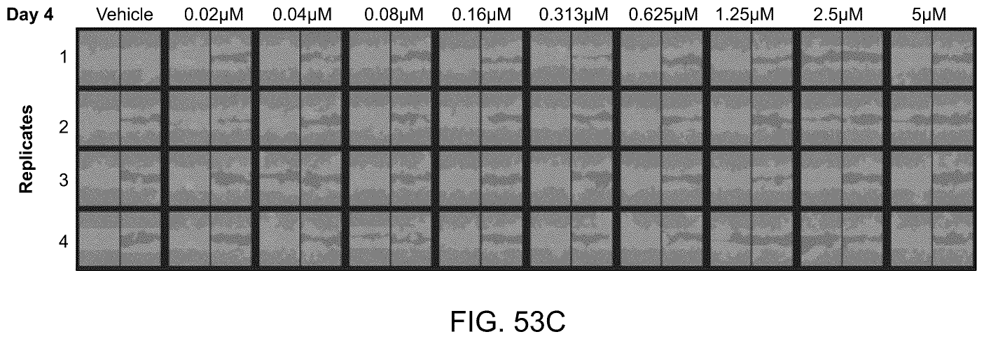

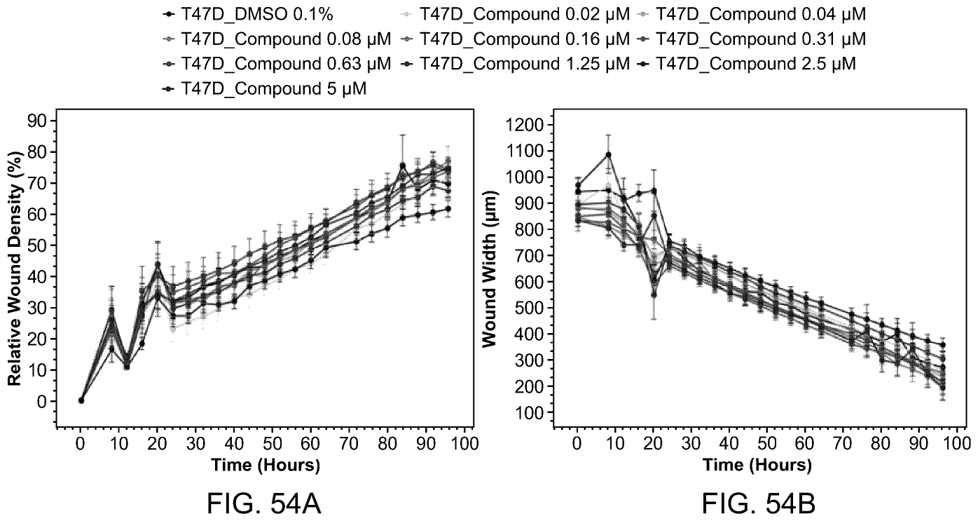

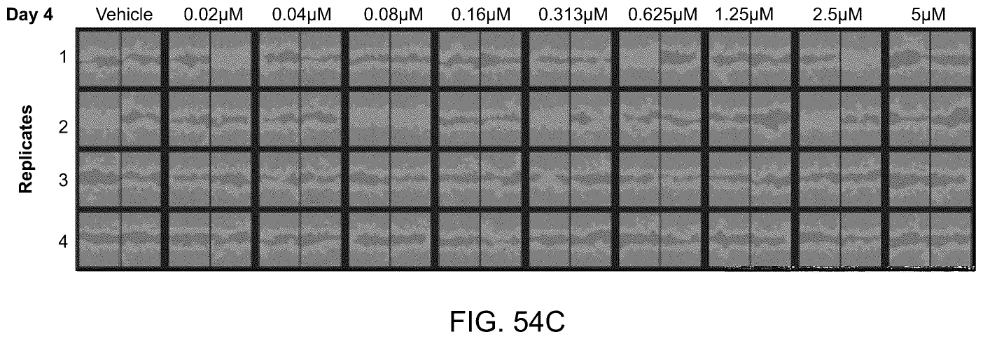

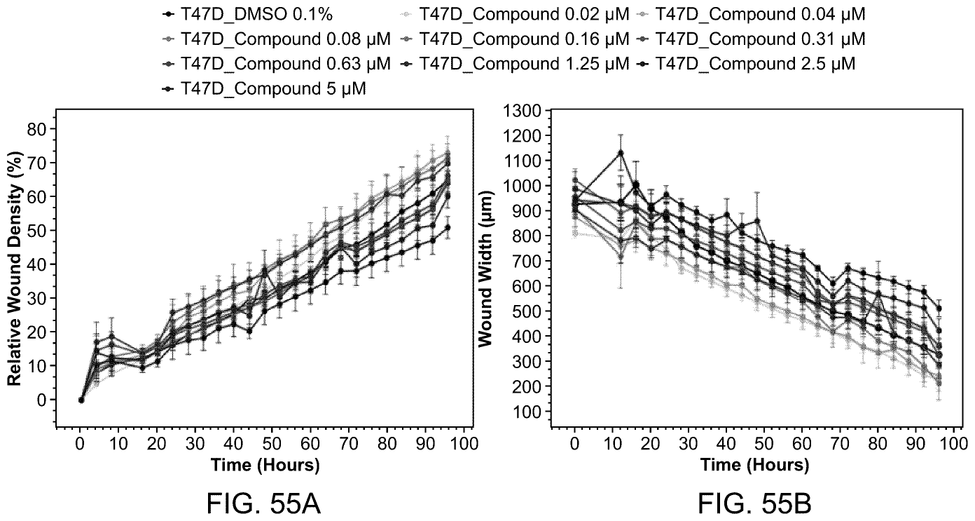

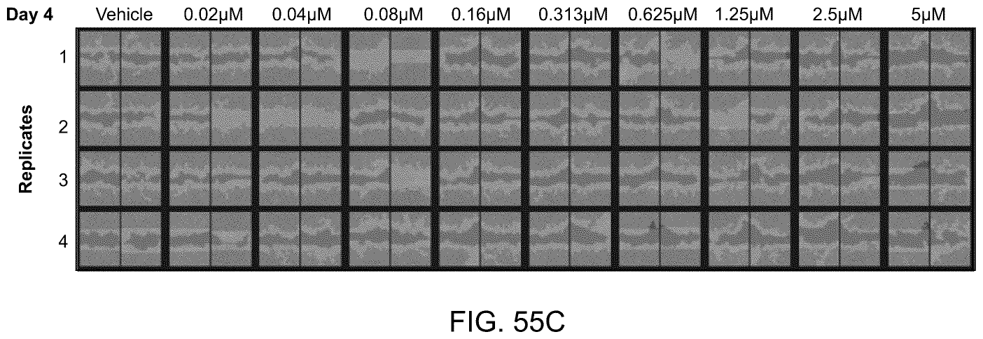

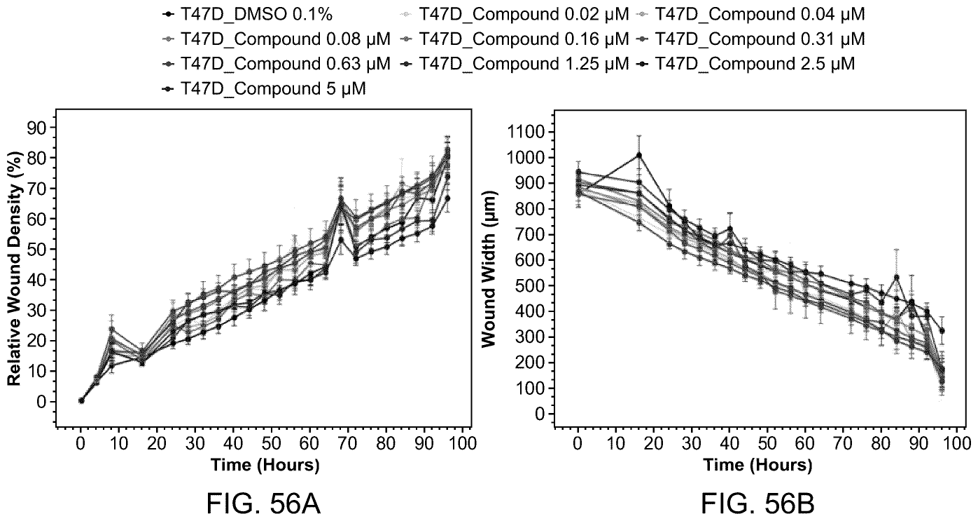

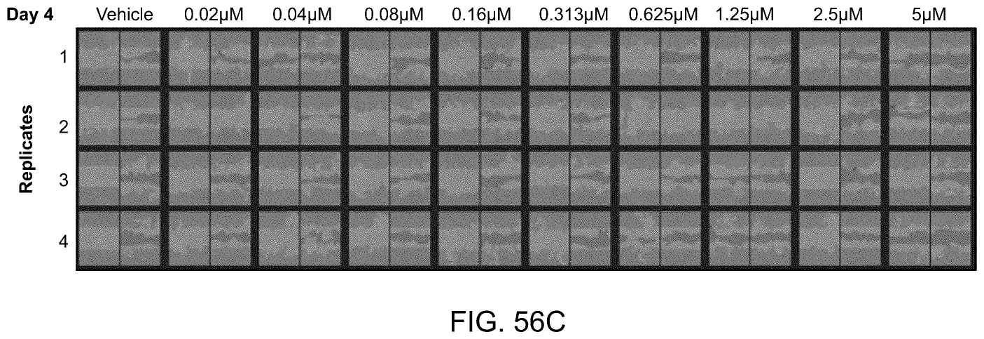

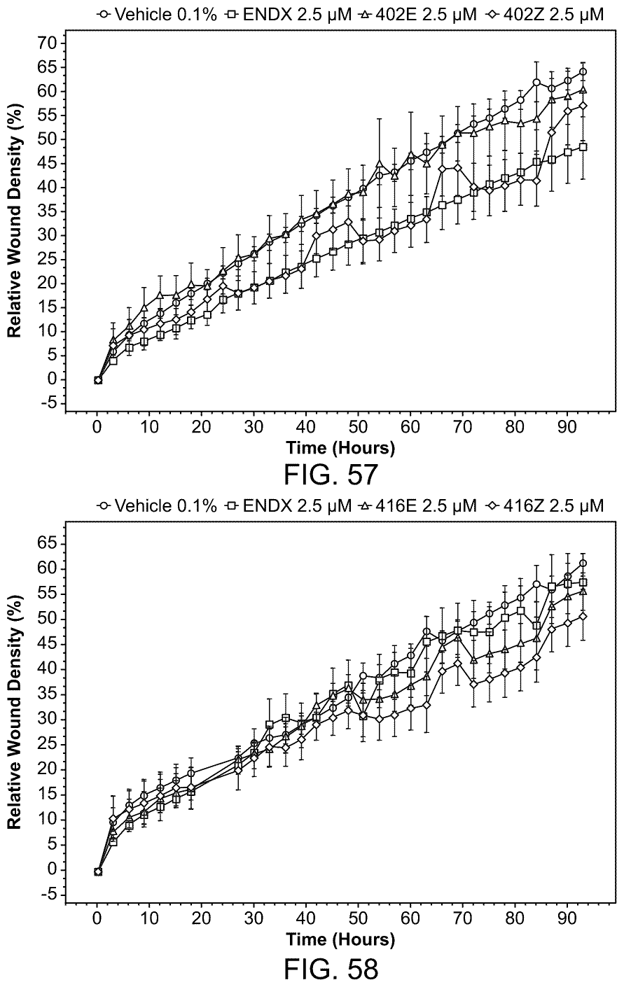

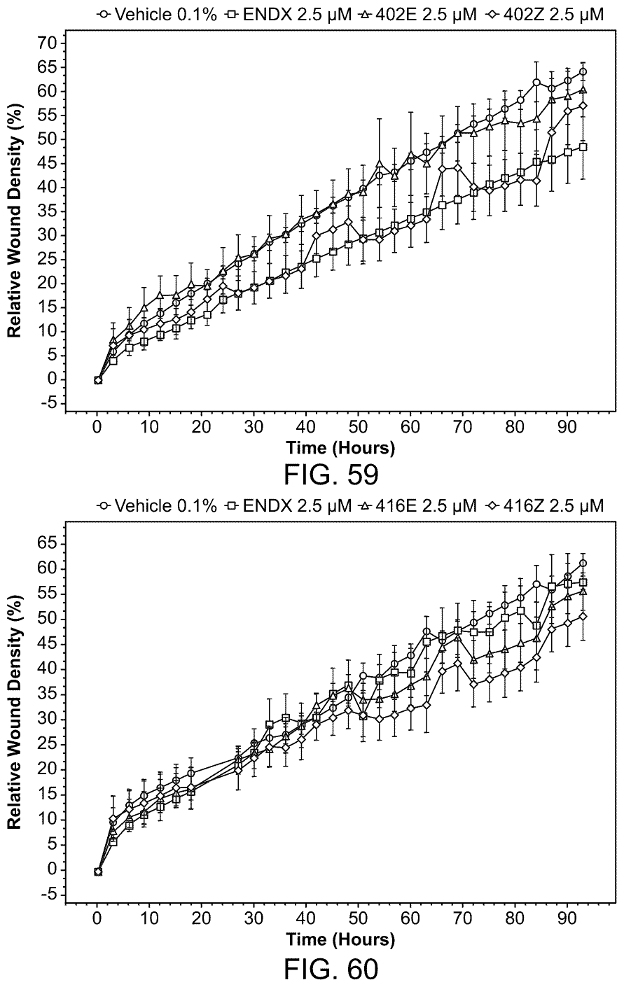

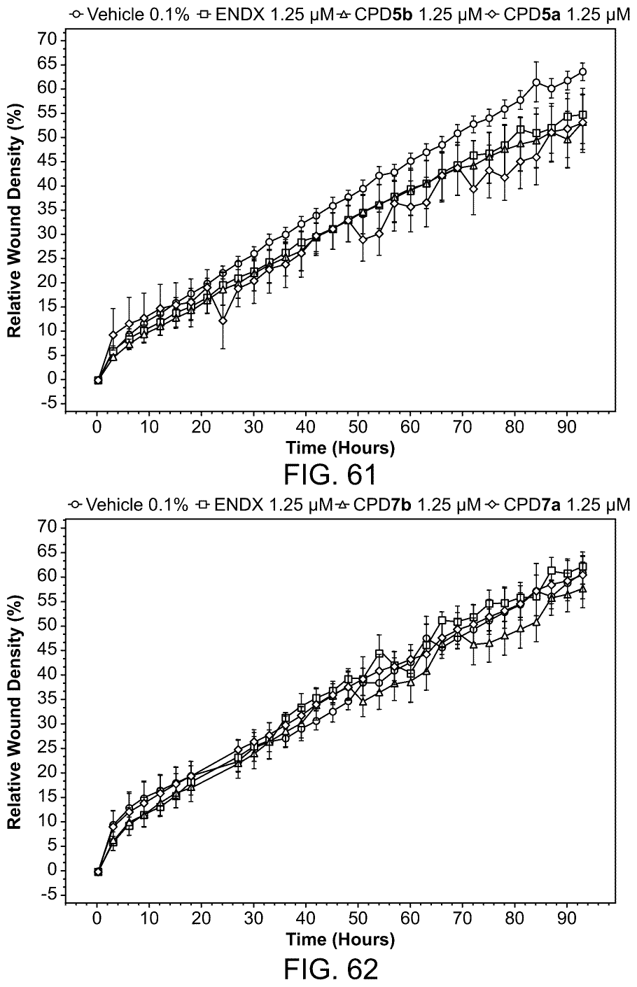

This example covers cell migration assays with endoxifen and multiple endoxifen-related compounds on T47D cancer cells. FIGS. 52A, 53A, 54A, 55A, and 56A are plots of relative wound density following treatment with vehicle or 0.02 to 6 μM endoxifen (FIG. 52A), Compound 5b (FIG. 53A), Compound 5a (FIG. 54A), Compound 7a (FIG. 55A), and Compound 7b (FIG. 56A) as a function of time (0 to 100 hours). FIGS. 52B, 53B, 54B, 55B, and 56B are plots of wound width following treatment with vehicle or 0.02 to 6 μM endoxifen (FIG. 52B), Compound 5b (FIG. 53B), Compound 5a (FIG. 54B), Compound 7a (FIG. 55B), and Compound 7b (FIG. 56B) as a function of time (0 to 100 hours). FIGS. 52C, 53C, 54C, 55C, and 56C are series of plots of individual replicates of relative wound density and wound width for endoxifen, Compound 5b, Compound 5a, Compound 7a, and Compound 7b, respectively. FIG. 57 is a plot of relative wound density over time (0 to 100 hours) for a wound treated with vehicle (DMSO), 5 μM endoxifen, 5 μM Compound 5b, and 5 μM Compound 5a. FIG. 58 is a plot of relative wound density over time (0 to 100 hours) for a wound treated with vehicle (DMSO), 5 μM endoxifen, 5 μM Compound 7a, and 5 μM Compound 7b. FIG. 59 is a plot of relative wound density over time (0 to 100 hours) for a wound treated with vehicle (DMSO), 2.5 μM endoxifen, 2.5 μM Compound 5b, and 2.5 μM Compound 5a. FIG. 60 is a plot of relative wound density over time (0 to 100 hours) for a wound treated with vehicle (DMSO), 2.5 μM endoxifen, 2.5 μM Compound 7a, and 2.5 μM Compound 7b. FIG. 61 is a plot of relative wound density over time (0 to 100 hours) for a wound treated with vehicle (DMSO), 1.25 μM endoxifen, 1.25 μM Compound 5b, and 1.25 μM Compound 5a. FIG. 62 is a plot of relative wound density over time (0 to 100 hours) for a wound treated with vehicle (DMSO), 1.25 μM endoxifen, 1.25 μM Compound 7a, and 1.25 μM Compound 7b. Numerous endoxifen-related compounds exhibited similar effects as endoxifen, with Compound 7b exhibiting greater potency than endoxifen at inhibiting the migration and proliferation of T47D cells at high doses (see FIGS. 58, 60, and 62).

### Example 21

### Cell Cycle Progression Assays

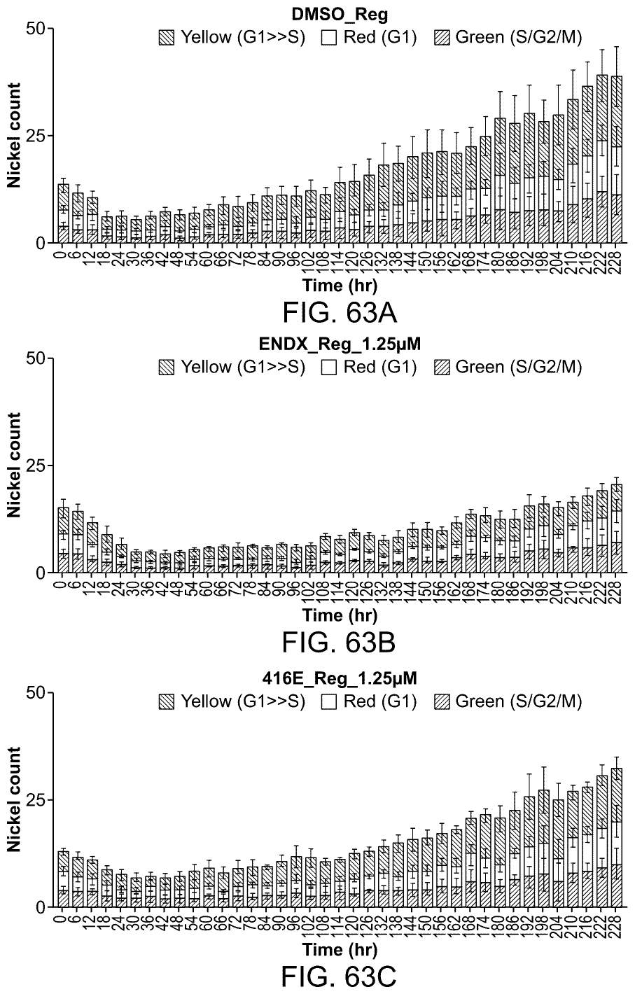

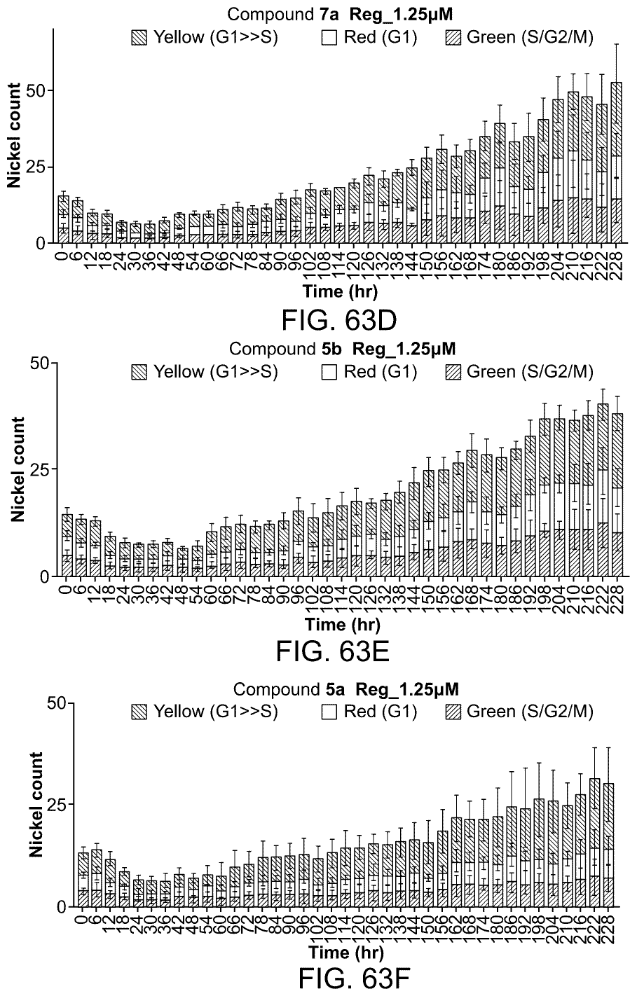

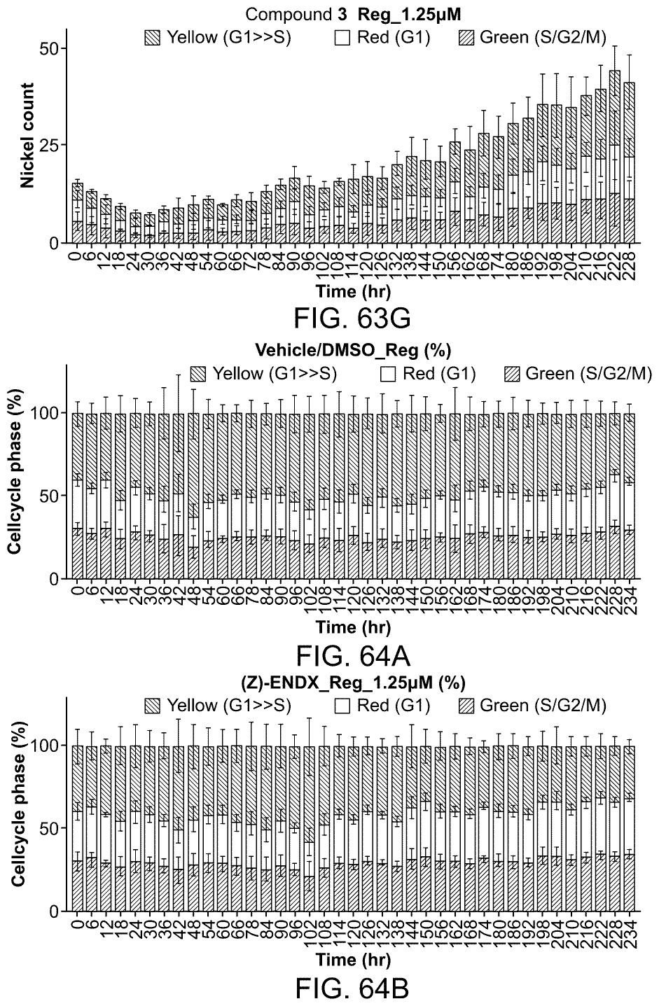

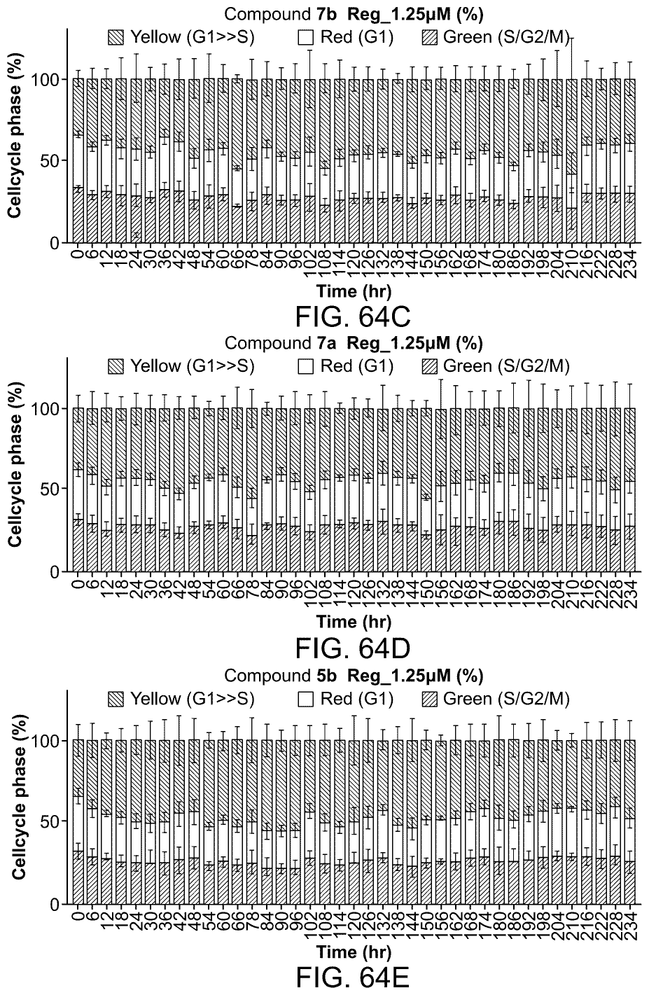

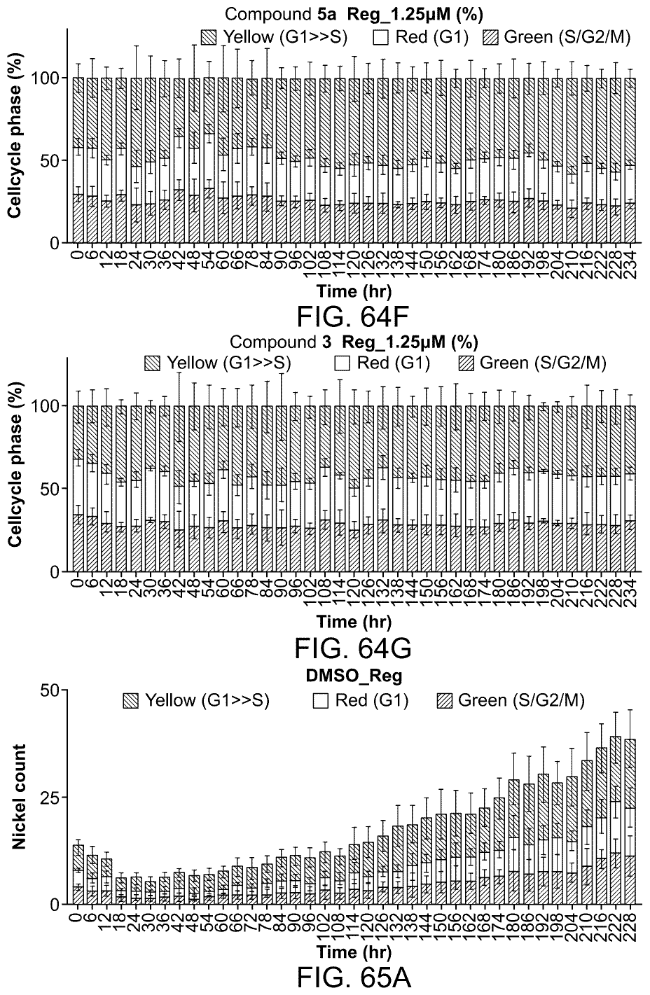

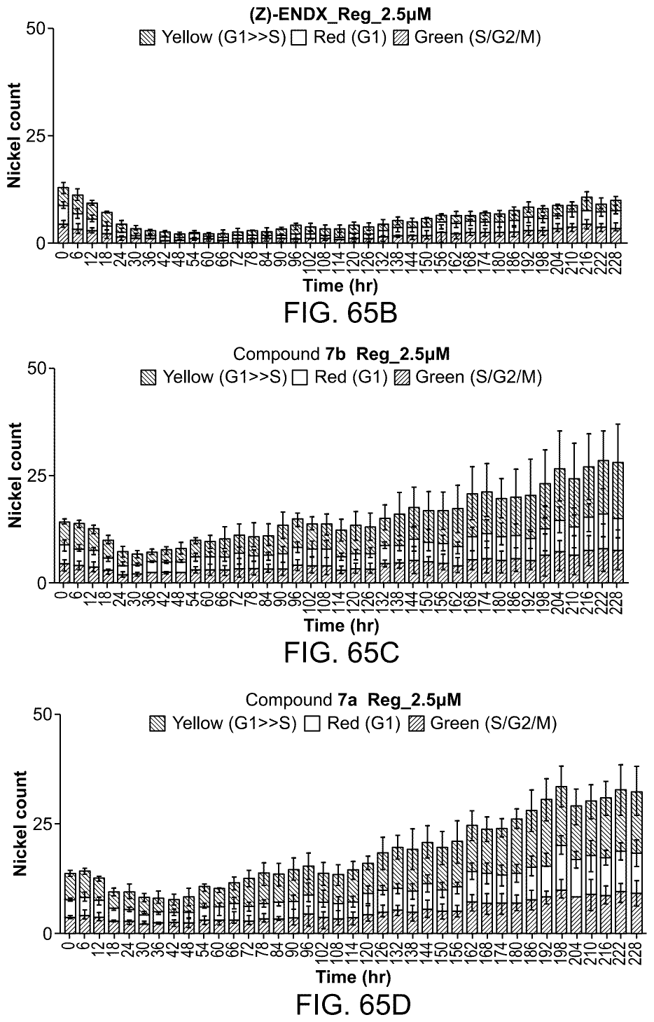

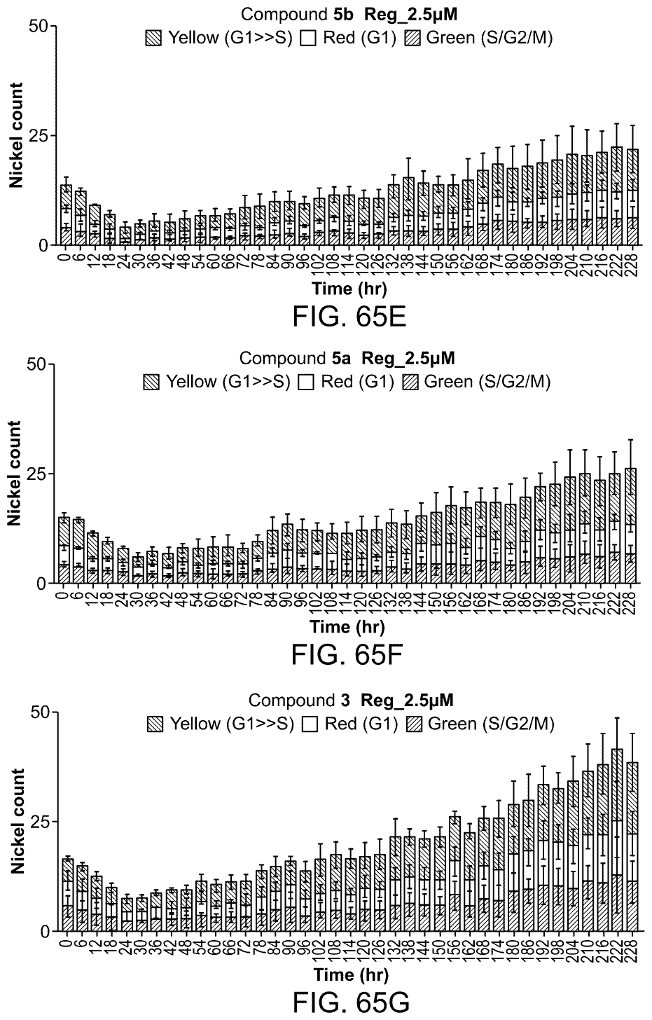

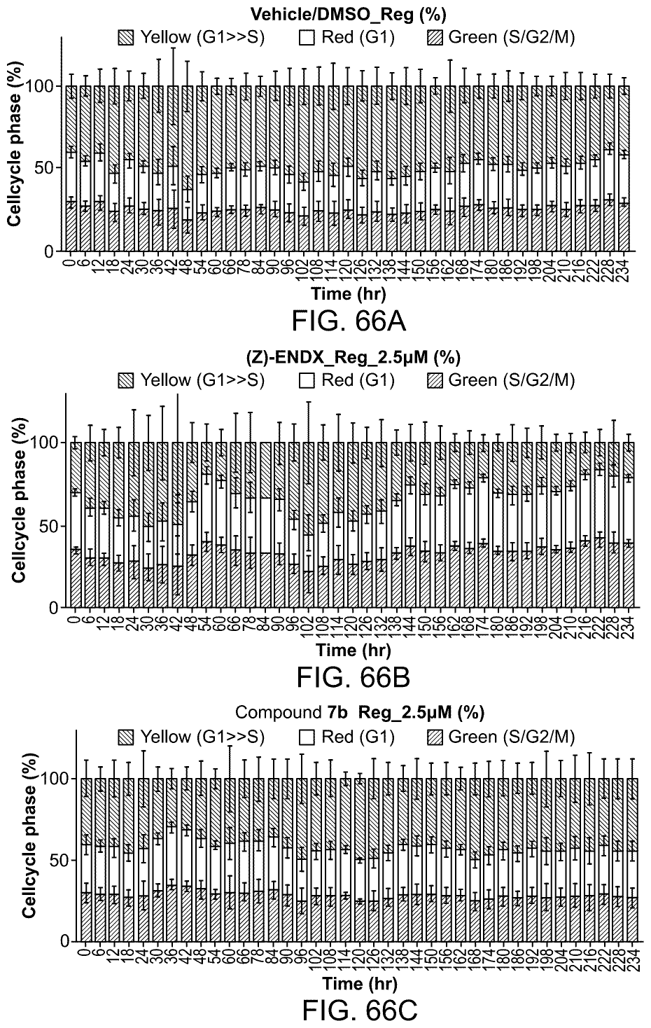

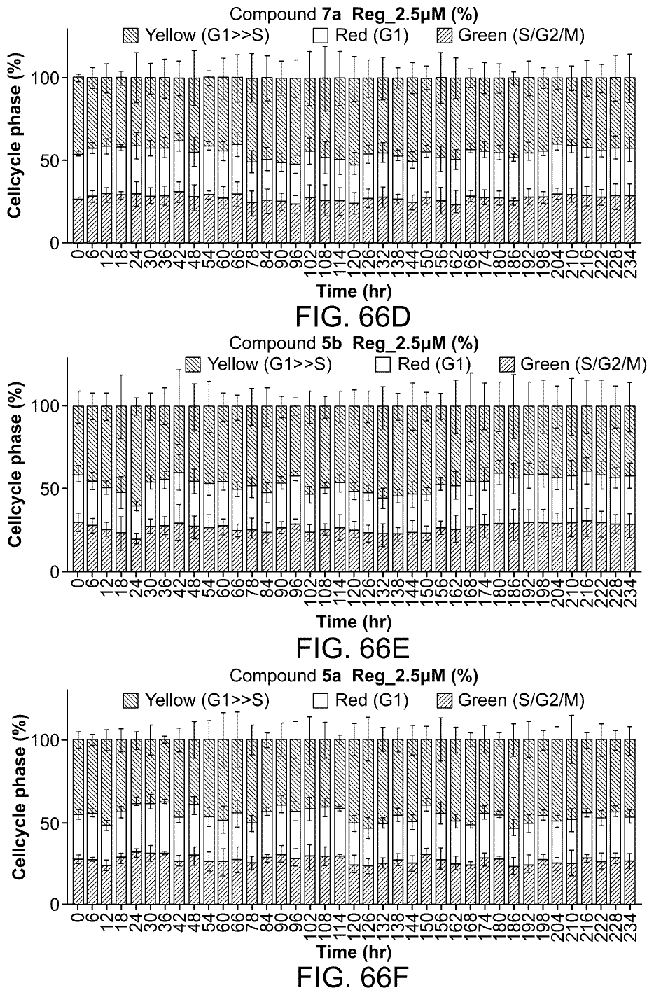

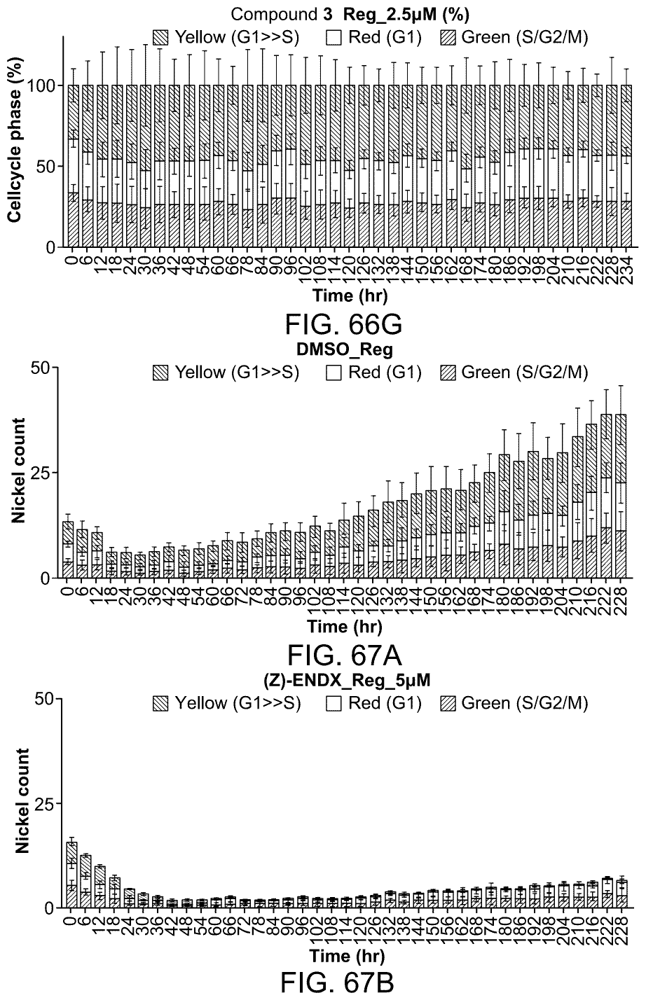

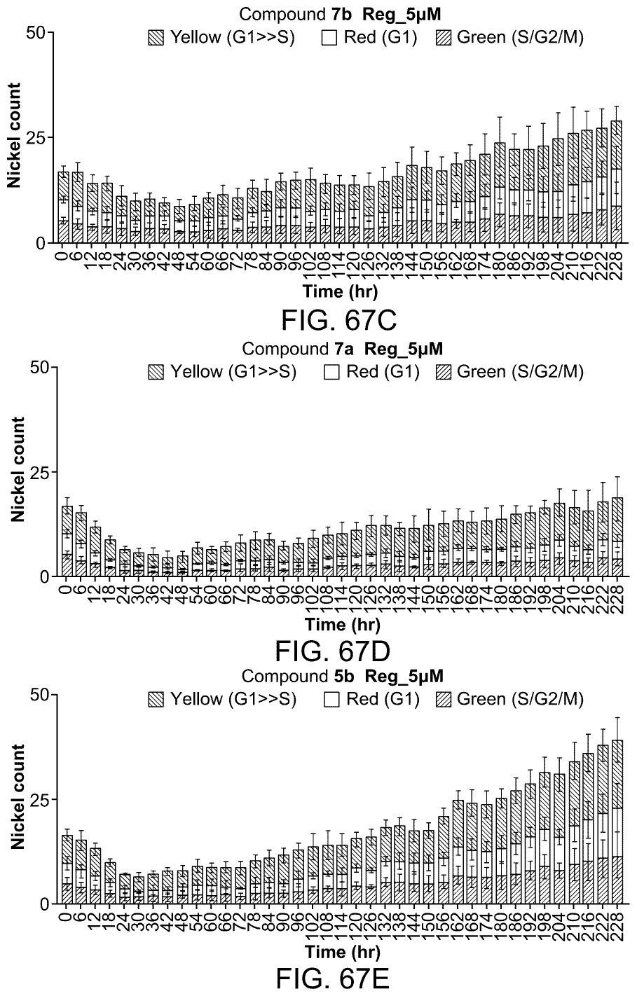

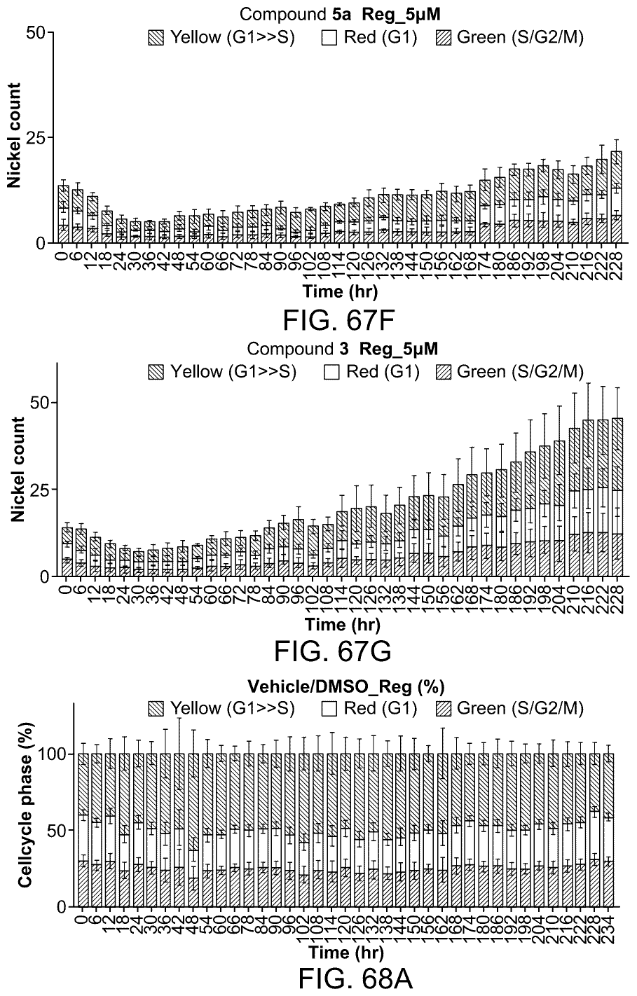

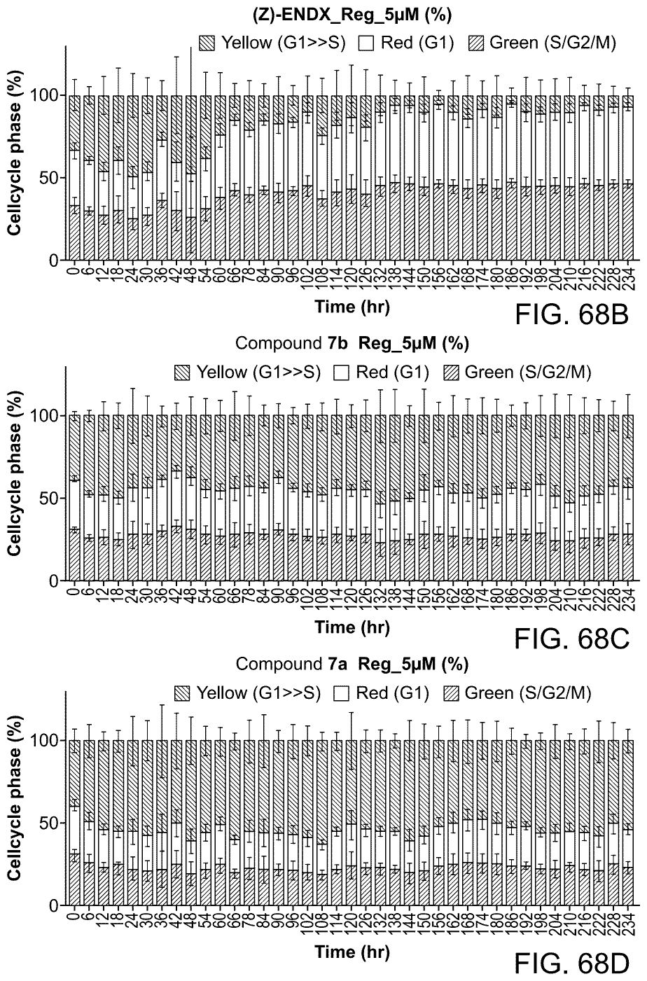

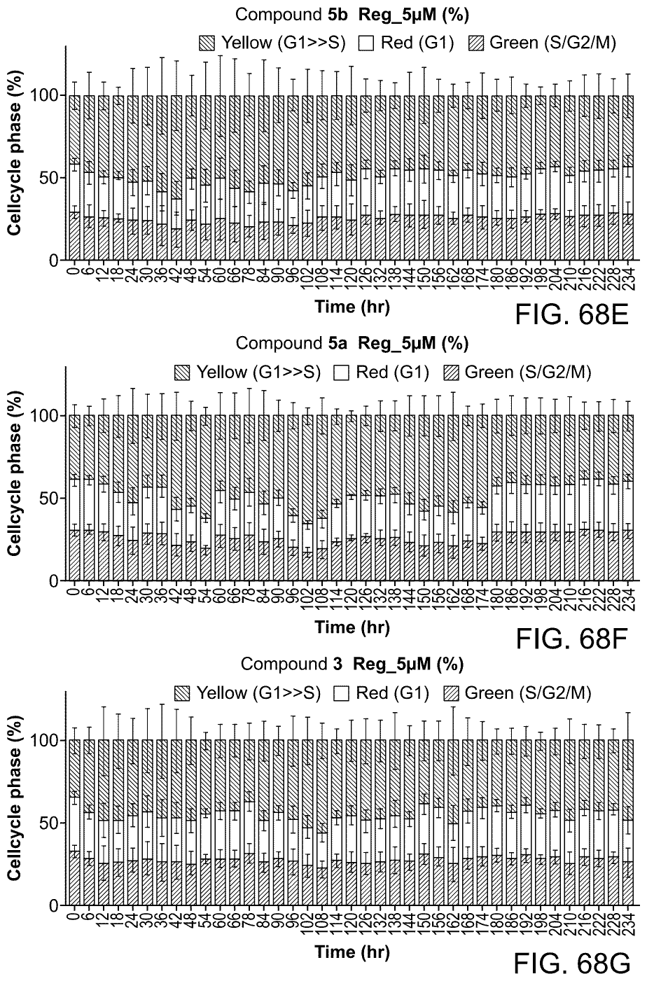

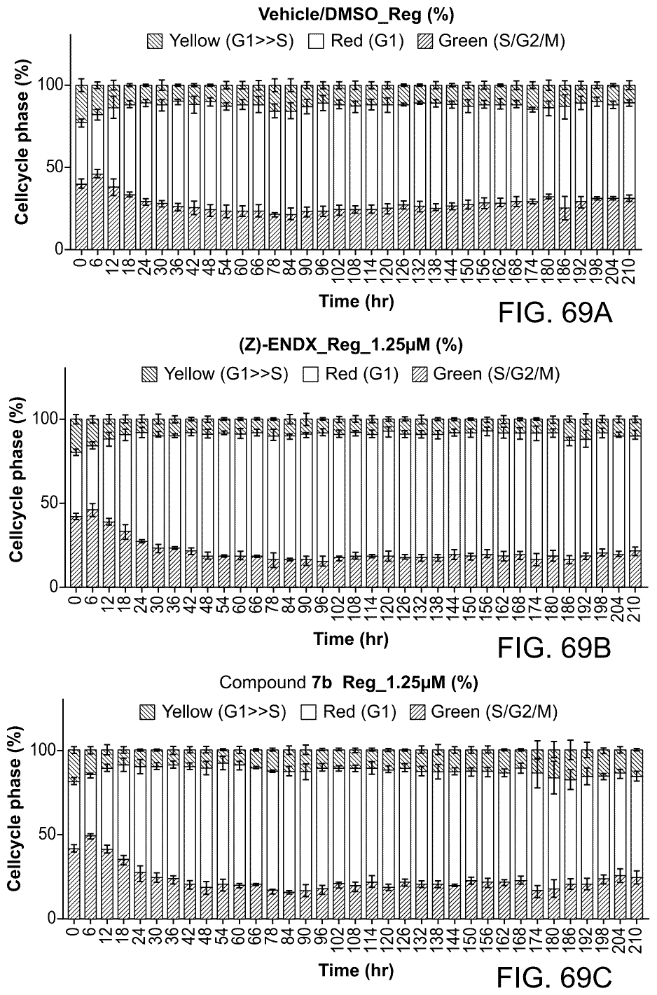

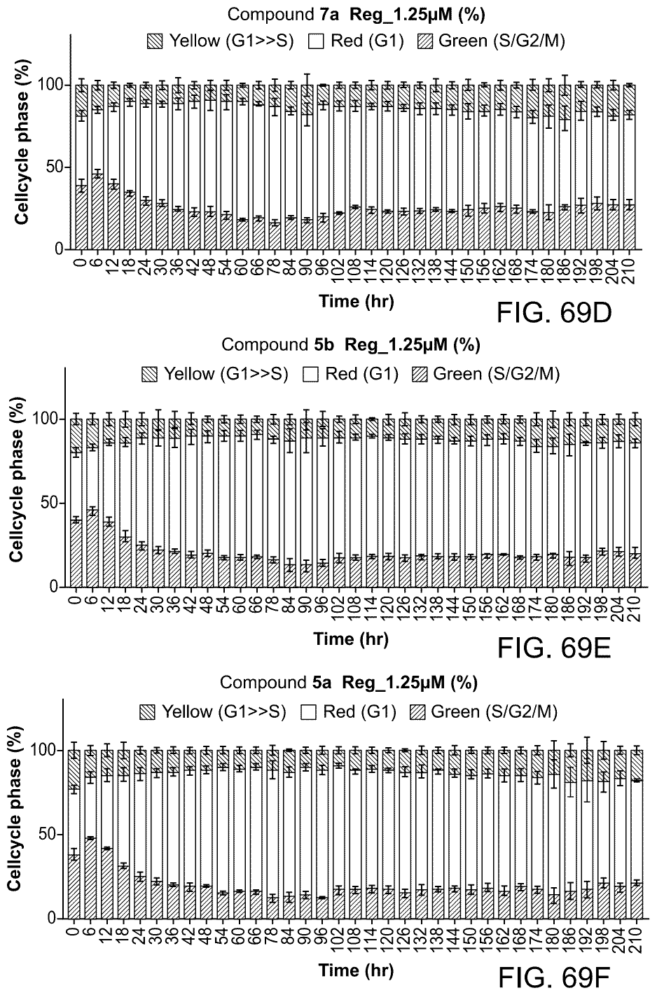

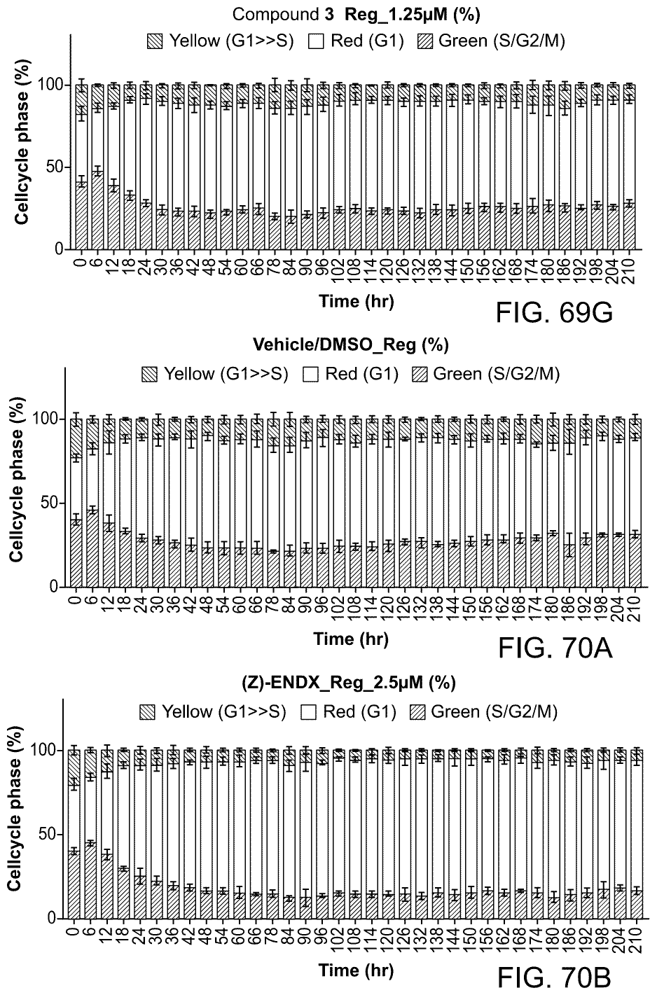

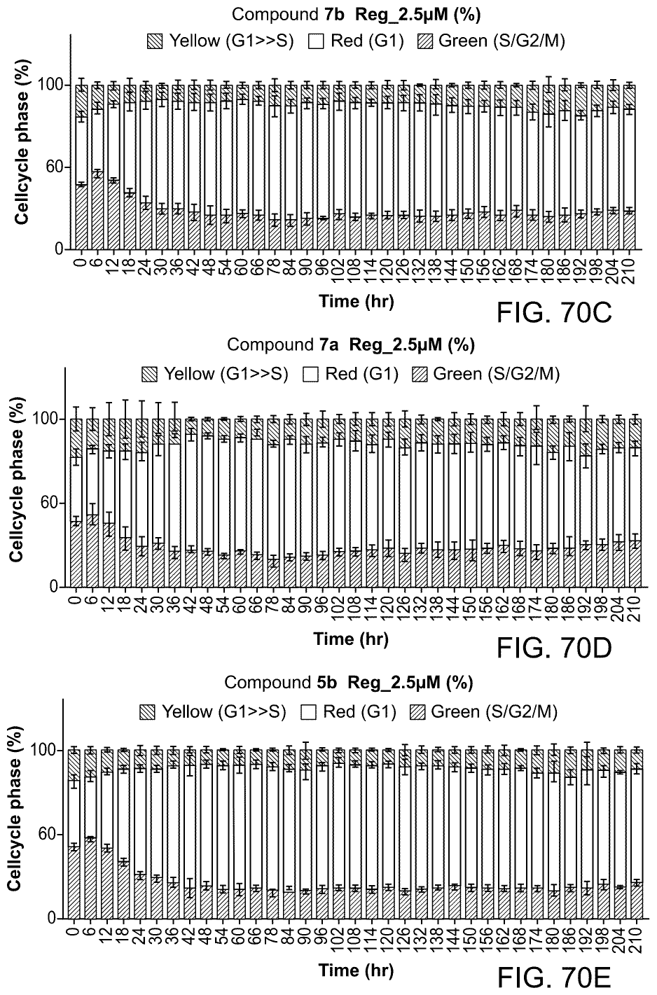

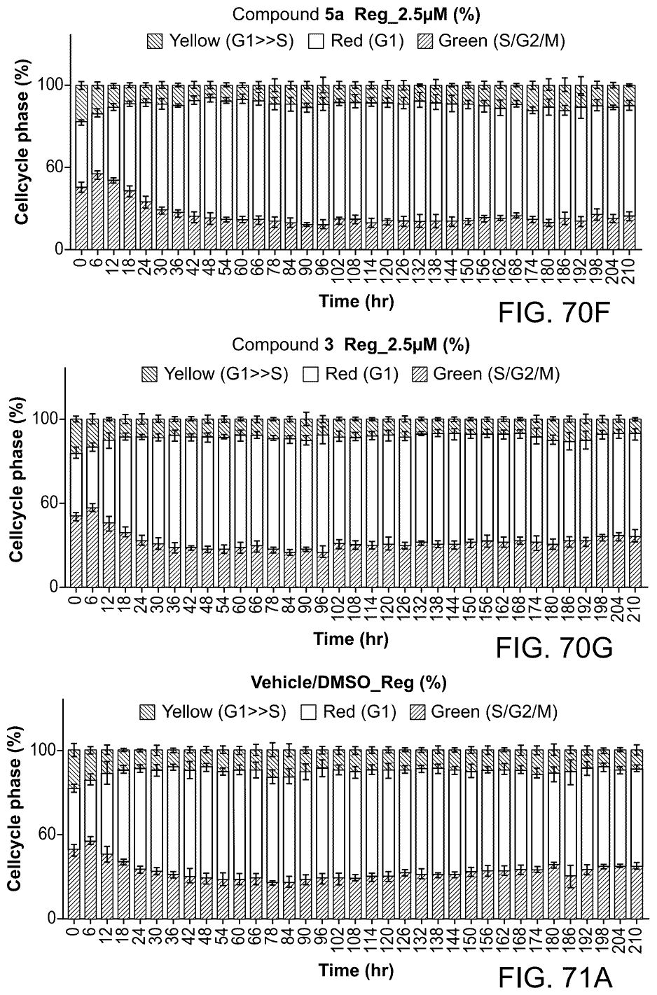

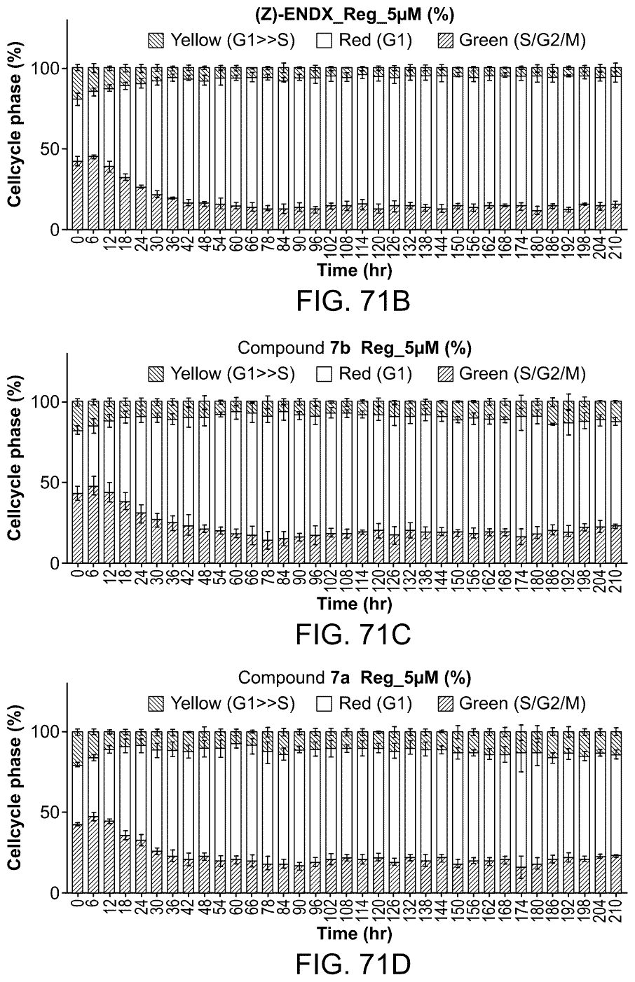

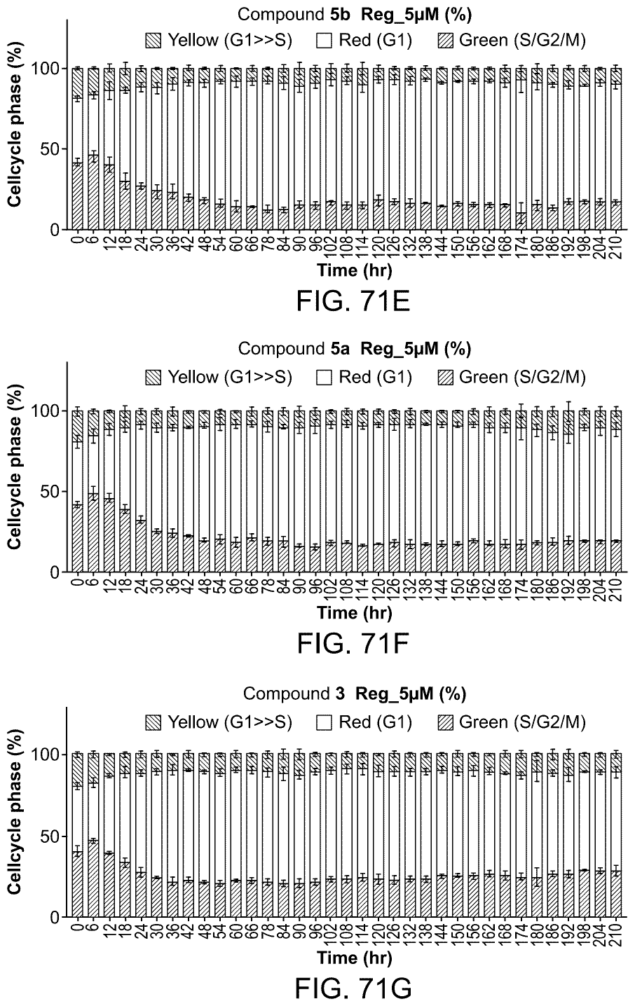

This example covers cell cycle progression assays with endoxifen and multiple endoxifen-related compounds on T47D cancer cells. FIGS. 63A-G are plots that show absolute cell counts for cells in different cell cycle phases (G1 through S; G1; and S, G2, and M) following treatment with vehicle (DMSO) or 1.25 μM endoxifen, Compound 7b, Compound 7a, Compound 5b, Compound 5a, and Compound 3, respectively. FIGS. 64A-G are plots that show percent cell counts for cells in different cell cycle phases following treatment with vehicle or 1.25 μM endoxifen, Compound 7b, Compound 7a, Compound 5b, Compound 5a, and Compound 3, respectively. FIGS. 65A-G are plots that show absolute cell counts for cells in different cell cycle phases following treatment with vehicle or 2.5 μM endoxifen, Compound 7b, Compound 7a, Compound 5b, Compound 5a, and Compound 3, respectively. FIGS. 66A-G are plots that show percent cell counts for cells in different cell cycle phases following treatment with vehicle or 2.5 μM endoxifen, Compound 7b, Compound 7a, Compound 5b, Compound 5a, and Compound 3, respectively. FIGS. 67A-G are plots that show absolute cell counts for cells in different cell cycle phases following treatment with vehicle or 5 μM endoxifen, Compound 7b, Compound 7a, Compound 5b, Compound 5a, and Compound 3, respectively. FIGS. 68A-G are plots that show percent cell counts for cells in different cell cycle phases following treatment with vehicle or 5 μM endoxifen, Compound 7b, Compound 7a, Compound 5b, Compound 5a, and Compound 3, respectively. FIGS. 69A-G are plots that show percent cell counts for cells in different cell cycle phases following treatment with vehicle or 1.25 μM endoxifen, Compound 7b, Compound 7a, Compound 5b, Compound 5a, and Compound 3, respectively. FIGS. 70A-G are plots that show percent cell counts for cells in different cell cycle phases following treatment with vehicle or 2.5 μM endoxifen, Compound 7b, Compound 7a, Compound 5b, Compound 5a, and Compound 3, respectively. FIGS. 71A-G are plots that show percent cell counts for cells in different cell cycle phases following treatment with vehicle or 5 μM endoxifen, Compound 7b, Compound 7a, Compound 5b, Compound 5a, and Compound 3, respectively. Collectively, these results show that numerous endoxifen-related compounds are active in inhibiting T47D cell cycles. At a concentration of 1.25 μM, Compounds 5a and 5b achieve similar cell cycle inhibition as endoxifen. At higher concentration, Compounds 5a and 5b exhibit slightly lower cell cycle inhibition activity than endoxifen. In T47D cells, at a concentration of 1.251M, Compounds 5a and 5b both exhibited comparable abilities to inhibit cell cycle progression as endoxifen. At higher concentrations, endoxifen demonstrated superior efficacy in inhibiting cell cycle progression, although Compounds 5a and 5b also exhibited high activity at these concentrations.

### Example 22

### Endoxifen-Related Compound Agonist and Antagonist Activities

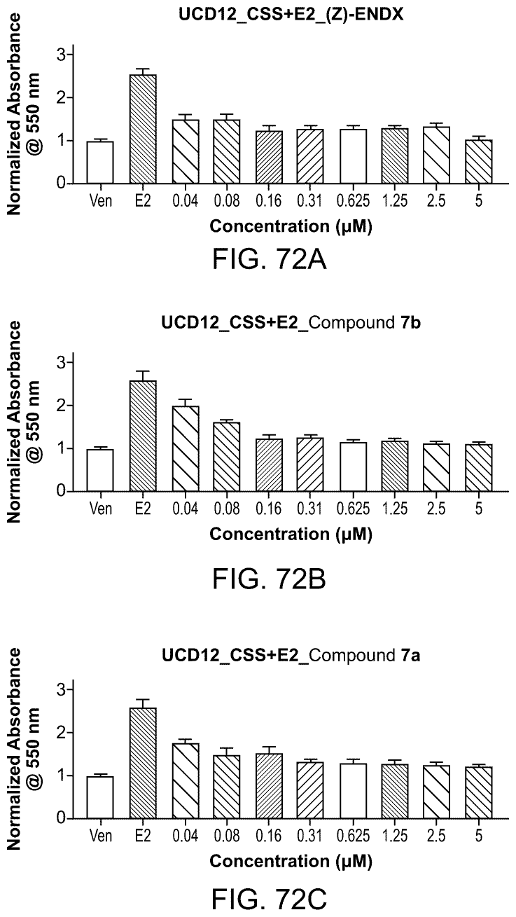

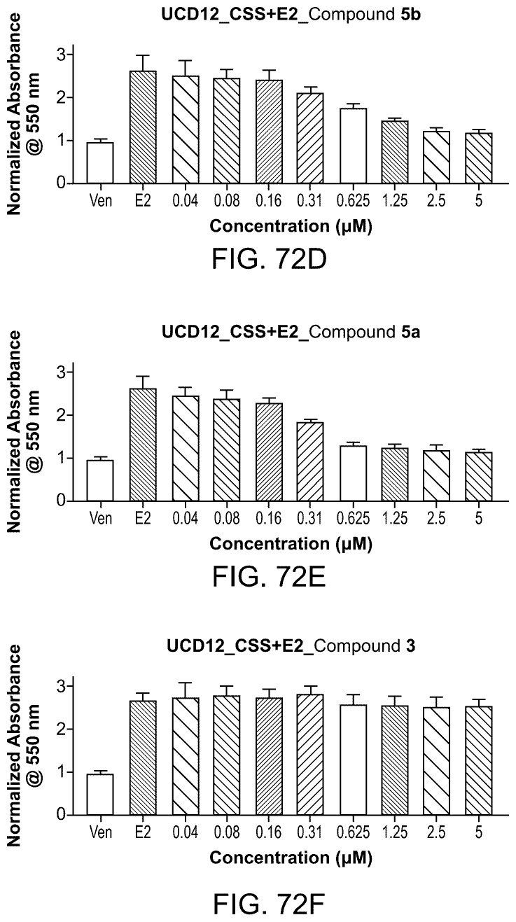

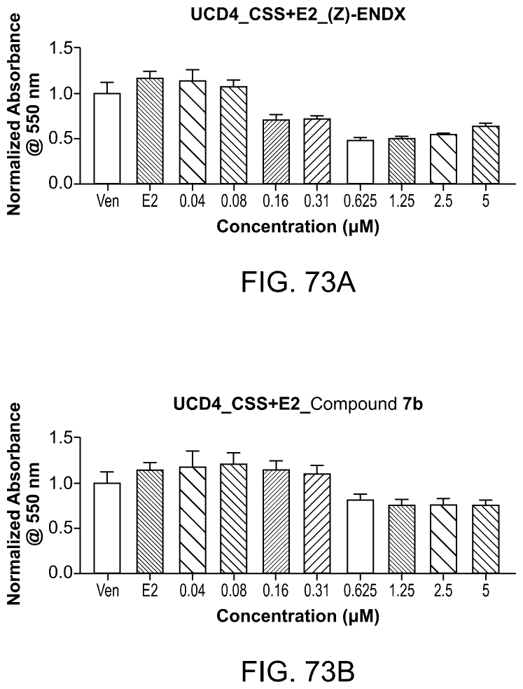

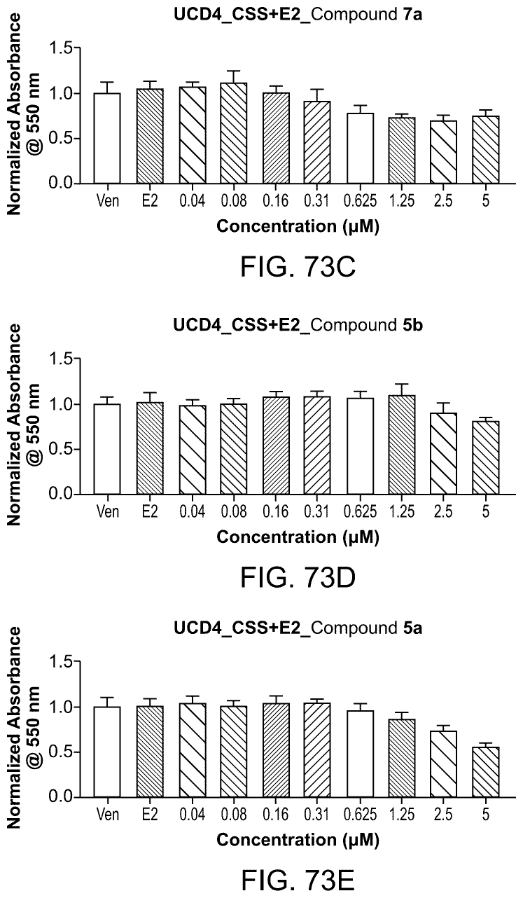

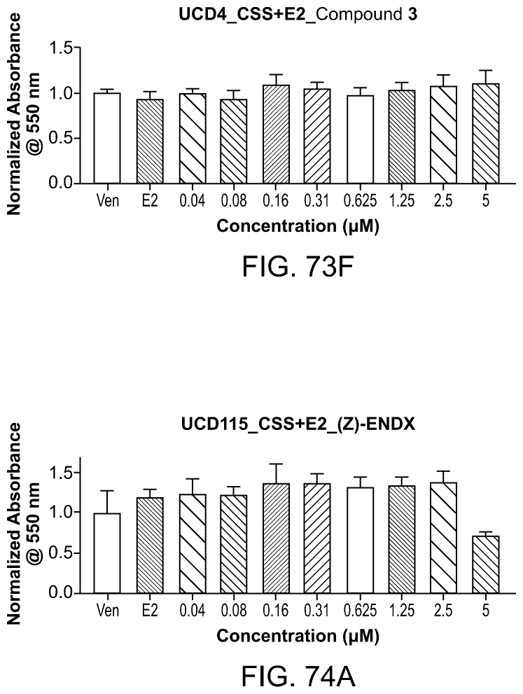

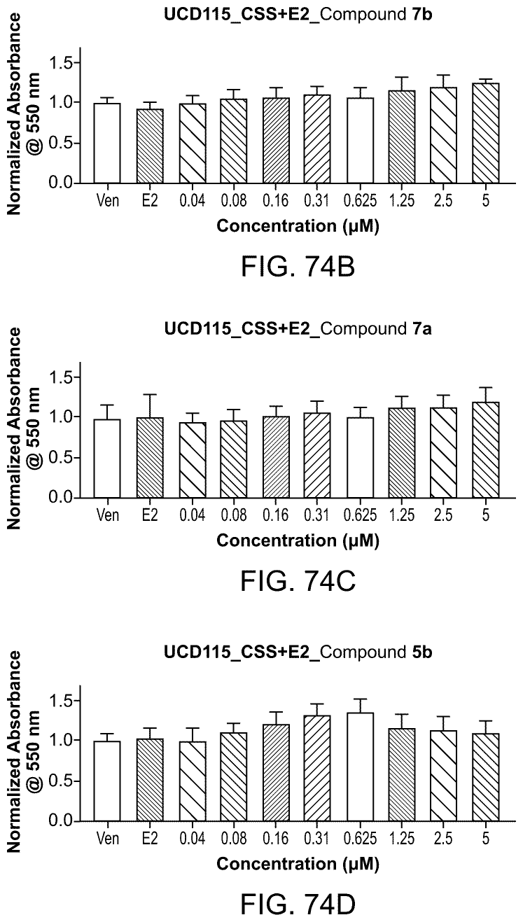

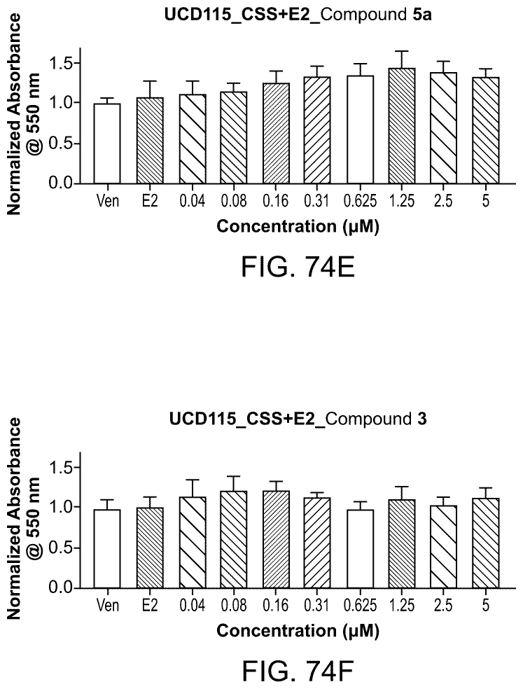

This example covers cell viability assays with endoxifen and endoxifen-related compounds. Luciferase assays were conducted on multiple T47D cancer cell lines (two ER+ and one ER−) with endoxifen and numerous endoxifen-related compounds to determine whether these compounds exhibit agonist or antagonist-behavior. The results of these analyses are summarized in FIGS. 72-74 for ER+, ER+, and ER− T47D cells, respectively. Within each figure, subfigure A (i.e., FIGS. 72A, 73A, and 74A) correspond to endoxifen, subfigure B corresponds to Compound 7b, subfigure C corresponds to Compound 7a, subfigure D corresponds to Compound 5b, subfigure E corresponds to Compound 5a, and subfigure F corresponds to Compound 3. These data show that the tested compounds exhibit antagonistic behavior akin to endoxifen. While endoxifen is the most potent antagonist from among the tested compounds, Compound 5a exhibited strong antagonistic behavior and a different concentration dependence than endoxifen, indicating it as a promising candidate for co-administration with endoxifen and as an agent for monotherapy.

### Example 23

### Endoxifen-Related Compound Agonist and Antagonist Activities

This example covers cell viability assays with endoxifen and endoxifen-related compounds. Luciferase assays were conducted on multiple T47D cancer cell lines (two ER+ and one ER−) with endoxifen and numerous endoxifen-related compounds to examine the abilities of these compounds to prevent estrogen-induced and ERα-mediated activation of an estrogen response element (ERE) luciferase reporter construct, and more generally to determine whether these compounds exhibit agonist or antagonist-behavior. The results of these analyses are summarized in FIGS. 72-74 for ER+, ER+, and ER− T47D cells, respectively. Within each figure, subfigure A (i.e., FIGS. 72A, 73A, and 74A) correspond to endoxifen, subfigure B corresponds to Compound 7b, subfigure C corresponds to Compound 7a, subfigure D corresponds to Compound 5b, subfigure E corresponds to Compound 5a, and subfigure F corresponds to Compound 3. These data show that the tested compounds exhibit antagonistic behavior akin to endoxifen. While endoxifen is the most potent antagonist from among the tested compounds, numerous compounds exhibited similar antagonist activities. In particular, in ER+ cells, the anti-proliferative effects of Compounds 7a and 7b closely resemble that of endoxifen in CSS+E2 media. However, ER− cells only exhibited a response to 5 μM endoxifen. Compound 5a exhibited strong antagonistic behavior and a different concentration dependence than endoxifen, indicating it as a promising candidate for co-administration with endoxifen and as an agent for monotherapy.

### Example 24

### Endoxifen-Related Compound Cell Migration Assays

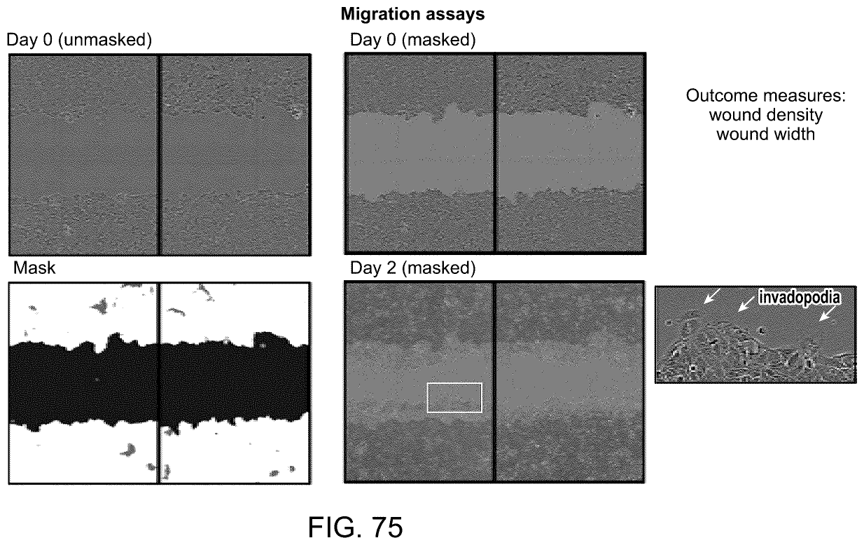

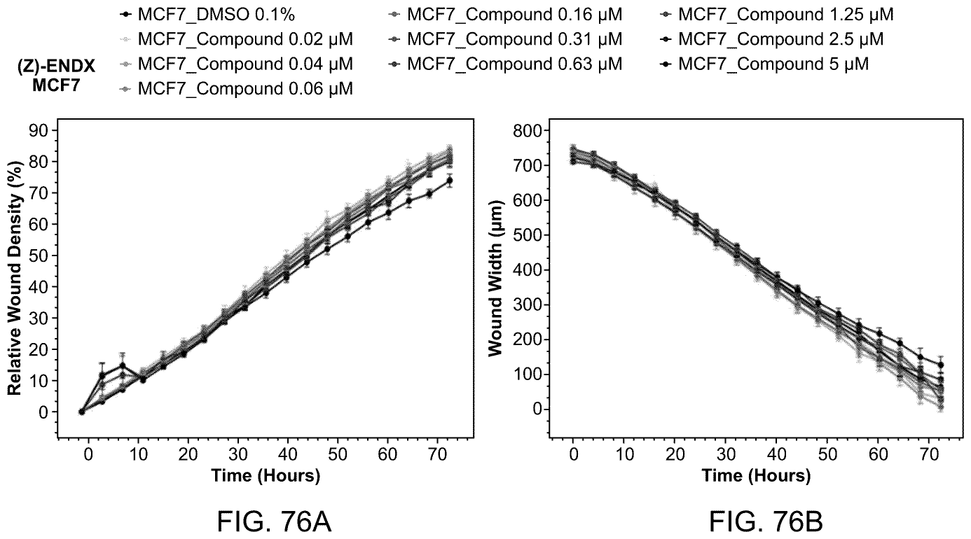

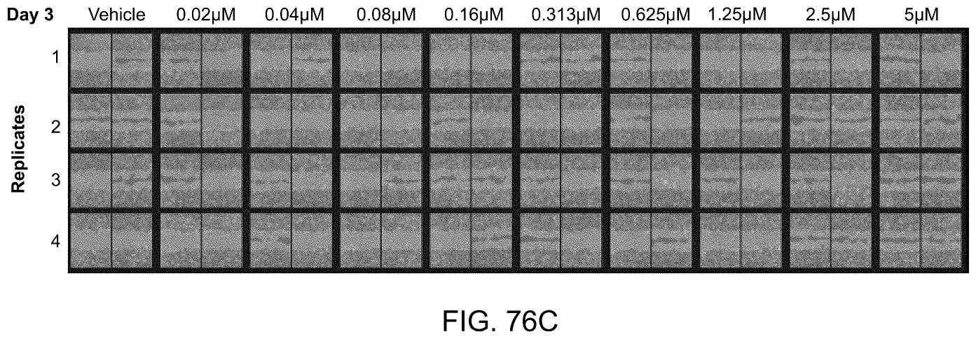

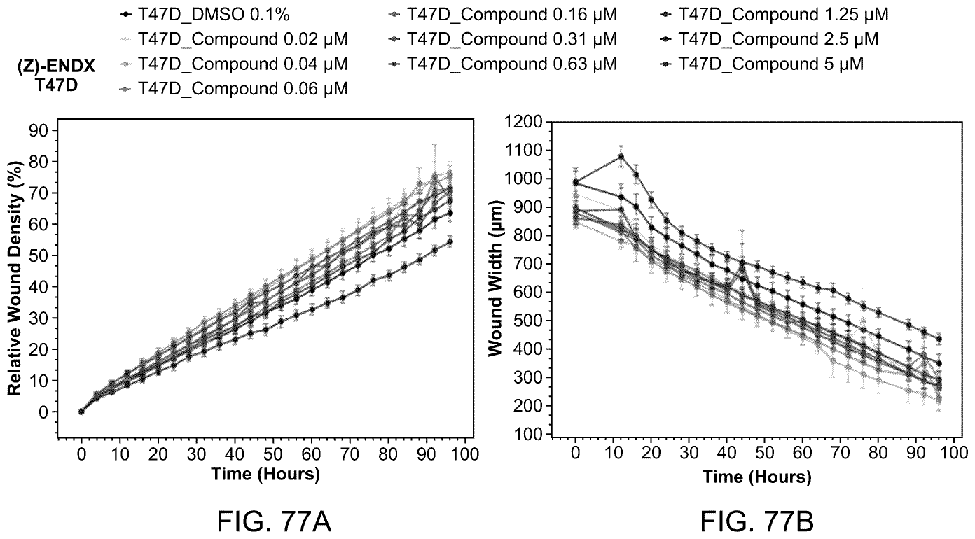

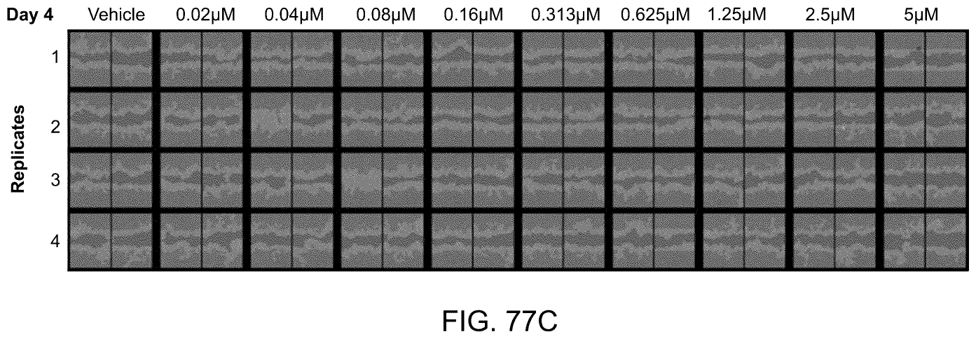

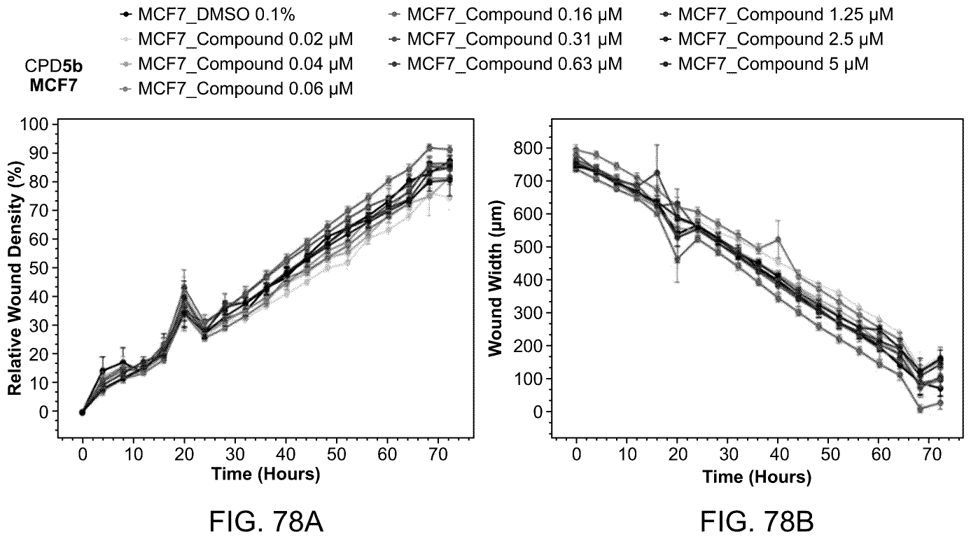

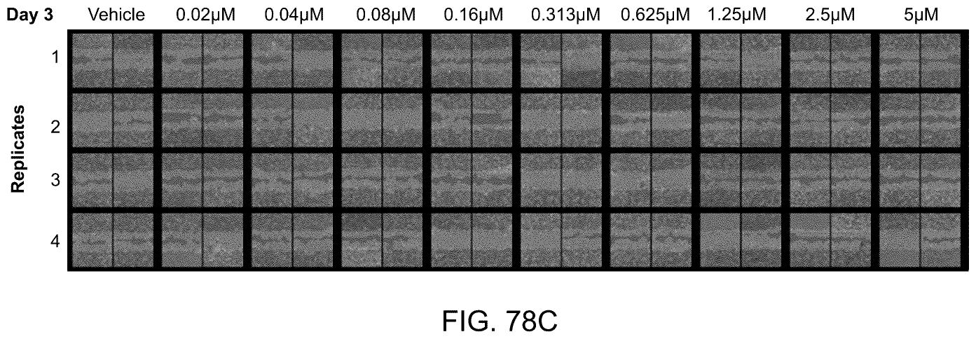

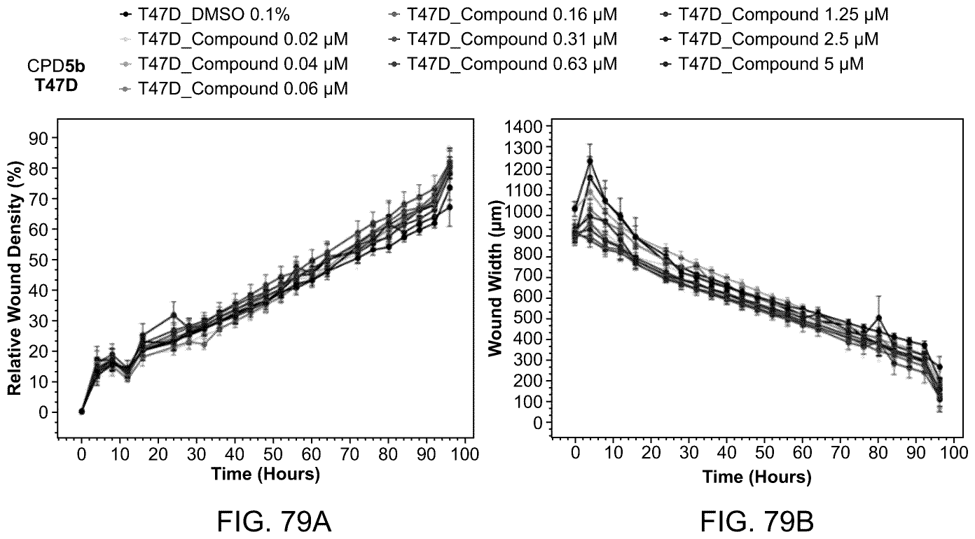

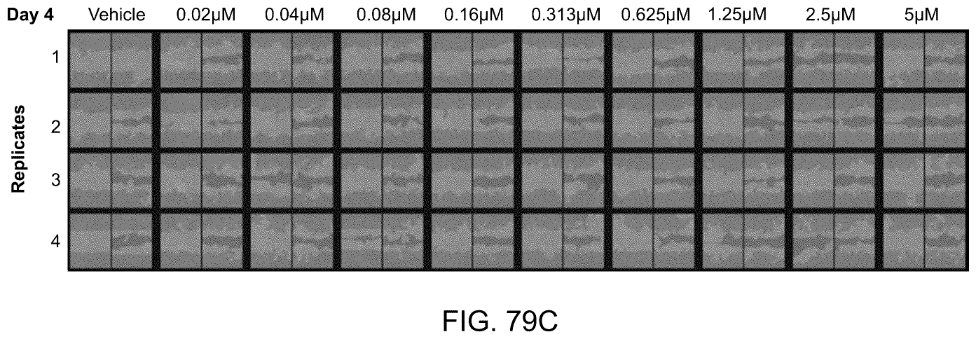

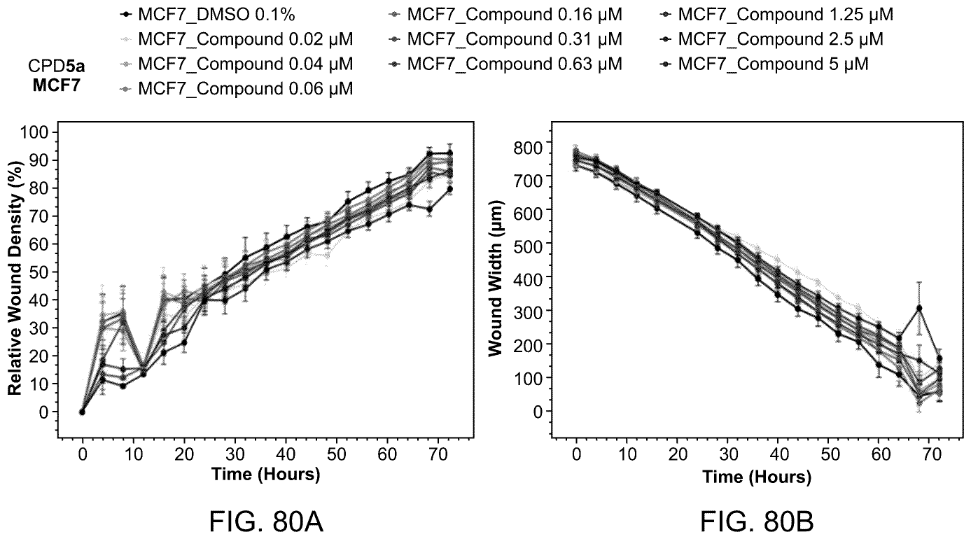

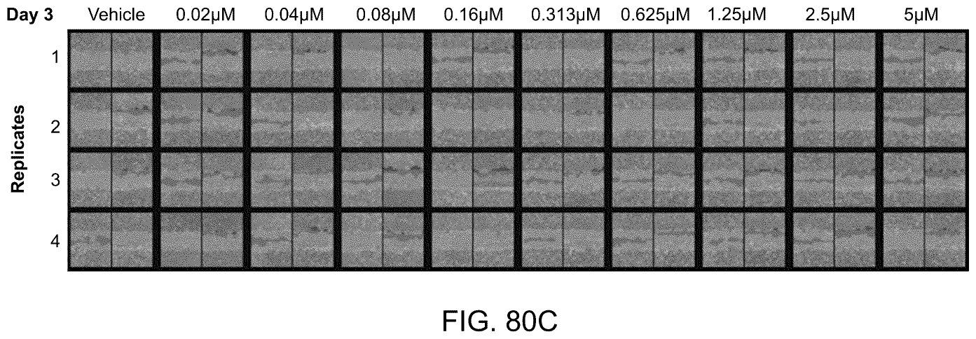

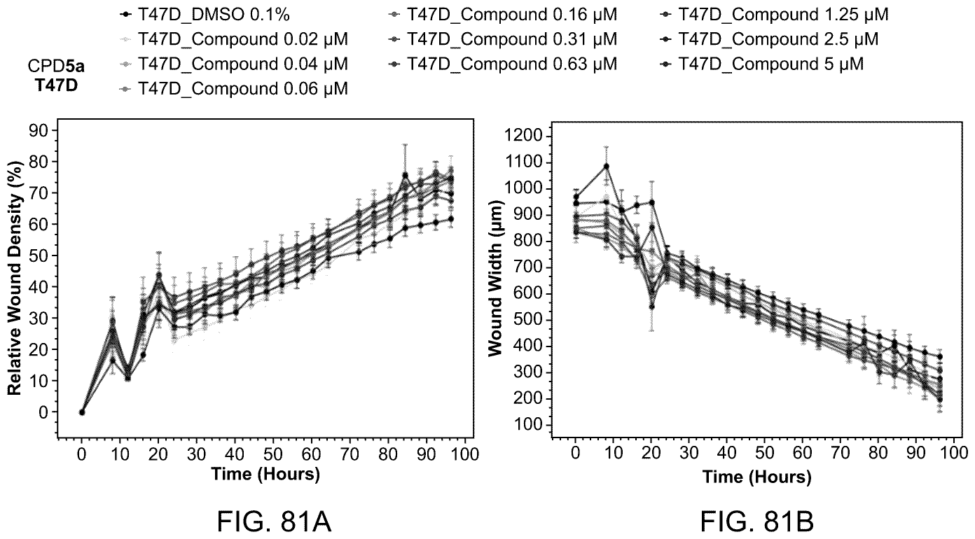

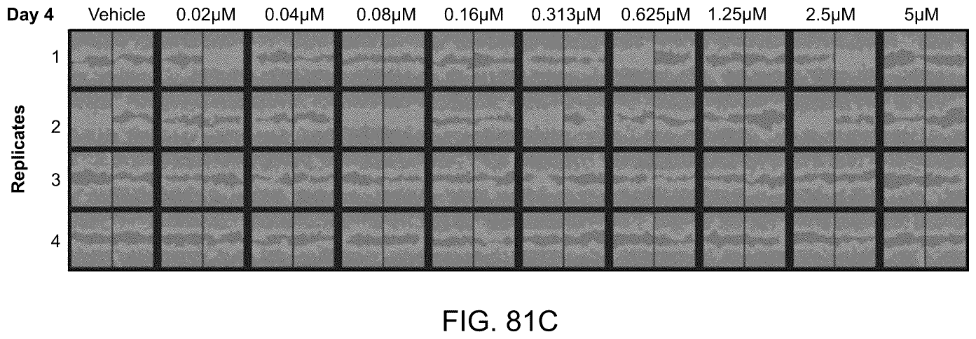

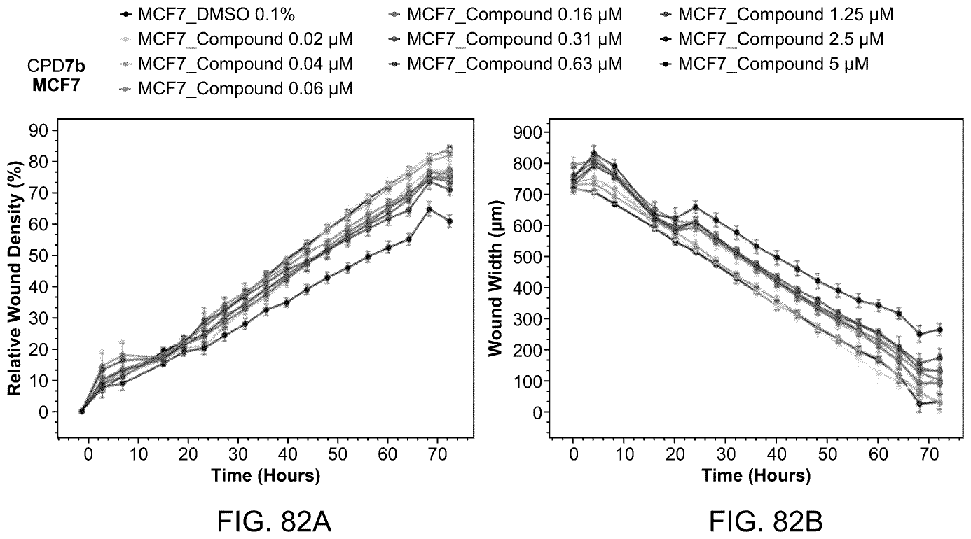

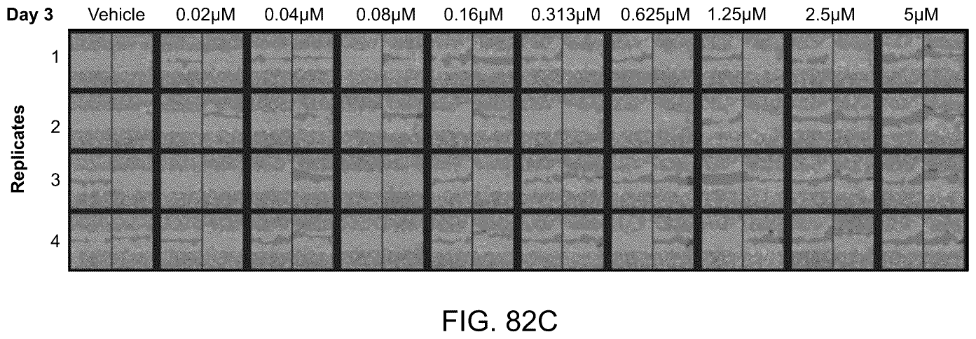

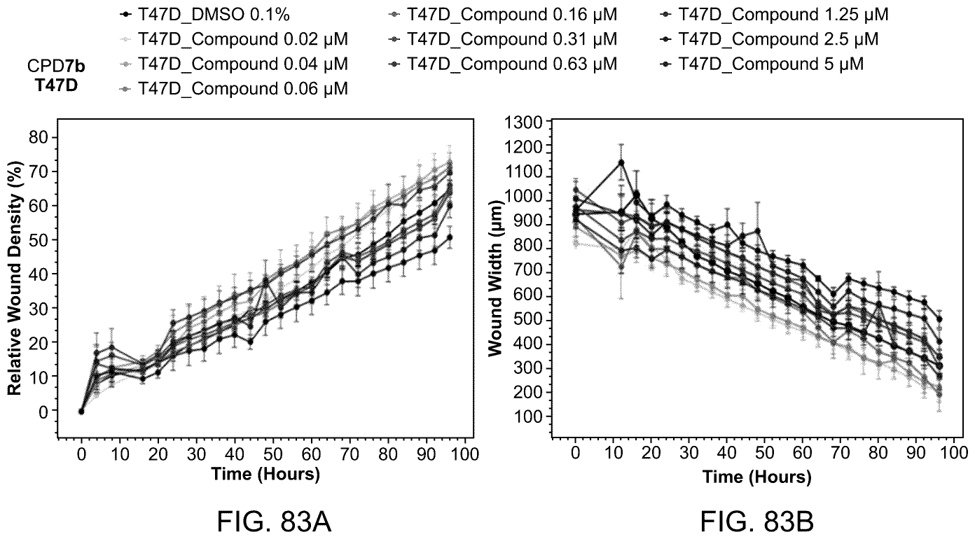

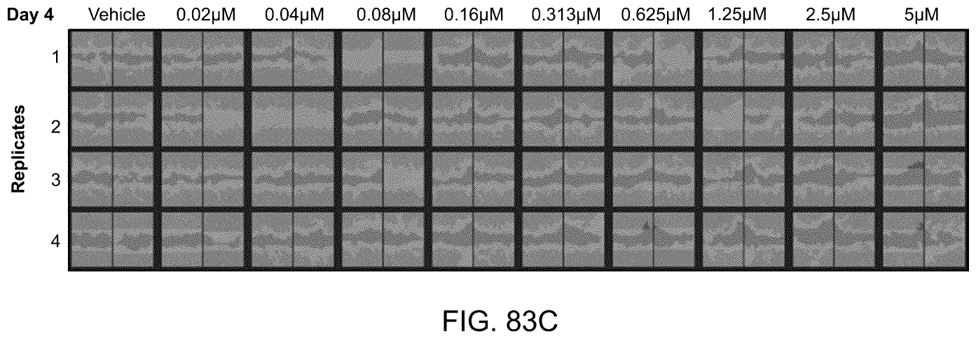

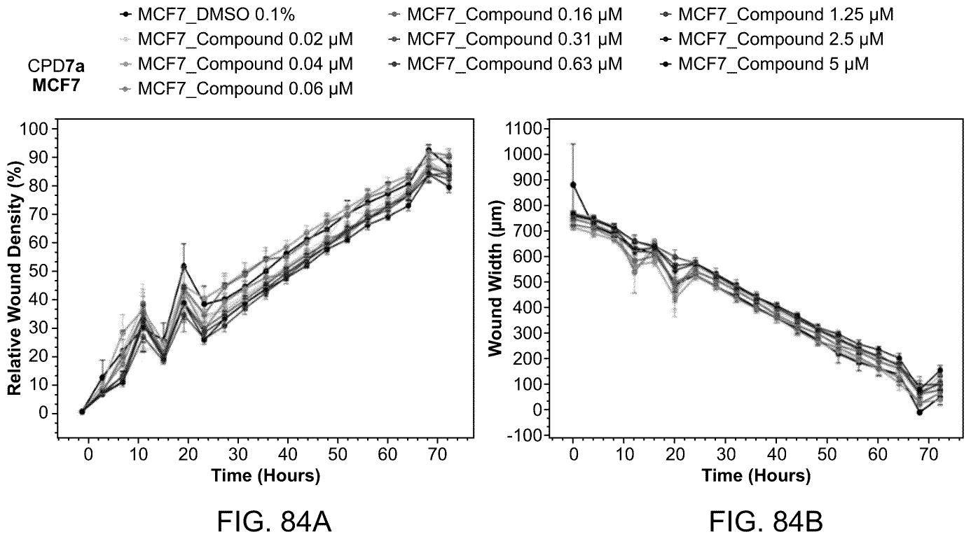

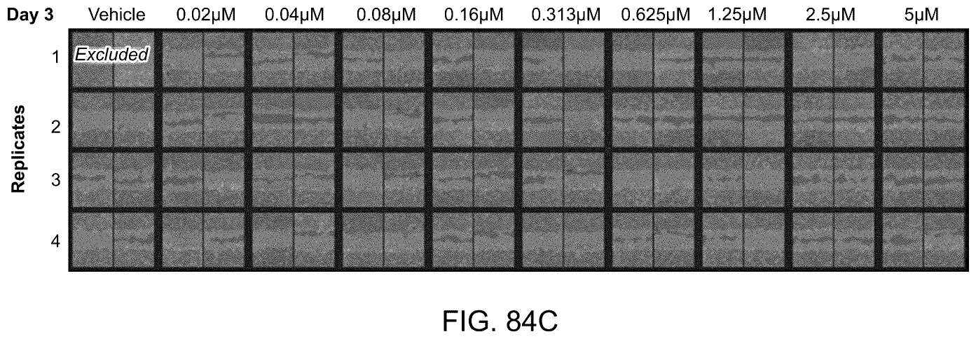

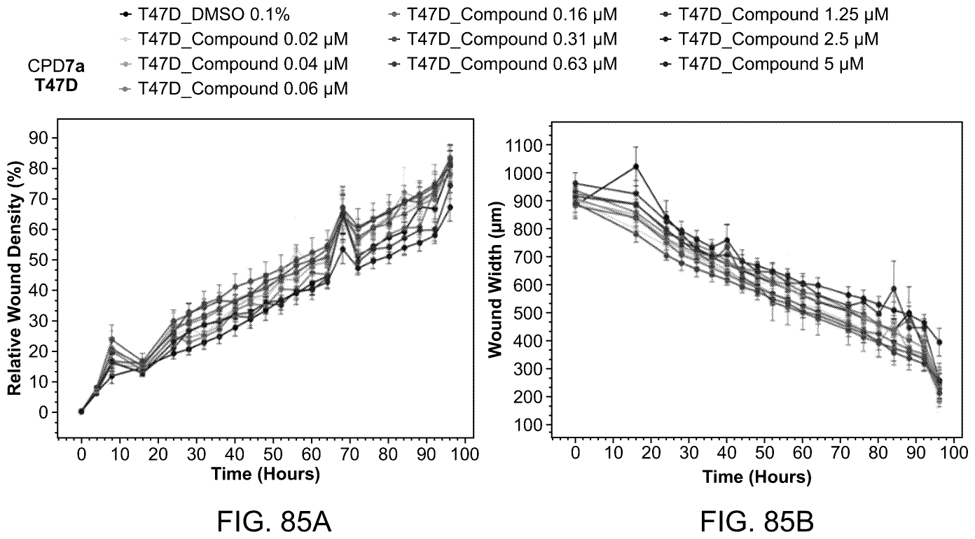

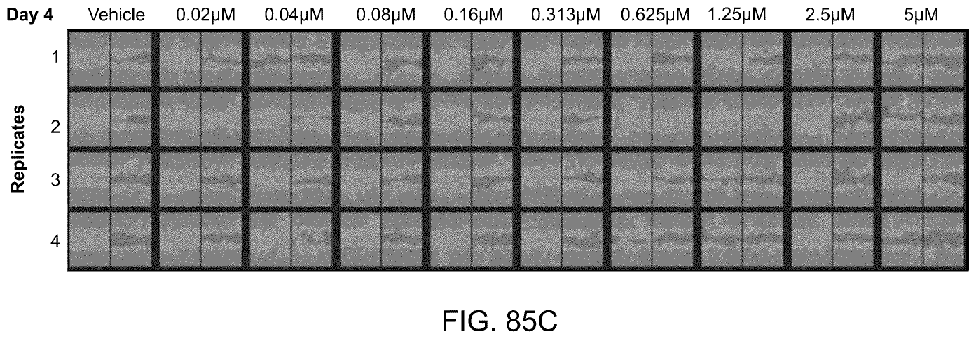

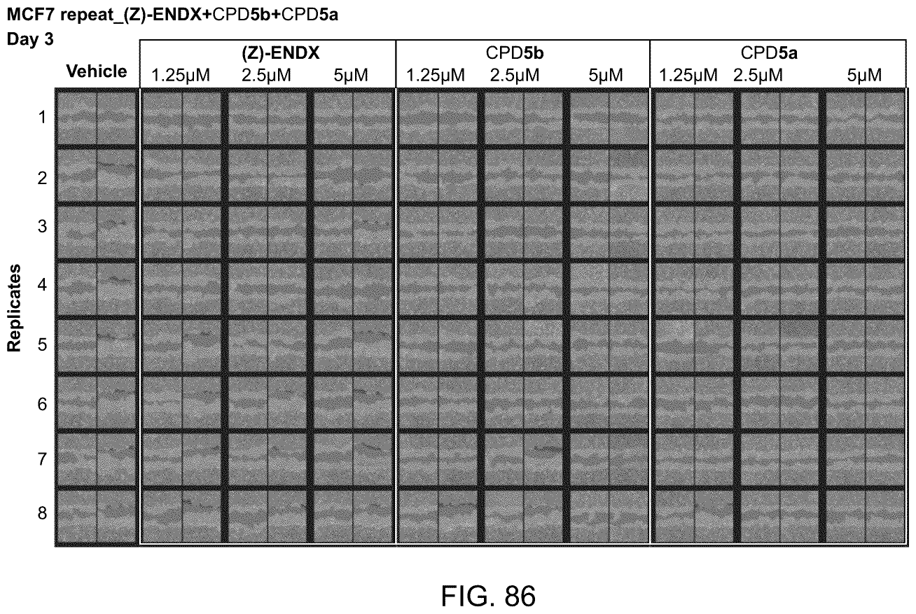

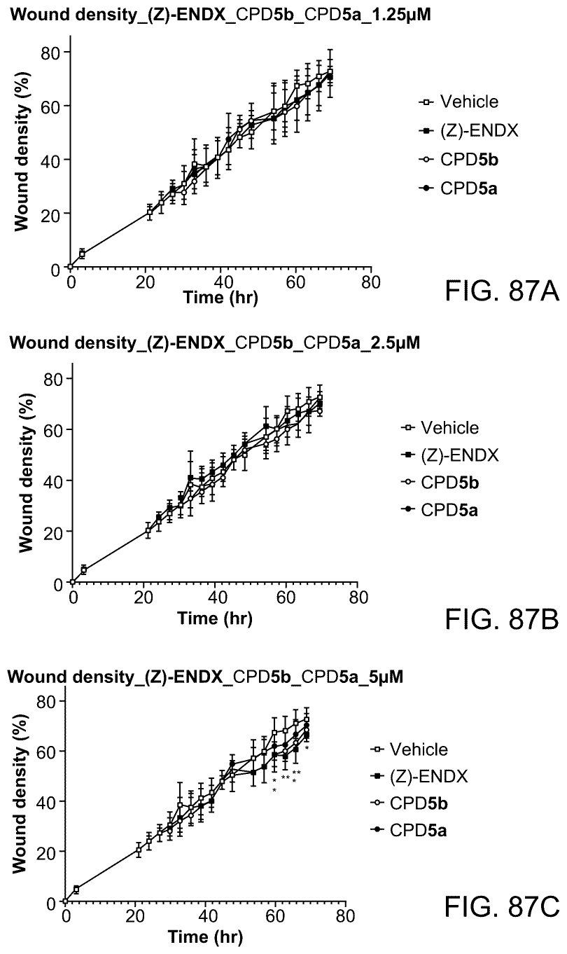

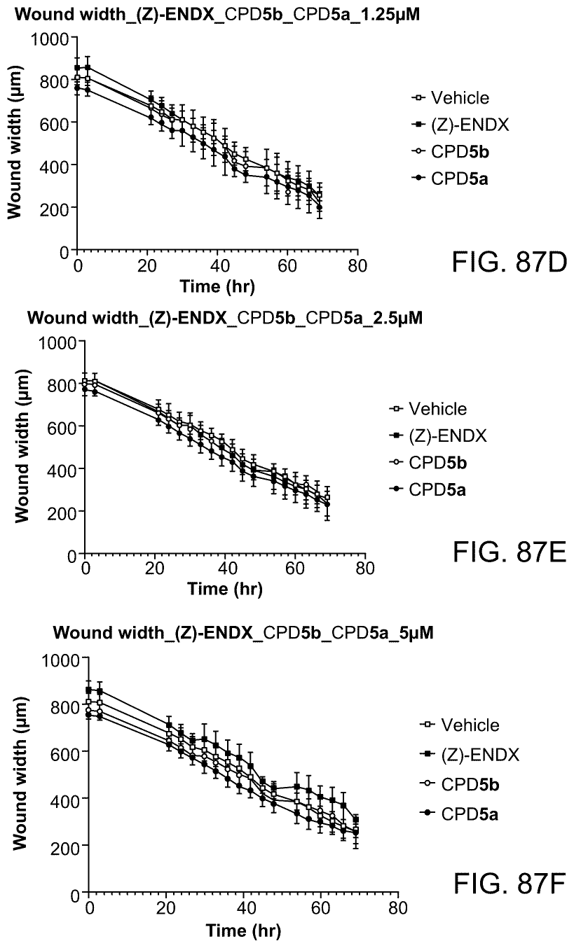

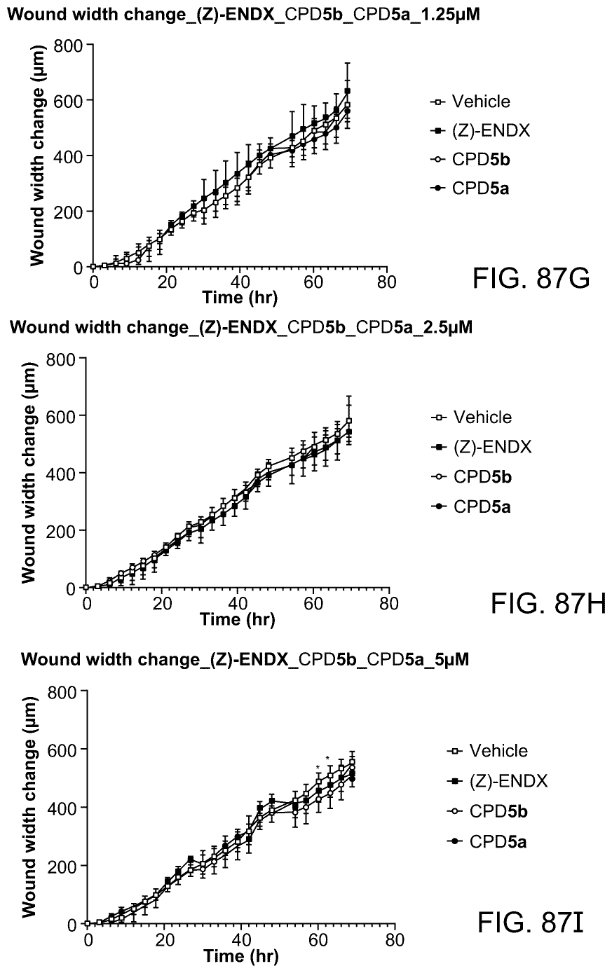

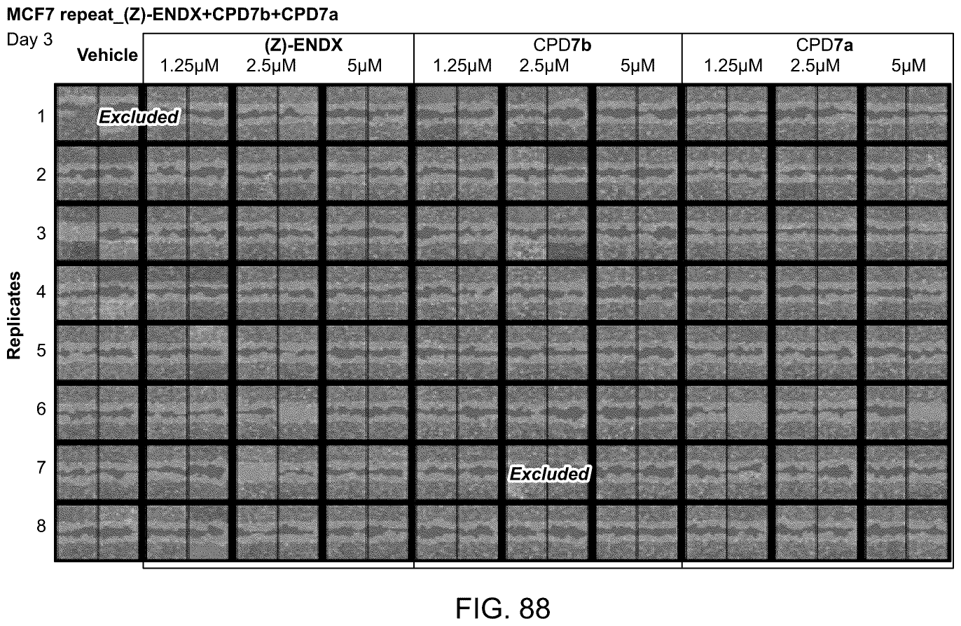

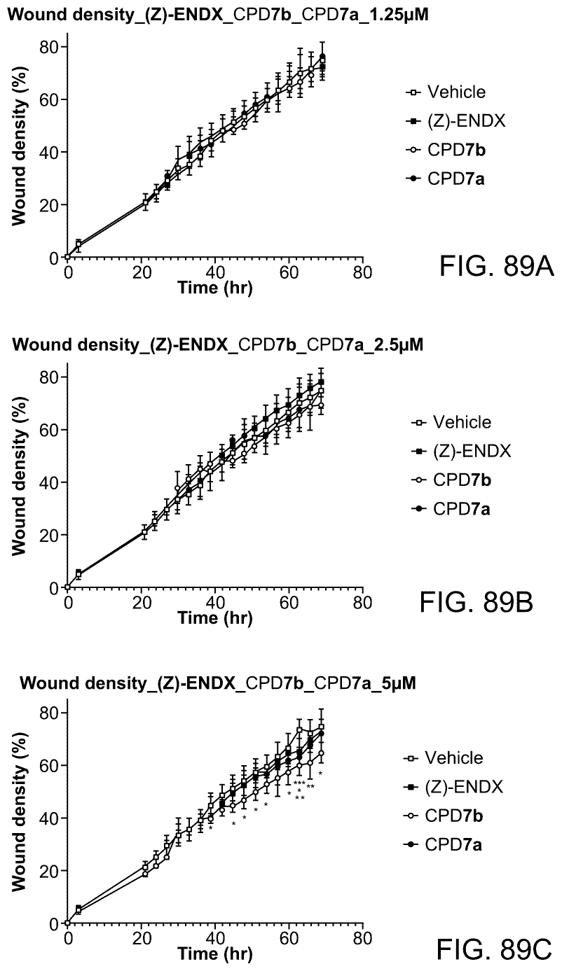

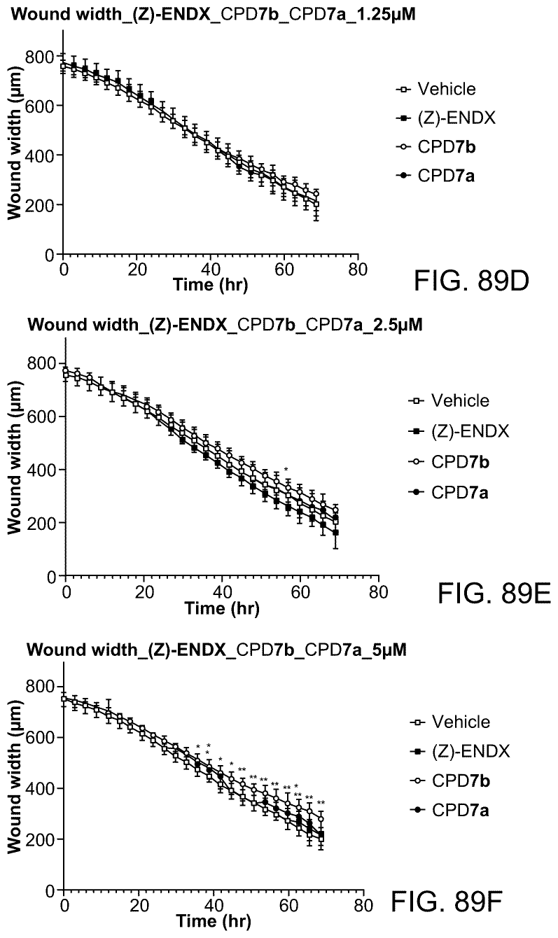

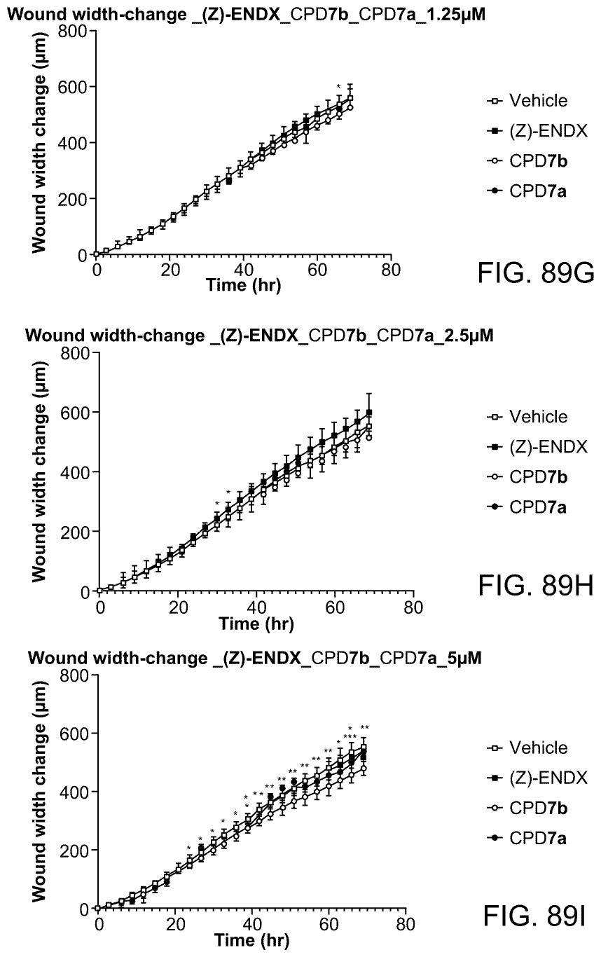

This example covers cell migration assays with endoxifen and endoxifen-related compounds. Scratch-wound density and width measurements were tracked for 70 to 100 hours in MCF7 and T47D samples following treatment with a compound. The results of these assays are displayed in FIGS. 75-89. FIG. 75 is a series of images of masked and unmasked wounds from the cell migration assays, with the rightmost figure showing invadopodia in cells within wound sites. In FIGS. 76-85, subfigure A is a plot of relative wound density, subfigure B is a plot of wound width, and subfigure C is a series of representative wound images from Day 3 or 4 of treatment. FIGS. 76-85 correspond to endoxifen treatment of MCF7 samples (FIG. 76), endoxifen treatment of T47D samples (FIG. 77), Compound 5b treatment of MCF7 samples (FIG. 78), Compound 5b treatment of T47D samples (FIG. 79), Compound 5a treatment of MCF7 samples (FIG. 80), Compound 5a treatment of T47D samples (FIG. 81), Compound 7b treatment of MCF7 samples (FIG. 82), Compound 7b treatment of T47D samples (FIG. 83), Compound 7a treatment of MCF7 samples (FIG. 84), and Compound 7a treatment of T47D samples (FIG. 85), respectively. FIGS. 86-89 show replicates of the wound density and width assays, with FIGS. 86 and 87A-I showing representative images and plots of wound density and width data on MCF7 samples treated with 1.25, 2.5, and 5 μM endoxifen, Compound 5a, and Compound 5b, and FIGS. 88 and 89A-I showing representative images and plots of wound density and width data on MCF7 samples treated with 1.25, 2.5, and 5 μM endoxifen, Compound 7a, and Compound 7b.

As can be seen from these figures, numerous endoxifen-related compounds exhibit comparable or enhanced wound healing relative to endoxifen. Migration inhibition was primarily detected at the highest tested dose (5 μM), which may be due to the fact that MCF7 and T47D cells are not migratory in nature under normal conditions. Accordingly, the scratch-wound assays, although conventionally employed to examine migration, in this case provided insight into a combination of cellular proliferation and cell migration. The results of these assays suggest that Compound 7b was more potent than endoxifen at inhibiting the migration and proliferation of MCF7 and T47D cells at the highest dose examined.

### Example 25

### Anti-Cancer Activities of Endoxifen-Related Compounds

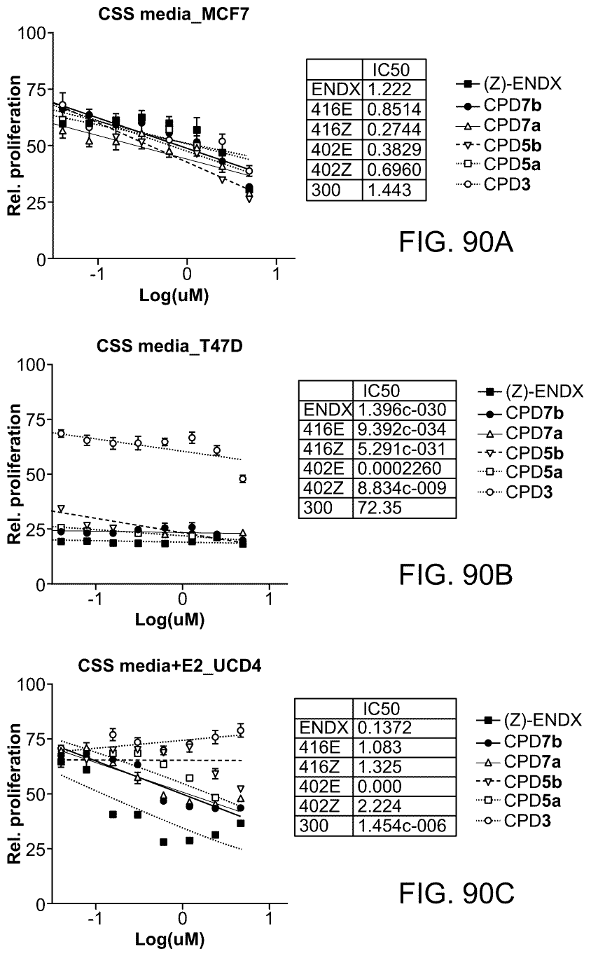

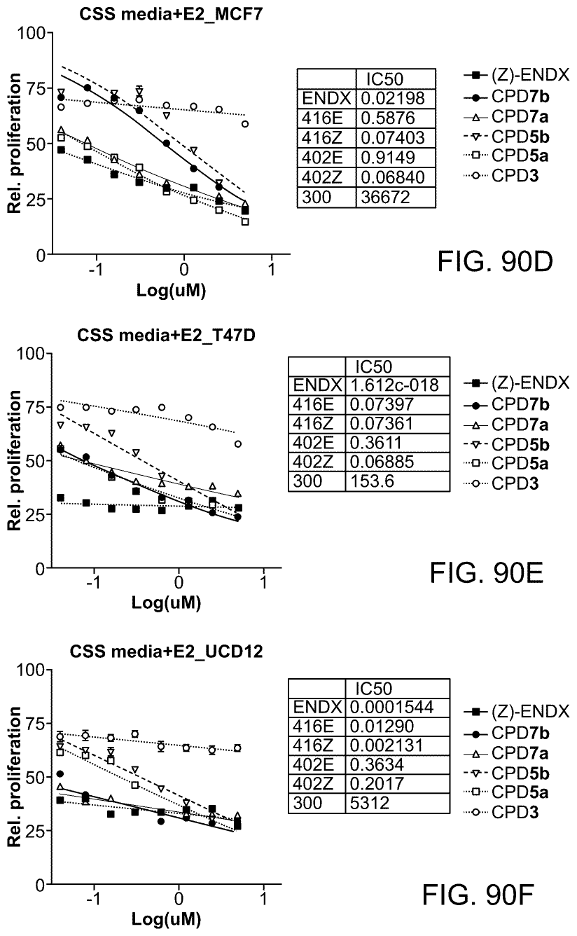

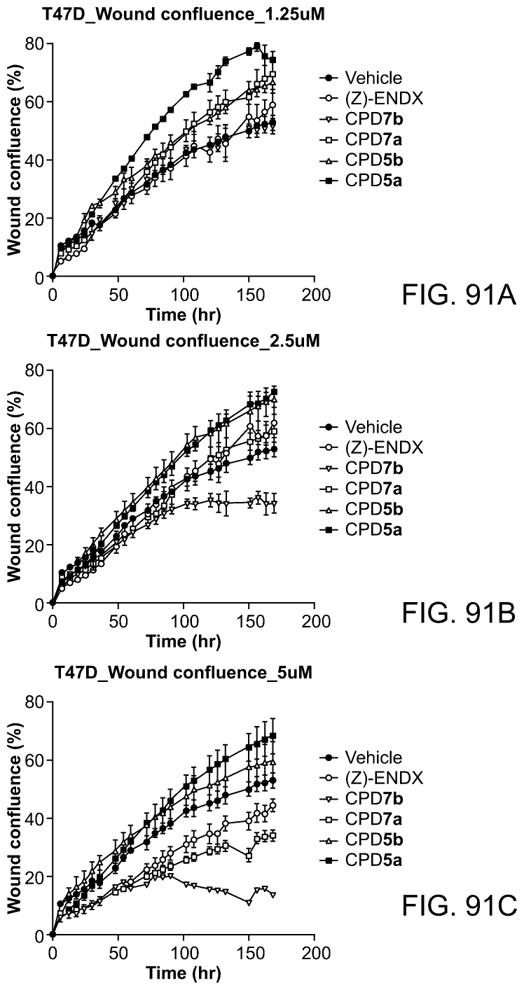

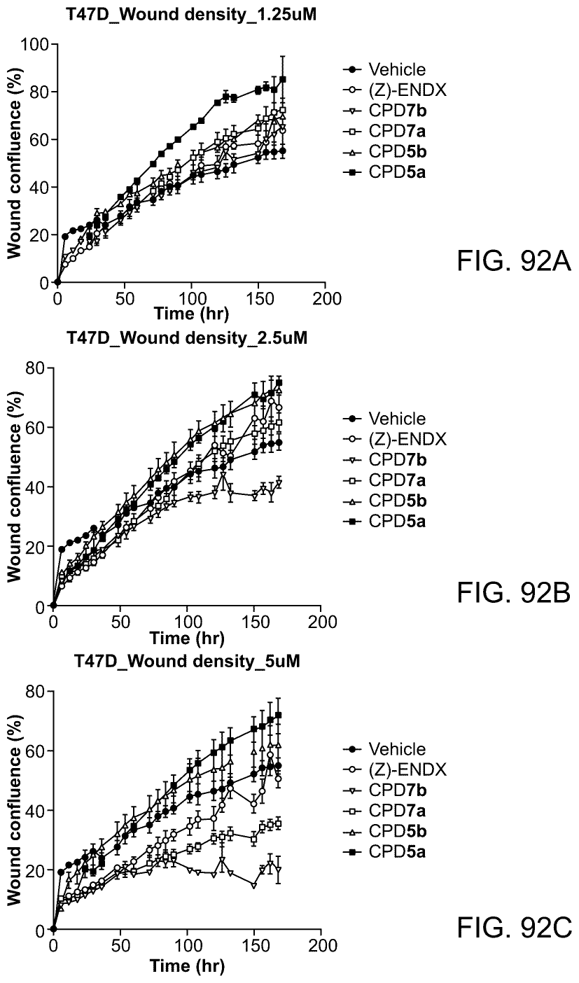

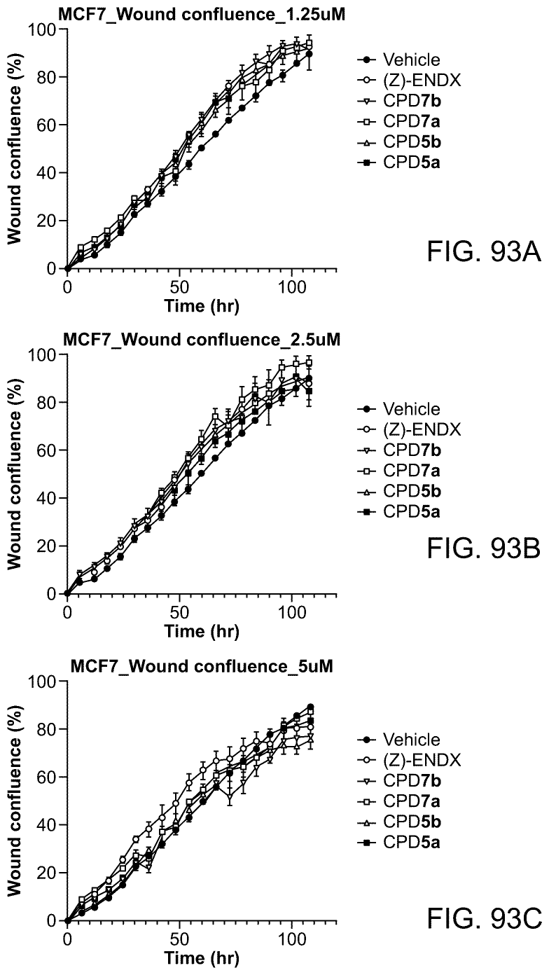

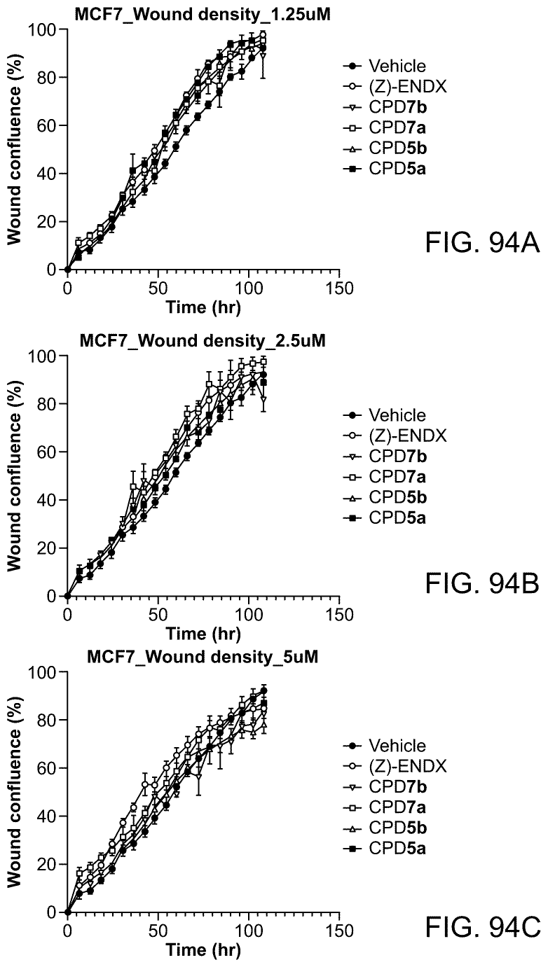

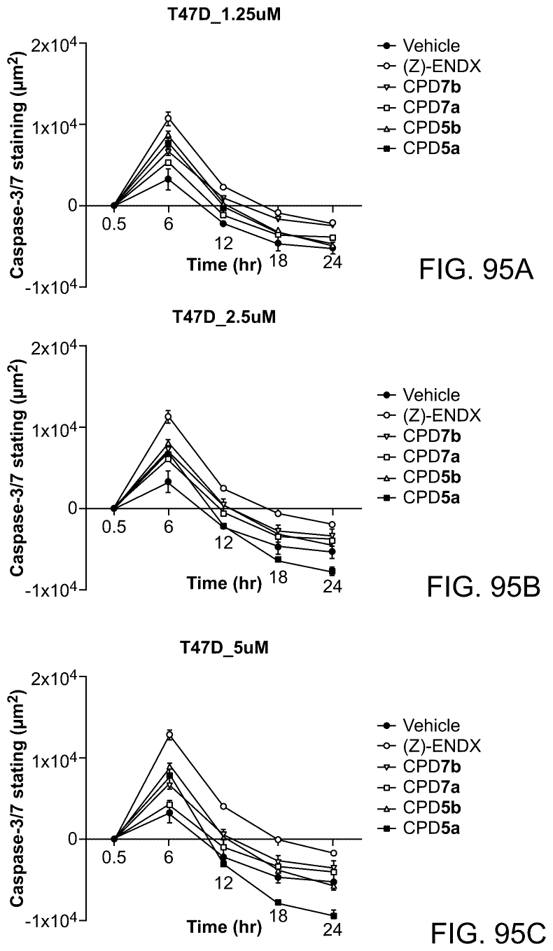

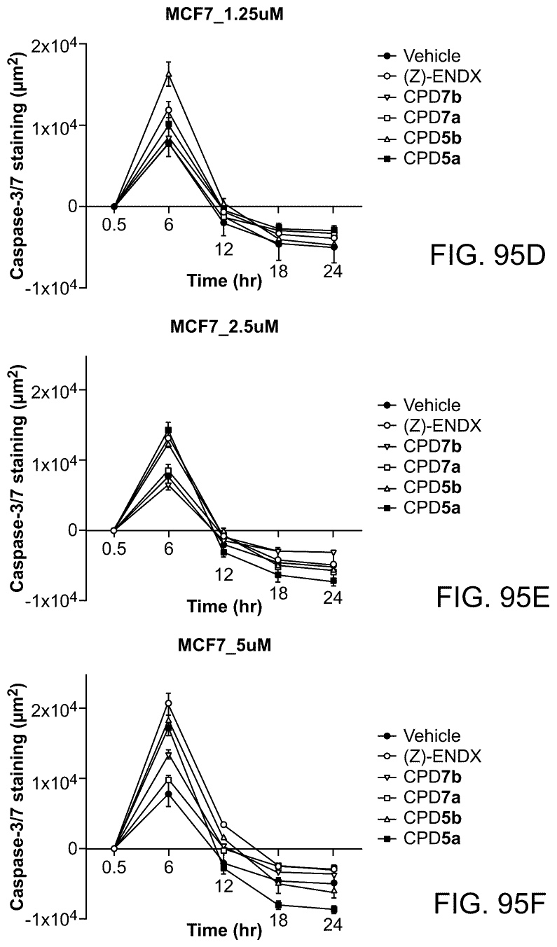

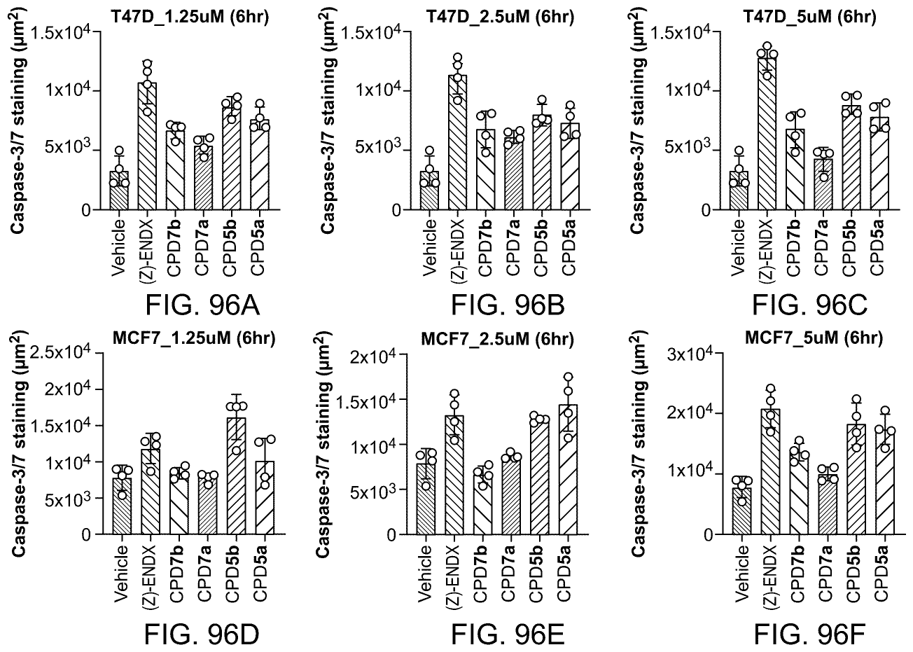

This example covers anti-invasion, apoptosis, and toxicity assays for various endoxifen-related compounds against multiple cancer cell lines. FIGS. 90A-F are plots that summarize results of proliferation assays for multiple cancer cell lines (MCF7, T47D, UCD4, and UCD12) in the presence of varying doses of endoxifen and multiple endoxifen-related compounds. FIGS. 91A-C are plots that summarize results of wound confluence measurements from scratch wound assays on T47D cells in the presence of 1.25 μM (FIG. 91A), 2.5 μM (FIG. 91B), and 5 μM (FIG. 91C) endoxifen and multiple endoxifen-related compounds. FIGS. 92A-C are plots that summarize results of wound density measurements from scratch wound assays on T47D cells in the presence of 1.25 μM (FIG. 92A), 2.5 μM (FIG. 92B), and 5 μM (FIG. 92C) endoxifen and multiple endoxifen-related compounds. FIGS. 93A-C are plots that summarize results of wound confluence measurements from scratch wound assays on MCF7 cells in the presence of 1.25 μM (FIG. 93A), 2.5 μM (FIG. 93B), and 5 μM (FIG. 93C) endoxifen and multiple endoxifen-related compounds. FIGS. 94A-C are plots that summarize results of wound density measurements from scratch wound assays on MCF7 cells in the presence of 1.25 μM (FIG. 94A), 2.5 μM (FIG. 94B), and 5 μM (FIG. 94C) endoxifen and multiple endoxifen-related compounds. FIGS. 95A-F are plots that summarize results of caspase-3/7 apoptosis assays on MCF7 and T47D cells in the presence of varying concentrations of endoxifen and multiple endoxifen-related compounds as a function of time. FIGS. 96A-F are plots that summarize aggregate results of caspase-3/7 apoptosis assays on MCF7 and T47D cells in the presence of varying concentrations of endoxifen and multiple endoxifen-related compounds. Multiple endoxifen-related compounds exhibited similar anti-cancer activities as endoxifen. A number of these compounds exhibited different relative anti-invasion, apoptosis, and IC₅₀ activities relative to endoxifen, indicating these compounds as viable candidates as endoxifen substitutes. Furthermore, specific compounds exhibited grater potency than endoxifen. For example, Compound 7b was more potent than endoxifen at inhibiting the invasion of T47D cells. While endoxifen was the most potent drug among those tested at inducing the apoptosis of T47D cells, for MCF7 cells, endoxifen and Compounds 5a and 5b exhibited equal potencies.

### Example 26

### Compound 3

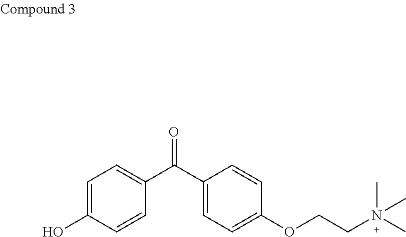

Compound S1 was suspended in 9 vol of THF and CH₃I (1.15 eq.) was added. The mixture was stirred 8-10 h at ambient temperature, the product was filtered, washed with THF (2 vol) and dried under reduced pressure to yield Compound 3.

### Example 27

### Alternative Synthesis of Compounds 5a and 5b

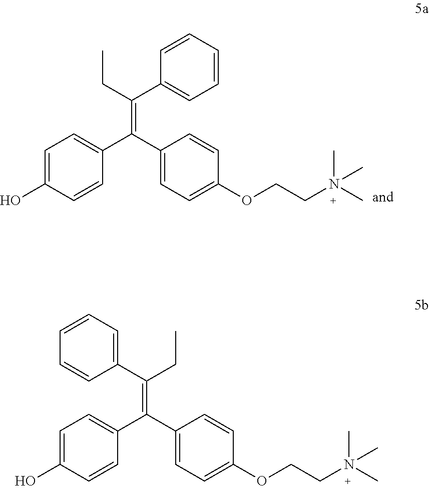

Either E or Z isomer of Endoxifen was suspended in 6 v of MTBE and 1.1 eq. of DIPEA was added. CH₃I (2.1 eq.) was added dropwise and the mixture was stirred at RT at least 10 h (formation of desired product was monitored by HPLC). The product was filtered, washed with MTBE (2 vol) and dried under vacuum at 40° C. until constant weight.

### Example 28

### Alternative Synthesis of Compounds 7a and 7b

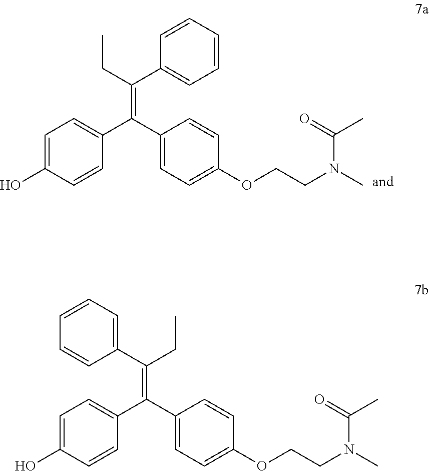 [SMILES: CC/C(=C(\c1ccc(O)cc1)c1ccc(OCCN(C)C(C)=O)cc1)c1ccccc1 ; CC/C(=C(/c1ccc(O)cc1)c1ccc(OCCN(C)C(C)=O)cc1)c1ccccc1]

The synthesis of both Compounds 7a and 7b was performed on 50 g scale of the corresponding endoxifen isomer. In particular the desired compound (7a or 7b) is suspended in EtOAc (10 vol) and TEA (1.2 eq.) is added. The Ac₂O (1.1 eq.) is added dropwise at room temperature over 15 minutes. The mixture is stirred for 16 hours, then the solid is filtered and washed with EtOAc (2 vol). The solid is dried under vacuum at 50° C. until constant weight.

### Example 29

### Anti-Cancer Activity of Compounds 5a, 5b, 7a and 7b

Approximately 80% of breast cancers express estrogen receptor alpha (ERα, ESR1) which drives cancer progression. Despite the array of targeted therapies, ERα+ breast cancer exhibits the highest recurrence and late distant relapse rates among subtypes. (Z)-Endoxifen (ENDX), an active metabolite of tamoxifen, is a potent orally bioavailable selective estrogen receptor modulator with a favorable safety profile now under development for breast health applications. Its synthesis yields endoxifen-related byproduct new chemical entities (NCE), including Compound 7b, Compound 7a, Compound 5b and Compound 5a. This study compared the anti-estrogenic effects of these compounds to (Z)-endoxifen in ERα+ breast cancer cell lines (MCF7, T47D, UCD12), including those with ESR1 mutations (Y537S, D538G) linked to endocrine resistance.

In 2D cultures with estrogen-containing media, (Z)-endoxifen and all byproducts showed significant anti-proliferative effects across all lines, achieving 20-90% inhibition at 0.02-5 μM (p<0.0001 vs. vehicle). The strongest effects were seen with (Z)-endoxifen in MCF7, AT402Z in T47D, and both (Z)-endoxifen and Compound 7a in UCD12 cells. Notably, under estrogen-deficient conditions, all byproducts exceeded (Z)-endoxifen's activity in MCF7 cells; Compound 7a and Compound 7b were more effective in T47D, and Compound 7a and Compound 5a in UCD12. (Z)-Endoxifen was the most potent in T47D cells with Y537S or D538G mutations, while (Z)-endoxifen and Compound 5a showed similar potency in MCF7 mutants. In 3D cultures at a clinically relevant dose (˜1 μM), all compounds significantly reduced cancer cell proliferation (˜75-90% inhibition; p<0.0001 vs. vehicle) with (Z)-endoxifen and Compound 5a demonstrating comparable, superior potency. Compound 7b consistently outperformed (Z)-endoxifen in migration and invasion inhibition assays in both MCF7 and T47D cells (p<0.0001), while UCD12 cells did not migrate in vitro, precluding their assessment. All compounds induced G1 cell cycle arrest, with Compound 5a and (Z)-endoxifen being the most effective. All compounds also significantly induced apoptosis in MCF7 cells at 1-5 μM (p<0.0001 vs. vehicle), with Compound 7a, Compound 5b, and Compound 5a surpassing (Z)-endoxifen (p<0.0001). In T47D cells, apoptosis was significantly induced by (Z)-endoxifen, Compound 7b, and Compound 7a (p<0.0001 vs. vehicle), with (Z)-endoxifen showing superior activity. RT-qPCR analysis revealed that at 0.1 μM, (Z)-endoxifen and Compound 7a were the most potent at

1-384. (canceled)

385. A compound of Formula (I): 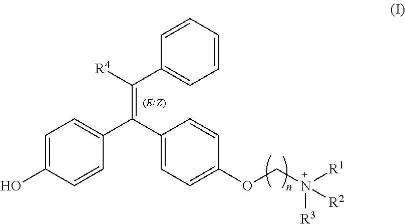
or a pharmaceutically acceptable salt, solvate, or tautomer thereof,
wherein:
R¹, R², R³ are each independently C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
R⁴ is C₁-C₆ alkyl substituted with 0 to 3 instances of R⁸, C₂-C₆ alkenyl substituted with 0 to 3 instances of R⁸, or C₂-C₆ alkynyl substituted with 0 to 3 instances of R⁸;
each instance of R⁵ is independently halogen, hydroxyl, C₁-C₃ alkoxy, C₁-C₃ alkylthio, C₁-C₃ alkyl, —C(═O)OH, or —C(═O)OCH3;
each instance of R⁶ and R⁷ are each independently hydrogen or C₁-C₃ alkyl, or an instance of R⁶ and an instance of R⁷ on the same nitrogen are taken together with the nitrogen to form C₂-C₇ heterocycloalkyl;
each instance of R⁸ is independently halogen, hydroxyl, C₁-C₃ alkoxy, C₁-C₃ alkylthio, C₁-C₃ alkyl, or C₃-C₈ cycloalkyl; and
n is an integer from 1 to 10.

386. The compound of claim 385, wherein R¹, R², and R³ are each independently C₁-C₆ alkyl.

387. The compound of claim 385, wherein R⁴ is C₁-C₆ alkyl.

388. The compound of claim 385, wherein at least 75% of the compound of Formula (I) is a compound of Formula (Ia):

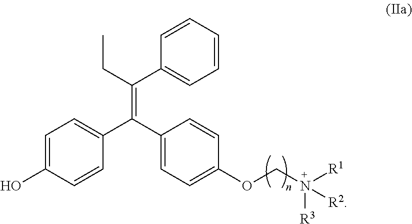

389. The compound of claim 385, wherein the compound has any one of the following structures: 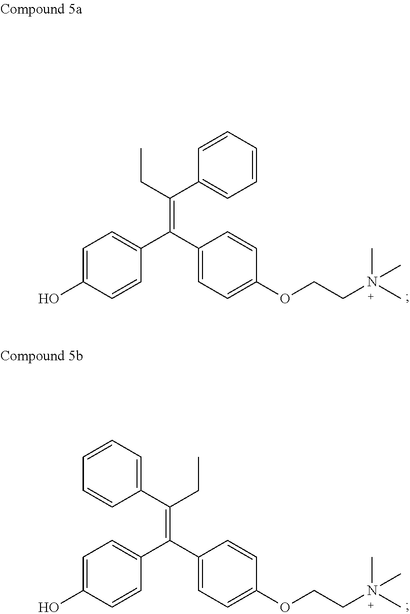
or a pharmaceutically acceptable salt, solvate, or tautomer thereof.

390. A pharmaceutical composition comprising a first compound of claim 385 and a pharmaceutically acceptable carrier or diluent.

391. The pharmaceutical composition of claim 390, further comprising a second compound selected from 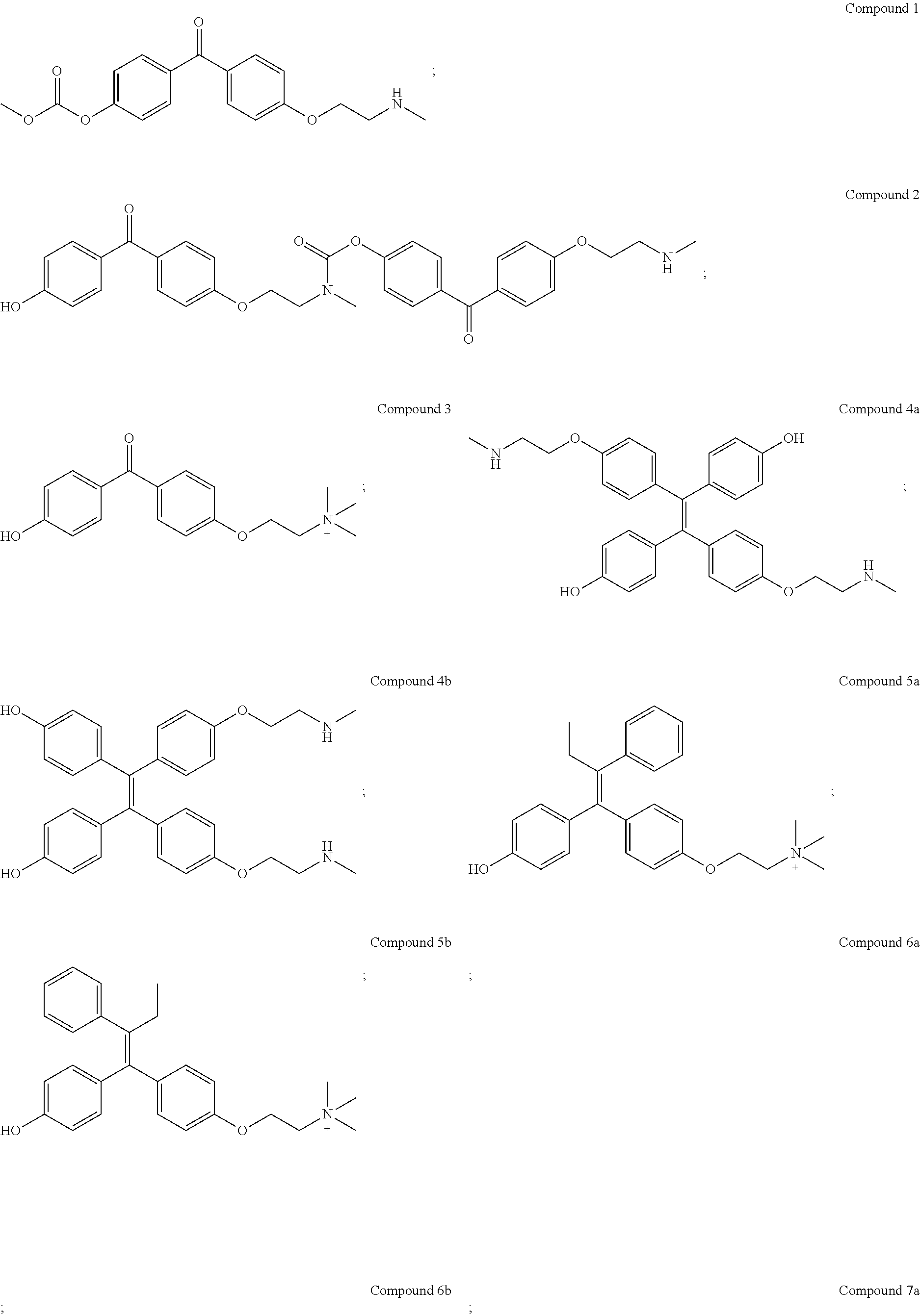 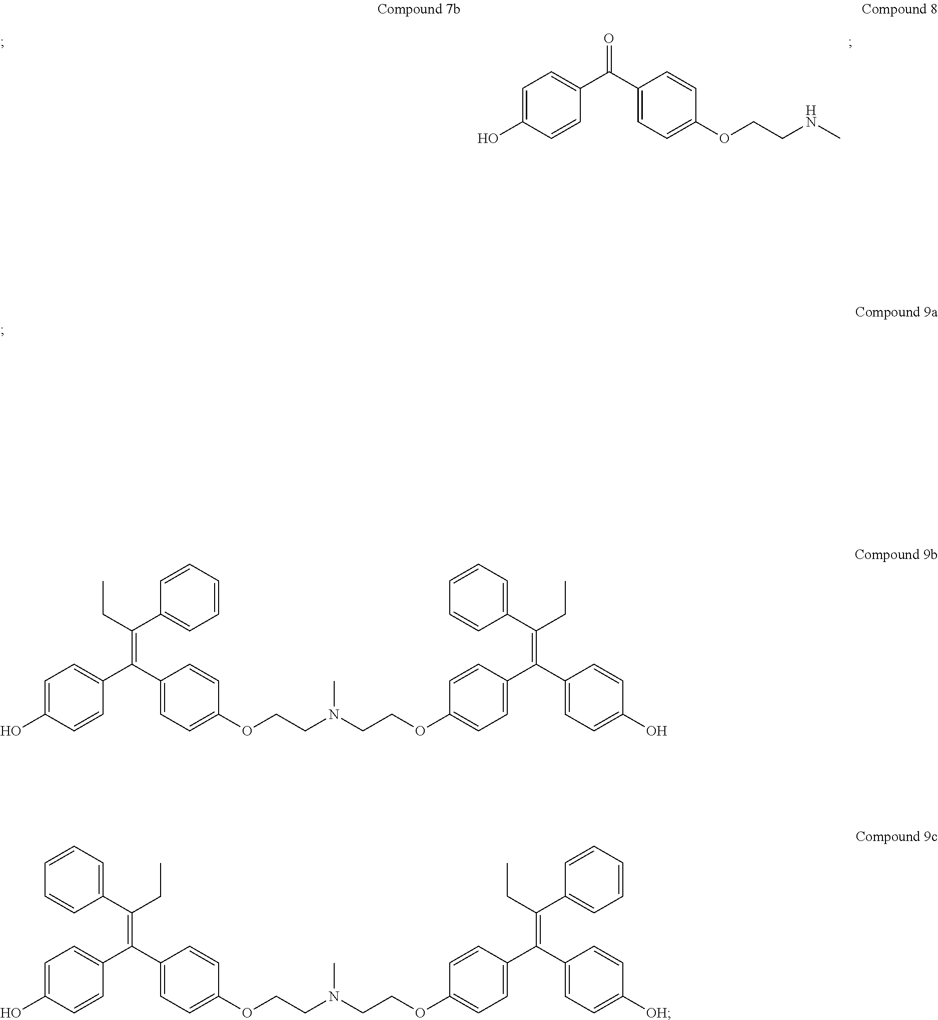 [SMILES: CNCCOc1ccc(C(=O)c2ccc(O)cc2)cc1 ; CC/C(=C(\c1ccc(O)cc1)c1ccc(OCCN(C)CCOc2ccc(/C(=C(/CC)c3ccccc3)c3ccc(O)cc3)cc2)cc1)c1ccccc1 ; CC/C(=C(/c1ccc(O)cc1)c1ccc(OCCN(C)CCOc2ccc(/C(=C(\CC)c3ccccc3)c3ccc(O)cc3)cc2)cc1)c1ccccc1]
or a pharmaceutically acceptable salt, solvate, or tautomer thereof.

392. The pharmaceutical composition of claim 390, further comprising endoxifen, or a pharmaceutically acceptable salt, solvate, or tautomer thereof.

393. The pharmaceutical composition of claim 390, wherein the first compound is: 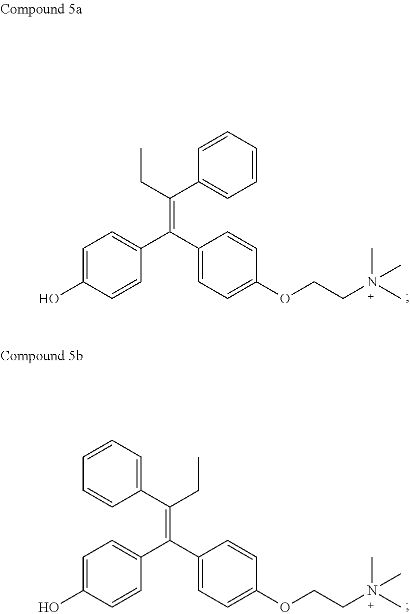
or a pharmaceutically acceptable salt, solvate, or tautomer thereof.

394. The pharmaceutical composition of claim 393, further comprising a second compound, wherein the second compound is: 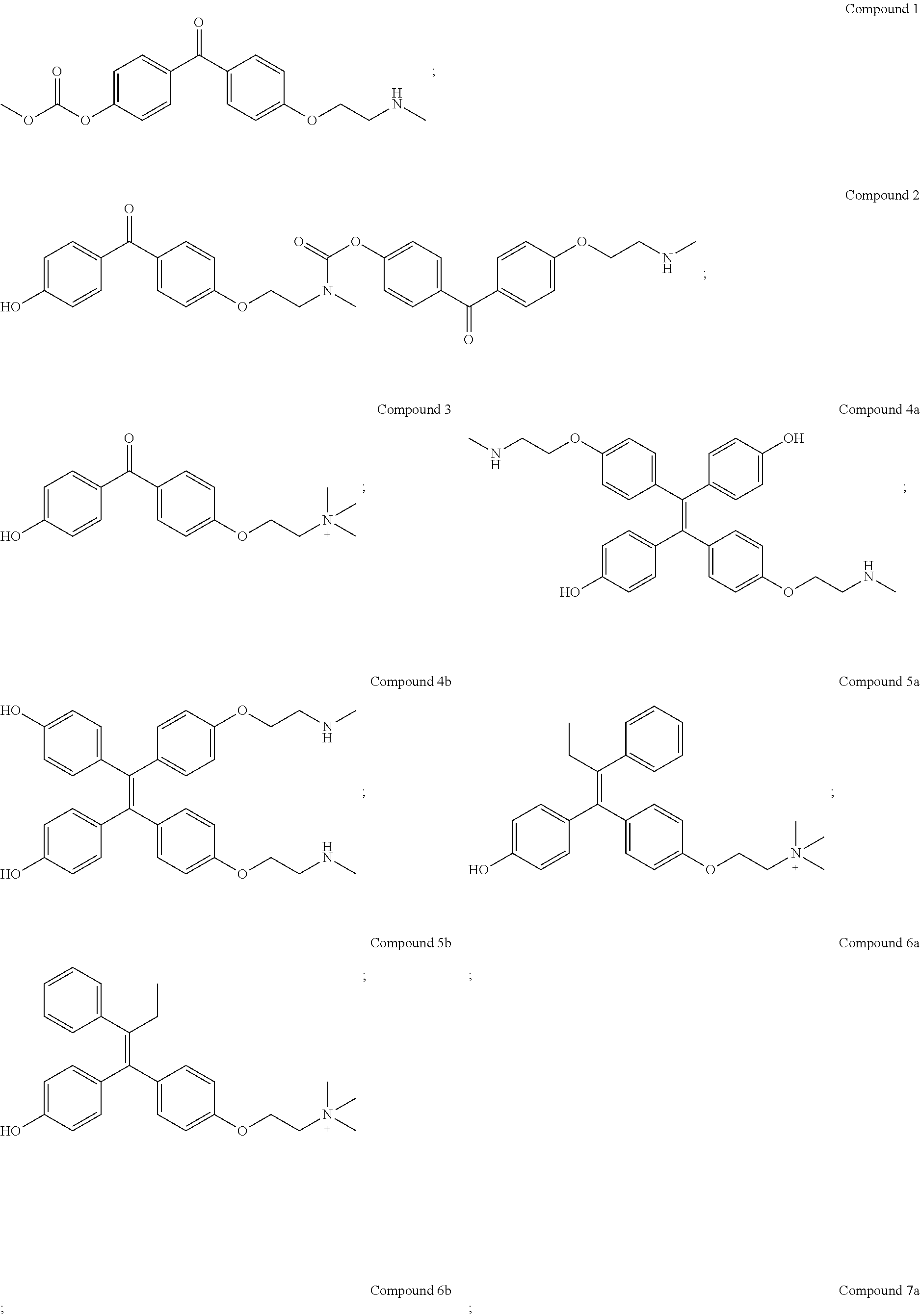 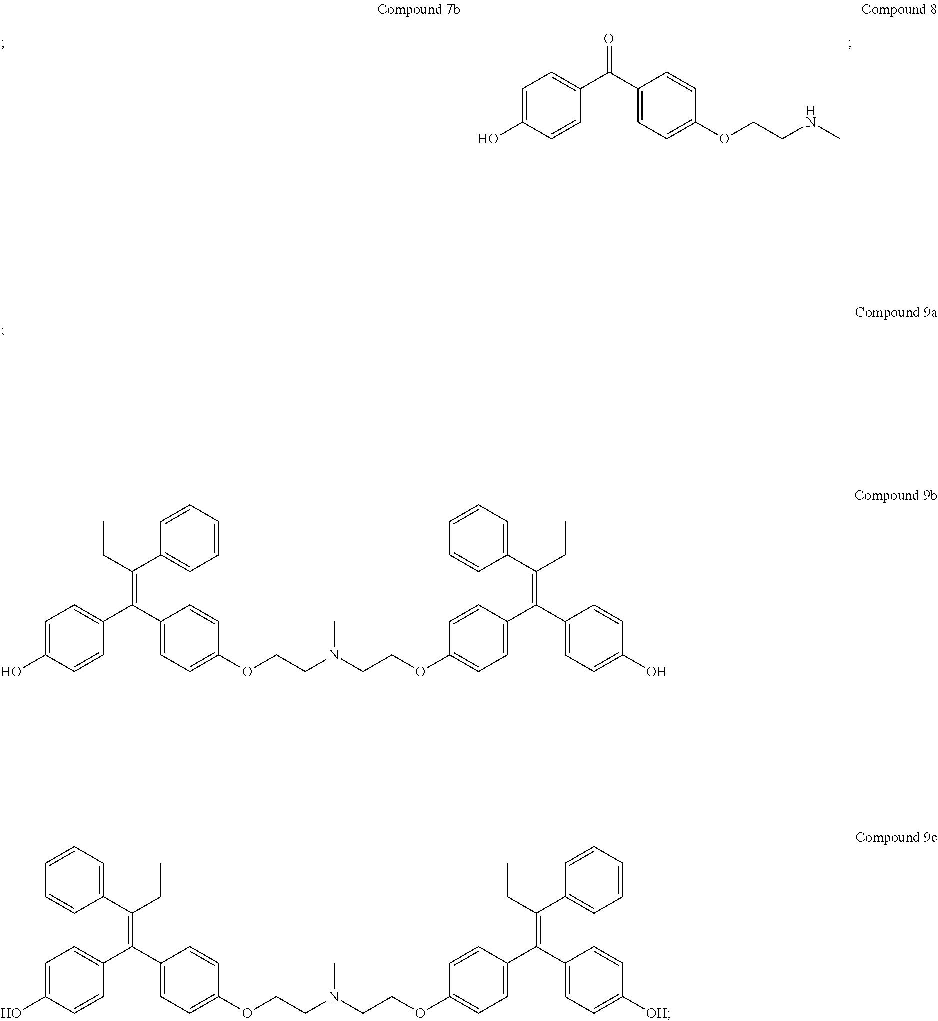 [SMILES: CNCCOc1ccc(C(=O)c2ccc(O)cc2)cc1 ; CC/C(=C(\c1ccc(O)cc1)c1ccc(OCCN(C)CCOc2ccc(/C(=C(/CC)c3ccccc3)c3ccc(O)cc3)cc2)cc1)c1ccccc1 ; CC/C(=C(/c1ccc(O)cc1)c1ccc(OCCN(C)CCOc2ccc(/C(=C(\CC)c3ccccc3)c3ccc(O)cc3)cc2)cc1)c1ccccc1]
or a pharmaceutically acceptable salt, solvate, or tautomer thereof.

395. The pharmaceutical composition of claim 390, wherein the pharmaceutical composition is a solid or a powder, or wherein the first compound is suspended in a liquid.

396. A method comprising administering pharmaceutical composition of claim 390 to a subject in need thereof, wherein the method treats a condition in the subject in need thereof, wherein the condition is cancer.

397. A compound of Formula (III): 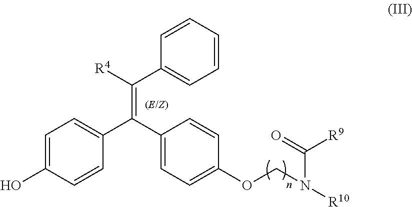
or a pharmaceutically acceptable salt, solvate, or tautomer thereof,
wherein:
R⁴ is C₁-C₆ alkyl substituted with 0 to 3 instances of R⁸, C₂-C₆ alkenyl substituted with 0 to 3 instances of R⁸, or C₂-C₆ alkynyl substituted with 0 to 3 instances of R⁸;
each instance of R⁶ and R⁸ are each independently hydrogen or C₁-C₃ alkyl, or an instance of R⁶ and an instance of R⁷ on the same nitrogen are taken together with the nitrogen to form C₂-C₇ heterocycloalkyl;
each instance of R⁸ is independently halogen, hydroxyl, C₁-C₃ alkoxy, C₁-C₃ alkylthio, C₁-C₃ alkyl, or C₃-C₈ cycloalkyl;
R⁹ is hydrogen, hydroxyl, C₁-C₆ alkyl substituted with 0 to 3 instances of R¹¹, C₂-C₆ alkenyl substituted with 0 to 3 instances of R¹¹, or C₂-C₆ alkynyl substituted with 0 to 3 instances of R¹¹;
R¹⁰ is hydrogen, C₁-C₆ alkyl substituted with 0 to 3 instances of R⁵, C₂-C₆ alkenyl substituted with 0 to 3 instances of R⁵, C₂-C₆ alkynyl substituted with 0 to 3 instances of R⁵, —C(═O)OR⁶, —C(═O)NR⁶R⁷, or —C(═O)SR⁶;
each instance of R¹¹ is independently halogen, hydroxyl, C₁-C₃ alkoxy, C₁-C₃ alkylthio, C₁-C₃ alkyl, or C₃-C₈ cycloalkyl, aryl, or —C(═O)OCH₃; and
n is an integer from 1 to 10.

398. The compound of claim 397, wherein the compound is a compound of Formula (III), and wherein R⁹ is hydrogen, hydroxyl, or C₁-C₃ alkyl.

399. The compound of claim 397, wherein R¹⁰ is hydrogen or C₁-C₃ alkyl.

400. The compound of claim 397, wherein at least 75% of the compound of Formula (III) is a compound of Formula (IV):

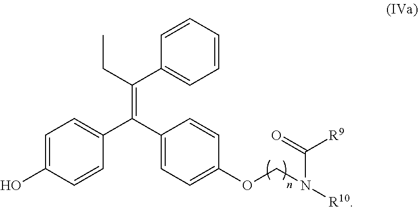

401. The compound of claim 397, wherein the compound has any one of the following structures: 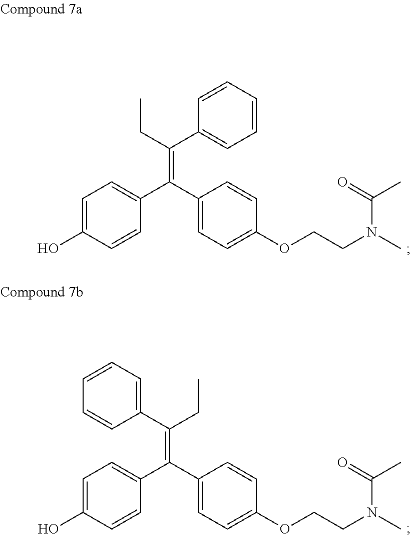 [SMILES: CC/C(=C(\c1ccc(O)cc1)c1ccc(OCCN(C)C(C)=O)cc1)c1ccccc1 ; CC/C(=C(/c1ccc(O)cc1)c1ccc(OCCN(C)C(C)=O)cc1)c1ccccc1]
or a pharmaceutically acceptable salt, solvate, or tautomer thereof.

402. A pharmaceutical composition comprising a first compound of claim 397 and a pharmaceutically acceptable carrier or diluent.

403. The pharmaceutical composition of claim 402, further comprising a second compound selected from 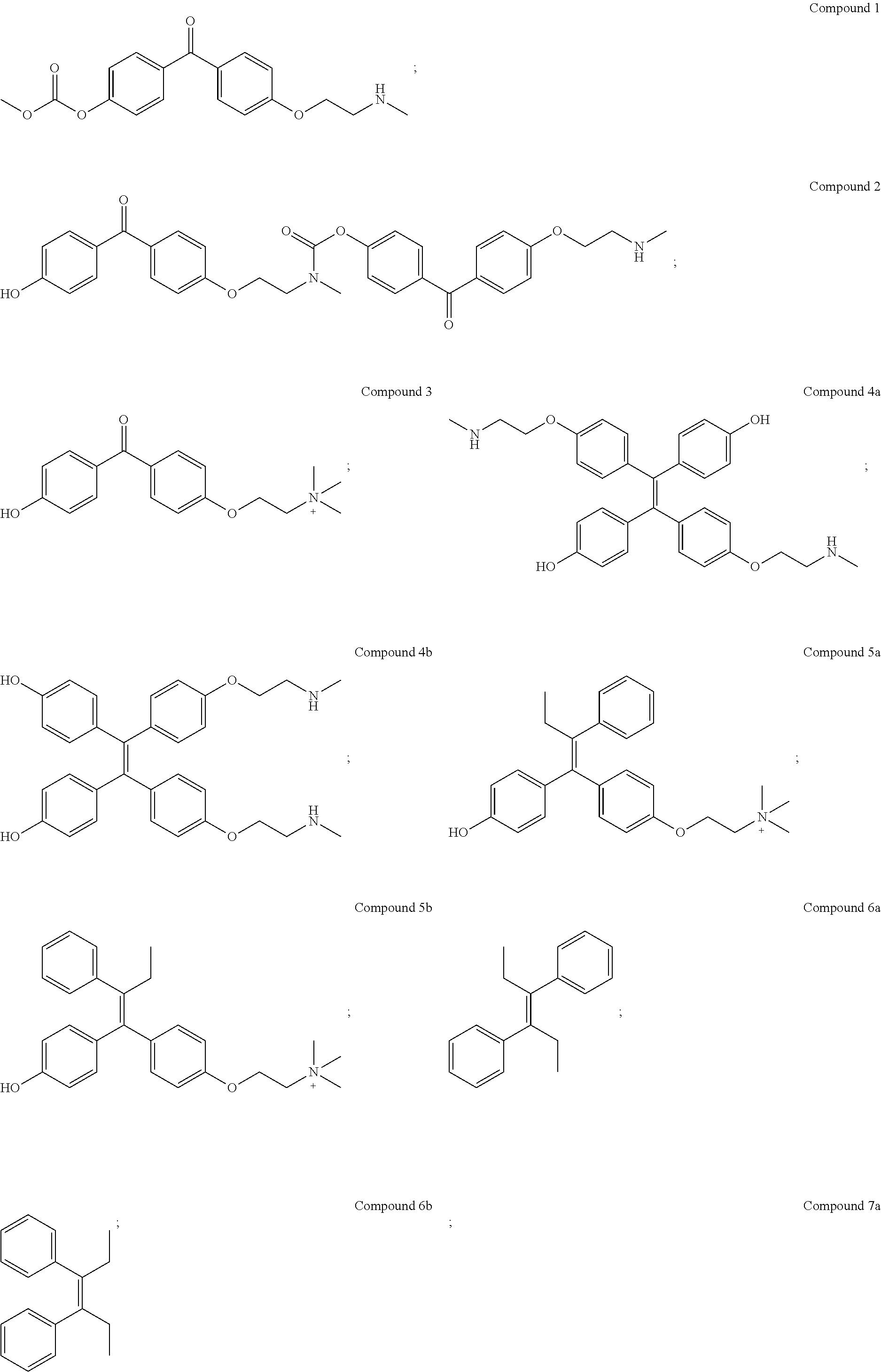 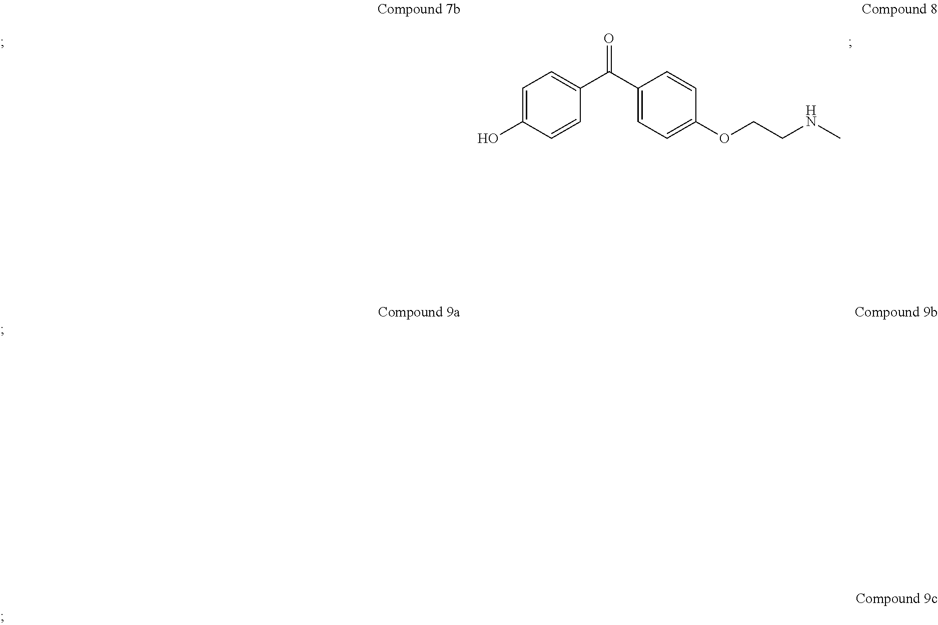 [SMILES: CNCCOc1ccc(C(=O)c2ccc(O)cc2)cc1]
or a pharmaceutically acceptable salt, solvate, or tautomer thereof.

404. The pharmaceutical composition of claim 402, further comprising endoxifen, or a pharmaceutically acceptable salt, solvate, or tautomer thereof.

405. The pharmaceutical composition of claim 402, wherein the first compound is: 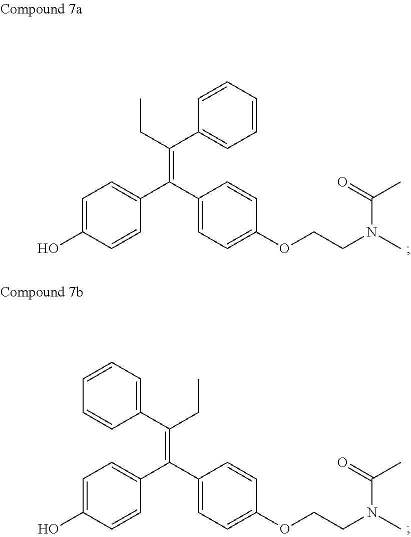 [SMILES: CC/C(=C(\c1ccc(O)cc1)c1ccc(OCCN(C)C(C)=O)cc1)c1ccccc1 ; CC/C(=C(/c1ccc(O)cc1)c1ccc(OCCN(C)C(C)=O)cc1)c1ccccc1]
or a pharmaceutically acceptable salt, solvate, or tautomer thereof.

406. The pharmaceutical composition of claim 402, further comprising a second compound, wherein the second compound is: 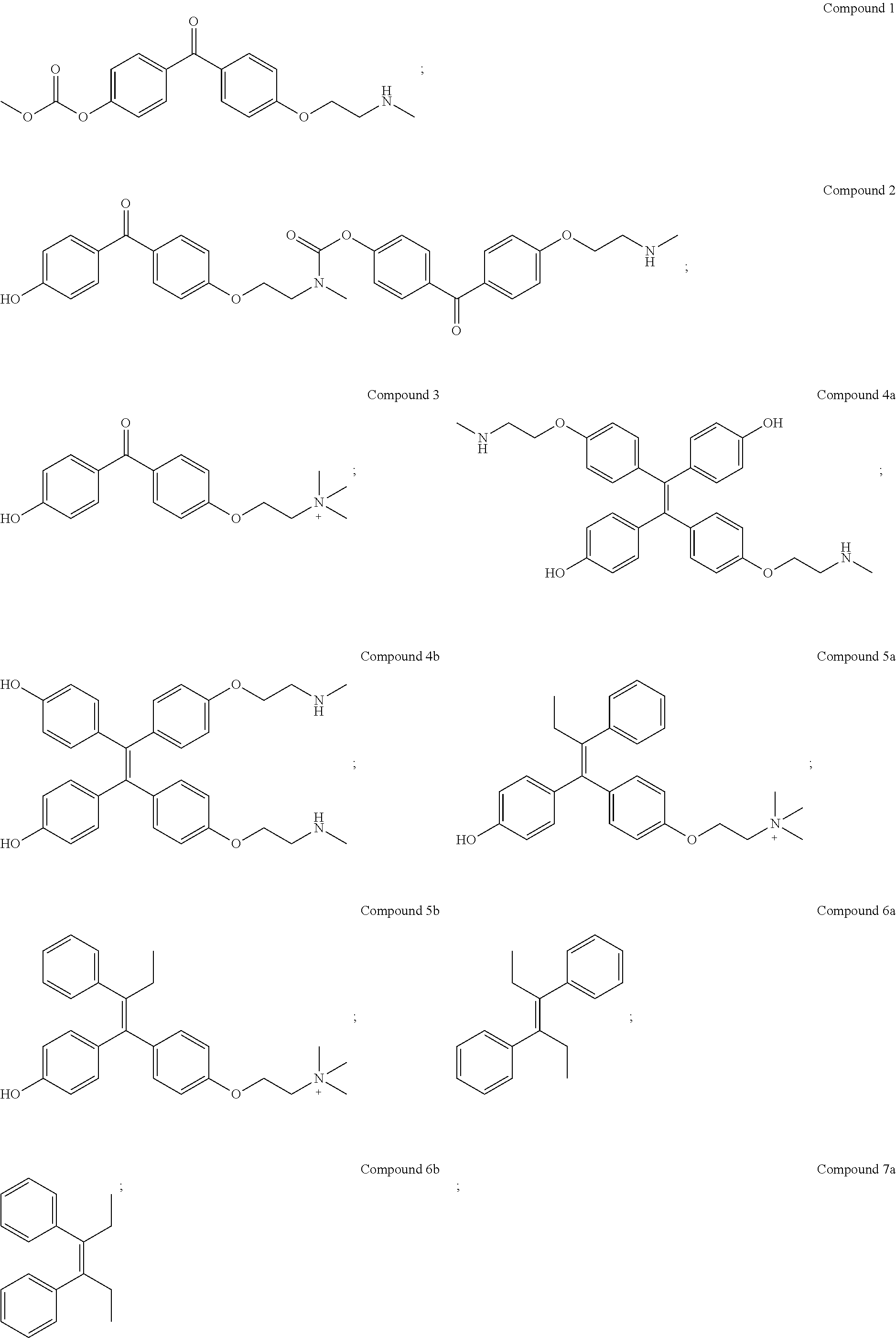 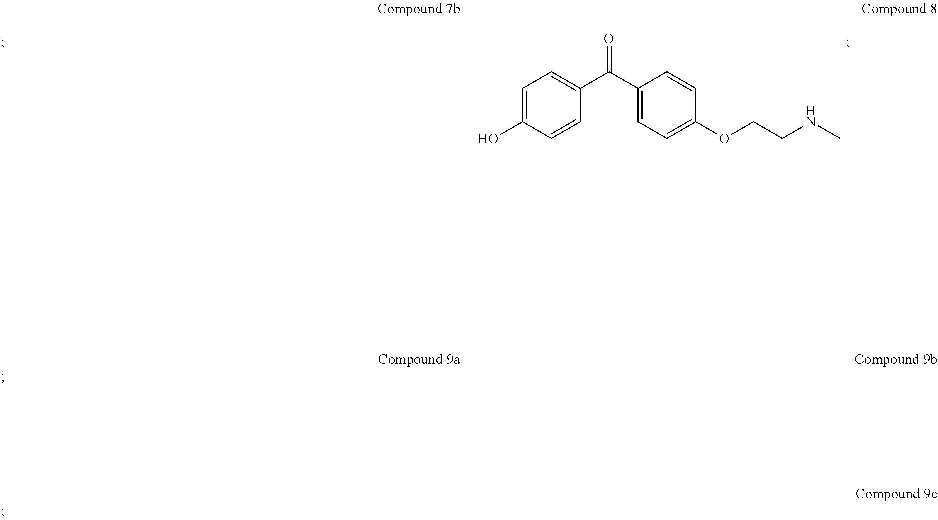 [SMILES: CNCCOc1ccc(C(=O)c2ccc(O)cc2)cc1]
or a pharmaceutically acceptable salt, solvate, or tautomer thereof.

407. The pharmaceutical composition of claim 402, wherein the pharmaceutical composition is a solid or a powder, or wherein the first compound is suspended in a liquid.

408. A method comprising administering pharmaceutical composition of claim 402 to a subject in need thereof, wherein the method treats a condition in the subject in need thereof, wherein the condition is cancer.
